SEQUENCE LISTING (203 sequences)

SEQ ID NO: 1 - RNA, 20 nt, Artificial Sequence
  CIITA sgRNA
gauauuggcauaagccuccc

SEQ ID NO: 2 - RNA, 20 nt, Artificial Sequence
  B2M sgRNA
ggccgagaugucucgcuccg

SEQ ID NO: 3 - DNA, 2520 bp, Artificial Sequence
  CD40 (humanCD40 signal peptide - anti-gp120 scFv - Linker - StrepTagII - Gly6 Linker- full CD40 protein - Gly6 Linker - HA tag - P2A skipping element- mCherry reporter)
caagtttaaacggatctctagcgatggtacggctccctctgcaatgtgtactttggggatgtcttctgactgccgtccatccccaagtacagctcgtacaaagtggagctgaggtcaaaaaaccgggtgcttcagtgaaagtctcatgtcaggctagtgggtatagatttagtcattttacagttcactgggttagacaagcacctggacagaggtttgaatggatgggttggataaacccatacaacggtaataaagagttcagcgcgaaatttcaagaccgggtaactttcacggctgacacaagcgctaacaccgcgtacatggaactgcgaagtttgaggagtgctgacaccgcagtatactactgtgcacgcgttggcgagtggggatgggatgactcaccctacgacaattattatatggacgtttggggtaaaggcaccactgttattgtcagctcaggcggtggagggtctggaggtggcggttccggtggtggaggtagcgagatcgttctcacgcaagccccaggtacgctgtcactttctcctggtgagcgagcaactttcagttgtagatccagccattccattcgcagtagaagagtggcctggtatcaacacaagccgggccaggcacctcgccttgttattcacggggtgtcaaaccgcgcttcagggatctcagatcgcttctccggcagtgggagtggaaccgacttcactttgactatcactcgagttgaacccgaggatttcgccttgtactattgccaagtttacggggcatcctcctatacatttggacagggcacaaagctggagcggaaaggatcagcgggtagtgcagcagggagcggcgagttctggtctcatccacaatttgaaaagggaggcggagggggtggtgaaccgccaacagcttgcagggagaaacaataccttattaatagtcagtgctgctcactgtgccagccgggtcagaagctggtcagcgactgtacggaattcactgaaacggagtgcctgccctgcggggaaagtgaattcttggatacgtggaatcgagaaactcattgtcatcaacacaagtactgtgacccgaacctgggtctgagggtgcagcaaaaaggcaccagtgagacggatacaatttgcacctgtgaagagggctggcactgtacctccgaggcgtgcgagagctgtgttctccaccgctcttgttctcccggatttggggttaaacaaatagcaactggggtttcagatacaatatgcgaaccatgcccggtcgggtttttttcaaatgtctctagcgcgtttgaaaagtgtcatccttggacaagctgcgagactaaggacctcgtcgttcagcaagcgggtacgaataaaactgatgttgtttgtgggcctcaggatcgacttagagcactggttgtcatccctattatatttgggattttgttcgctatcctgcttgtgcttgtaaagaaggttgctaagaagcccactaacaaggcgcctcacccgaagcaggaacctcaagaaattaattttccagacgatctgccagggtcaaacacggctgcacccgtacaggaaacactgcacgggtgtcagcccgtgactcaagaagacggaaaagagagccgaataagcgtgcaggagcgacaaggcggtggcggcggcgggtacccctacgacgtgccagattatgcaggctctggtgctactaatttttctctgctgaaacaggcaggggacgtcgaagagaaccctggccccatggtttctaagggggaagaagacaacatggcaattatcaaggaatttatgcgatttaaggtgcatatggaaggcagcgtgaatggtcacgagttcgaaatagaaggagaaggcgaaggtaggccatacgaaggcacgcagactgcgaaattgaaggtgacaaaagggggcccgctgccatttgcgtgggatattttgagcccccaattcatgtacggcagtaaagcttacgtgaaacacccggctgatatcccagactatctgaagctttcttttccggagggctttaagtgggagcgggttatgaattttgaagatggtggagtcgtcacagtaacccaagattccagtttgcaggacggtgaatttatatataaggtgaaattgaggggaacgaatttcccaagcgatggacctgtaatgcaaaaaaaaacaatggggtgggaagcgtcatctgagcgcatgtatcccgaggatggcgcacttaaaggcgaaattaagcaaagattgaaattgaaggacggcggacactatgacgctgaagtaaagactacttataaagccaaaaaaccggtccaactccctggcgcgtataacgtgaacataaagctggacatcacaagccataacgaagactataccattgtagaacagtatgaacgcgccgagggccgccacagtacgggtggtatggatgaactctacaagtgaagggcccgtttctgctagcaag

SEQ ID NO: 4 - protein, 824 aa, Artificial Sequence
  humanCD40 signal peptide - anti-gp120 scFv - Linker - StrepTagII - Gly6 Linker- full CD40 protein - Gly6 Linker - HA tag - P2A skipping element- mCherry reporter
MVRLPLQCVLWGCLLTAVHPQVQLVQSGAEVKKPGASVKVSCQASGYRFSHFTVHWVRQAPGQRFEWMGWINPYNGNKEFSAKFQDRVTFTADTSANTAYMELRSLRSADTAVYYCARVGEWGWDDSPYDNYYMDVWGKGTTVIVSSGGGGSGGGGSGGGGSEIVLTQAPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKGSAGSAAGSGEFWSHPQFEKGGGGGGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVKKVAKKPTNKAPHPKQEPQEINFPDDLPGSNTAAPVQETLHGCQPVTQEDGKESRISVQERQGGGGGGYPYDVPDYAGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 5 - DNA, 2085 bp, Artificial Sequence
  IgG ECD (humanCD40 signal peptide - anti-gp120 scFv - Linker - StrepTagII- Gly6 Linker- IgG Hinge- Gly6 Linker- CD40 Transmembrane Domain - CD40 Intracellular Domain - Gly6 Linker - HA tag - P2A skipping element- mCherry reporter)
caagtttaaacggatctctagcgatggtacggctccctctgcaatgtgtactttggggatgtcttctgactgccgtccatccccaagtacagctcgtacaaagtggagctgaggtcaaaaaaccgggtgcttcagtgaaagtctcatgtcaggctagtgggtatagatttagtcattttacagttcactgggttagacaagcacctggacagaggtttgaatggatgggttggataaacccatacaacggtaataaagagttcagcgcgaaatttcaagaccgggtaactttcacggctgacacaagcgctaacaccgcgtacatggaactgcgaagtttgaggagtgctgacaccgcagtatactactgtgcacgcgttggcgagtggggatgggatgactcaccctacgacaattattatatggacgtttggggtaaaggcaccactgttattgtcagctcaggcggtggagggtctggaggtggcggttccggtggtggaggtagcgagatcgttctcacgcaagccccaggtacgctgtcactttctcctggtgagcgagcaactttcagttgtagatccagccattccattcgcagtagaagagtggcctggtatcaacacaagccgggccaggcacctcgccttgttattcacggggtgtcaaaccgcgcttcagggatctcagatcgcttctccggcagtgggagtggaaccgacttcactttgactatcactcgagttgaacccgaggatttcgccttgtactattgccaagtttacggggcatcctcctatacatttggacagggcacaaagctggagcggaaaggatcagcgggtagtgcagcagggagcggcgagttctggtctcatccacaatttgaaaagggaggcggaggtggaggtgaacccaagtcatgtgataagacacatacttgtcccccctgcccgggaggtggtggagggggtgctctcgtggttatccctatcatattcggcatactttttgcaatcctccttgtcctggtaaaaaaggtagcgaaaaagcccaagaaggttgctaagaagcccactaacaaggcgcctcacccgaagcaggaacctcaagaaattaattttccagacgatctgccagggtcaaacacggctgcacccgtacaggaaacactgcacgggtgtcagcccgtgactcaagaagacggaaaagagagccgaataagcgtgcaggagcgacaaggcggtggcggcggcgggtacccctacgacgtgccagattatgcaggctctggtgctactaatttttctctgctgaaacaggcaggggacgtcgaagagaaccctggccccatggtttctaagggggaagaagacaacatggcaattatcaaggaatttatgcgatttaaggtgcatatggaaggcagcgtgaatggtcacgagttcgaaatagaaggagaaggcgaaggtaggccatacgaaggcacgcagactgcgaaattgaaggtgacaaaagggggcccgctgccatttgcgtgggatattttgagcccccaattcatgtacggcagtaaagcttacgtgaaacacccggctgatatcccagactatctgaagctttcttttccggagggctttaagtgggagcgggttatgaattttgaagatggtggagtcgtcacagtaacccaagattccagtttgcaggacggtgaatttatatataaggtgaaattgaggggaacgaatttcccaagcgatggacctgtaatgcaaaaaaaaacaatggggtgggaagcgtcatctgagcgcatgtatcccgaggatggcgcacttaaaggcgaaattaagcaaagattgaaattgaaggacggcggacactatgacgctgaagtaaagactacttataaagccaaaaaaccggtccaactccctggcgcgtataacgtgaacataaagctggacatcacaagccataacgaagactataccattgtagaacagtatgaacgcgccgagggccgccacagtacgggtggtatggatgaactctacaagtgaagggcccgtttctgctagcaag

SEQ ID NO: 6 - protein, 679 aa, Artificial Sequence
  IgG ECD (humanCD40 signal peptide - anti-gp120 scFv - Linker - StrepTagII- Gly6 Linker- IgG Hinge- Gly6 Linker- CD40 Transmembrane Domain - CD40 Intracellular Domain - Gly6 Linker - HA tag - P2A skipping element- mCherry reporter)
MVRLPLQCVLWGCLLTAVHPQVQLVQSGAEVKKPGASVKVSCQASGYRFSHFTVHWVRQAPGQRFEWMGWINPYNGNKEFSAKFQDRVTFTADTSANTAYMELRSLRSADTAVYYCARVGEWGWDDSPYDNYYMDVWGKGTTVIVSSGGGGSGGGGSGGGGSEIVLTQAPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKGSAGSAAGSGEFWSHPQFEKGGGGGGEPKSCDKTHTCPPCPGGGGGGALVVIPIIFGILFAILLVLVKKVAKKPKKVAKKPTNKAPHPKQEPQEINFPDDLPGSNTAAPVQETLHGCQPVTQEDGKESRISVQERQGGGGGGYPYDVPDYAGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 7 - DNA, 2082 bp, Artificial Sequence
  IgG ECD + TM (humanCD40 signal peptide - anti-gp120 scFv - Linker - StrepTagII- Gly6 Linker- IgG Hinge- Gly6 Linker- IgG2 Transmembrane Domain - Gly6 Linker - CD40 Intracellular Domain - Gly6 Linker - HA tag - P2A skipping element- mCherry reporter)
caagtttaaacggatctctagcgatggtacggctccctctgcaatgtgtactttggggatgtcttctgactgccgtccatccccaagtacagctcgtacaaagtggagctgaggtcaaaaaaccgggtgcttcagtgaaagtctcatgtcaggctagtgggtatagatttagtcattttacagttcactgggttagacaagcacctggacagaggtttgaatggatgggttggataaacccatacaacggtaataaagagttcagcgcgaaatttcaagaccgggtaactttcacggctgacacaagcgctaacaccgcgtacatggaactgcgaagtttgaggagtgctgacaccgcagtatactactgtgcacgcgttggcgagtggggatgggatgactcaccctacgacaattattatatggacgtttggggtaaaggcaccactgttattgtcagctcaggcggtggagggtctggaggtggcggttccggtggtggaggtagcgagatcgttctcacgcaagccccaggtacgctgtcactttctcctggtgagcgagcaactttcagttgtagatccagccattccattcgcagtagaagagtggcctggtatcaacacaagccgggccaggcacctcgccttgttattcacggggtgtcaaaccgcgcttcagggatctcagatcgcttctccggcagtgggagtggaaccgacttcactttgactatcactcgagttgaacccgaggatttcgccttgtactattgccaagtttacggggcatcctcctatacatttggacagggcacaaagctggagcggaaaggatcagcgggtagtgcagcagggagcggcgagttctggtctcatccacaatttgaaaagggagggggaggcggaggagaaccaaaaagctgtgacaaaacgcacacctgccctccctgcccaggtctgtggactactataaccatctttatcacgctttttttgctttcagtttgctatagcgcaactataacctttttcgggggcggcggtggtggtaagaaggttgctaagaagcccactaacaaggcgcctcacccgaagcaggaacctcaagaaattaattttccagacgatctgccagggtcaaacacggctgcacccgtacaggaaacactgcacgggtgtcagcccgtgactcaagaagacggaaaagagagccgaataagcgtgcaggagcgacaaggcggtggcggcggcgggtacccctacgacgtgccagattatgcaggctctggtgctactaatttttctctgctgaaacaggcaggggacgtcgaagagaaccctggccccatggtttctaagggggaagaagacaacatggcaattatcaaggaatttatgcgatttaaggtgcatatggaaggcagcgtgaatggtcacgagttcgaaatagaaggagaaggcgaaggtaggccatacgaaggcacgcagactgcgaaattgaaggtgacaaaagggggcccgctgccatttgcgtgggatattttgagcccccaattcatgtacggcagtaaagcttacgtgaaacacccggctgatatcccagactatctgaagctttcttttccggagggctttaagtgggagcgggttatgaattttgaagatggtggagtcgtcacagtaacccaagattccagtttgcaggacggtgaatttatatataaggtgaaattgaggggaacgaatttcccaagcgatggacctgtaatgcaaaaaaaaacaatggggtgggaagcgtcatctgagcgcatgtatcccgaggatggcgcacttaaaggcgaaattaagcaaagattgaaattgaaggacggcggacactatgacgctgaagtaaagactacttataaagccaaaaaaccggtccaactccctggcgcgtataacgtgaacataaagctggacatcacaagccataacgaagactataccattgtagaacagtatgaacgcgccgagggccgccacagtacgggtggtatggatgaactctacaagtgaagggcccgtttctgctagcaag

SEQ ID NO: 8 - protein, 678 aa, Artificial Sequence
  IgG ECD + TM (humanCD40 signal peptide - anti-gp120 scFv - Linker - StrepTagII- Gly6 Linker- IgG Hinge- Gly6 Linker- IgG2 Transmembrane Domain - Gly6 Linker - CD40 Intracellular Domain - Gly6 Linker - HA tag - P2A skipping element- mCherry reporter)
MVRLPLQCVLWGCLLTAVHPQVQLVQSGAEVKKPGASVKVSCQASGYRFSHFTVHWVRQAPGQRFEWMGWINPYNGNKEFSAKFQDRVTFTADTSANTAYMELRSLRSADTAVYYCARVGEWGWDDSPYDNYYMDVWGKGTTVIVSSGGGGSGGGGSGGGGSEIVLTQAPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKGSAGSAAGSGEFWSHPQFEKGGGGGGEPKSCDKTHTCPPCPGLWTTITIFITLFLLSVCYSATITFFGGGGGGKKVAKKPTNKAPHPKQEPQEINFPDDLPGSNTAAPVQETLHGCQPVTQEDGKESRISVQERQGGGGGGYPYDVPDYAGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 9 - DNA, 3225 bp, Artificial Sequence
  full CD40+FKBP12v36 (humanCD40 signal peptide - anti-gp120 scFv - Linker - StrepTagII - Gly6 Linker- full CD40 protein - Gly6 Linker- FkBP12v36 - GlySer Linker - FkBP12v36- Gly6 Linker - HA tag - P2A skipping element- mCherry reporter)
caagtttaaacggatctctagcgatggtacggctccctctgcaatgtgtactttggggatgtcttctgactgccgtccatccccaagtacagctcgtacaaagtggagctgaggtcaaaaaaccgggtgcttcagtgaaagtctcatgtcaggctagtgggtatagatttagtcattttacagttcactgggttagacaagcacctggacagaggtttgaatggatgggttggataaacccatacaacggtaataaagagttcagcgcgaaatttcaagaccgggtaactttcacggctgacacaagcgctaacaccgcgtacatggaactgcgaagtttgaggagtgctgacaccgcagtatactactgtgcacgcgttggcgagtggggatgggatgactcaccctacgacaattattatatggacgtttggggtaaaggcaccactgttattgtcagctcaggcggtggagggtctggaggtggcggttccggtggtggaggtagcgagatcgttctcacgcaagccccaggtacgctgtcactttctcctggtgagcgagcaactttcagttgtagatccagccattccattcgcagtagaagagtggcctggtatcaacacaagccgggccaggcacctcgccttgttattcacggggtgtcaaaccgcgcttcagggatctcagatcgcttctccggcagtgggagtggaaccgacttcactttgactatcactcgagttgaacccgaggatttcgccttgtactattgccaagtttacggggcatcctcctatacatttggacagggcacaaagctggagcggaaaggatcagcgggtagtgcagcagggagcggcgagttctggtctcatccacaatttgaaaagggaggcggagggggtggtgaaccgccaacagcttgcagggagaaacaataccttattaatagtcagtgctgctcactgtgccagccgggtcagaagctggtcagcgactgtacggaattcactgaaacggagtgcctgccctgcggggaaagtgaattcttggatacgtggaatcgagaaactcattgtcatcaacacaagtactgtgacccgaacctgggtctgagggtgcagcaaaaaggcaccagtgagacggatacaatttgcacctgtgaagagggctggcactgtacctccgaggcgtgcgagagctgtgttctccaccgctcttgttctcccggatttggggttaaacaaatagcaactggggtttcagatacaatatgcgaaccatgcccggtcgggtttttttcaaatgtctctagcgcgtttgaaaagtgtcatccttggacaagctgcgagactaaggacctcgtcgttcagcaagcgggtacgaataaaactgatgttgtttgtgggcctcaggatcgacttagagcactggttgtcatccctattatatttgggattttgttcgctatcctgcttgtgcttgtaaagaaggttgctaagaagcccactaacaaggcgcctcacccgaagcaggaacctcaagaaattaattttccagacgatctgccagggtcaaacacggctgcacccgtacaggaaacactgcacgggtgtcagcccgtgactcaagaagacggaaaagagagccgaataagcgtgcaggagcgacaaggaggtggcggaggaggcggggtgcaagtagaaaccatatctcctggtgacggacggacgtttcccaagcgaggacaaacgtgcgtggtacactacaccgggatgctggaagacgggaaaaaagttgatagcagcagggaccgaaataagccattcaaatttatgctgggaaagcaggaagtgatccgaggctgggaagaaggagttgcccagatgtcagtaggccagcgggctaaactgacaatctcacccgactatgcatacggggcaacgggacacccgggaattattcctcctcacgctacgctggtctttgacgttgaattgttgaaactggagggcgggggcggatcaggtggtggtggatcaggcggaggtggaagtggggttcaagtggaaacaatttctccgggagatggcagaacctttcccaaacgcggccagacctgcgtggtacattacactggcatgctggaggatggtaaaaaagtagactcaagccgagacaggaacaagccttttaaatttatgttgggcaaacaagaggttattcgcgggtgggaagaaggggtcgcgcagatgtccgtcggccaacgagctaaactcacgataagtccggattatgcatatggggctaccggacacccaggcattatcccaccccacgcgacgcttgtctttgatgttgaacttttgaaattggaaggcggcgggggaggtggctacccctacgacgtgccagattatgcaggctctggtgctactaatttttctctgctgaaacaggcaggggacgtcgaagagaaccctggccccatggtttctaagggggaagaagacaacatggcaattatcaaggaatttatgcgatttaaggtgcatatggaaggcagcgtgaatggtcacgagttcgaaatagaaggagaaggcgaaggtaggccatacgaaggcacgcagactgcgaaattgaaggtgacaaaagggggcccgctgccatttgcgtgggatattttgagcccccaattcatgtacggcagtaaagcttacgtgaaacacccggctgatatcccagactatctgaagctttcttttccggagggctttaagtgggagcgggttatgaattttgaagatggtggagtcgtcacagtaacccaagattccagtttgcaggacggtgaatttatatataaggtgaaattgaggggaacgaatttcccaagcgatggacctgtaatgcaaaaaaaaacaatggggtgggaagcgtcatctgagcgcatgtatcccgaggatggcgcacttaaaggcgaaattaagcaaagattgaaattgaaggacggcggacactatgacgctgaagtaaagactacttataaagccaaaaaaccggtccaactccctggcgcgtataacgtgaacataaagctggacatcacaagccataacgaagactataccattgtagaacagtatgaacgcgccgagggccgccacagtacgggtggtatggatgaactctacaagtgaagggcccgtttctgctagcaag

SEQ ID NO: 10 - protein, 1059 aa, Artificial Sequence
  full CD40+FKBP12v36 (humanCD40 signal peptide - anti-gp120 scFv - Linker - StrepTagII - Gly6 Linker- full CD40 protein - Gly6 Linker- FkBP12v36 - GlySer Linker - FkBP12v36- Gly6 Linker - HA tag - P2A skipping element- mCherry reporter)
MVRLPLQCVLWGCLLTAVHPQVQLVQSGAEVKKPGASVKVSCQASGYRFSHFTVHWVRQAPGQRFEWMGWINPYNGNKEFSAKFQDRVTFTADTSANTAYMELRSLRSADTAVYYCARVGEWGWDDSPYDNYYMDVWGKGTTVIVSSGGGGSGGGGSGGGGSEIVLTQAPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKGSAGSAAGSGEFWSHPQFEKGGGGGGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVKKVAKKPTNKAPHPKQEPQEINFPDDLPGSNTAAPVQETLHGCQPVTQEDGKESRISVQERQGGGGGGGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGGGGSGGGGSGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGGGYPYDVPDYAGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 11 - DNA, 1692 bp, Artificial Sequence
  full CD40 no scFv (human CD40 signal peptide - full CD40 protein - Gly6 Linker - HA tag - P2A skipping element- mCherry Reporter)
caagtttaaacggatctctagcgatggtacggctccctctgcaatgtgtactttggggatgtcttctgactgccgtccatcccgaaccgccaacagcttgcagggagaaacaataccttattaatagtcagtgctgctcactgtgccagccgggtcagaagctggtcagcgactgtacggaattcactgaaacggagtgcctgccctgcggggaaagtgaattcttggatacgtggaatcgagaaactcattgtcatcaacacaagtactgtgacccgaacctgggtctgagggtgcagcaaaaaggcaccagtgagacggatacaatttgcacctgtgaagagggctggcactgtacctccgaggcgtgcgagagctgtgttctccaccgctcttgttctcccggatttggggttaaacaaatagcaactggggtttcagatacaatatgcgaaccatgcccggtcgggtttttttcaaatgtctctagcgcgtttgaaaagtgtcatccttggacaagctgcgagactaaggacctcgtcgttcagcaagcgggtacgaataaaactgatgttgtttgtgggcctcaggatcgacttagagcactggttgtcatccctattatatttgggattttgttcgctatcctgcttgtgcttgtaaagaaggttgctaagaagcccactaacaaggcgcctcacccgaagcaggaacctcaagaaattaattttccagacgatctgccagggtcaaacacggctgcacccgtacaggaaacactgcacgggtgtcagcccgtgactcaagaagacggaaaagagagccgaataagcgtgcaggagcgacaaggcggtggcggcggcgggtacccctacgacgtgccagattatgcaggctctggtgctactaatttttctctgctgaaacaggcaggggacgtcgaagagaaccctggccccatggtttctaagggggaagaagacaacatggcaattatcaaggaatttatgcgatttaaggtgcatatggaaggcagcgtgaatggtcacgagttcgaaatagaaggagaaggcgaaggtaggccatacgaaggcacgcagactgcgaaattgaaggtgacaaaagggggcccgctgccatttgcgtgggatattttgagcccccaattcatgtacggcagtaaagcttacgtgaaacacccggctgatatcccagactatctgaagctttcttttccggagggctttaagtgggagcgggttatgaattttgaagatggtggagtcgtcacagtaacccaagattccagtttgcaggacggtgaatttatatataaggtgaaattgaggggaacgaatttcccaagcgatggacctgtaatgcaaaaaaaaacaatggggtgggaagcgtcatctgagcgcatgtatcccgaggatggcgcacttaaaggcgaaattaagcaaagattgaaattgaaggacggcggacactatgacgctgaagtaaagactacttataaagccaaaaaaccggtccaactccctggcgcgtataacgtgaacataaagctggacatcacaagccataacgaagactataccattgtagaacagtatgaacgcgccgagggccgccacagtacgggtggtatggatgaactctacaagtgaagggcccgtttctgctagcaag

SEQ ID NO: 12 - protein, 548 aa, Artificial Sequence
  full CD40 no scFv (human CD40 signal peptide - full CD40 protein - Gly6 Linker - HA tag - P2A skipping element- mCherry Reporter)
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVKKVAKKPTNKAPHPKQEPQEINFPDDLPGSNTAAPVQETLHGCQPVTQEDGKESRISVQERQGGGGGGYPYDVPDYAGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 13 - DNA, 7736 bp, Artificial Sequence
  MRT3_PTT3_mouseCD79a_IntracellularCD40 (mouseCD79alpha-mouseIntracellularCD40-P2A skipping element-T2A skipping element-eGFP Reporter)
agatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacggatctctagcgatgcctggtggcctggaagccctgcgcgccctgcccttgttgctttttctctcttacgcttgccttggtccaggttgtcaagcactcagggtcgaaggagggccccccagcctcacagtgaatctcggtgaggaagcccggttgacctgtgagaacaacggtagaaaccccaacatcacctggtggttttctttgcaatcaaacatcacctggcctcctgtcccactgggccccggccaaggaacaactggacaactgttctttcccgaggtgaataagaaccaccgaggactctattggtgccaagttatagagaacaacatccttaagcgaagctgcgggacttatcttagggttcgcaaccctgtccccagaccattccttgacatgggggaagggaccaagaatcgaataatcacagcagagggtattattctgttgttttgcgctgtggtgccaggtacacttctgctttttcggaagcggtggcaaaacgagaagttcggcgtggatatgcctgacgattacgaggatgagaacctgtacgaggggcttaatctggatgattgtagtatgtacgaagacatcagcagaggcctgcaagggacataccaagacgtagggaacctgcacatcggtgatgctcaactggagaaaccaaagaaagtggtcaagaagccaaaggacaatgagattctgccccccgctgcaagaaggcaagatccccaggagatggaggactatccaggccacaacacagccgccccagtgcaggaaactctgcacggttgccaacctgttacacaggaggatgggaaggaaagccgcatcagtgtgcaagagcgacaagtgacagactcaatagccttgcggcccctcgttggctccggcgctaccaattttagcctgttgaagcaggctggggacgtggaagagaaccccggaccaggaagtggcgagggaagggggagtcttcttacctgtggggatgttgaagagaatcctggccccatggtatccaagggagaggagctgttcacaggcgttgtcccaattctcgtggaattggatggggacgtaaatggtcacaagtttagcgtctctggcgagggcgagggtgacgcaacatatggtaaattgactctgaagtttatttgcaccacaggtaaacttcctgtgccttggcctacacttgtcactactctgacctatggcgtgcaatgtttctcccgctaccctgaccatatgaaacaacacgatttttttaaatctgccatgcccgaagggtatgtgcaggaacggactattttctttaaggacgacggcaattataaaactcgagctgaggtcaaatttgaaggtgacaccttggtcaacagaatagaactcaagggcattgattttaaagaggacgggaacatactgggtcacaaattggagtacaattataactcccacaacgtttatataatggctgataagcagaaaaatggaattaaggtcaacttcaaaatccgacataacatagaggatgggtcagtacagcttgccgaccattatcagcaaaacactcctataggcgatggacccgtgcttttgcctgacaatcattatctgtctactcaaagcgctctttctaaagatcctaacgaaaaacgggatcatatggtgctcttggagtttgtaactgccgccggcatcacattgggaatggacgaactctataaatgaccctctagagggcccgtttctgctagcaagcttgctagcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgaccaattctcatgtttgacagcttatcatcgcagatccgggcaacgttgttgccattgctgcaggcgcagaactggtaggtatggc

SEQ ID NO: 14 - protein, 576 aa, Artificial Sequence
  MRT3_PTT3_mouseCD79a_IntracellularCD40 (mouseCD79alpha-mouseIntracellularCD40-P2A skipping element-T2A skipping element-eGFP Reporter)
MPGGLEALRALPLLLFLSYACLGPGCQALRVEGGPPSLTVNLGEEARLTCENNGRNPNITWWFSLQSNITWPPVPLGPGQGTTGQLFFPEVNKNHRGLYWCQVIENNILKRSCGTYLRVRNPVPRPFLDMGEGTKNRIITAEGIILLFCAVVPGTLLLFRKRWQNEKFGVDMPDDYEDENLYEGLNLDDCSMYEDISRGLQGTYQDVGNLHIGDAQLEKPKKVVKKPKDNEILPPAARRQDPQEMEDYPGHNTAAPVQETLHGCQPVTQEDGKESRISVQERQVTDSIALRPLVGSGATNFSLLKQAGDVEENPGPGSGEGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 15 - DNA, 7760 bp, Artificial Sequence
  MRT4_PTT3_mouseCD79b_IntracellularCD40 (mouseCD79beta-mouseIntracelluarCD40-P2A skipping element-T2A skipping element- eGFP Reporter)
agatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacggatctctagcgatggctacactggttctcagctcaatgccatgtcattggcttttgtttttgctgttgctgttcagtggggagccagtgcctgctatgacatcctccgatttgcccttgaactttcagggatcaccctgcagccaaatatggcagcacccacgctttgcagctaaaaagcgctccagcatggtcaaatttcattgttatacaaaccacagcggggcattgacctggttcagaaagaggggcagccaacagcctcaagaattggtcagtgaggaaggtcgcatagtgcagacacaaaatggctccgtatataccctcactatccaaaacattcagtacgaagacaacggaatctatttttgtaagcagaagtgcgacagtgccaatcacaatgtaacagatagctgtggcaccgagcttctggttcttggcttctcaacacttgatcagttgaagcgacgaaataccctgaaagacggtattatactcattcaaactttgctcataatcctgttcataatagtacctatcttcctgctccttgataaggacgacgggaaagcaggcatggaagaagatcacacctacgaggggttgaatatcgaccagacagccacctacgaagacattgtgactctccgaaccggggaggtgaaatggagtgtaggagaacaccccggtcaagaaaagaaagtggtcaagaagccaaaggacaatgagattctgccccccgctgcaagaaggcaagatccccaggagatggaggactatccaggccacaacacagccgccccagtgcaggaaactctgcacggttgccaacctgttacacaggaggatgggaaggaaagccgcatcagtgtgcaagagcgacaagtgacagactcaatagccttgcggcccctcgttggctccggcgctaccaattttagcctgttgaagcaggctggggacgtggaagagaaccccggaccaggaagtggcgagggaagggggagtcttcttacctgtggggatgttgaagagaatcctggccccatggtatccaagggagaggagctgttcacaggcgttgtcccaattctcgtggaattggatggggacgtaaatggtcacaagtttagcgtctctggcgagggcgagggtgacgcaacatatggtaaattgactctgaagtttatttgcaccacaggtaaacttcctgtgccttggcctacacttgtcactactctgacctatggcgtgcaatgtttctcccgctaccctgaccatatgaaacaacacgatttttttaaatctgccatgcccgaagggtatgtgcaggaacggactattttctttaaggacgacggcaattataaaactcgagctgaggtcaaatttgaaggtgacaccttggtcaacagaatagaactcaagggcattgattttaaagaggacgggaacatactgggtcacaaattggagtacaattataactcccacaacgtttatataatggctgataagcagaaaaatggaattaaggtcaacttcaaaatccgacataacatagaggatgggtcagtacagcttgccgaccattatcagcaaaacactcctataggcgatggacccgtgcttttgcctgacaatcattatctgtctactcaaagcgctctttctaaagatcctaacgaaaaacgggatcatatggtgctcttggagtttgtaactgccgccggcatcacattgggaatggacgaactctataaatgaccctctagagggcccgtttctgctagcaagcttgctagcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggggtgacgcccccagaataaaatttggacggggggttcagtggtggcattgtgctatgacaccaatataaccctcacaaaccccttgggcaataaatactagtgtaggatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgaccaattctcatgtttgacagcttatcatcgcagatccgggcaacgttgttgccattgctgcaggcgcagaactggtaggtatggc

SEQ ID NO: 16 - protein, 584 aa, Artificial Sequence
  MRT4_PTT3_mouseCD79b_IntracellularCD40 (mouseCD79beta-mouseIntracelluarCD40-P2A skipping element-T2A skipping element- eGFP Reporter)
MATLVLSSMPCHWLLFLLLLFSGEPVPAMTSSDLPLNFQGSPCSQIWQHPRFAAKKRSSMVKFHCYTNHSGALTWFRKRGSQQPQELVSEEGRIVQTQNGSVYTLTIQNIQYEDNGIYFCKQKCDSANHNVTDSCGTELLVLGFSTLDQLKRRNTLKDGIILIQTLLIILFIIVPIFLLLDKDDGKAGMEEDHTYEGLNIDQTATYEDIVTLRTGEVKWSVGEHPGQEKKVVKKPKDNEILPPAARRQDPQEMEDYPGHNTAAPVQETLHGCQPVTQEDGKESRISVQERQVTDSIALRPLVGSGATNFSLLKQAGDVEENPGPGSGEGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 17 - DNA, 7638 bp, Artificial Sequence
  MRT5_Ptt3_humanCD79a_IntracellularCD40 (humanCD79alpha-humanIntracellularCD40-P2A skipping element-mCherry Reporter)
agatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacggatctctagcgatgccgggtggacccggtgtcttgcaagccctccccgctaccatctttcttttgtttctcctctcagcagtttatttgggtcccggctgccaagccctctggatgcacaaagtcccggcctctcttatggtatctcttggtgaggatgcgcactttcagtgtccacataactcctctaataatgctaacgttacatggtggagggtccttcacggaaattatacttggcctccagagtttttggggcctggcgaggatccaaatggaacgctgattatacagaacgtgaataaatctcacggagggatctatgtttgtcgagttcaagaggggaatgaatcatatcagcaaagctgcgggacttatttgcgggtacgacaaccaccacctagaccattcctggatatgggagaaggaaccaaaaatagaattatcacagctgagggcataatcctcctcttttgtgcagtagttccaggtaccctcctgctctttagaaagcgatggcaaaatgagaaactcgggttggatgctggagatgaatatgaggatgagaatctgtacgaaggtctgaatctggatgattgtagtatgtacgaggatatttcaagaggtctgcaaggtacctatcaagatgtcggctccttgaatatcggcgacgtacagctcgagaaaccgaaaaaagtggctaagaaaccaactaataaagctccccaccccaaacaggagccacaagagataaatttcccagatgaccttccaggatcaaacacagcagcaccagttcaagaaacactgcacggctgccaacctgttacacaggaagacgggaaagagagcagaatttccgtccaagaacgacagggctctggtgctactaatttttctctgctgaaacaggcaggggacgtcgaagagaaccctggccccatggtttctaagggggaagaagacaacatggcaattatcaaggaatttatgcgatttaaggtgcatatggaaggcagcgtgaatggtcacgagttcgaaatagaaggagaaggcgaaggtaggccatacgaaggcacgcagactgcgaaattgaaggtgacaaaagggggcccgctgccatttgcgtgggatattttgagcccccaattcatgtacggcagtaaagcttacgtgaaacacccggctgatatcccagactatctgaagctttcttttccggagggctttaagtgggagcgggttatgaattttgaagatggtggagtcgtcacagtaacccaagattccagtttgcaggacggtgaatttatatataaggtgaaattgaggggaacgaatttcccaagcgatggacctgtaatgcaaaaaaaaacaatggggtgggaagcgtcatctgagcgcatgtatcccgaggatggcgcacttaaaggcgaaattaagcaaagattgaaattgaaggacggcggacactatgacgctgaagtaaagactacttataaagccaaaaaaccggtccaactccctggcgcgtataacgtgaacataaagctggacatcacaagccataacgaagactataccattgtagaacagtatgaacgcgccgagggccgccacagtacgggtggtatggatgaactctacaagtgaagggcccgtttctgctagcaagcttgctagcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgaccaattctcatgtttgacagcttatcatcgcagatccgggcaacgttgttgccattgctgcaggcgcagaactggtaggtatggc

SEQ ID NO: 18 - protein, 547 aa, Artificial Sequence
  MRT5_Ptt3_humanCD79a_IntracellularCD40 (humanCD79alpha-humanIntracellularCD40-P2A skipping element-mCherry Reporter)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKPKKVAKKPTNKAPHPKQEPQEINFPDDLPGSNTAAPVQETLHGCQPVTQEDGKESRIESVQERQGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 19 - DNA, 7647 bp, Artificial Sequence
  MRT6_Ptt3_humanCD79b_IntracellularCD40 (humanCD79beta-humanIntracellularCD40-P2A skipping element-mCherry Reporter)
agatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacggatctctagcgatggcccgactcgcactgtcaccagtaccgtcacattggatggtggcattgttgcttcttctgagtgctgaacccgtcccagccgctcggagtgaggatcggtaccggaatccgaaaggctcagcatgttcccgaatctggcaatcacctaggtttattgctagaaaacgcggcttcacagtcaaaatgcactgctacatgaacagcgcaagtggtaacgttagctggctttggaagcaggagatggacgaaaacccgcaacagctcaaattggaaaagggacgcatggaggaatcccaaaatgaatcactggcaacacttacgatccaaggtattagattcgaggataacggaatctacttctgtcagcagaaatgcaataatacgagcgaagtctatcaaggctgtggtacagagctgagagtgatggggtttagcactctggcccaacttaaacaaagaaacaccttgaaggacggaataatcatgatccaaactttgctcatcattctttttatcatagtgcctattttcttgttgctcgataaggacgacagtaaagcgggaatggaagaagatcatacatatgagggccttgacattgaccaaactgcgacgtatgaagacatagttaccttgcggacgggcgaagttaaatggagtgttggcgagcatcctggtcaggaaaaaaaagtggctaagaaaccaactaataaagctccccaccccaaacaggagccacaagagataaatttcccagatgaccttccaggatcaaacacagcagcaccagttcaagaaacactgcacggctgccaacctgttacacaggaagacgggaaagagagcagaatttccgtccaagaacgacagggctctggtgctactaatttttctctgctgaaacaggcaggggacgtcgaagagaaccctggccccatggtttctaagggggaagaagacaacatggcaattatcaaggaatttatgcgatttaaggtgcatatggaaggcagcgtgaatggtcacgagttcgaaatagaaggagaaggcgaaggtaggccatacgaaggcacgcagactgcgaaattgaaggtgacaaaagggggcccgctgccatttgcgtgggatattttgagcccccaattcatgtacggcagtaaagcttacgtgaaacacccggctgatatcccagactatctgaagctttcttttccggagggctttaagtgggagcgggttatgaattttgaagatggtggagtcgtcacagtaacccaagattccagtttgcaggacggtgaatttatatataaggtgaaattgaggggaacgaatttcccaagcgatggacctgtaatgcaaaaaaaaacaatggggtgggaagcgtcatctgagcgcatgtatcccgaggatggcgcacttaaaggcgaaattaagcaaagattgaaattgaaggacggcggacactatgacgctgaagtaaagactacttataaagccaaaaaaccggtccaactccctggcgcgtataacgtgaacataaagctggacatcacaagccataacgaagactataccattgtagaacagtatgaacgcgccgagggccgccacagtacgggtggtatggatgaactctacaagtgaagggcccgtttctgctagcaagcttgctagcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgaccaattctcatgtttgacagcttatcatcgcagatccgggcaacgttgttgccattgctgcaggcgcagaactggtaggtatggc

SEQ ID NO: 20 - protein, 549 aa, Artificial Sequence
  MRT6_Ptt3_humanCD79b_IntracellularCD40 (humanCD79beta-humanIntracellularCD40-P2A skipping element-mCherry Reporter)
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQEKKVAKKPTNKAPHPKQEPQEINFPDDLPGSNTAAPVQETLHGCQPVTQEDGKESRISVQERQGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 21 - protein, 546 aa, Artificial Sequence
  Anti-gp120 scFv - Linker - StrepTagII - Gly6 Linker- full CD40 protein - Gly6 Linker - HA tag
QVQLVQSGAEVKKPGASVKVSCQASGYRFSHFTVHWVRQAPGQRFEWMGWINPYNGNKEFSAKFQDRVTFTADTSANTAYMELRSLRSADTAVYYCARVGEWGWDDSPYDNYYMDVWGKGTTVIVSSGGGGSGGGGSGGGGSEIVLTQAPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKGSAGSAAGSGEFWSHPQFEKGGGGGGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVKKVAKKPTNKAPHPKQEPQEINFPDDLPGSNTAAPVQETLHGCQPVTQEDGKESRISVQERQGGGGGGYPYDVPDYA

SEQ ID NO: 22 - protein, 401 aa, Artificial Sequence
  Anti-gp120 scFv - Linker - StrepTagII- Gly6 Linker- IgG Hinge- Gly6 Linker- CD40 Transmembrane Domain - CD40 Intracellular Domain - Gly6 Linker - HA tag
QVQLVQSGAEVKKPGASVKVSCQASGYRFSHFTVHWVRQAPGQRFEWMGWINPYNGNKEFSAKFQDRVTFTADTSANTAYMELRSLRSADTAVYYCARVGEWGWDDSPYDNYYMDVWGKGTTVIVSSGGGGSGGGGSGGGGSEIVLTQAPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKGSAGSAAGSGEFWSHPQFEKGGGGGGEPKSCDKTHTCPPCPGGGGGGALVVIPIIFGILFAILLVLVKKVAKKPKKVAKKPTNKAPHPKQEPQEINFPDDLPGSNTAAPVQETLHGCQPVTQEDGKESRISVQERQGGGGGGYPYDVPDYA

SEQ ID NO: 23 - protein, 400 aa, Artificial Sequence
  Anti-gp120 scFv - Linker - StrepTagII- Gly6 Linker- IgG Hinge- Gly6 Linker- IgG2 Transmembrane Domain - Gly6 Linker - CD40 Intracellular Domain - Gly6 Linker - HA tag
QVQLVQSGAEVKKPGASVKVSCQASGYRFSHFTVHWVRQAPGQRFEWMGWINPYNGNKEFSAKFQDRVTFTADTSANTAYMELRSLRSADTAVYYCARVGEWGWDDSPYDNYYMDVWGKGTTVIVSSGGGGSGGGGSGGGGSEIVLTQAPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKGSAGSAAGSGEFWSHPQFEKGGGGGGEPKSCDKTHTCPPCPGLWTTITIFITLFLLSVCYSATITFFGGGGGGKKVAKKPTNKAPHPKQEPQEINFPDDLPGSNTAAPVQETLHGCQPVTQEDGKESRISVQERQGGGGGGYPYDVPDYA

SEQ ID NO: 24 - protein, 782 aa, Artificial Sequence
  Anti-gp120 scFv - Linker - StrepTagII - Gly6 Linker- full CD40 protein - Gly6 Linker- FkBP12v36 - GlySer Linker - FkBP12v36- Gly6 Linker - HA tag
QVQLVQSGAEVKKPGASVKVSCQASGYRFSHFTVHWVRQAPGQRFEWMGWINPYNGNKEFSAKFQDRVTFTADTSANTAYMELRSLRSADTAVYYCARVGEWGWDDSPYDNYYMDVWGKGTTVIVSSGGGGSGGGGSGGGGSEIVLTQAPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKGSAGSAAGSGEFWSHPQFEKGGGGGGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVKKVAKKPTNKAPHPKQEPQEINFPDDLPGSNTAAPVQETLHGCQPVTQEDGKESRISVQERQGGGGGGGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGGGGSGGGGSGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGGGYPYDVPDYAG

SEQ ID NO: 25 - protein, 294 aa, Artificial Sequence
  MouseCD79alpha-IntracellularCD40
MPGGLEALRALPLLLFLSYACLGPGCQALRVEGGPPSLTVNLGEEARLTCENNGRNPNITWWFSLQSNITWPPVPLGPGQGTTGQLFFPEVNKNHRGLYWCQVIENNILKRSCGTYLRVRNPVPRPFLDMGEGTKNRIITAEGIILLFCAVVPGTLLLFRKRWQNEKFGVDMPDDYEDENLYEGLNLDDCSMYEDISRGLQGTYQDVGNLHIGDAQLEKPKKVVKKPKDNEILPPAARRQDPQEMEDYPGHNTAAPVQETLHGCQPVTQEDGKESRISVQERQVTDSIALRPLV

SEQ ID NO: 26 - protein, 302 aa, Artificial Sequence
  MouseCD79beta-IntracelluarCD40
MATLVLSSMPCHWLLFLLLLFSGEPVPAMTSSDLPLNFQGSPCSQIWQHPRFAAKKRSSMVKFHCYTNHSGALTWFRKRGSQQPQELVSEEGRIVQTQNGSVYTLTIQNIQYEDNGIYFCKQKCDSANHNVTDSCGTELLVLGFSTLDQLKRRNTLKDGIILIQTLLIILFIIVPIFLLLDKDDGKAGMEEDHTYEGLNIDQTATYEDIVTLRTGEVKWSVGEHPGQEKKVVKKPKDNEILPPAARRQDPQEMEDYPGHNTAAPVQETLHGCQPVTQEDGKESRISVQERQVTDSIALRPLV

SEQ ID NO: 27 - protein, 289 aa, Artificial Sequence
  HumanCD79alpha-IntracellularCD40
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKPKKVAKKPTNKAPHPKQEPQEINFPDDLPGSNTAAPVQETLHGCQPVTQEDGKESRIESVQERQ

SEQ ID NO: 28 - protein, 291 aa, Artificial Sequence
  HumanCD79beta-IntracellularCD40
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQEKKVAKKPTNKAPHPKQEPQEINFPDDLPGSNTAAPVQETLHGCQPVTQEDGKESRISVQERQ

SEQ ID NO: 29 - protein, 20 aa, Homo sapiens
MVRLPLQCVLWGCLLTAVHP

SEQ ID NO: 30 - protein, 262 aa, Artificial Sequence
  Anti-gp120 scFv
QVQLVQSGAEVKKPGASVKVSCQASGYRFSHFTVHWVRQAPGQRFEWMGWINPYNGNKEFSAKFQDRVTFTADTSANTAYMELRSLRSADTAVYYCARVGEWGWDDSPYDNYYMDVWGKGTTVIVSSGGGGSGGGGSGGGGSEIVLTQAPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKGSAGSAAGSGEF

SEQ ID NO: 31 - protein, 255 aa, Artificial Sequence
  CD40
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVKKVAKKPTNKAPHPKQEPQEINFPDDLPGSNTAAPVQETLHGCQPVTQEDGKESRISVQERQ

SEQ ID NO: 32 - protein, 173 aa, Artificial Sequence
  Extracellular CD40 protein
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 33 - protein, 20 aa, Artificial Sequence
  CD40 Transmembrane Domain
ALVVIPIIFGILFAILLVLV

SEQ ID NO: 34 - protein, 62 aa, Artificial Sequence
  CD40 Intracellular Domain
KKVAKKPTNKAPHPKQEPQEINFPDDLPGSNTAAPVQETLHGCQPVTQEDGKESRISVQERQ

SEQ ID NO: 35 - protein, 63 aa, Homo sapiens
KKVAKKPTNKAPHPKQEPQEINFPDDLPGSNTAAPVQETLHGCQPVTQEDGKESRIESVQERQ

SEQ ID NO: 36 - protein, 74 aa, Mus musculus
KKVVKKPKDNEILPPAARRQDPQEMEDYPGHNTAAPVQETLHGCQPVTQEDGKESRISVQERQVTDSIALRPLV

SEQ ID NO: 37 - protein, 15 aa, Artificial Sequence
  IgG hinge
EPKSCDKTHTCPPCP

SEQ ID NO: 38 - protein, 26 aa, Artificial Sequence
  IgG2 Transmembrane domain
GLWTTITIFITLFLLSVCYSATITFF

SEQ ID NO: 39 - protein, 107 aa, Artificial Sequence
  FkBP12v36
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 40 - protein, 106 aa, Artificial Sequence
  FkBP12 domain
MGVQVETISPGDGRTFPKRGQTCWHYTGMLEDGKKFDSSRDRNPFKFMLGKQEVIRGWEEGVAQMSVGQRAKLTISPDYAYGATGHPGIIPPHATLVFDVELLKLE

SEQ ID NO: 41 - protein, 100 aa, Artificial Sequence
  FRB segment of mTOR
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 42 - protein, 236 aa, Artificial Sequence
  mCherry reporter
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 43 - protein, 239 aa, Artificial Sequence
  eGFP reporter
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 44 - protein, 220 aa, Mus musculus
MPGGLEALRALPLLLFLSYACLGPGCQALRVEGGPPSLTVNLGEEARLTCENNGRNPNITWWFSLQSNITWPPVPLGPGQGTTGQLFFPEVNKNHRGLYWCQVIENNILKRSCGTYLRVRNPVPRPFLDMGEGTKNRIITAEGIILLFCAVVPGTLLLFRKRWQNEKFGVDMPDDYEDENLYEGLNLDDCSMYEDISRGLQGTYQDVGNLHIGDAQLEKP

SEQ ID NO: 45 - protein, 228 aa, Mus musculus
MATLVLSSMPCHWLLFLLLLFSGEPVPAMTSSDLPLNFQGSPCSQIWQHPRFAAKKRSSMVKFHCYTNHSGALTWFRKRGSQQPQELVSEEGRIVQTQNGSVYTLTIQNIQYEDNGIYFCKQKCDSANHNVTDSCGTELLVLGFSTLDQLKRRNTLKDGIILIQTLLIILFIIVPIFLLLDKDDGKAGMEEDHTYEGLNIDQTATYEDIVTLRTGEVKWSVGEHPGQE

SEQ ID NO: 46 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 47 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 48 - protein, 6 aa, Artificial Sequence
  linker
GGGGGG

SEQ ID NO: 49 - protein, 15 aa, Artificial Sequence
  linker
GGGGSGGGGSGGGGS

SEQ ID NO: 50 - protein, 8 aa, Artificial Sequence
  StrepTagII
WSHPQFEK

SEQ ID NO: 51 - protein, 9 aa, Artificial Sequence
  HA tag
YPYDVPDYA

SEQ ID NO: 52 - protein, 6 aa, Artificial Sequence
  His tag
HHHHHH

SEQ ID NO: 53 - protein, 15 aa, Artificial Sequence
  Avi tag
GLNDIFEAQKIEWHE

SEQ ID NO: 54 - protein, 5 aa, Artificial Sequence
  sFLAG
DYKDE

SEQ ID NO: 55 - protein, 9 aa, Artificial Sequence
  Tev
ENLYFQGGS

SEQ ID NO: 56 - protein, 8 aa, Artificial Sequence
  Flag tag
DYKDDDDK

SEQ ID NO: 57 - protein, 8 aa, Artificial Sequence
  Xpress tag
DLYDDDDK

SEQ ID NO: 58 - protein, 26 aa, Artificial Sequence
  Calmodulin tag
KRRWKKNFIAVSAANRFKKISSSGAL

SEQ ID NO: 59 - protein, 10 aa, Artificial Sequence
  Myc tag
EQKLISEEDL

SEQ ID NO: 60 - protein, 13 aa, Artificial Sequence
  Softag 1
SLAELLNAGLGGS

SEQ ID NO: 61 - protein, 8 aa, Artificial Sequence
  Softag 3
TQDPSRVG

SEQ ID NO: 62 - protein, 8 aa, Artificial Sequence
  STREP tag
WRHPQFGG

SEQ ID NO: 63 - protein, 14 aa, Artificial Sequence
  V5 tag
GKPIPNPLLGLDST

SEQ ID NO: 64 - skipped

SEQ ID NO: 65 - protein, 5 aa, Artificial Sequence
  Gly Ser linker
  MISC_FEATURE (1)..(5): (GlyGlyGlyGlySer) is repeated n times wherein n is an integerof 1, 2, 3, 4, 5, 6, 7, 8, 9 or 10
GGGGS

SEQ ID NO: 66 - protein, 9 aa, Artificial Sequence
  Gly Ser linker
  MISC_FEATURE (1)..(4): (GlyGlyGlySer) is repeated n times wherein n is an integerof 1, 2, 3, 4, 5, 6, 7, 8, 9 or 10
  MISC_FEATURE (5)..(9): (GlyGlyGlyGlySer) is repeated n times wherein n is an integerof 1, 2, 3, 4, 5, 6, 7, 8, 9 or 10
GGGSGGGGS

SEQ ID NO: 67 - protein, 7 aa, Artificial Sequence
  Gly Ser linker
  MISC_FEATURE (1)..(4): (GlyGlyGlySer) is repeated n times wherein n is an integerof 1, 2, 3, 4, 5, 6, 7, 8, 9 or 10
  MISC_FEATURE (5)..(7): (GlyGlySer) is repeated n times wherein n is an integerof 1, 2, 3, 4, 5, 6, 7, 8, 9 or 10
GGGSGGS

SEQ ID NO: 68 - protein, 9 aa, Artificial Sequence
  Gly Ser linker
  MISC_FEATURE (1)..(4): (GlyGlyGlySer) is repeated n times wherein n is an integerof 1, 2, 3, 4, 5, 6, 7, 8, 9 or 10
GGGSGGGGS

SEQ ID NO: 69 - protein, 20 aa, Artificial Sequence
  Gly Ser linker
GGGGSGGGGSGGGGSGGGGS

SEQ ID NO: 70 - protein, 10 aa, Artificial Sequence
  Gly Ser linker
GGGGSGGGGS

SEQ ID NO: 71 - protein, 5 aa, Artificial Sequence
  Gly Ser linker
GGGGS

SEQ ID NO: 72 - protein, 8 aa, Artificial Sequence
  Gly Ser linker
GGGSGGGS

SEQ ID NO: 73 - protein, 4 aa, Artificial Sequence
  Gly Ser linker
GGGS

SEQ ID NO: 74 - protein, 6 aa, Artificial Sequence
  Gly Ser linker
GGSGGS

SEQ ID NO: 75 - protein, 11 aa, Artificial Sequence
  Gly Ser linker
GGSGGGSGGSG

SEQ ID NO: 76 - protein, 10 aa, Artificial Sequence
  Gly Ser linker
GGSGGGSGSG

SEQ ID NO: 77 - protein, 8 aa, Artificial Sequence
  Gly Ser linker
GGSGGGSG

SEQ ID NO: 78 - protein, 120 aa, Artificial Sequence
  mouse palivizumab variable heavy chain
QVELQESGPGILQPSQTLSLTCSFSGFSLSTSGMSVGWIRQPSGEGLEWLADIWWDDKKDYNPSLKSRLTISKDTSSNQVFLKITGVDTADTATYYCARSMITNWYFDVWGAGTTVTVSS

SEQ ID NO: 79 - protein, 106 aa, Artificial Sequence
  mouse palivizumab variable light chain
DIQLTQSPAIMSASPGEKVTMTCSASSSVGYMHWYQQKLSTSPKLQIYDTSKLASGVPGRFSGSGSGNSYSLTISSIQAEDVATYYCFRGSGYPFTFGQGTKLEIK

SEQ ID NO: 80 - protein, 106 aa, Artificial Sequence
  human palivizumab variable light chain
DIQMTQSPSTLSASVGDRVTITCKCQLSVGYMHWYQQKPGKAPKLLIYDTSKLASGVPSRFSGSGSGTEFTLTISSLQPDFATYYCFQGSGYPFTFGGGTKLEIKR

SEQ ID NO: 81 - protein, 115 aa, Artificial Sequence
  human palivizumab variable heavy chain
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTT

SEQ ID NO: 82 - protein, 12 aa, Artificial Sequence
  anti-RSV antibody CDRH1
GASINSDNYYWT

SEQ ID NO: 83 - protein, 16 aa, Artificial Sequence
  anti-RSV antibody CDRH2
HISYTGNTYYTPSLKS

SEQ ID NO: 84 - protein, 15 aa, Artificial Sequence
  anti-RSV antibody CDRH3
CGAYVLISNCGWFDS

SEQ ID NO: 85 - protein, 11 aa, Artificial Sequence
  anti-RSV antibody CDRL1
QASQDISTYLN

SEQ ID NO: 86 - protein, 7 aa, Artificial Sequence
  anti-RSV antibody CDRL2
GASNLET

SEQ ID NO: 87 - protein, 9 aa, Artificial Sequence
  anti-RSV antibody CDRL3
QQYQYLPYT

SEQ ID NO: 88 - protein, 8 aa, Artificial Sequence
  10E8 anti-HIV antibody CDRH1
GFDFDNAW

SEQ ID NO: 89 - protein, 10 aa, Artificial Sequence
  10E8 anti-HIV antibody CDRH2
ITGPGEGWSV

SEQ ID NO: 90 - protein, 20 aa, Artificial Sequence
  10E8 anti-HIV antibody CDRH3
TGKYYDFWSGYPPGEEYFQD

SEQ ID NO: 91 - protein, 11 aa, Artificial Sequence
  10E8 anti-HIV antibody CDRL1
TGDSLRSHYAS

SEQ ID NO: 92 - protein, 7 aa, Artificial Sequence
  10E8 anti-HIV antibody CDRL2
GKNNRPS

SEQ ID NO: 93 - protein, 12 aa, Artificial Sequence
  10E8 anti-HIV antibody CDRL3
SSRDKSGSRLSV

SEQ ID NO: 94 - protein, 8 aa, Artificial Sequence
  VRC01 antibody CDRH1
GYEFIDCT

SEQ ID NO: 95 - protein, 8 aa, Artificial Sequence
  VRC01 antibody CDRH2
KPRGGAVN

SEQ ID NO: 96 - protein, 14 aa, Artificial Sequence
  VRC01 antibody CDRH3
RGKNCDYNWDFEHW

SEQ ID NO: 97 - protein, 4 aa, Artificial Sequence
  VRC01 antibody CDRL1
QYGS

SEQ ID NO: 98 - protein, 5 aa, Artificial Sequence
  VRC01 antibody CDRL3
QQYEF

SEQ ID NO: 99 - protein, 30 aa, Artificial Sequence
  anti-Dengue virus antibody (ab55) CDRH1
EVQLHQSGAELVKPGASVKLSCTVSGFNIK

SEQ ID NO: 100 - protein, 13 aa, Artificial Sequence
  anti-Dengue virus antibody (ab55) CDRH2
WVKQRPEQGLEWI

SEQ ID NO: 101 - protein, 31 aa, Artificial Sequence
  anti-Dengue virus antibody (ab55) CDRH3
ATIKADTSSNTAYLQLISLTSEDTAVYYCAF

SEQ ID NO: 102 - protein, 23 aa, Artificial Sequence
  anti-Dengue virus antibody (ab55) CDRL1
DIQMTQSPASLSVSVGETVTITC

SEQ ID NO: 103 - protein, 15 aa, Artificial Sequence
  anti-Dengue virus antibody (ab55) CDRL2
WYQQKQGKSPQLLVY

SEQ ID NO: 104 - protein, 32 aa, Artificial Sequence
  anti-Dengue virus antibody (ab55) CDRL3
GVPSRFSGSGSGTQYSLKINSLQSEDFGTYYC

SEQ ID NO: 105 - protein, 9 aa, Artificial Sequence
  anti-Dengue virus antibody (DB2-3) CDRH1
YTFTDYAIT

SEQ ID NO: 106 - protein, 18 aa, Artificial Sequence
  anti-Dengue virus antibody (DB2-3) CDRH2
GLISTYYGDSFYNQKFKG

SEQ ID NO: 107 - protein, 10 aa, Artificial Sequence
  anti-Dengue virus antibody (DB2-3) CDRH3
TIRDGKAMDY

SEQ ID NO: 108 - protein, 16 aa, Artificial Sequence
  anti-Dengue virus antibody (DB2-3) CDRL1
RSSQSLVHSNGNTYLH

SEQ ID NO: 109 - protein, 7 aa, Artificial Sequence
  anti-Dengue virus antibody (DB2-3) CDRL2
KVSNRFS

SEQ ID NO: 110 - protein, 9 aa, Artificial Sequence
  anti-Dengue virus antibody (DB2-3) CDRL3
SQSTHVPYT

SEQ ID NO: 111 - protein, 118 aa, Artificial Sequence
  anti-pertussis antibody variable heavy chain
QVQLQQPGSELVRPGASVKLSCKASGYKFTSYWMHWVKQRPGQGLEWIGNIFPGSGSTNYDEKFNSKATLTVDTSSNTAYMQLSSLTSEDSAVYYCTRWLSGAYFDYWGQGTTVTVSS

SEQ ID NO: 112 - protein, 106 aa, Artificial Sequence
  anti-pertussis antibody variable light chain
QIVLTQSPALMSASPGEKVTMTCSASSSVSFMYWYQQKPRSSPKPWIYLTSNLPSGVPARFSGSGSGTSYSLTISSMEAEDAATYYCQQWSSHPPTFGSGTKLEIK

SEQ ID NO: 113 - protein, 6 aa, Artificial Sequence
  anti-hepatitis C antibody CDRH1
SYGMHW

SEQ ID NO: 114 - protein, 18 aa, Artificial Sequence
  anti-hepatitis C antibody CDRH2
VIWLDGSNTYYADSVKGR

SEQ ID NO: 115 - protein, 17 aa, Artificial Sequence
  anti-hepatitis C antibody CDRH3
ARDIFTVARGVIIYFDY

SEQ ID NO: 116 - protein, 11 aa, Artificial Sequence
  anti-hepatitis C antibody CDRL1
RASQSVSSYLA

SEQ ID NO: 117 - protein, 7 aa, Artificial Sequence
  anti-hepatitis C antibody CDRL2
DASNRAT

SEQ ID NO: 118 - protein, 8 aa, Artificial Sequence
  anti-hepatitis C antibody CDRL3
QQRSNWVT

SEQ ID NO: 119 - protein, 8 aa, Artificial Sequence
  anti-influenza virus antibody CDRH1
GMTSNSLA

SEQ ID NO: 120 - protein, 8 aa, Artificial Sequence
  anti-influenza virus antibody CDRH2
IIPVFETP

SEQ ID NO: 121 - protein, 16 aa, Artificial Sequence
  anti-influenza virus antibody CDRH3
ATSAGGIVNYYLSFNI

SEQ ID NO: 122 - protein, 6 aa, Artificial Sequence
  anti-influenza virus antibody CDRL1
QTITTW

SEQ ID NO: 123 - protein, 9 aa, Artificial Sequence
  anti-influenza virus antibody CDRL3
QQYSTYSGT

SEQ ID NO: 124 - protein, 10 aa, Artificial Sequence
  anti-EBV antibody AMM01 CDRH1
YTFIHFGISW

SEQ ID NO: 125 - protein, 16 aa, Artificial Sequence
  anti-EBV antibody AMM01 CDRH2
IDTNNGNTNYAQSLQG

SEQ ID NO: 126 - protein, 15 aa, Artificial Sequence
  anti-EBV antibody AMM01 CDRH3
RALEMGHRSGFPFDY

SEQ ID NO: 127 - protein, 11 aa, Artificial Sequence
  anti-EBV antibody AMM01 CDRL1
GGHNIGAKNVH

SEQ ID NO: 128 - protein, 7 aa, Artificial Sequence
  anti-EBV antibody AMM01 CDRL2
YDSDRPS

SEQ ID NO: 129 - protein, 14 aa, Artificial Sequence
  anti-EBV antibody AMM01 CDRL3
CQVWDSGRGHPLYV

SEQ ID NO: 130 - protein, 22 aa, Artificial Sequence
  porcine teschovirus-1 P2A
  MISC_FEATURE (1)..(3): (GlySerGly) residues can be added to the 5′ end of the peptide to improve cleavage efficiency
GSGATNFSLLKQAGDVEENPGP

SEQ ID NO: 131 - protein, 21 aa, Artificial Sequence
  Thosea asigna virus T2A
  MISC_FEATURE (1)..(3): (GlySerGly) residues can be added to the 5′ end of the peptide to improve cleavage efficiency
GSGEGRGSLLTCGDVEENPGP

SEQ ID NO: 132 - protein, 24 aa, Artificial Sequence
  equine rhinitis A virus E2A
  MISC_FEATURE (1)..(3): (GlySerGly) residues can be added to the 5′ end of the peptide to improve cleavage efficiency
GSGQCTNYALLKLAGDVESNPGPP

SEQ ID NO: 133 - protein, 25 aa, Artificial Sequence
  foot-and-mouth disease virus F2A
  MISC_FEATURE (1)..(3): (GlySerGly) residues can be added to the 5′ end of the peptide to improve cleavage efficiency
GSGVKQTLNFDLLKLAGDVESNPGP

SEQ ID NO: 134 - RNA, 20 nt, Artificial Sequence
  gRNA molecules for CIITA deletion
ucgaguuggauguggaaggu

SEQ ID NO: 135 - RNA, 20 nt, Artificial Sequence
  gRNA molecules for CIITA deletion
uuuucauccccacuucacac

SEQ ID NO: 136 - RNA, 20 nt, Artificial Sequence
  gRNA molecules for CIITA deletion
ccucgggggagagagaggug

SEQ ID NO: 137 - RNA, 20 nt, Artificial Sequence
  gRNA molecules for CIITA deletion
ugggcucaggugcuuccuca

SEQ ID NO: 138 - RNA, 20 nt, Artificial Sequence
  gRNA molecules for CIITA deletion
ucaaaguagagcacauagga

SEQ ID NO: 139 - RNA, 20 nt, Artificial Sequence
  gRNA molecules for CIITA deletion
ccaucaaaaguccuuuuugg

SEQ ID NO: 140 - RNA, 20 nt, Artificial Sequence
  gRNA molecules for CIITA deletion
gugucuacacuuagccuuuc

SEQ ID NO: 141 - RNA, 20 nt, Artificial Sequence
  gRNA molecules for CIITA deletion
gggugaaauuucccaacuuu

SEQ ID NO: 142 - RNA, 20 nt, Artificial Sequence
  gRNA molecules for CIITA deletion
ccggccuuuuuaccuugggg

SEQ ID NO: 143 - RNA, 20 nt, Artificial Sequence
  gRNA molecules for CIITA deletion
ucugcagccuucccagagga

SEQ ID NO: 144 - RNA, 20 nt, Artificial Sequence
  gRNA molecules for TRAC deletion
uugcuccaggccacagcacu

SEQ ID NO: 145 - RNA, 20 nt, Artificial Sequence
  gRNA molecules for TRAC deletion
ucgaccagcuugacaucaca

SEQ ID NO: 146 - RNA, 20 nt, Artificial Sequence
  gRNA molecules for TRAC deletion
agaaucaaaaucggugaaua

SEQ ID NO: 147 - RNA, 20 nt, Artificial Sequence
  gRNA molecules for TRAC deletion
caugugcaaacgccuucaac

SEQ ID NO: 148 - RNA, 20 nt, Artificial Sequence
  gRNA molecules for TRAC deletion
aaaguuuagguucguaucug

SEQ ID NO: 149 - RNA, 20 nt, Artificial Sequence
  gRNA molecules for TRAC deletion
uuugagaaucaaaaucggug

SEQ ID NO: 150 - RNA, 20 nt, Artificial Sequence
  gRNA molecules for TRAC deletion
auucucaaacaaauguguca

SEQ ID NO: 151 - RNA, 20 nt, Artificial Sequence
  gRNA molecules for TRAC deletion
cuuuuagaaaguuccuguga

SEQ ID NO: 152 - RNA, 20 nt, Artificial Sequence
  gRNA molecules for TRAC deletion
aaagcuuuucucgaccagcu

SEQ ID NO: 153 - RNA, 20 nt, Artificial Sequence
  gRNA molecules for TRAC deletion
gagucucucagcugguacac

SEQ ID NO: 154 - RNA, 20 nt, Artificial Sequence
  gRNA molecules for TRBC deletion
cagaggaccugaaaaacgug

SEQ ID NO: 155 - RNA, 20 nt, Artificial Sequence
  gRNA molecules for TRBC deletion
agguccucuggaaagggaag

SEQ ID NO: 156 - RNA, 20 nt, Artificial Sequence
  gRNA molecules for TRBC deletion
agccaucagaagcagagauc

SEQ ID NO: 157 - RNA, 20 nt, Artificial Sequence
  gRNA molecules for TRBC deletion
ggugugggagaucucugcuu

SEQ ID NO: 158 - RNA, 20 nt, Artificial Sequence
  gRNA molecules for TRBC deletion
gcccuauccuggguccacuc

SEQ ID NO: 159 - RNA, 20 nt, Artificial Sequence
  gRNA molecules for TRBC deletion
uuccccuguuuucuuucaga

SEQ ID NO: 160 - RNA, 20 nt, Artificial Sequence
  gRNA molecules for TRBC deletion
uuucagacuguggcuucacc

SEQ ID NO: 161 - RNA, 20 nt, Artificial Sequence
  gRNA molecules for TRBC deletion
aggccucggcgcugacgauc

SEQ ID NO: 162 - RNA, 20 nt, Artificial Sequence
  gRNA molecules for TRBC deletion
caggccccacucaccugcuc

SEQ ID NO: 163 - RNA, 20 nt, Artificial Sequence
  gRNA molecules for TRBC deletion
aggccccacucaccugcucu

SEQ ID NO: 164 - RNA, 20 nt, Artificial Sequence
  gRNA molecules for B2M deletion
uggccuggaggcuauccagc

SEQ ID NO: 165 - RNA, 20 nt, Artificial Sequence
  gRNA molecules for B2M deletion
ccgauauuccucagguacuc

SEQ ID NO: 166 - RNA, 20 nt, Artificial Sequence
  gRNA molecules for B2M deletion
gaguaccugaggaauaucgg

SEQ ID NO: 167 - RNA, 20 nt, Artificial Sequence
  gRNA molecules for B2M deletion
cucacgucauccagcagaga

SEQ ID NO: 168 - RNA, 20 nt, Artificial Sequence
  gRNA molecules for B2M deletion
cauucucugcuggaugacgu

SEQ ID NO: 169 - RNA, 20 nt, Artificial Sequence
  gRNA molecules for B2M deletion
acuuuccauucucugcugga

SEQ ID NO: 170 - RNA, 20 nt, Artificial Sequence
  gRNA molecules for B2M deletion
cugaauugcuaugugucugg

SEQ ID NO: 171 - RNA, 20 nt, Artificial Sequence
  gRNA molecules for B2M deletion
auccauccgacauugaaguu

SEQ ID NO: 172 - RNA, 20 nt, Artificial Sequence
  gRNA molecules for B2M deletion
aauucucucuccauucuuca

SEQ ID NO: 173 - RNA, 20 nt, Artificial Sequence
  gRNA molecules for B2M deletion
agcaaggacuggucuuucua

SEQ ID NO: 174 - RNA, 25 nt, Artificial Sequence
  gRNA molecule for RFX5 deletion
aaaauguuuuaauguauauguguua

SEQ ID NO: 175 - RNA, 24 nt, Artificial Sequence
  gRNA molecule for RFX5 deletion
aauguuuuaauguauauguguuau

SEQ ID NO: 176 - RNA, 25 nt, Artificial Sequence
  gRNA molecule for RFX5 deletion
uauuauaauuucuuaaaaucugcug

SEQ ID NO: 177 - RNA, 25 nt, Artificial Sequence
  gRNA molecule for RFX5 deletion
uauaauuucuuaaaaucugcugugg

SEQ ID NO: 178 - RNA, 25 nt, Artificial Sequence
  gRNA molecule for RFX5 deletion
ucccugaaagaaaaucaguguuucu

SEQ ID NO: 179 - RNA, 25 nt, Artificial Sequence
  gRNA molecule for RFX5 deletion
cccugaaagaaaaucaguguuucuu

SEQ ID NO: 180 - RNA, 25 nt, Artificial Sequence
  gRNA molecule for RFX5 deletion
cccugacagaagcuacccuauguag

SEQ ID NO: 181 - RNA, 25 nt, Artificial Sequence
  gRNA molecule for RFX5 deletion
agcuacccuauguagaggacaaagu

SEQ ID NO: 182 - RNA, 25 nt, Artificial Sequence
  gRNA molecule for RFX5 deletion
aaguaggucuucaauaaauauuagu

SEQ ID NO: 183 - RNA, 25 nt, Artificial Sequence
  gRNA molecule for RFX5 deletion
auuaguugguuuacugcuuuuccca

SEQ ID NO: 184 - RNA, 25 nt, Artificial Sequence
  gRNA molecules for RFXAP deletion
cgcgggcguacacacgcugacgcgc

SEQ ID NO: 185 - RNA, 25 nt, Artificial Sequence
  gRNA molecules for RFXAP deletion
cguacacacgcugacgcgcaggcug

SEQ ID NO: 186 - RNA, 25 nt, Artificial Sequence
  gRNA molecules for RFXAP deletion
gcugacgcgcaggcugcggucgcgc

SEQ ID NO: 187 - RNA, 25 nt, Artificial Sequence
  gRNA molecules for RFXAP deletion
caggcugcggucgcgcaggcgcagu

SEQ ID NO: 188 - RNA, 25 nt, Artificial Sequence
  gRNA molecules for RFXAP deletion
ggcugcggucgcgcaggcgcagucg

SEQ ID NO: 189 - RNA, 25 nt, Artificial Sequence
  gRNA molecules for RFXAP deletion
cgcaggcgcagucggggcgccuucc

SEQ ID NO: 190 - RNA, 25 nt, Artificial Sequence
  gRNA molecules for RFXAP deletion
cgcagucggggcgccuucccgguau

SEQ ID NO: 191 - RNA, 25 nt, Artificial Sequence
  gRNA molecules for RFXAP deletion
uuuaccccagcguguccugagucuu

SEQ ID NO: 192 - RNA, 25 nt, Artificial Sequence
  gRNA molecules for RFXAP deletion
agucuuugguucgcgaagugccguu

SEQ ID NO: 193 - DNA, 20 bp, Artificial Sequence
  gRNA sequences for RFXANK deletion
cggctcctccatagcgaccg

SEQ ID NO: 194 - DNA, 20 bp, Artificial Sequence
  gRNA sequences for RFXANK deletion
cctccatagcgaccgcggcc

SEQ ID NO: 195 - RNA, 20 nt, Artificial Sequence
  gRNA sequences for RFXANK deletion
gcgaccgcggccgagcgaac

SEQ ID NO: 196 - RNA, 20 nt, Artificial Sequence
  gRNA sequences for RFXANK deletion
cgaccgcggccgagcgaacc

SEQ ID NO: 197 - RNA, 20 nt, Artificial Sequence
  gRNA sequences for RFXANK deletion
cggccgagcgaaccgggaaa

SEQ ID NO: 198 - RNA, 20 nt, Artificial Sequence
  gRNA sequences for RFXANK deletion
agcgaaccgggaaacggcac

SEQ ID NO: 199 - RNA, 20 nt, Artificial Sequence
  gRNA sequences for RFXANK deletion
gaaccgggaaacggcaccgg

SEQ ID NO: 200 - RNA, 20 nt, Artificial Sequence
  gRNA sequences for RFXANK deletion
gggaaacggcaccggaagcc

SEQ ID NO: 201 - RNA, 20 nt, Artificial Sequence
  gRNA sequences for RFXANK deletion
gaaacggcaccggaagcccg

SEQ ID NO: 202 - RNA, 20 nt, Artificial Sequence
  gRNA sequences for RFXANK deletion
aaacggcaccggaagcccgg

SEQ ID NO: 203 - protein, 65 aa, Artificial Sequence
  CD40
VFIKKVAKKPTNKAPHPKQEPQEINFPDDLPGSNTAAPVQETLHGCQPVTQEDGKESRISVQERQ

(19) United States
(12) Patent Application Publication
(10) Pub. No.: US 2024/0052013 A1
(43) Pub. Date: Feb. 15, 2024

(54) SYSTEMS AND METHODS TO LINK CD40 SIGNALING TO ANTIGEN BINDING

(71) Applicants: Fred Hutchinson Cancer Center, Seattle, WA (US); University of Washington, Seattle, WA (US)

(72) Inventors: Justin J. Taylor, Seattle, WA (US); Laila Shehata, Seattle, WA (US); Marti Tooley, Seattle, WA (US)

(73) Assignees: Fred Hutchinson Cancer Center, Seattle, WA (US); University of Washington, Seattle, WA (US)

(21) Appl. No.: 18/264,749

(22) PCT Filed: Feb. 8, 2022
(86) PCT No.: PCT/US2022/015690

§ 371 Date: Aug. 8, 2023

Related U.S. Application Data
(60) Provisional application No. 63/147,041, filed on Feb. 8, 2021.

(51) Int. Cl. C07K 14/705; C07K 16/10; C07K 16/08; C07K 16/12; C07K 16/24; C12N 5/0781; A61K 39/00

(52) U.S. Cl. CPC C07K 14/70578; C07K 16/1027; C07K 16/10; C07K 16/1018; C07K 16/081; C07K 16/085; C07K 16/087; C07K 16/1282; C07K 16/241; C07K 14/70503; C12N 5/0635; A61K 39/4612; A61K 39/4622; A61K 39/4637; C07K 2319/03; C07K 2317/622; C07K 2319/02; A61K 2239/26; A61K 2239/22

(57) ABSTRACT

Systems and methods to link CD40 signaling to antigen binding, independently of CD40 ligand binding are described. The systems and methods include fusion proteins including an extracellular antigen binding domain linked to an intracellular CD40 signaling domain.

### CROSS-REFERENCE TO RELATED APPLICATION

This application is a U.S. National Phase Patent Application based on International Patent Application No. PCT/US2022/015690, filed on Feb. 8, 2022, which claims priority to U.S. Provisional Patent Application No. 63/147,041 filed on Feb. 8, 2021, both of which are incorporated herein by reference in their entirety as if fully set forth herein.

### REFERENCE TO SEQUENCE LISTING

The Sequence Listing associated with this application is provided in text format in lieu of a paper copy and is hereby incorporated by reference into the specification. The name of the text file containing the Sequence Listing is 2XT8521_ST25.txt. The text file is 202 KB, was created on Aug. 3, 2023, and is being submitted electronically via Patent Center.

### FIELD OF THE DISCLOSURE

The present disclosure describes systems and methods to link CD40 signaling to antigen binding, independently of interactions with CD40 ligand. The systems and methods include fusion proteins that, when expressed by a cell, include an extracellular antigen binding domain linked to an intracellular CD40 signaling domain.

### BACKGROUND OF THE DISCLOSURE

Vaccines are designed to increase the immunity of a subject against a particular infection by stimulating B cells to produce antibodies against the targeted infectious agent. Routine pediatric vaccination is a long-established clinical intervention with comparatively low risk and high efficacy. Unfortunately, however, vaccinations are not available for all infectious agents. As one example, every year in the United States, millions of children visit a doctor or emergency room due to infections with Respiratory Syncytial Virus (RSV).

In addition to combating infections, antibodies can also be useful as treatments for other conditions such as autoimmune diseases, and cancer. However, these antibody-based therapies typically require repeated injections of the antibodies to maintain efficacy.

Technologies have been developed to genetically modify B cells to express selected antibodies that bind targeted antigens (see WO2019/079772). Potent B cell responses, however, typically require CD40 expressed on the surface of B cells to interact with CD40 ligand (CD40L) expressed on the surface of CD4+ T cells. If T cells have not encountered the targeted antigen, such interactions may not sufficiently occur. Absent the required T cell interactions, B cells expressing antibodies may fail to adequately expand, survive, and differentiate.

B cell's natural dependence on T cell interactions also hampers the development of universal donor B cells as broadly applicable therapeutics. This is because T cells interact with B cells by binding to peptides presented on the B cell surface within major histocompatibility complex (MHC) class II molecules. Within the human population there are hundreds of different MHC alleles, with the most frequent allele only being present in 10% of the population. Unfortunately, the host immune system will reject any cell that expresses a different MHC allele than the host.

### SUMMARY OF THE DISCLOSURE

The current disclosure describes B cells that receive CD40 activation signals independently of CD40 ligand (CD40L) binding. This advance is achieved by genetically modifying B cells to express fusion proteins that link extracellular antigen binding domains to CD40 signaling domains. In this manner, CD40 activation signals are triggered by antigen binding, independently of interactions with CD40L expressed by T cells.

The current disclosure provides two main approaches to link CD40 signaling to antigen binding. The first includes linking CD40 signaling domains to engineered extracellular antigen binding domains, such as scFv. In this approach, CD40 signaling is triggered when antigen binds the engineered antigen binding domain.

The second approach includes linking CD40 signaling to B cell receptor (BCR) signaling. When bound by antigen, BCR naturally transmit B cell activation signals through the intracellular proteins CD79α and CD79β. The current disclosure provides linking an intracellular CD40 signaling domain to CD79α and/or CD79β, such that when CD79α and CD79β signaling is triggered by antigen binding, CD40 signaling is also triggered.

When a subject donates his or her own B cells for genetic engineering (or is otherwise immunologically matched to a donor), no modifications regarding the B cell's MHC alleles are required. However, another advance described herein is deleting MHC class II molecules from the genetically-engineered B cells so that the B cells can be used as universal donor cells for all individuals. MHC class II molecule expression can be deleted by eliminating activity of one or more transcription factors required for MHC class II molecule expression. Such transcription factors include CIITA, TRAC, TRBC, B2M, RFX5, and RFXAP.

### BRIEF DESCRIPTION OF THE DRAWINGS

Some of the drawings submitted herein may be better understood in color. Applicant considers the color versions of the drawings as part of the original submission and reserves the right to present color images of the drawings in later proceedings.

FIG. 1. Schematic of T cell dependent B cell activation, showing a T cell (left), a B cell (right) and engagement of several interaction molecules. Adapted from Janeway et al, Immunologie (Berlin, 2002)

FIG. 2. The CD40-CD40L complex showing the trimeric form of CD40L wedged between two monomers of CD40. Adapted from An et al., “Crystallographic and Mutational Analysis of the CD40-CD154 Complex and Its Implications for Receptor Activation”, J. Biol. Chem. 2011 Apr. 1; 286(13): 11226-11235 and PDB Accession 3QD6.

FIG. 3. A mapping of protein interaction with CD40 intracellularly. The grey boxes underlining the CD40 sequence (SEQ ID NO: 203) from left to right include the transmembrane region (up to residue 215), box-1 binding site (residues 222-229), TRAF binding sites (residues 231-238 and 250-254), and box-2 binding site (residues 260-270). Adapted from An et al., J. Biol. Chem. 2011 Apr. 1; 286(13): 11226-11235

FIG. 4. Schematic depicting B cell activities following activation.

FIG. 5. Schematics of exemplary fusion proteins disclosed herein. These depicted constructs each include an engineered extracellular binding domain (scFv), an extracellular tag (Strep), an extracellular spacer (CD40 or IgG segment), a transmembrane domain (CD40 or IgG segment), a CD40 intracellular signaling domain, and an intracellular tag (HA). Construct (+) additionally includes an FKBP12 dimerization sequence.

FIGS. 6A-6D. (6A) Schematic of a natural BCR complex depicting an extracellular antigen binding domain and CD79α/β BCR signaling domains. (6B-6D) Schematics of BCR-CD40 constructs disclosed herein. (6B) Intracellular CD40 signaling domain linked to CD79α and CD79β subunits. (6C) Intracellular CD40 signaling domain linked to CD79α. (6D) Intracellular CD40 signaling domain linked to CD79β.

FIG. 7. Human B cells were purified from PBMC and stimulated for 2 days in vitro in the presence of CD40L, IL-2, IL-10, IL-15 and CpG prior to electroporation with a plasmid encoding construct #1. Cells were placed back in culture with media containing CD40L, IL-2, IL-10, IL-15 and CpG for an additional 24 hours. The media was then removed and replaced with media containing insulin and transferrin with or without 15 ug/mL tetramerized HIV Env (SF162) and incubated for an additional 24 hours. Cells were harvested and stained intracellularly for phosphorlyated-p38 (p-p38), phospho-STAT3 (p-STAT3), and the HA tag expressed by the construct. The % of P-p38+ and P-STAT+ cells that expressed the construct (HA+, five-point start) or did not express the construct (HA−, triangle) are displayed. Bottom Panel: As a positive control for CD40 signaling, control cells were incubated in the presence (pentagon) or absence of CD40L (triangle) throughout the culture period.

FIG. 8. RAMOS B cells were transfected with constructs encoding mCherry and wild-type CD79α, wild-type CD79β, CD79α with the CD40 signaling domain (CD79β/CD40), and/or CD79β with the CD40 signaling domain (CD79β/CD40) prior to incubation with 5 ug/mL anti-human Ig for 48 hours. After incubation, cells were permeabilized and stained for phosphorylated-p38 and phosphorylated-STAT3. Cells that were + and − for the constructs were determined based upon the presence or absence of mCherry.

FIG. 9. RAMOS B cells were electroporated with gRNA targeting B2M and/or CIITA 48 hours prior to the analysis of MHCI and MHCII expression using flow cytometry. sgRNA sequences for CIITA (SEQ ID NO: 1) and B2M (SEQ ID NO: 2) are provided.

FIGS. 10A-10H. Sequence schematics in which coding sequence is aligned with amino acid sequence.

(10A) Schematic of Construct 1 (2520 bp). The coding sequence encodes the amino acid sequence including a human CD40 signal peptide—anti-gp120 scFV—a StrepTagII®—Gly linker CD40 full protein—Gly linker —HA tag—P2A skipping element—mCherry reporter.

(10B) Schematic of Construct 2 (2085 bp). The coding sequence encodes the amino acid sequence including human CD40 signal peptide—anti-gp120 scFv—StrepTagII®—Gly linker—IgG hinge—Gly linker—CD40 transmembrane domain and CD40 intracellular domain—Gly linker—HA tag—P2A skipping element—mCherry reporter.

(10C) Schematic of Construct 3 (2082 bp). The coding sequence encodes the amino acid sequence including human CD40 signal peptide—anti-gp120 scFv—StrepTagII®—Gly linker; IgG hinge—Gly linker—IgG2 transmembrane domain; Gly linker—CD40 intracellular domain—Gly linker—HA tag—P2A skipping element—mCherry reporter.

(10D) Schematic of Construct 4 (3225 bp). The coding sequence encodes the amino acid sequence including human CD40 signal peptide—anti-gp120 scFv—StrepTagII®—Gly linker—full CD40 protein—Gly linker—FkBP12v36—GlySer linker—FkBP12v36—Gly linker—HA tag—P2A skipping element—mCherry reporter.

(10E) Schematic of Construct 5 (1692 bp). The coding sequence encodes the amino acid sequence including human CD40 signal peptide—full CD40 protein—Gly linker—HA tag—P2A skipping element—mCherry Reporter.

(10F) Schematic of MRT3_PTT3_mouse CD79α_IntracellularCD40 (7736 bp) in which CD40 is linked to mouse CD70α. The coding sequence encodes the aligned amino acid sequence including mouse CD79α—intracellular CD40-P2A skipping element —T2A skipping element —mCherry reporter.

(10G) Schematic of MRT4_PTT3_mouse CD79β_Intracellular CD40 (7760 bp) in which CD40 is linked to mouse CD79β. The coding sequence encodes the aligned amino acid sequence including mouse CD79β—intracellular CD40-P2A skipping element—T2A skipping element —mCherry reporter.

(10H) Schematic of MRT5_Ptt3_humanCD79α_Intracellular CD40 (7638 bp) in which CD40 is linked to human CD79α. The coding sequence encodes the aligned amino acid sequence including human CD79α—intracellular CD40-P2A skipping element—mCherry reporter.

FIG. 11. Additional sequences supporting the disclosure. Sequences include:

coding sequence of Construct 1 (SEQ ID NO: 3) and protein sequence of Construct 1 including a human CD40 signal peptide (underlined), anti-gp120 scFV, a StrepTagII® (bold), Gly linker (double underlined), the CD40 full protein (bold and underlined), a Gly linker (double underlined), HA tag (italicized), P2A skipping element (bold and italicized), and an mCherry reporter (italicized and underlined) (SEQ ID NO: 4);
coding sequence of Construct 2 (SEQ ID NO: 5) and protein sequence of Construct 2 including human CD40 signal peptide (underlined), anti-gp120 scFv, StrepTagII® (bold), Gly linker (double underlined), IgG hinge (bold, underlined, and italicized), Gly linker (double underlined), CD40 transmembrane domain and CD40 intracellular domain (bold and underlined), Gly linker (double underlined), HA tag (italicized), P2A skipping element (bold and italicized), and an mCherry reporter (underlined and italicized (SEQ ID NO: 6);
coding sequence of Construct 3 (SEQ ID NO: 7) and protein sequence of Construct 3 including human CD40 signal peptide (underlined); anti-gp120 scFv, StrepTagII® (bold), Gly linker, IgG hinge (bold, underlined, and italicized), Gly linker (double underlined), IgG2 transmembrane domain, Gly linker (double underlined), CD40 intracellular domain (bold and underlined), Gly linker (double underlined), HA tag (italicized), P2A skipping element (bold and italicized), and an mCherry reporter (underlined and italicized) (SEQ ID NO: 8);
coding sequence of Construct 4 (SEQ ID NO: 9) and sequence of Construct 4 including human CD40 signal peptide (underlined), anti-gp120 scFv, StrepTagII® (bold), Gly linker (double underlined), full CD40 protein (bold and underlined), Gly linker (double underlined), FkBP12v36 (bold, underlined, and italicized), GlySer linker, FkBP12v36 (bold, underlined, and italicized), Gly linker (double underlined), HA tag (italicized), P2A skipping element (bold and italicized), and mCherry reporter (underlined and italicized (SEQ ID NO: 10);
coding sequence of Construct 5 (SEQ ID NO: 11) and protein sequence of Construct 5 including human CD40 signal peptide (underlined), full CD40 protein (bold and underlined), Gly linker (double underlined), HA tag (italicized), P2A skipping element (bold and italicized), and an mCherry Reporter (italicized and underlined (SEQ ID NO: 12);
coding sequence of MRT3 (SEQ ID NO: 13) and protein sequence of MRT3 including mouse CD79α (underlined), mouse intracellular CD40 (italicized), P2A skipping element (double underlined), T2A skipping element (bold), and an eGFP reporter (SEQ ID NO: 14);
coding sequence of MRT4 (SEQ ID NO: 15) and protein sequence of MRT4 including mouse CD79β (underlined), mouse intracellular CD40 (italicized), P2A skipping element (double underlined), T2A skipping element (bold), and an eGFP reporter (SEQ ID NO: 16);
coding sequence of MRT5 (SEQ ID NO: 17) and protein sequence of MRT5 including human CD79α (underlined), human intracellular CD40 (italicized), P2A skipping element (double underlined), and an mCherry reporter (SEQ ID NO: 18);
coding sequence of MRT6 (SEQ ID NO: 19) and protein sequence of MRT6 including human CD79β (underlined), human intracellular CD40 (italicized), P2A skipping element (double underlined), and an mCherry reporter (SEQ ID NO: 20);
Anti-gp120 scFv—Linker—StrepTagII—Gly6 Linker—full CD40 protein—Gly6 Linker—HA tag (SEQ ID NO: 21);
Anti-gp120 scFv—Linker—StrepTagII—Gly6 Linker—IgG Hinge—Gly6 Linker—CD40 Transmembrane Domain—CD40 Intracellular Domain—Gly6 Linker—HA tag (SEQ ID NO: 22);
Anti-gp120 scFv—Linker—StrepTagII—Gly6 Linker—IgG Hinge—Gly6 Linker—IgG2 Transmembrane Domain—Gly6 Linker—CD40 Intracellular Domain—Gly6 Linker—HA tag (SEQ ID NO: 23);
Anti-gp120 scFv—Linker—StrepTagII—Gly6 Linker—full CD40 protein—Gly6 Linker-FkBP12v36—GlySer Linker—FkBP12v36—Gly6 Linker—HA tag (SEQ ID NO: 24);
MouseCD79α-IntracellularCD40 (SEQ ID NO: 25);
MouseCD79β-IntracelluarCD40 (SEQ ID NO: 26);
HumanCD79α-IntracellularCD40 (SEQ ID NO: 27);
HumanCD79β-IntracellularCD40 (SEQ ID NO: 28); and
human CD40 signal peptide (SEQ ID NO: 29); anti-gp120 scFv (SEQ ID NO: 30); full CD40 protein (SEQ ID NO: 31); CD40 extracellular domain (SEQ ID NO: 32); CD40 transmembrane domain (SEQ ID NO: 33); CD40 intracellular domain (SEQ ID NO: 34); human intracellular CD40 domain (SEQ ID NO: 35); murine intracellular CD40 domain (SEQ ID NO: 36); IgG hinge (SEQ ID NO: 37); IgG2 transmembrane domain (SEQ ID NO: 38); FkBP12v36 (SEQ ID NO: 39); FKBP12 domain (SEQ ID NO: 40); FRB segment of mTOR (SEQ ID NO: 41); mCherry reporter (SEQ ID NO: 42); eGFP Reporter (SEQ ID NO: 43); murine CD79α (SEQ ID NO: 44); murine CD79β (SEQ ID NO: 45); human CD79α (SEQ ID NO: 46); human CD79β (SEQ ID NO: 47); linker (SEQ ID NO: 48); linker (SEQ ID NO: 49); StrepTagII (SEQ ID NO: 50); HA tag (SEQ ID NO: 51); His tag (SEQ ID NO: 52); Avi tag (SEQ ID NO: 53); sFLAG (SEQ ID NO: 54); Tev (SEQ ID NO: 55); Flag tag (SEQ ID NO: 56); Xpress tag (SEQ ID NO: 57); Calmodulin tag (SEQ ID NO: 58); Myc tag (SEQ ID NO: 59); Softag 1 (SEQ ID NO: 60); Softag 3 (SEQ ID NO: 61); STREP® tag (SEQ ID NO: 62); and V5 tag (SEQ ID NO: 63).

### DETAILED DESCRIPTION

Vaccines are designed to increase the immunity of a subject against a particular infection by stimulating B cells to produce antibodies against the targeted infectious agent. Routine pediatric vaccination is a long-established clinical intervention with comparatively low risk and high efficacy. Unfortunately, however, vaccinations are not available for all infectious agents. As one example, every year in the United States, millions of children visit a doctor or emergency room due to infections with Respiratory Syncytial Virus (RSV).

In addition to combating infections, antibodies can also be useful as treatments for other conditions such as autoimmune diseases. However, these antibody-based therapies typically require repeated injections of the antibodies to maintain protection.

Technologies have been developed to genetically modify B cells to express selected antibodies (see, e.g., WO2019/079772, which is incorporated by reference herein for its teachings regarding the same). Potent B cell responses, however, typically require these modified B cells to interact with CD4+ T cells to receive activation signals from the T cells. If the T cells have not encountered the antigen, such interactions may not sufficiently occur. Absent the required T cell interactions, B cells expressing selected antibodies may fail to adequately expand, survive, and differentiate.

B cell's natural dependence on T cell interactions also hampers the development of universal donor B cells as broadly applicable therapeutics. This is because T cells interact with B cells by binding to proteins presented on the B cell surface within major histocompatibility complex (MHC) class II molecules. In humans, MHC molecules are referred to as human leukocyte antigen (HLA) molecules. More than 600 HLA alleles are known, and more than 10,000 alleles are estimated (Lim et al., Cell. 168(4):724-740, 2017). The frequency of expression of HLA alleles varies by population and ethnic group (see, for instance, U.S. Pat. No. 8,268,964). Unfortunately, the most frequent allele is only present in 10% of the population, and T cells will reject any cell that expresses a different MHC allele than the host.

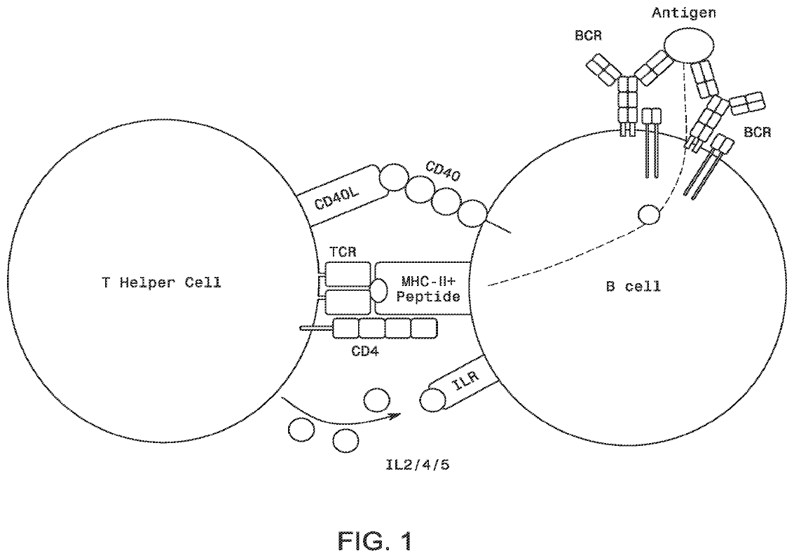

The most important T cell activation signal comes through the binding of CD40 expressed by B cells with CD40L expressed by T cells. Such stimulation of CD40 by CD40L binding promotes cytokine production (e.g., IL-2, IL-4, IL-21, IL-10, IFN-g, BAFF/BLyS), isotype switching, GC formation, somatic hypermutation, and formation of long-lived plasma B cells and memory B cells. FIG. 1 provides a schematic depicting B-cell/T-cell interactions including CD40/CD40L binding.

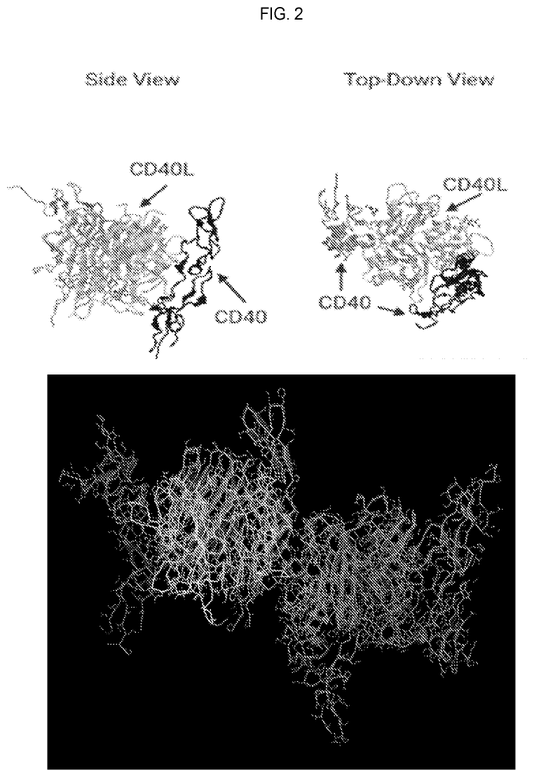

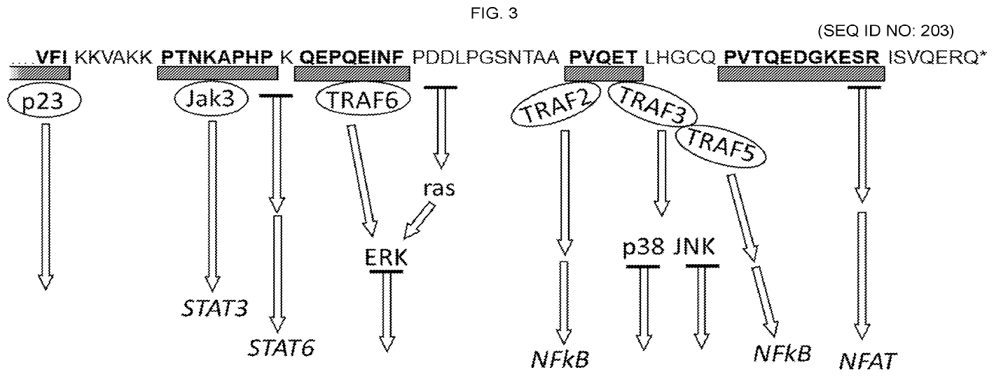

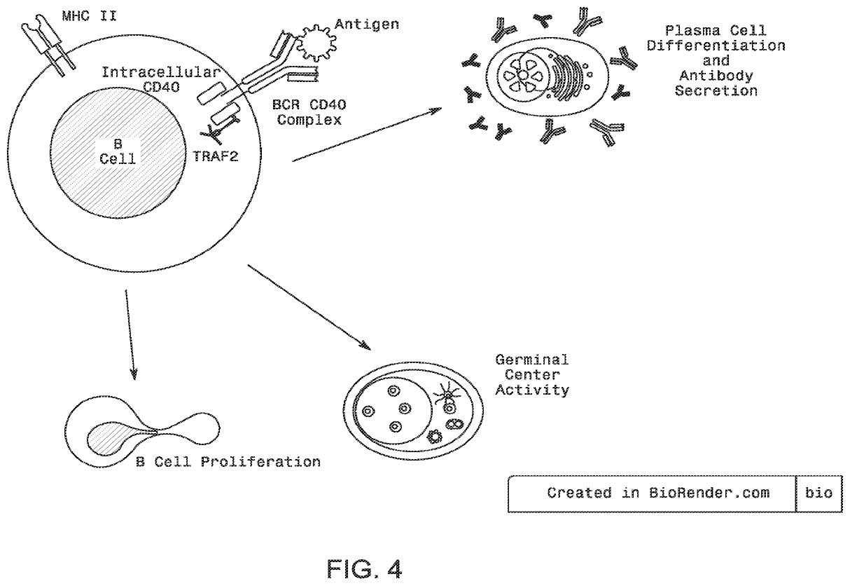

CD40's binding partner, CD40L (CD154) is found mainly on activated mature T cells but also appears on granulocytes, macrophages, and platelets. CD40/CD40L binding interactions are depicted in FIG. 2. CD40 binds asymmetrically to two subunits of CD40L's trimeric complex. Though CD40L is a trimer, it does not lead to trimerization of CD40. This is evident in the 2:3 molar ratio of CD40:CD40L that is consistently isolated. CD40L is endocytosed and then degraded by the B cell once CD40 interacts with CD40L. This interaction also promotes the release of soluble CD40 which will bind to CD40L on T cells and through proteolytic cleavage down regulate the mRNA expression of CD40L. CD40 activation by its ligand activates several pathways due to phosphorylation of TRAF pathways such as NFkB, MAPK and P13k. It is important to note that CD40 does not have a kinase domain and does not phosphorylate. TRAF5 activation is key for B cell proliferation and regulation of surface receptors. FIG. 3 depicts intracellular CD40 signaling pathways that can result in phosphorylation of p38, Erk, and Stat3, for example. Stimulation of CD40 signaling pathways can ultimately result in activation of B cell activities as described previously and as depicted in FIG. 4.

The current disclosure describes B cells that receive sufficient activation signals independently of CD40L binding. “Independently of CD40L binding” and “independently of interaction with CD40L” (used interchangeably herein) means that CD40L binding is not required for B cell activation. Because the B cells do not require CD40L binding for activation, they can be engineered into universal donor cells by deleting non-host compatible MHC molecules.

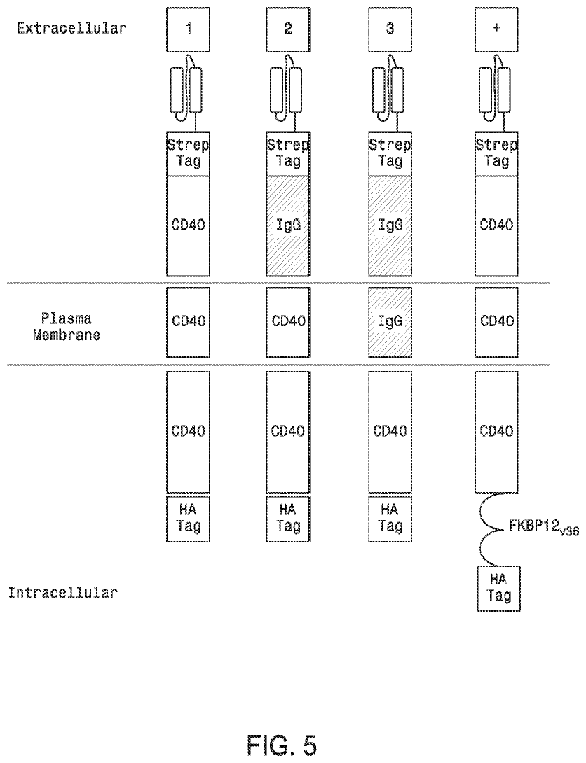

The current disclosure provides B cells that receive sufficient activation signals independently of CD40L binding by disclosing fusion proteins that allow CD40 activation signals following antigen binding to a B cell. One example of such a fusion protein includes, when expressed, an extracellular antigen binding domain linked through a transmembrane domain to an intracellular CD40 signaling domain. Examples of this approach are depicted in FIG. 5, showing fusion proteins including an scFv binding domain, one or more tag sequences, one or more spacer sequences, one or more multimerization molecules, a transmembrane domain, and an intracellular CD40 signaling domain. Such proteins can be expressed with signal peptides, reporter proteins (e.g., mCherry, truncated EGFR (tEGFR)) and skipping elements (e.g., IRES or self-cleaving peptides).

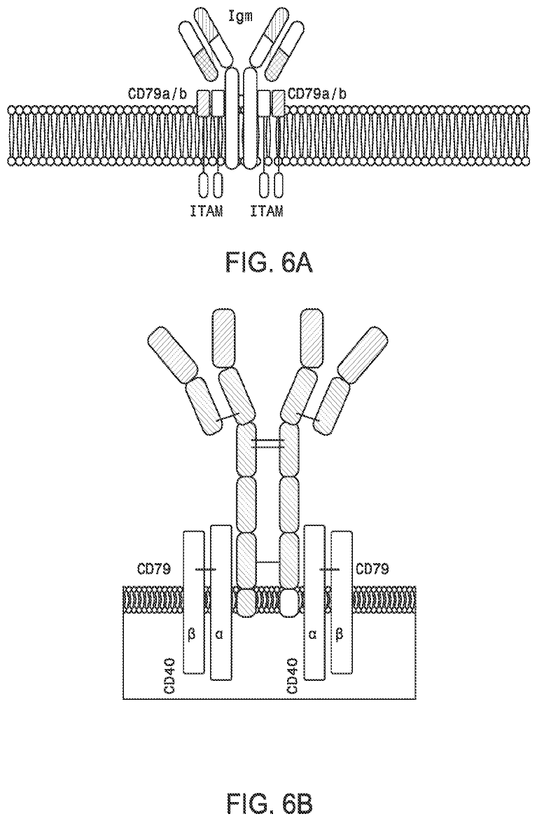

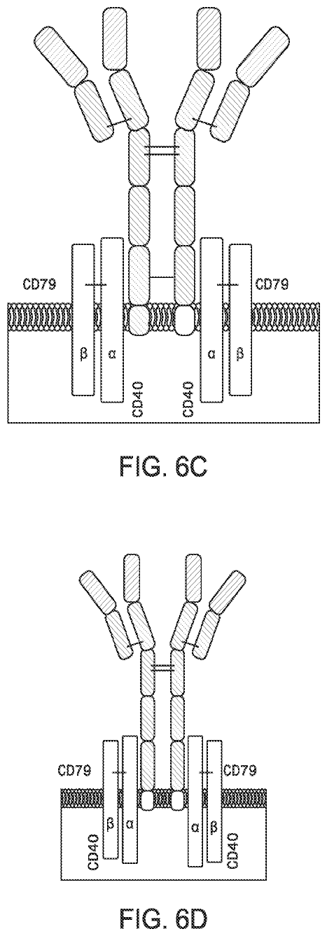

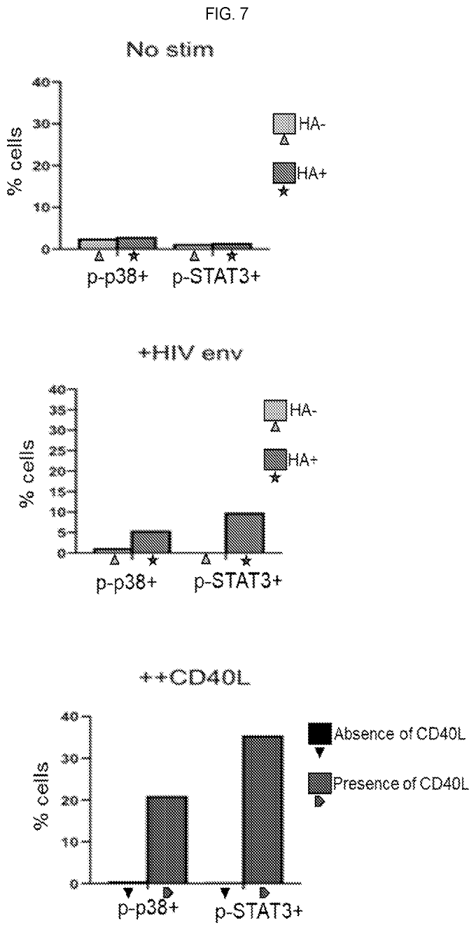

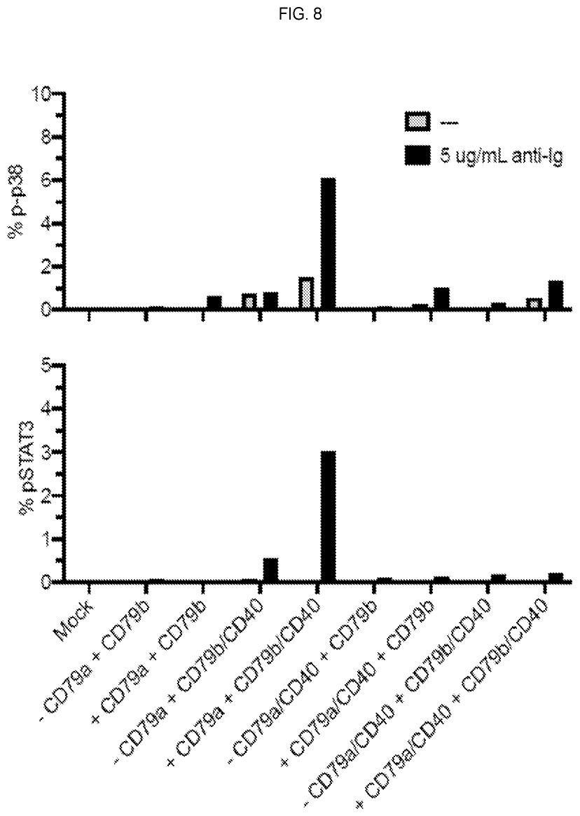

Another approach disclosed herein utilizes antigen-binding based B cell receptor (BCR) signaling to trigger CD40 signaling. BCR naturally transmit B cell activation signals through the intracellular proteins CD79α and CD79β when bound by an antigen (see FIG. 6A). More particularly, BCR on B cells electrostatically associate CD79α and CD79β. CD79α and CD79β are intrinsically disordered but have a secondary structure tendency with immunoreceptor tyrosine-based activation motif (ITAM) regions for downstream molecule interactions with kinases. These proteins are phosphorylated due to crosslinking from BCR aggregation from interaction with antigen. Upon phosphorylation, both the α and β subunit change conformational shape in degree of helical turn. The a subunit increases in helicy while the β subunit loses helical tightness. The sequence between the α and β subunit are only 20% similar and the β subunits exact function has yet to be fully elucidated. The ITAM regions are highly sensitive to mutations especially in the N-terminal residues. If the regions are disrupted, calcium mobilization and phosphorylation of subsequent proteins like Syk and Src will be affected.

As shown in FIGS. 6B-6D, fusion proteins can be generated wherein the intracellular signaling portion of CD40 is linked to the intracellular signaling portion of the BCR itself. An intracellular CD40 signaling domain can be linked to a CD79α subunit and a CD79β subunit (FIG. 6B), a CD79α subunit and a CD79α subunit (FIG. 6C), or a CD79β subunit and a CD79β subunit (FIG. 6D). Using this second approach the antigen binds to the BCR which transmits both BCR and CD40 signals substantially simultaneously. As with other fusion proteins described herein, these embodiments can similarly be expressed with one or more signal peptides, reporter proteins (e.g., mCherry, tEGFR), skipping elements (e.g., IRES or self-cleaving peptides), and/or multimerization molecules.

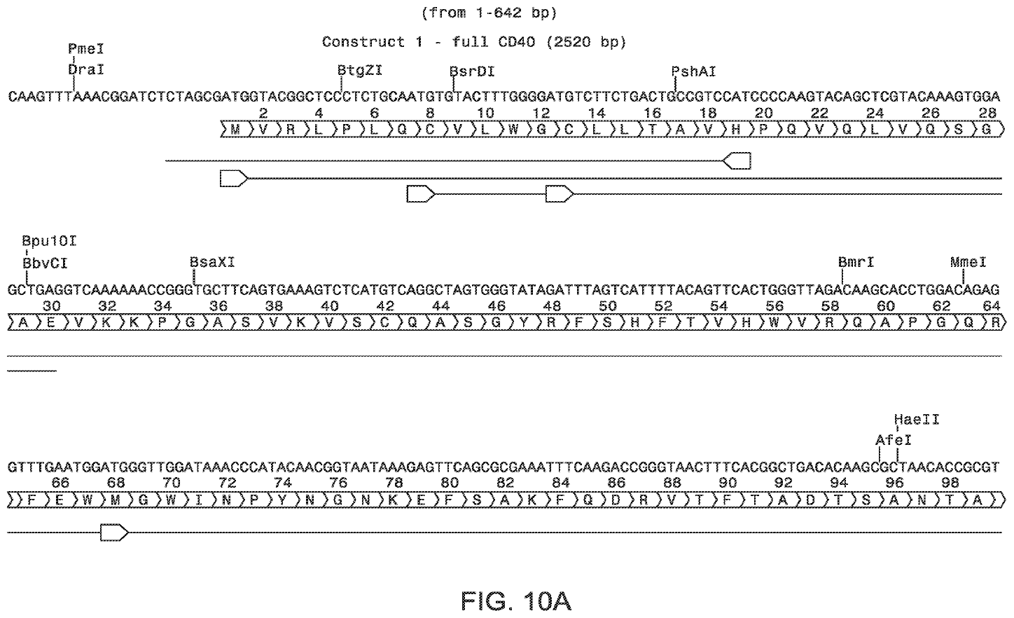

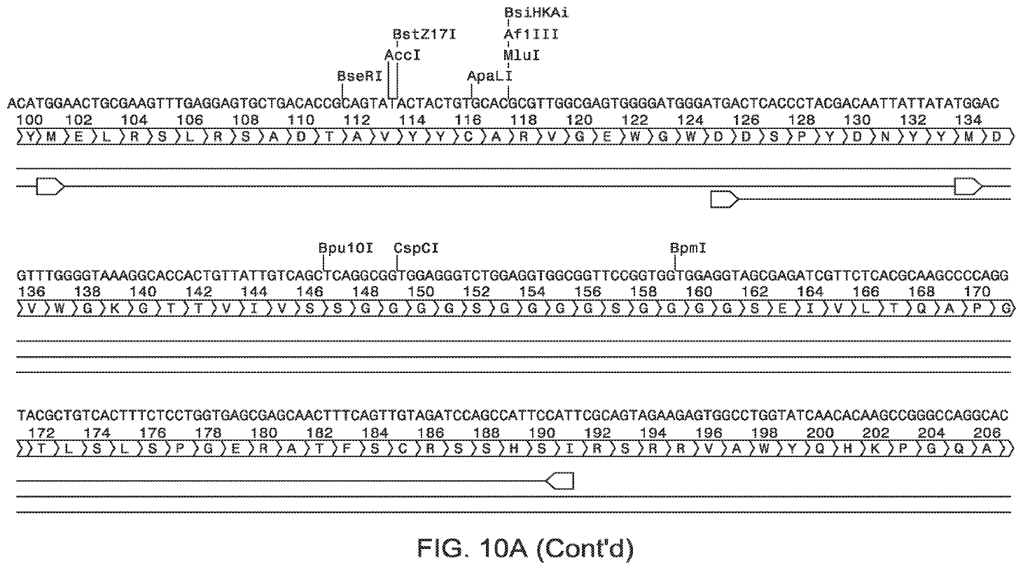

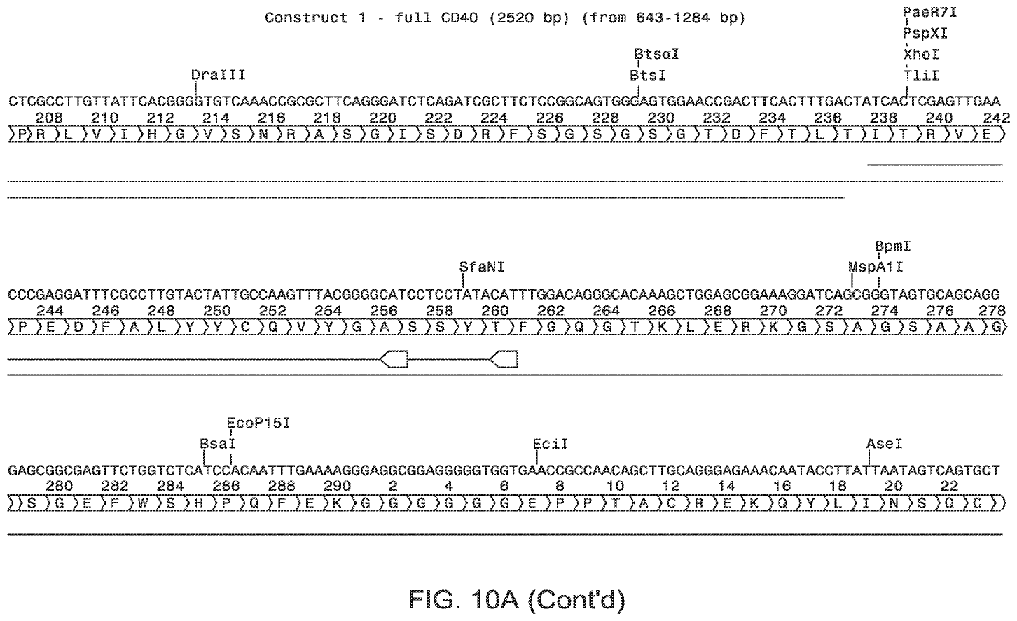

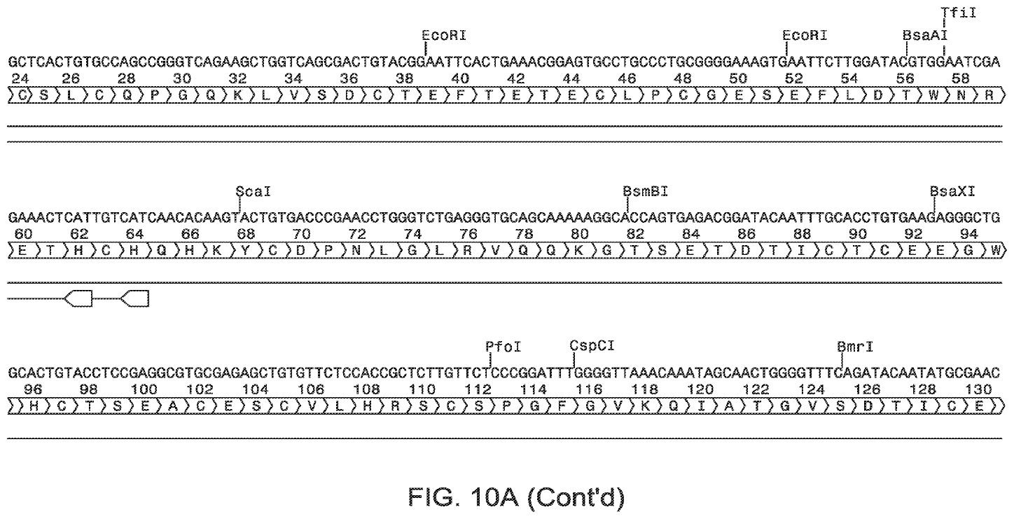

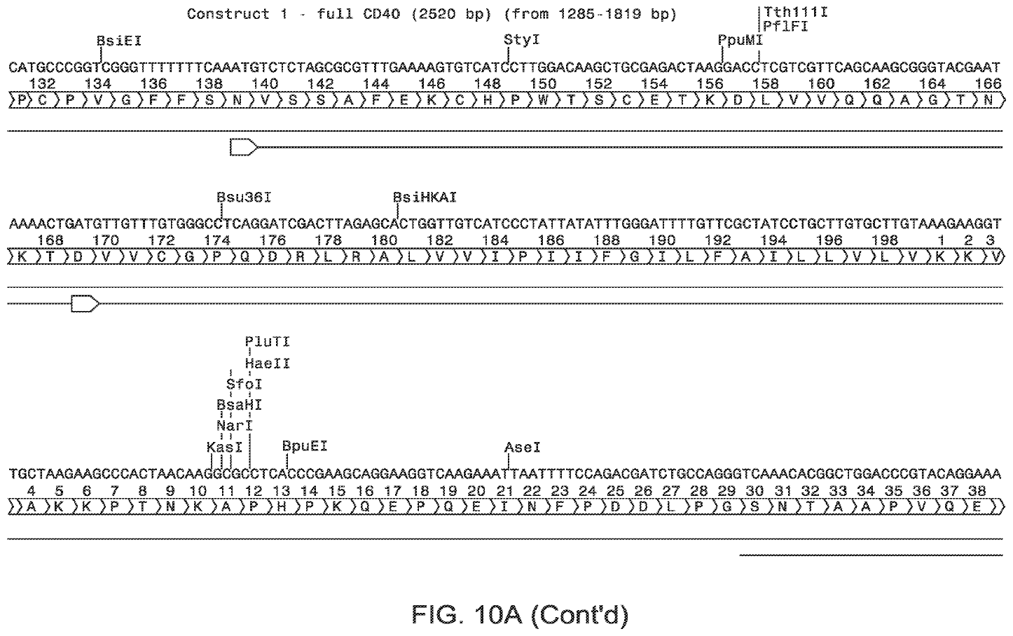

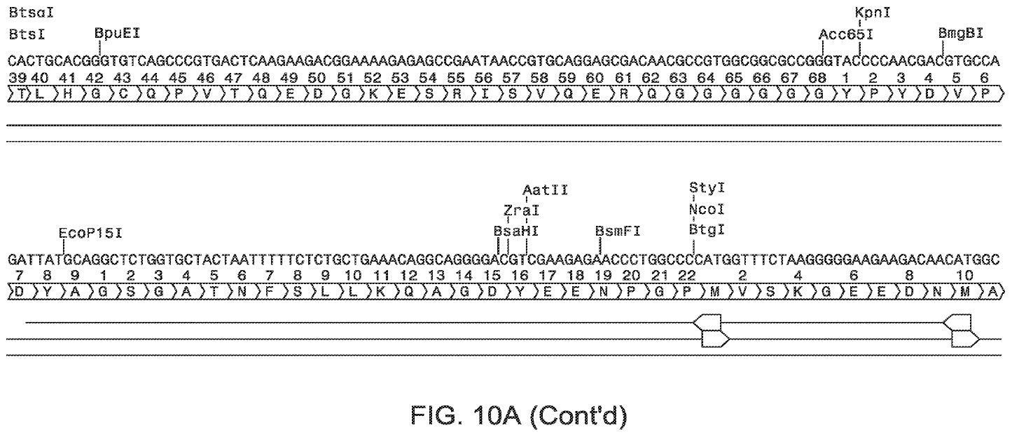

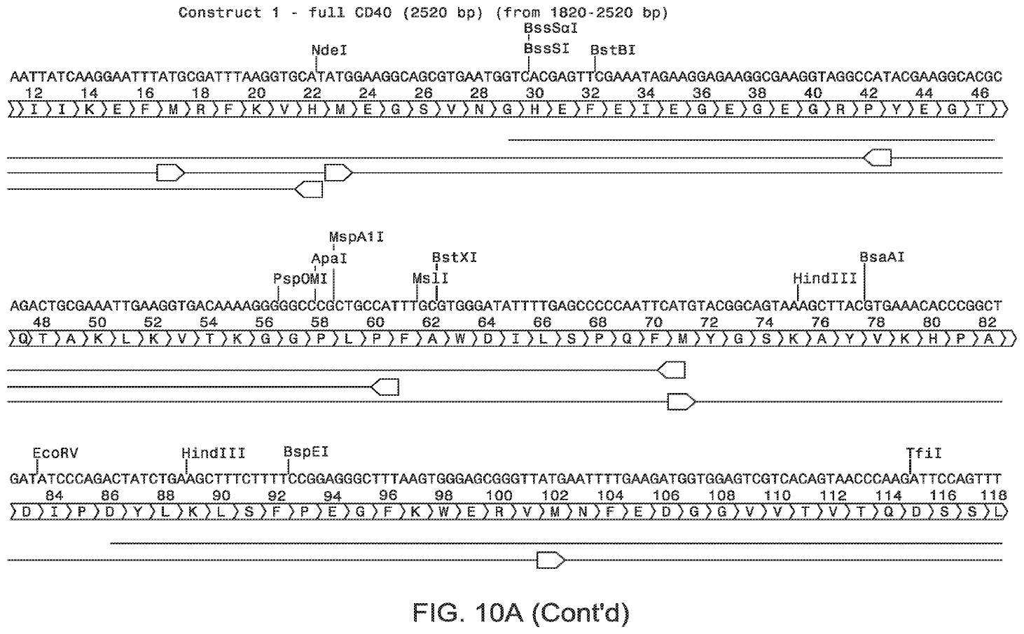

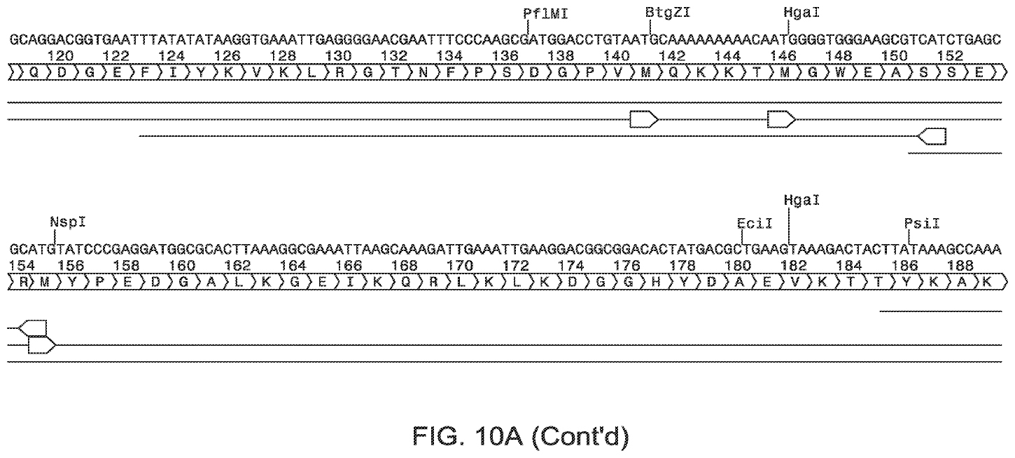

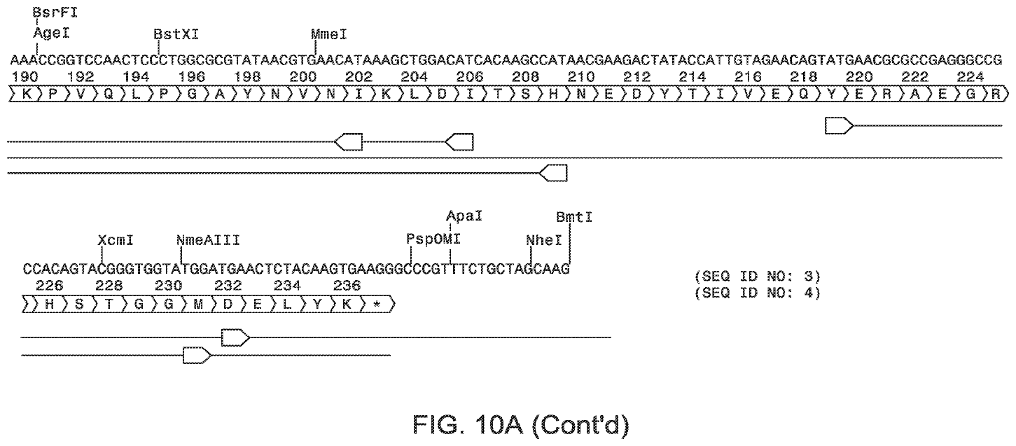

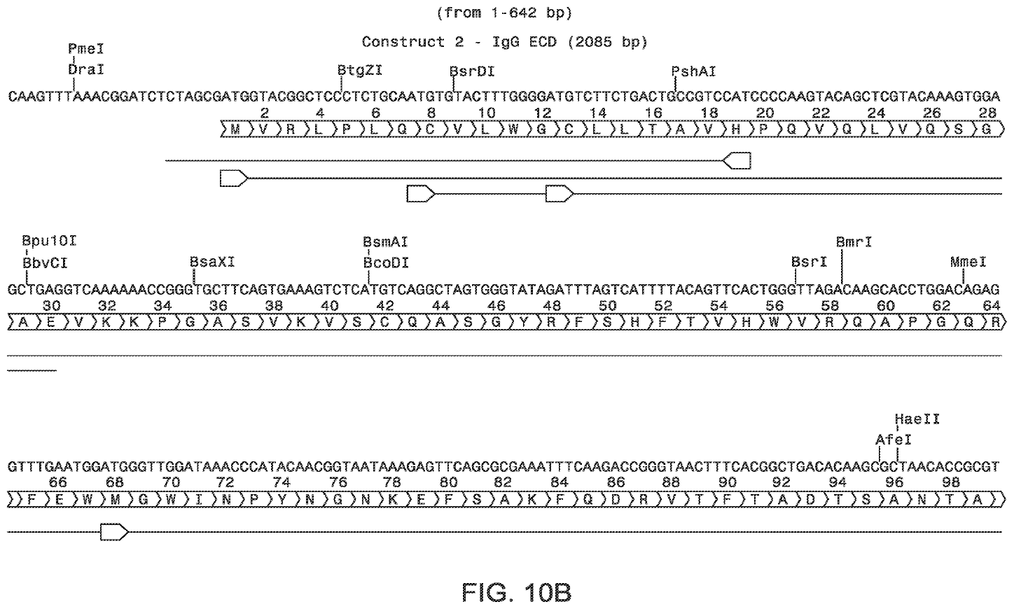

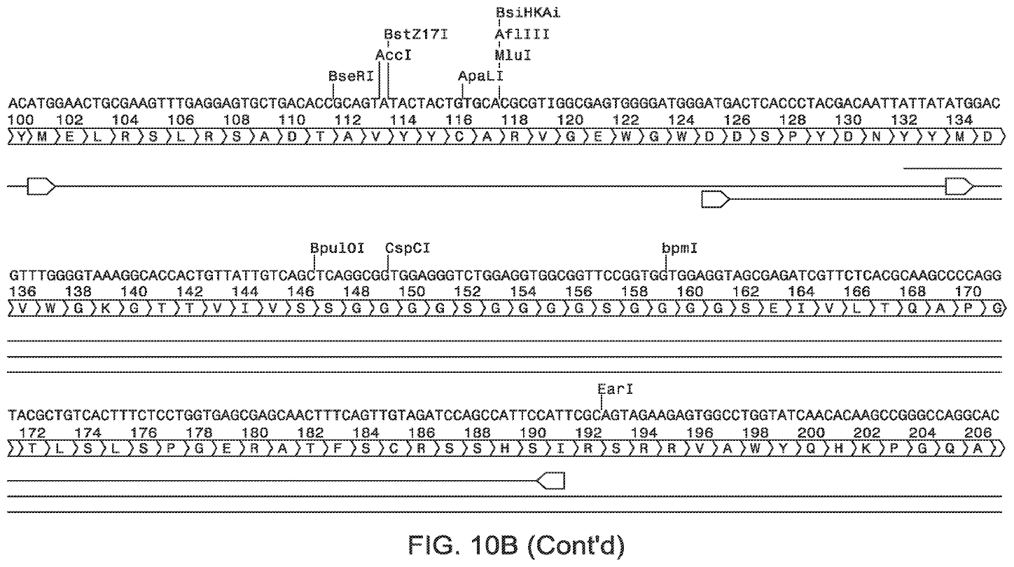

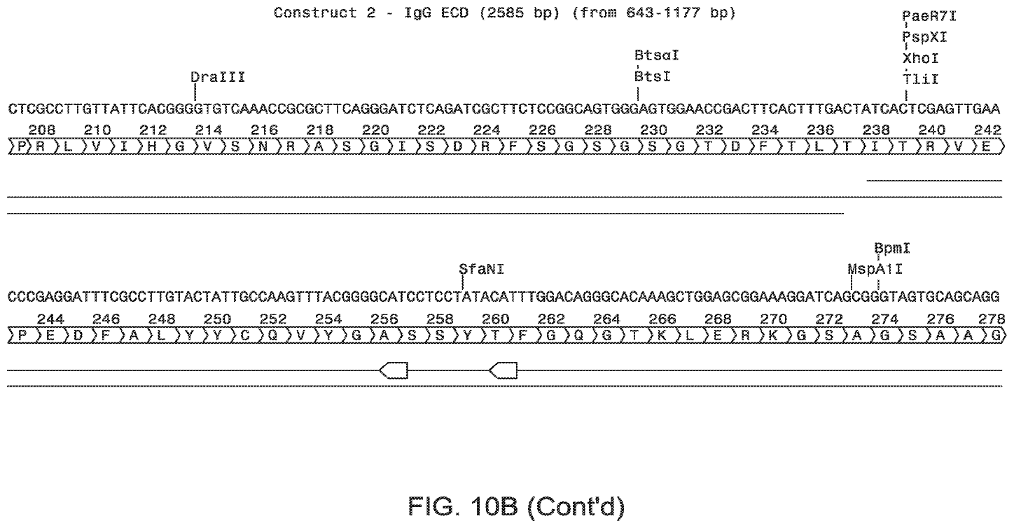

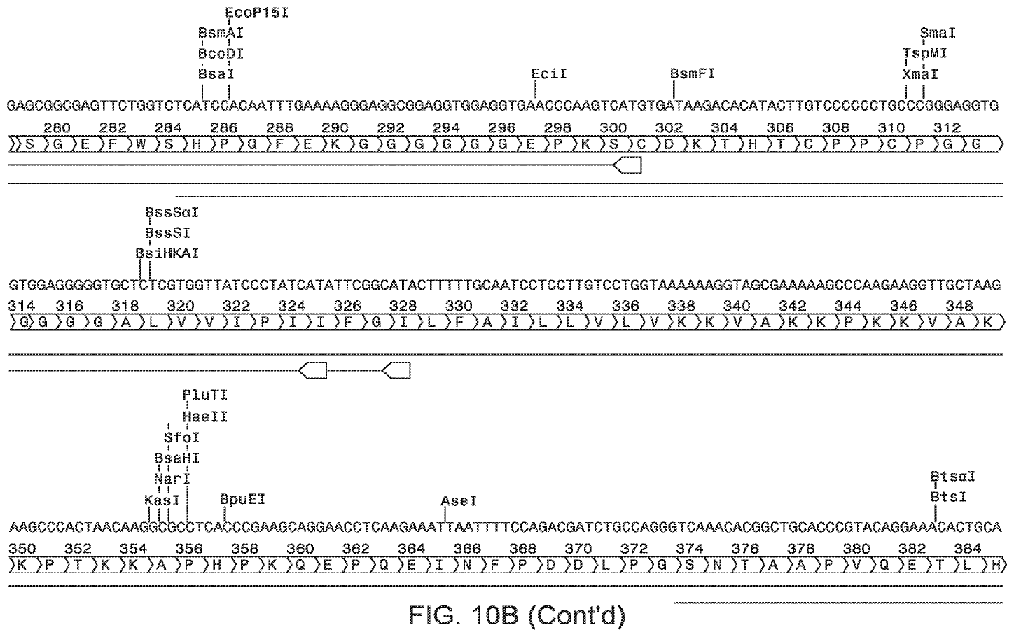

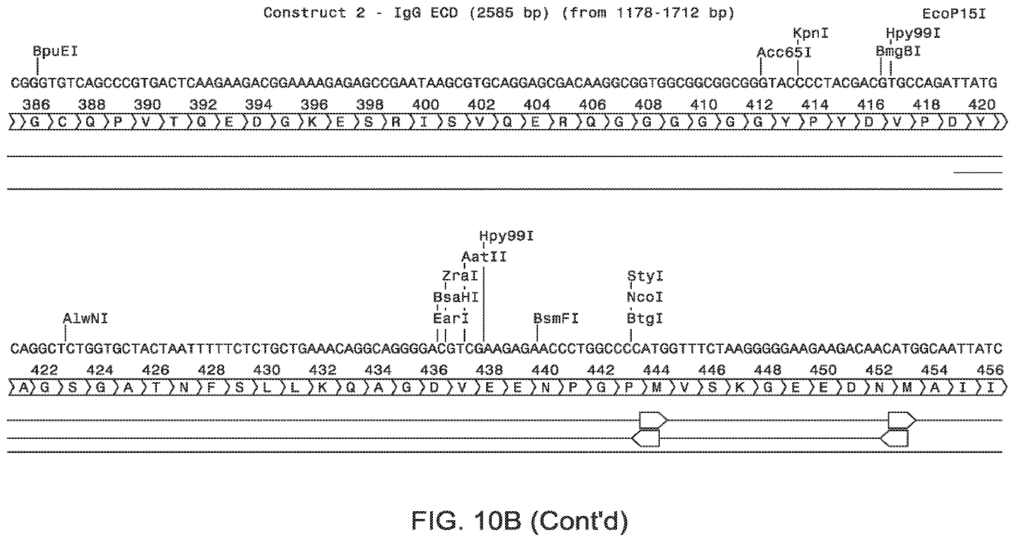

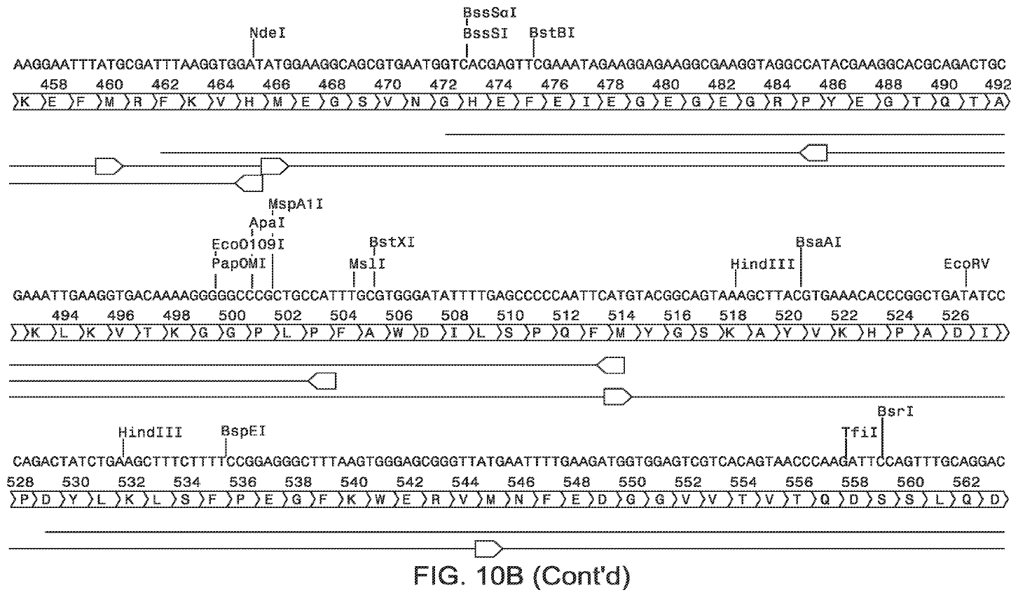

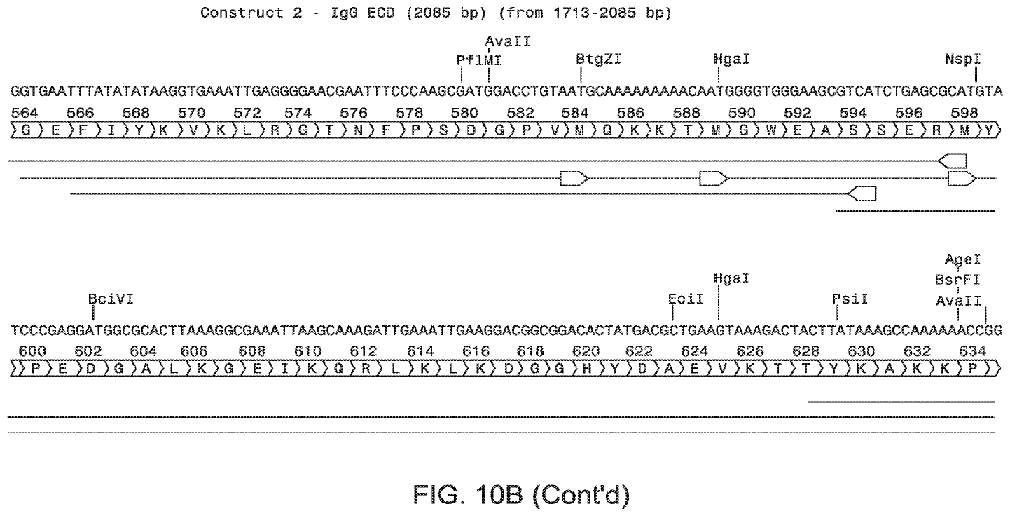

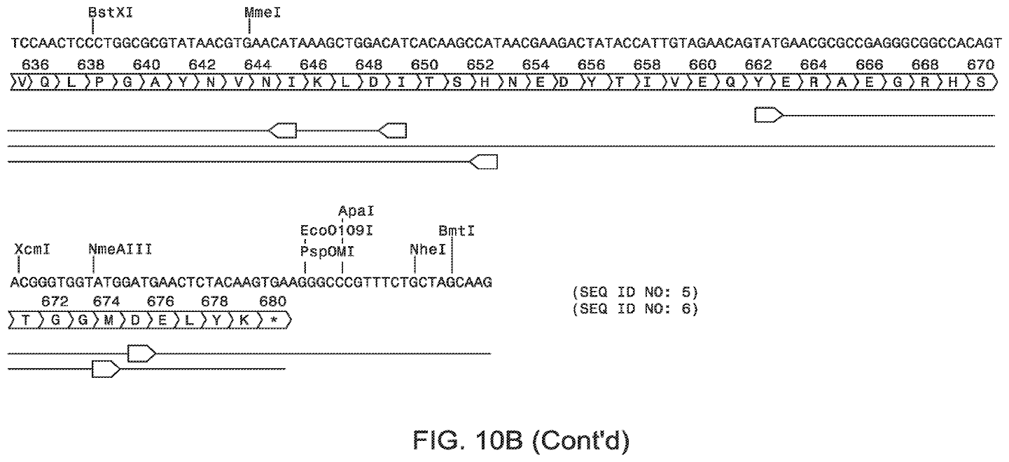

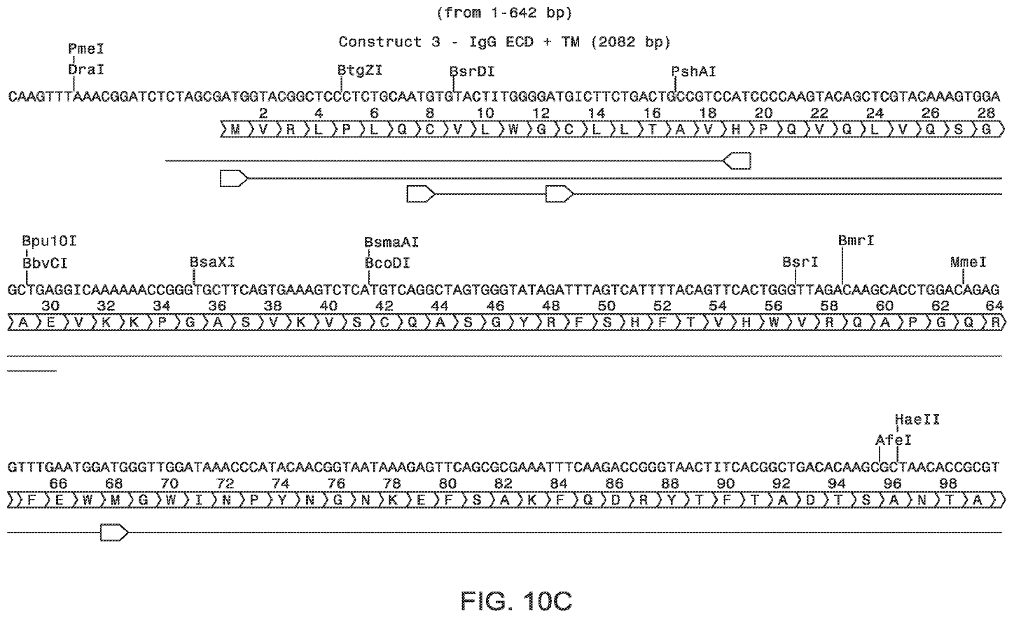

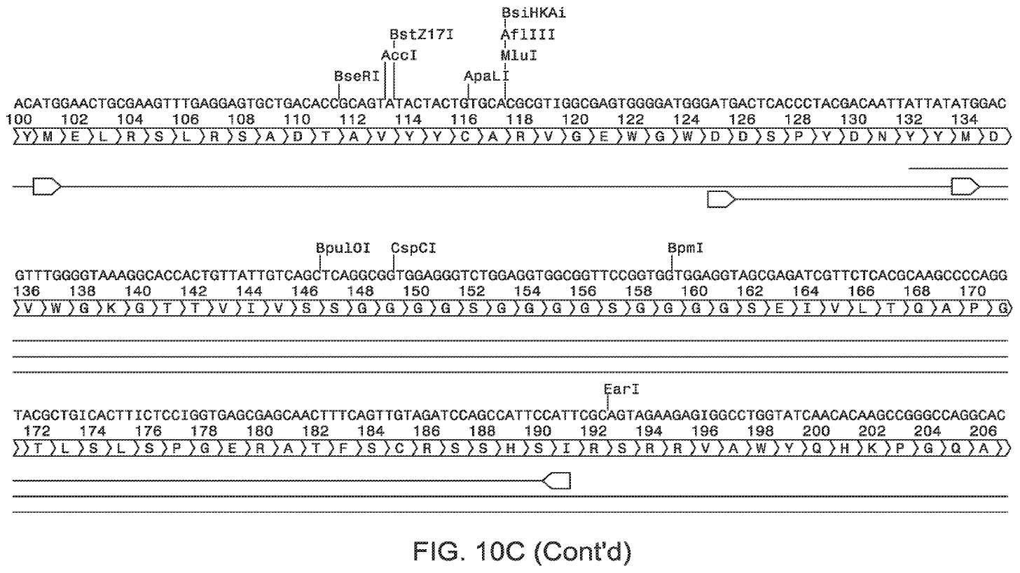

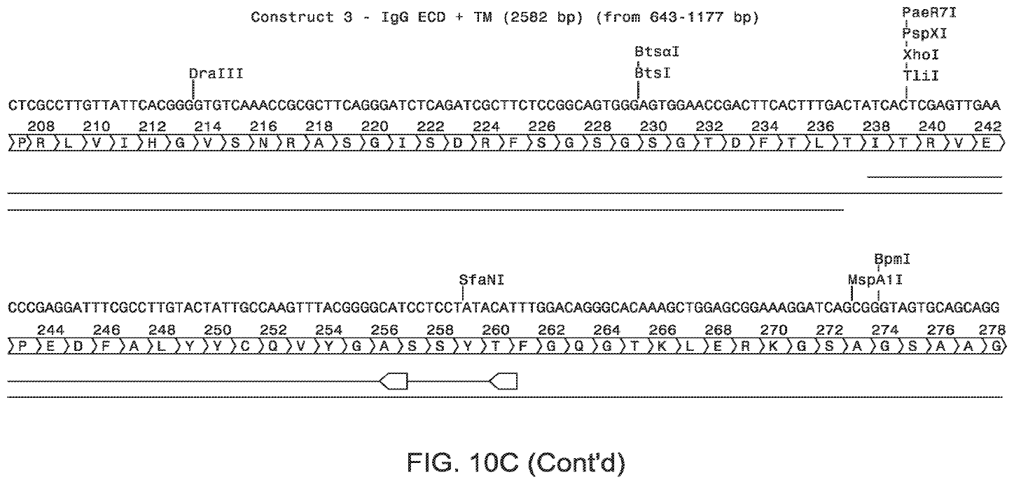

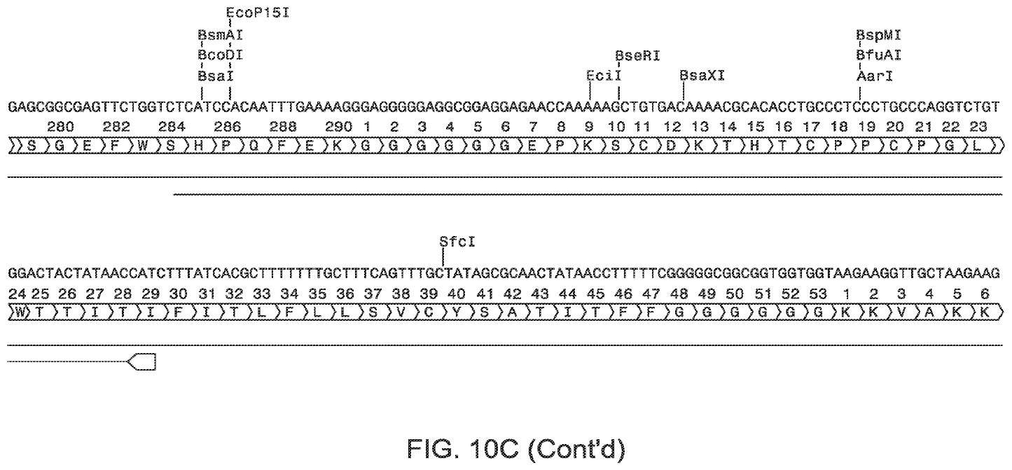

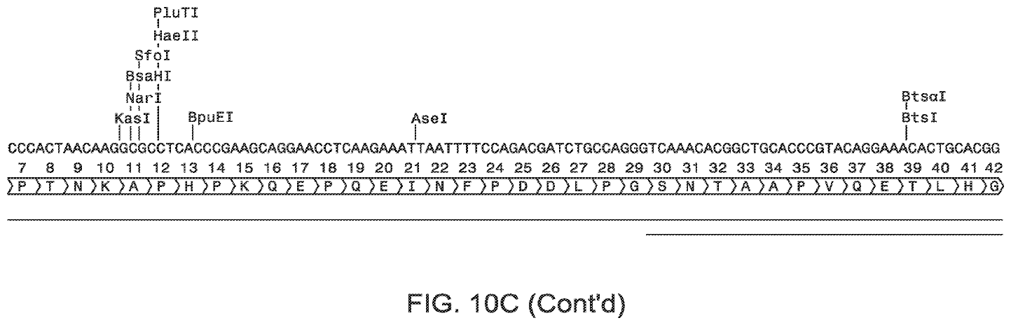

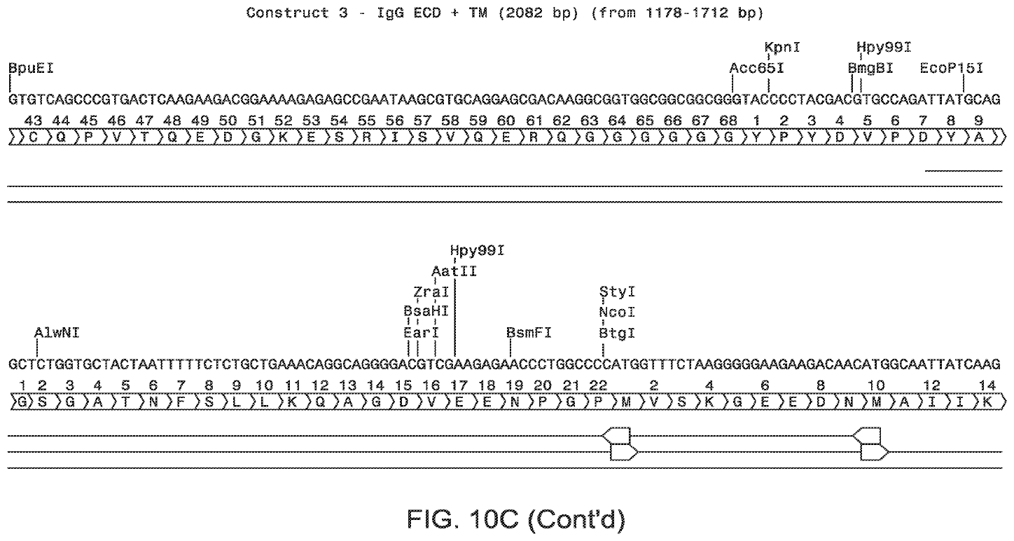

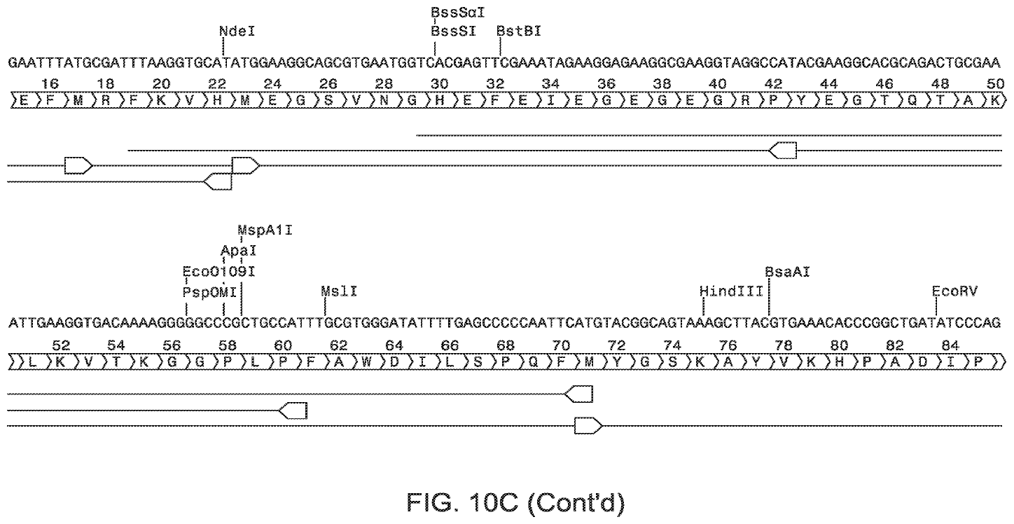

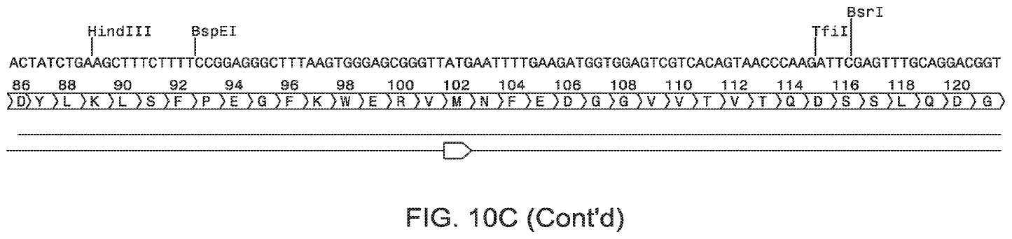

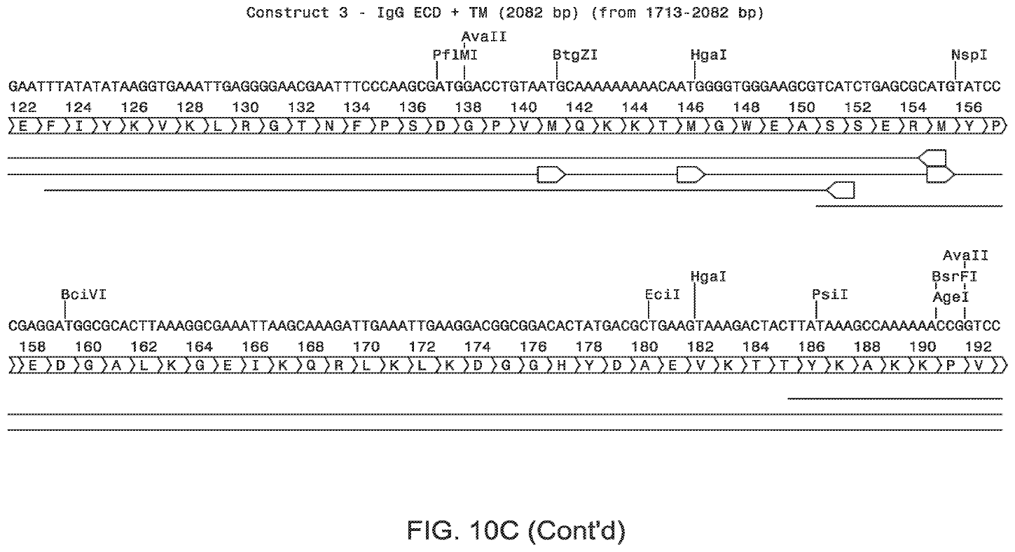

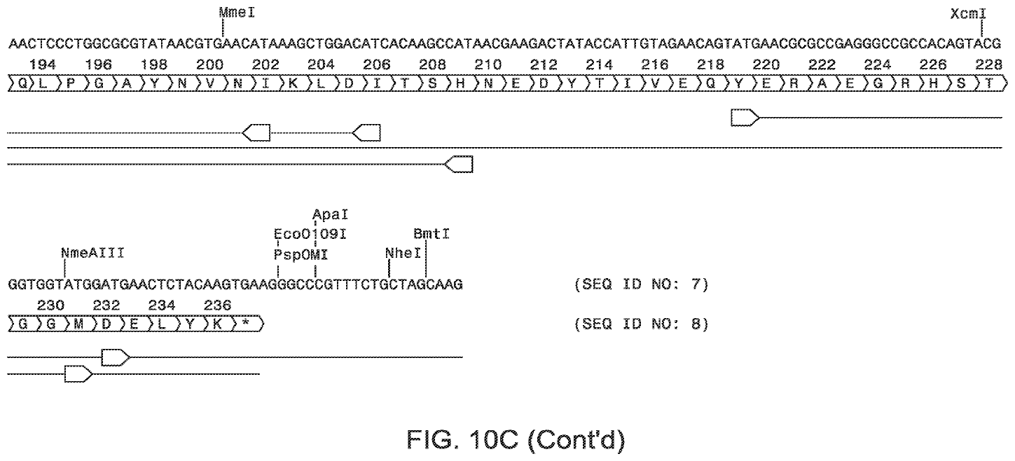

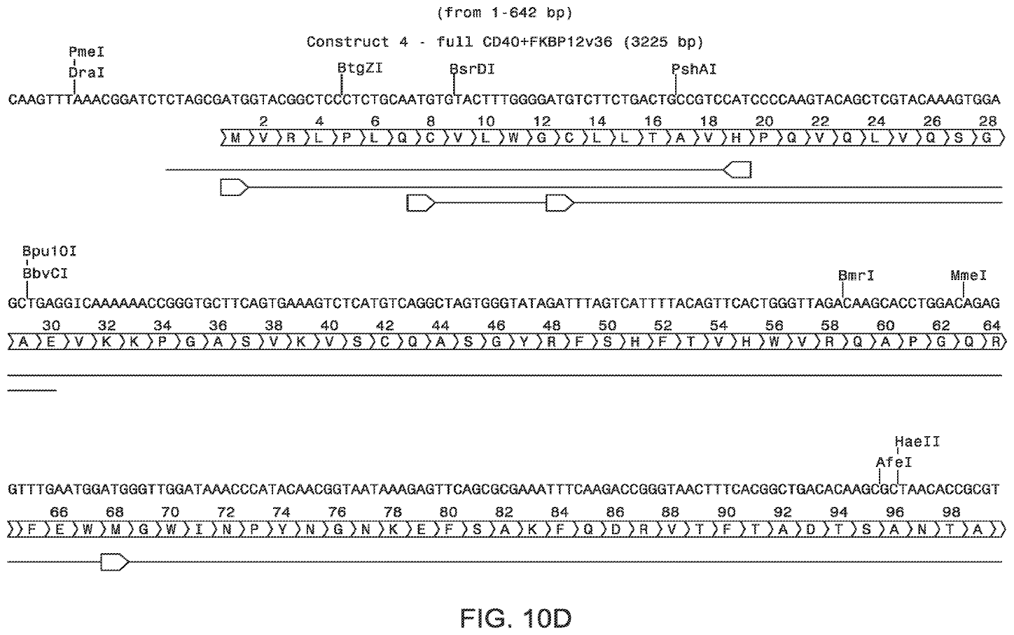

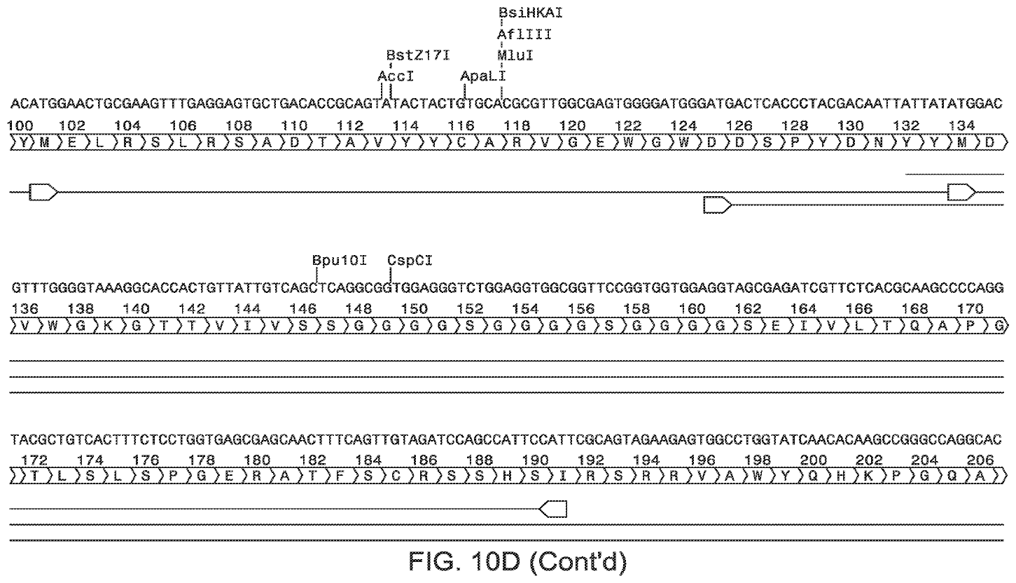

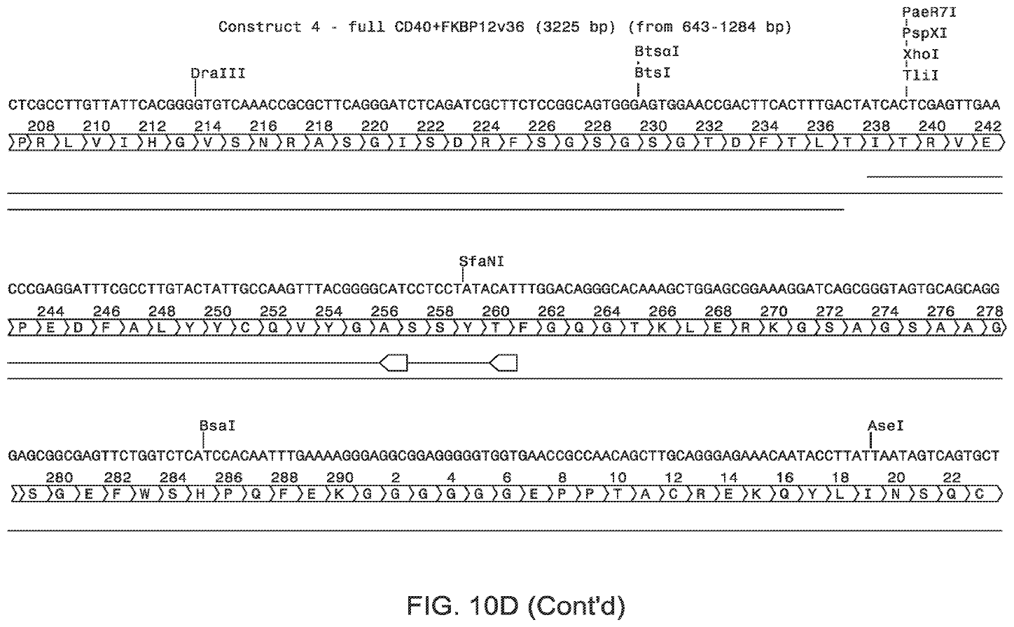

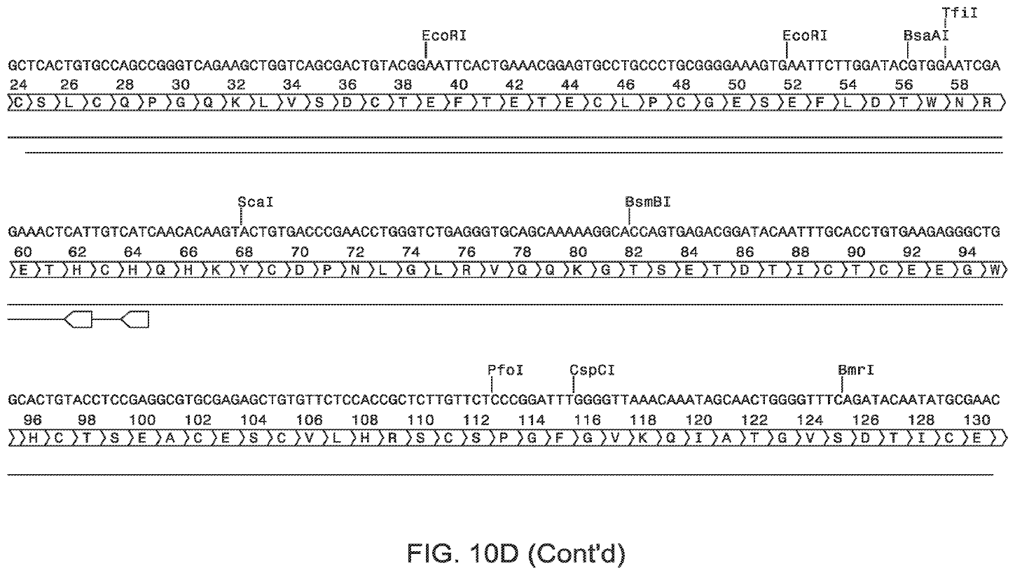

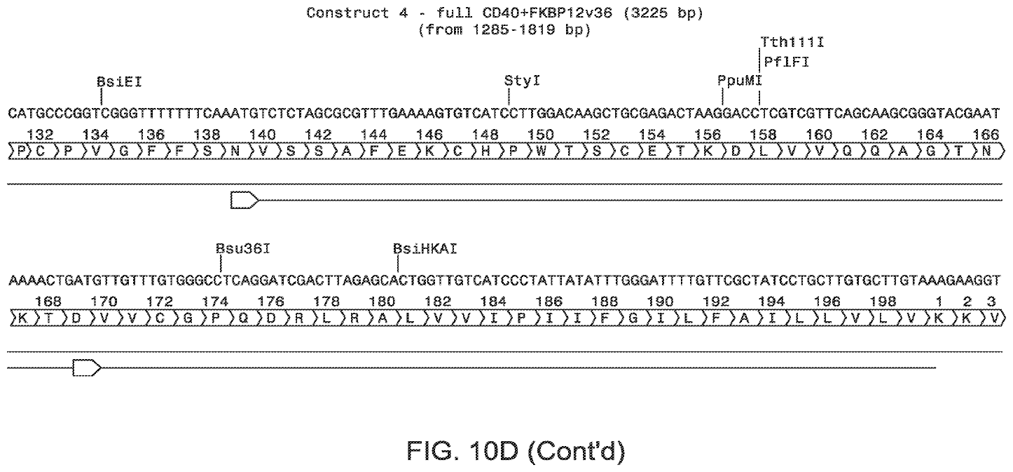

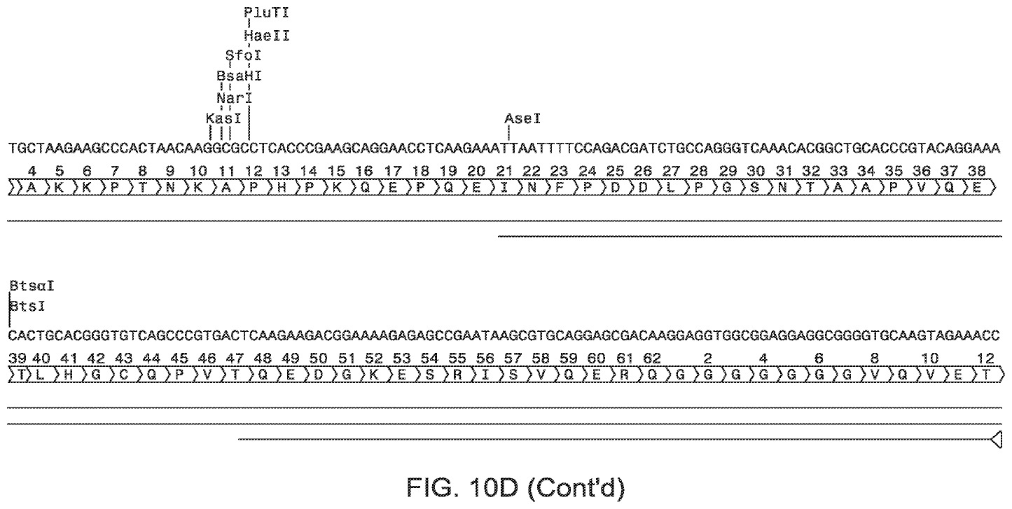

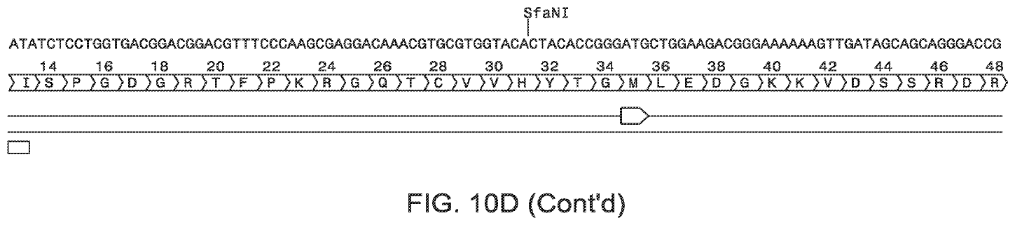

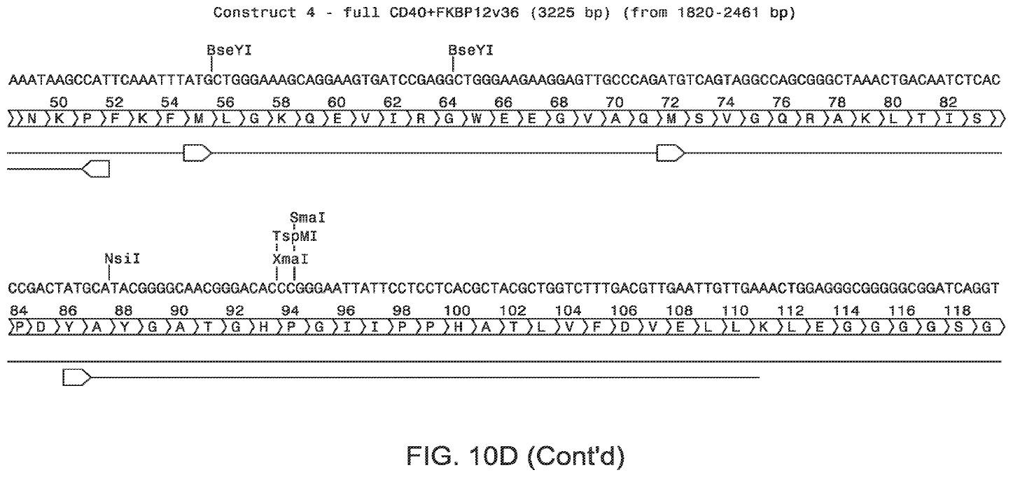

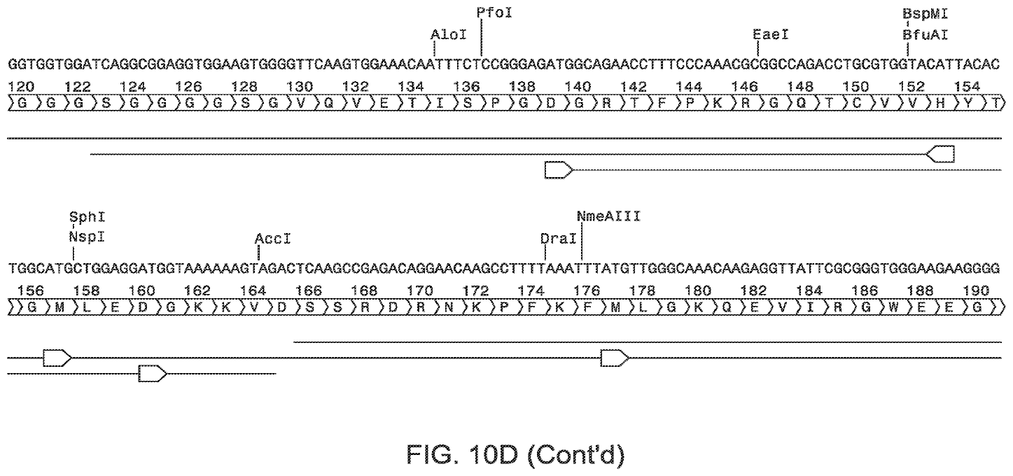

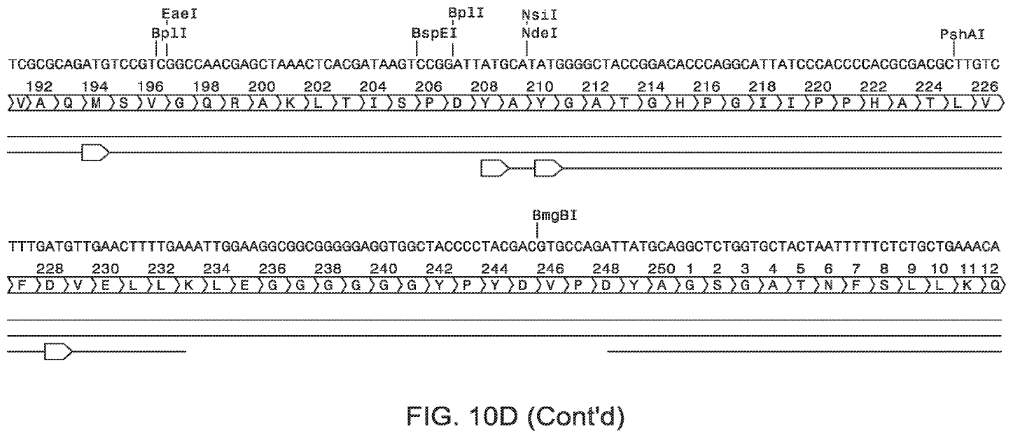

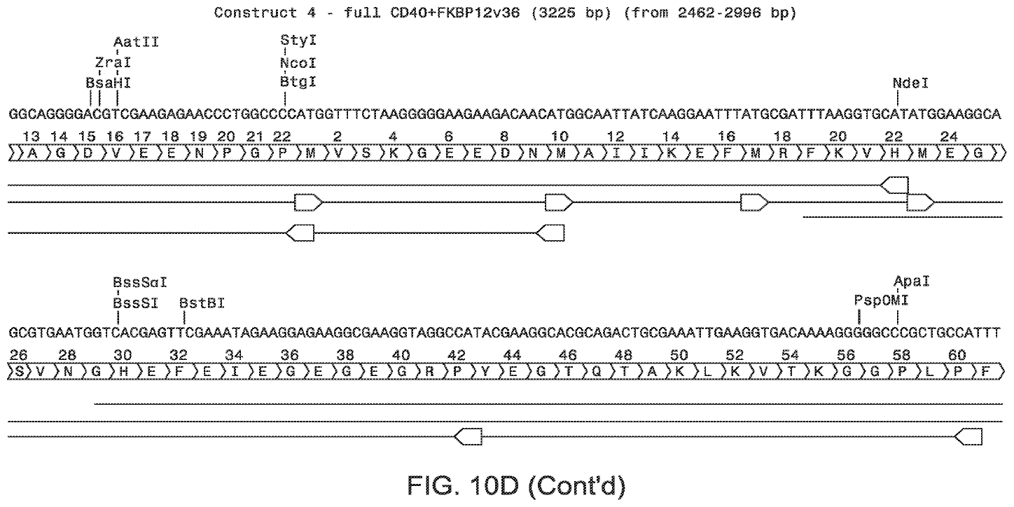

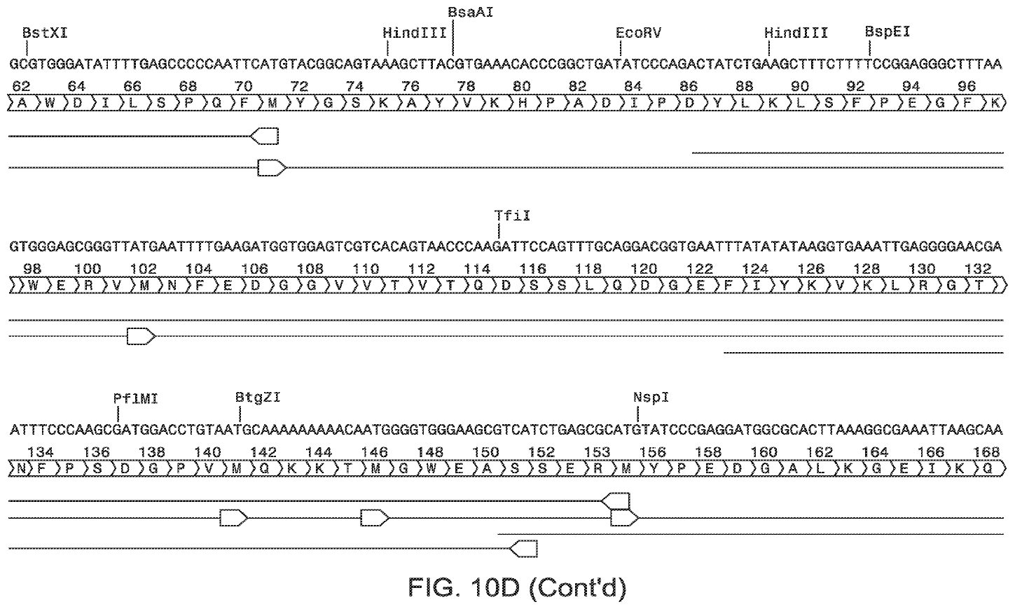

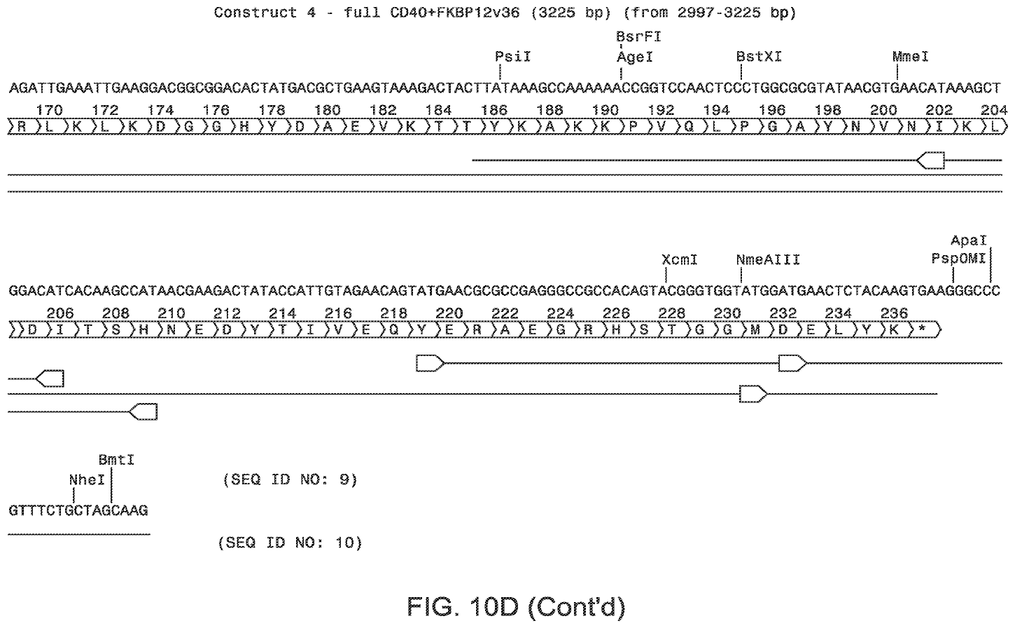

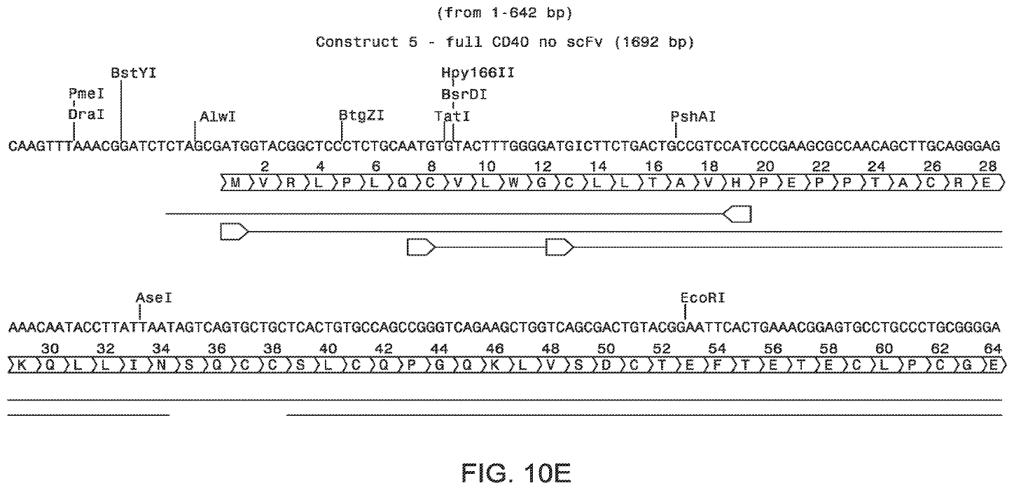

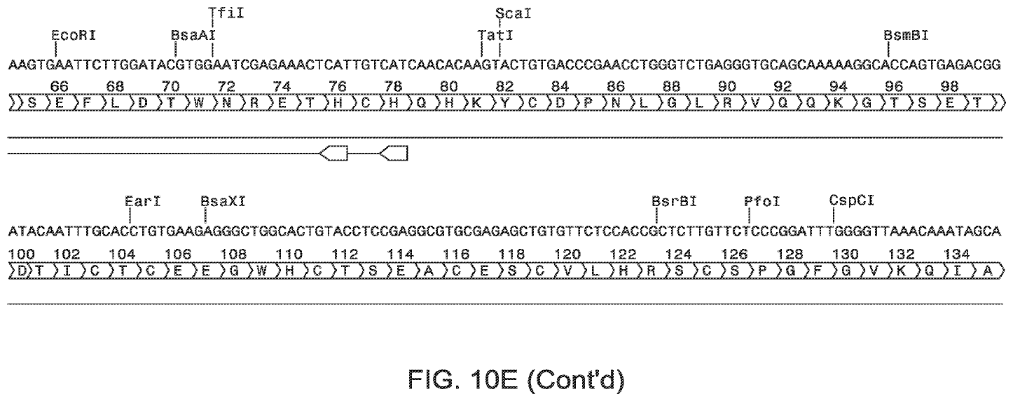

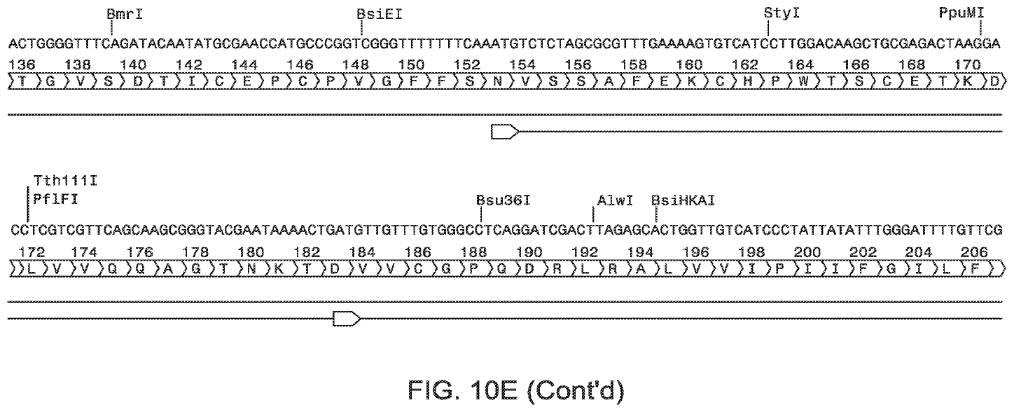

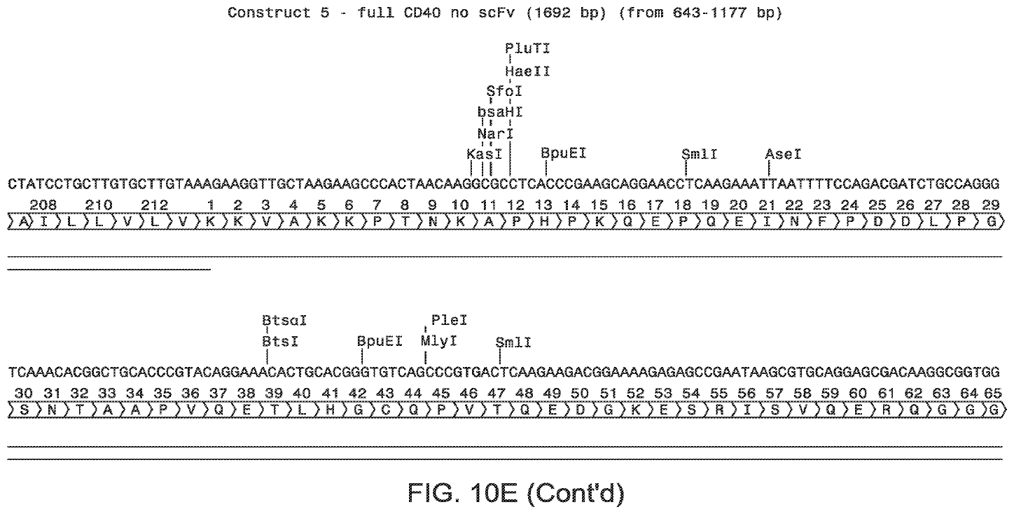

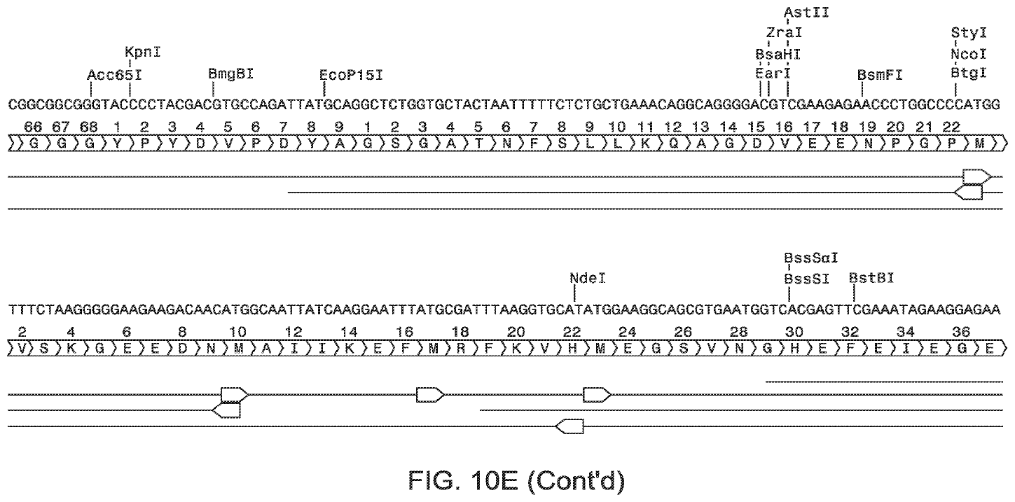

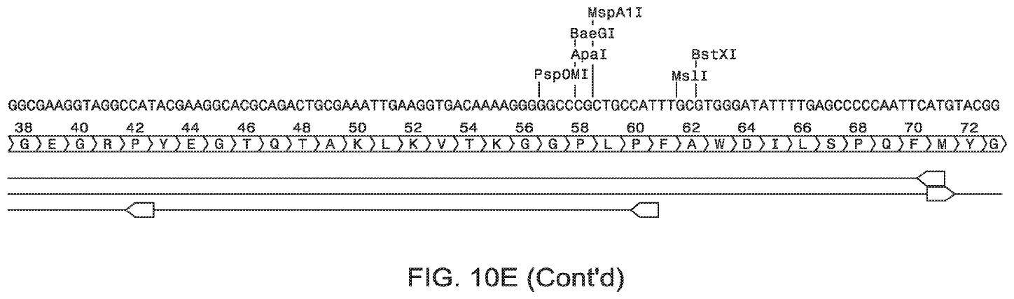

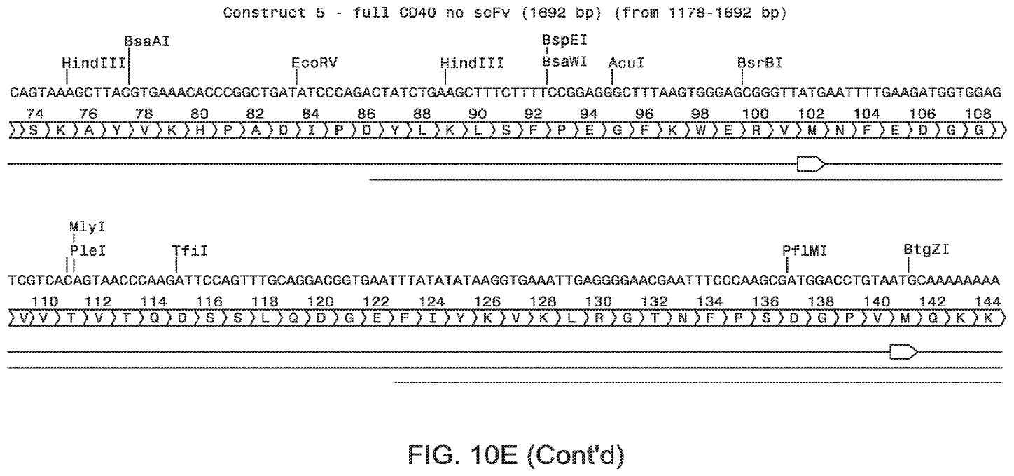

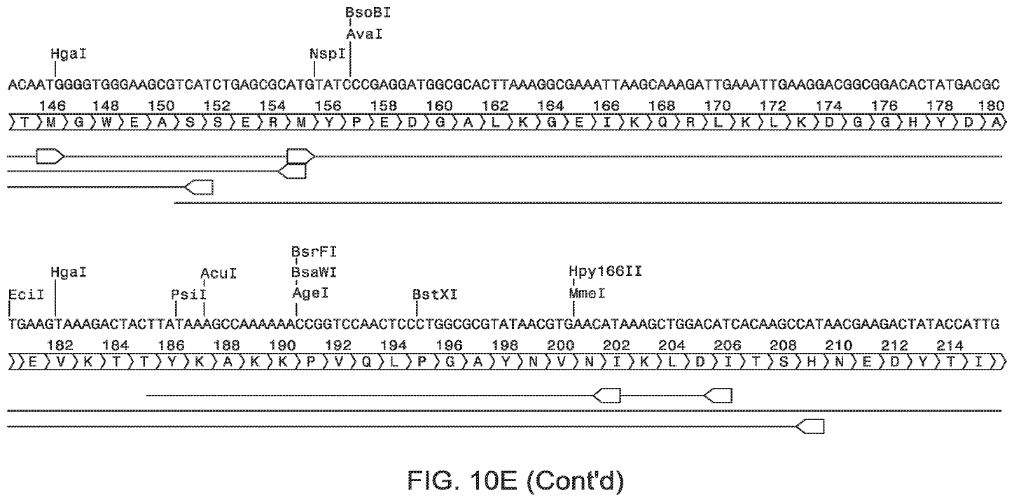

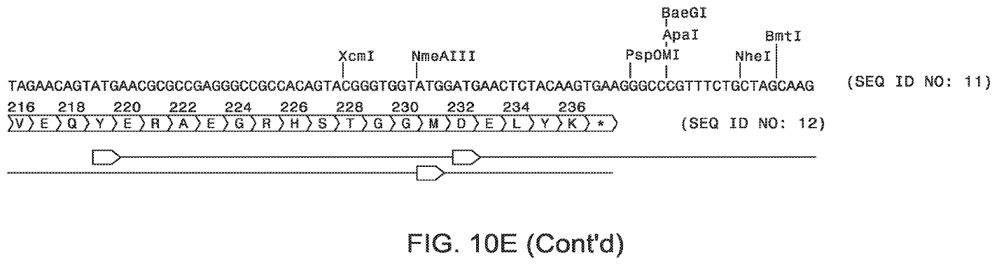

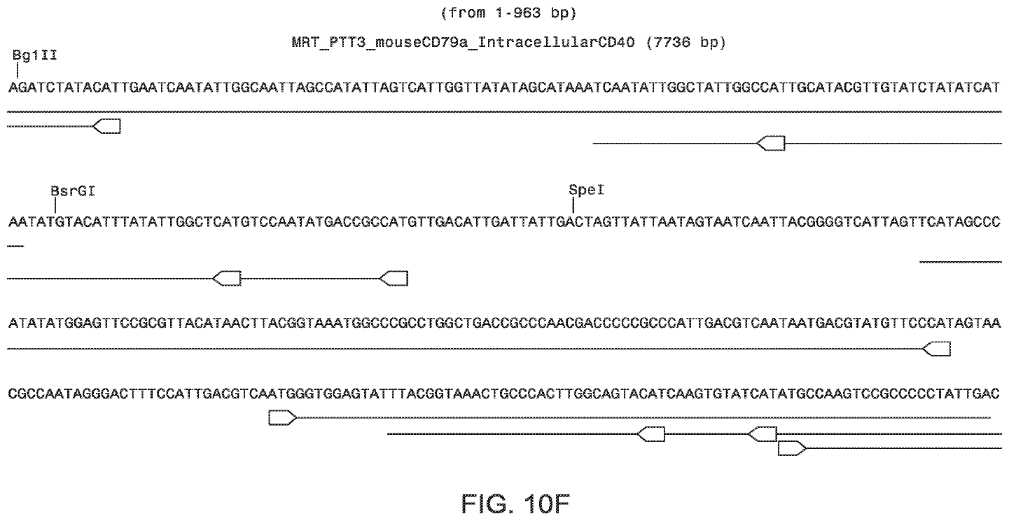

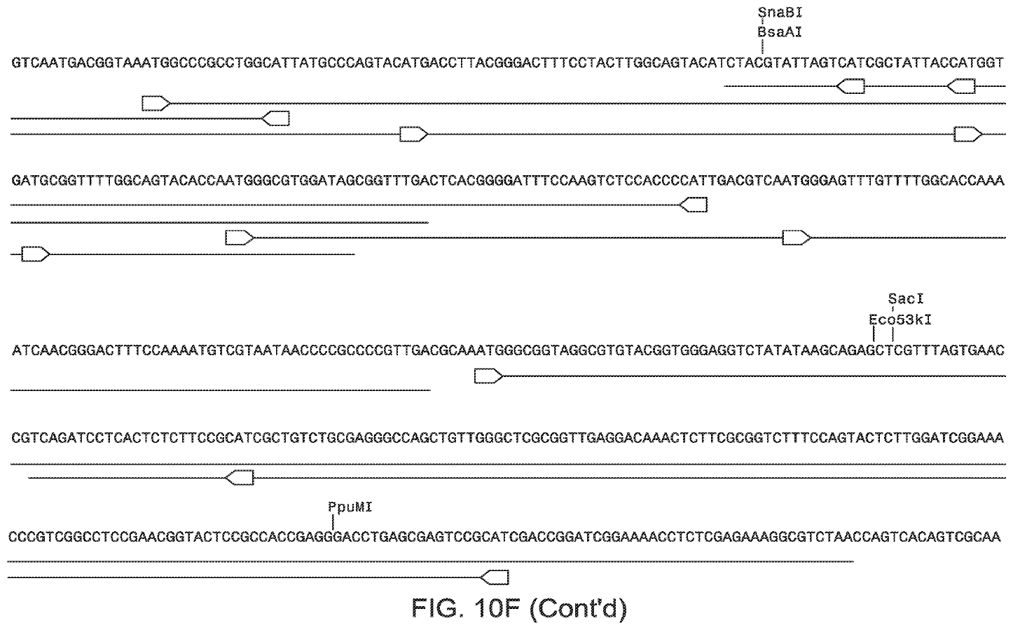

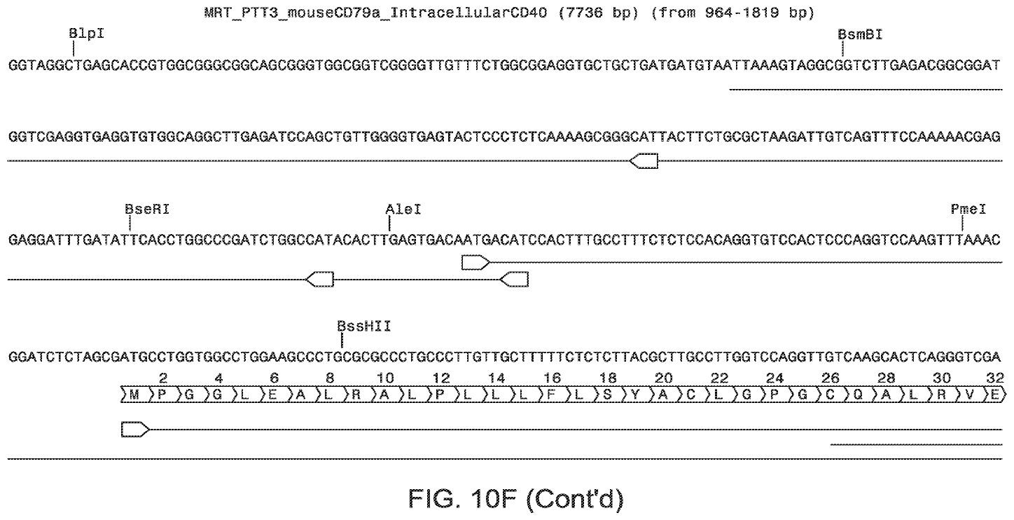

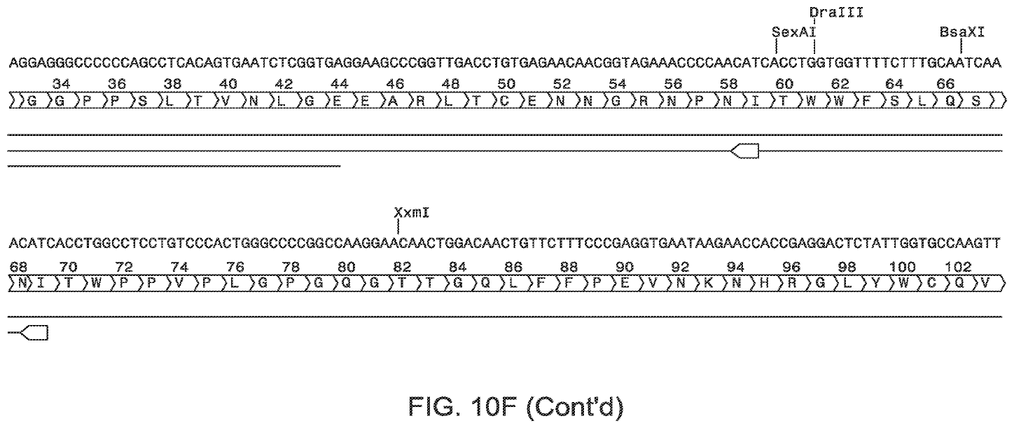

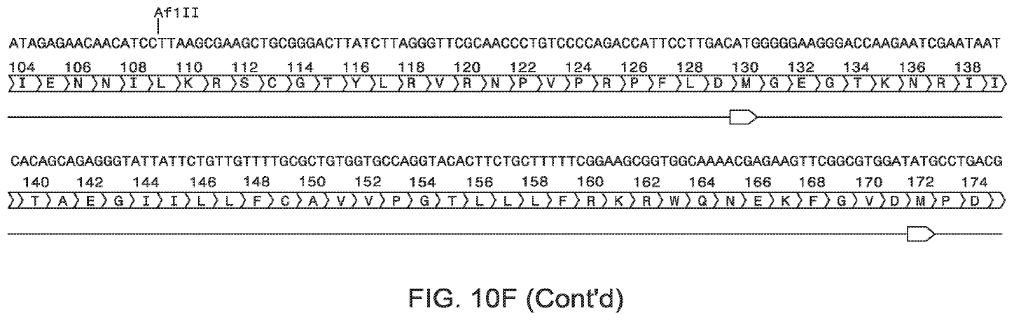

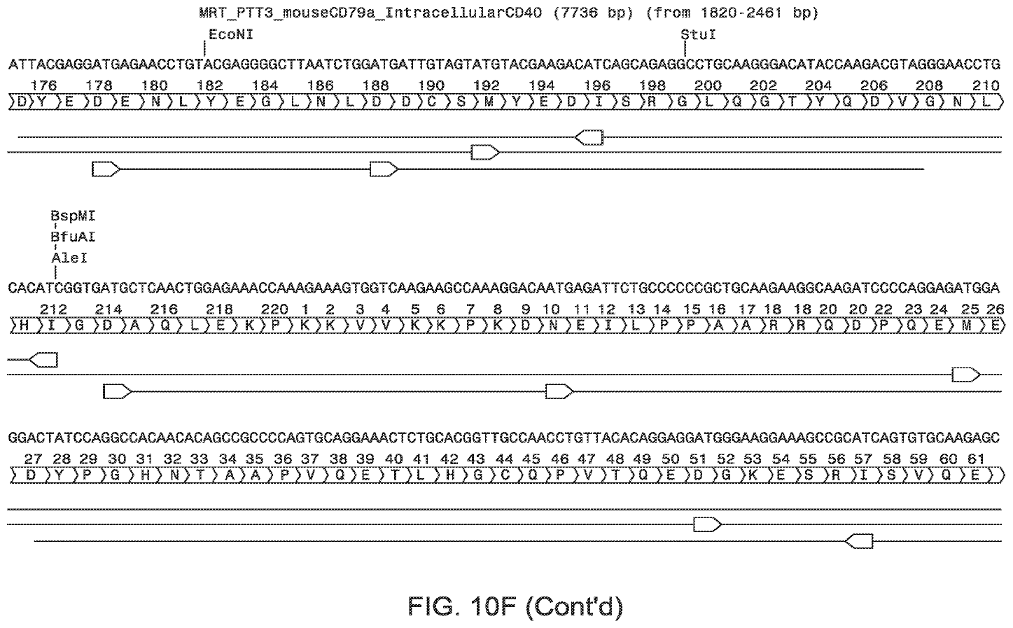

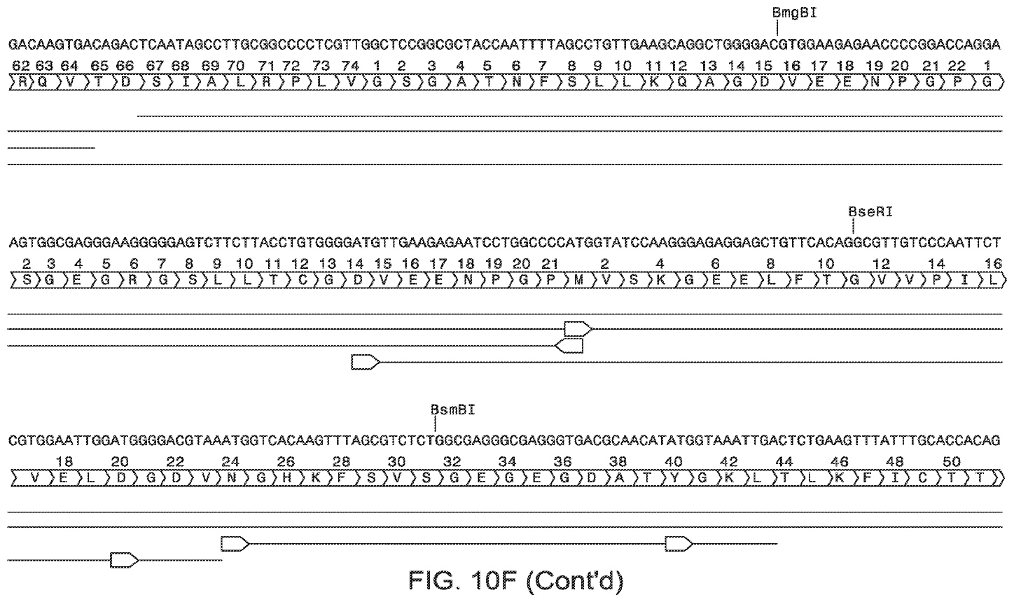

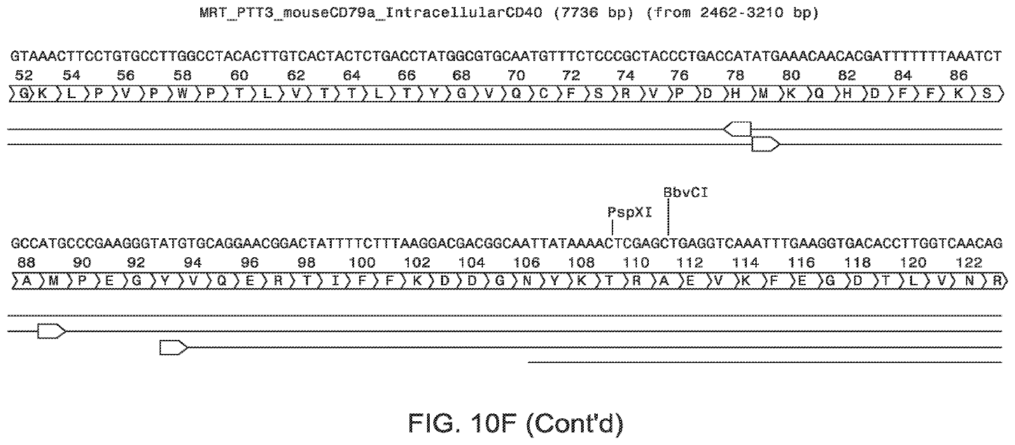

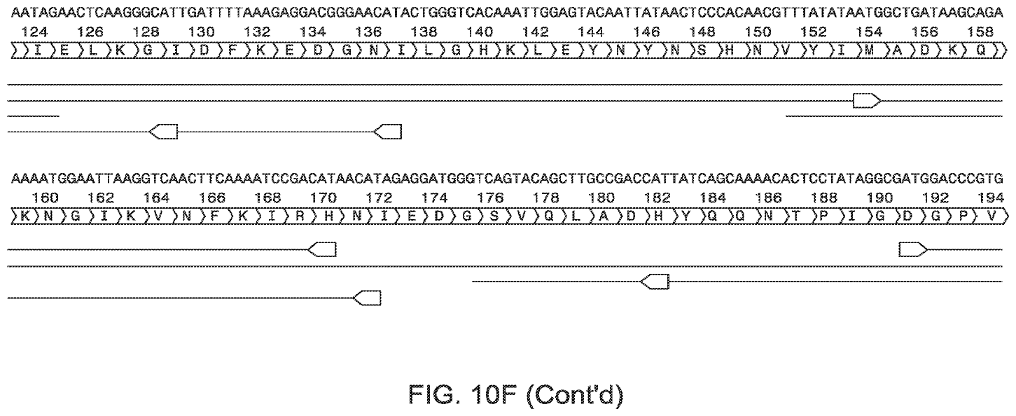

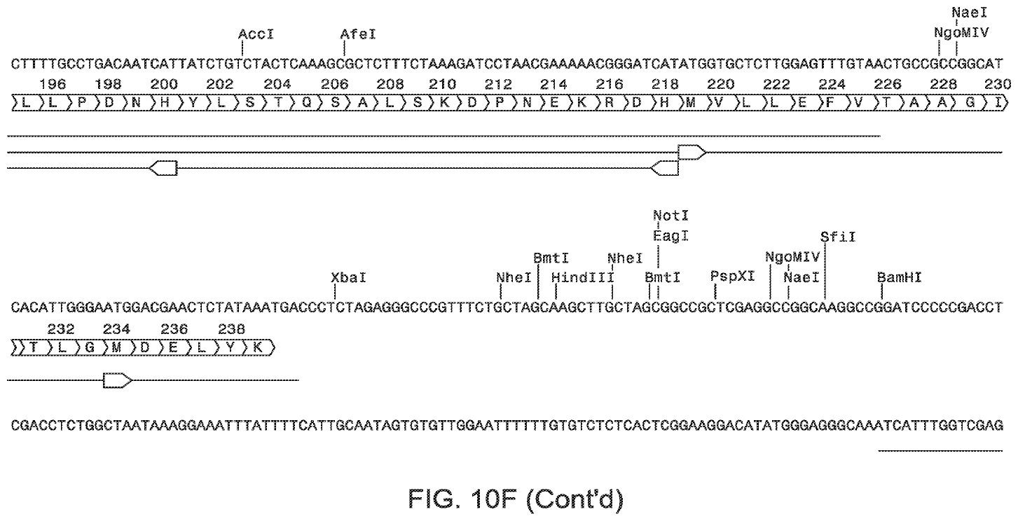

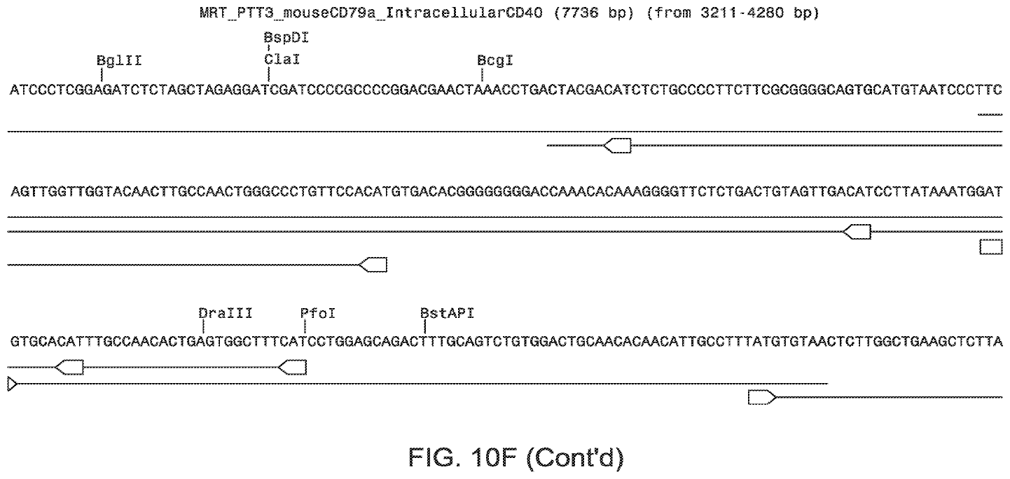

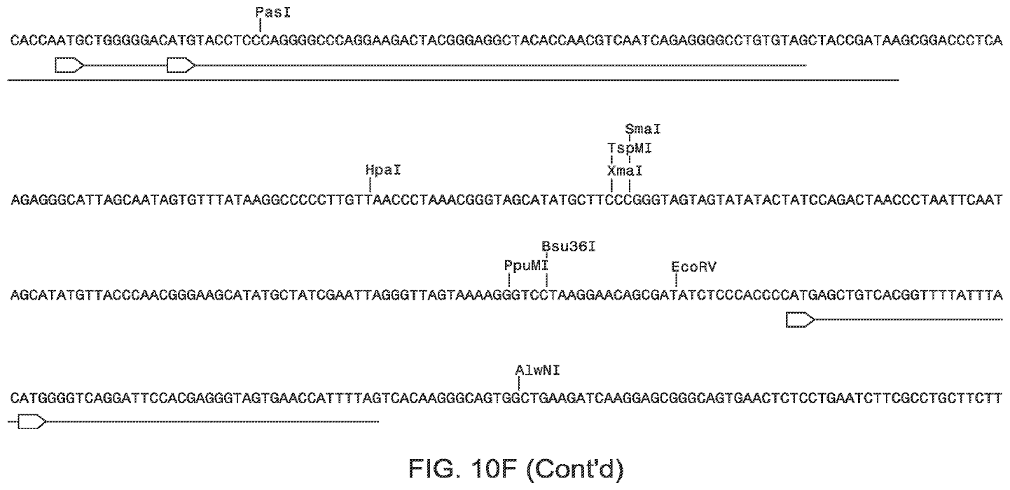

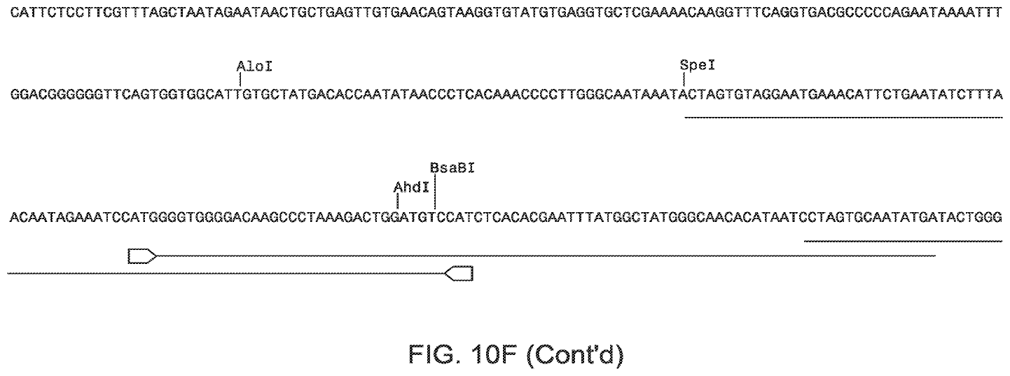

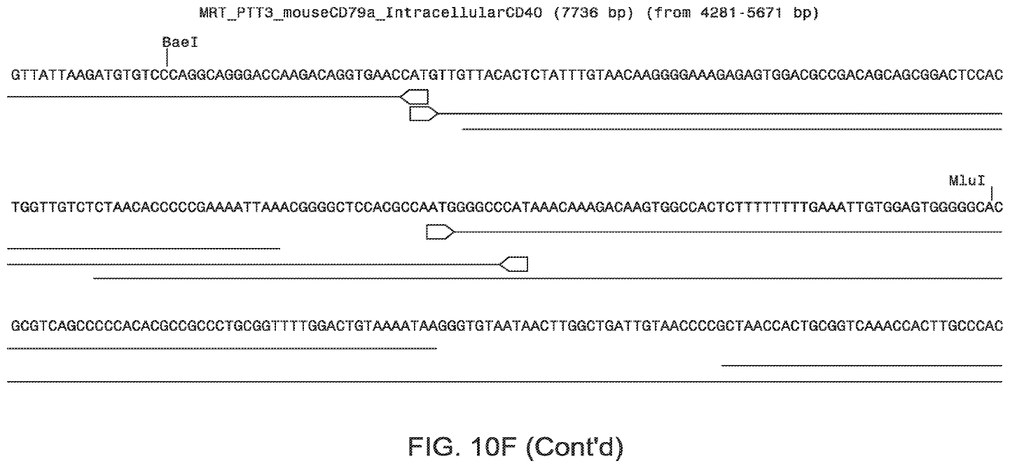

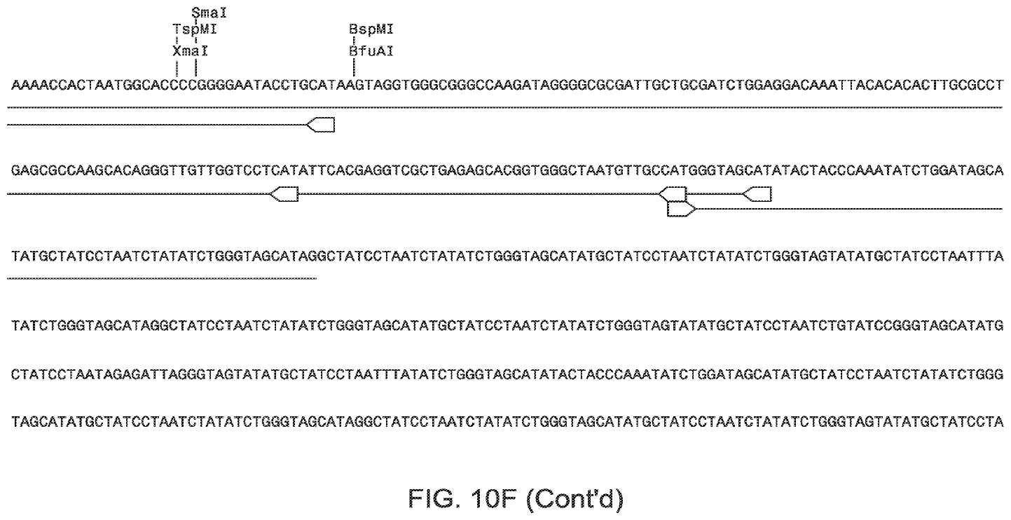

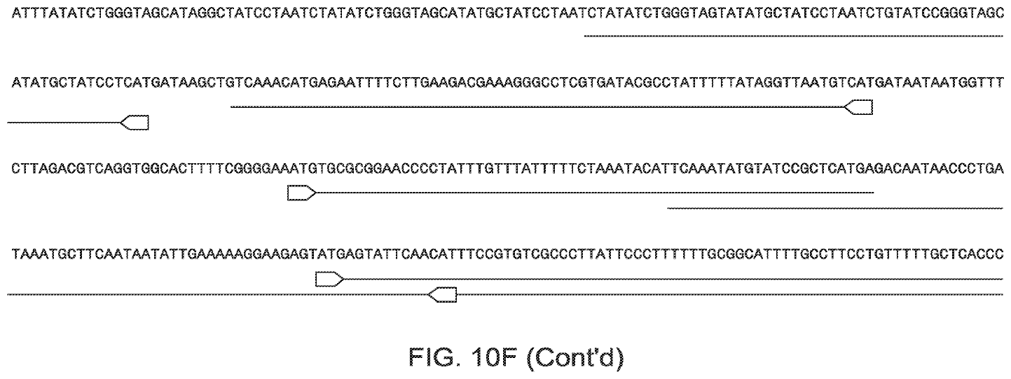

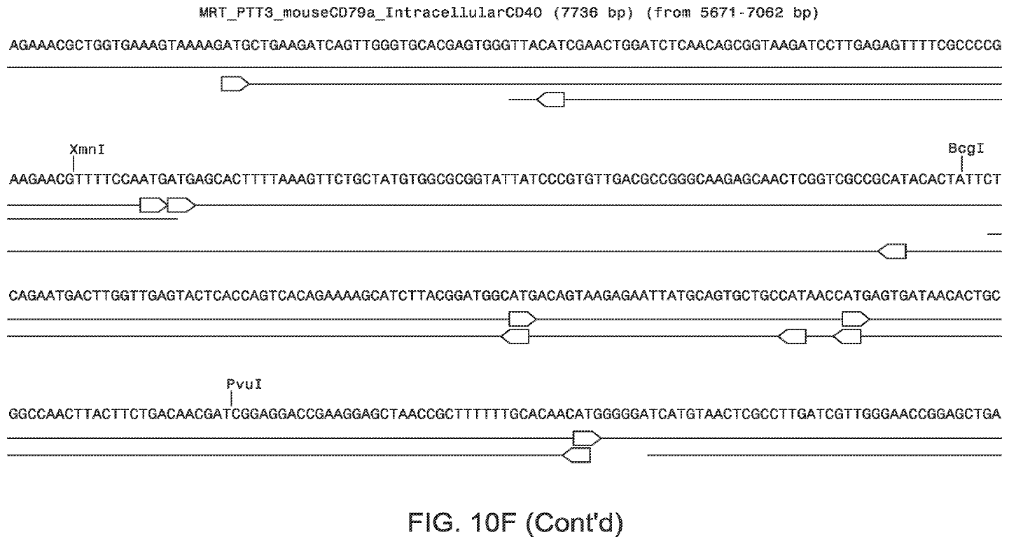

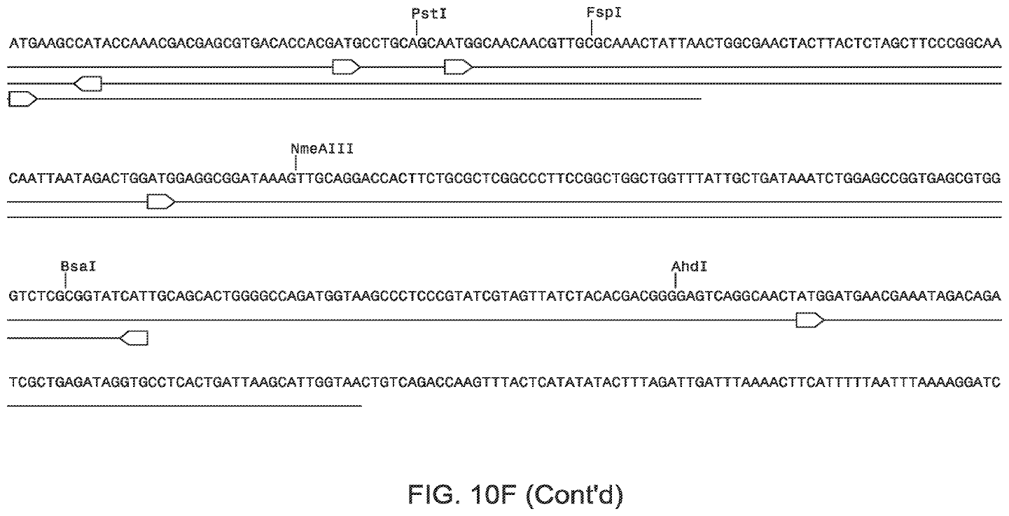

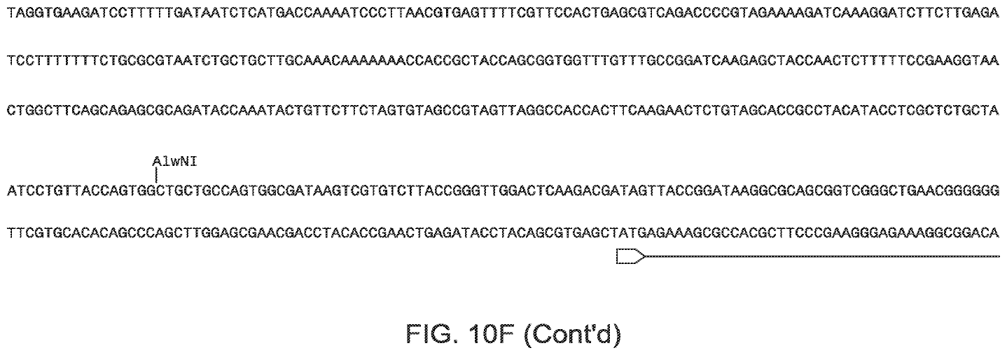

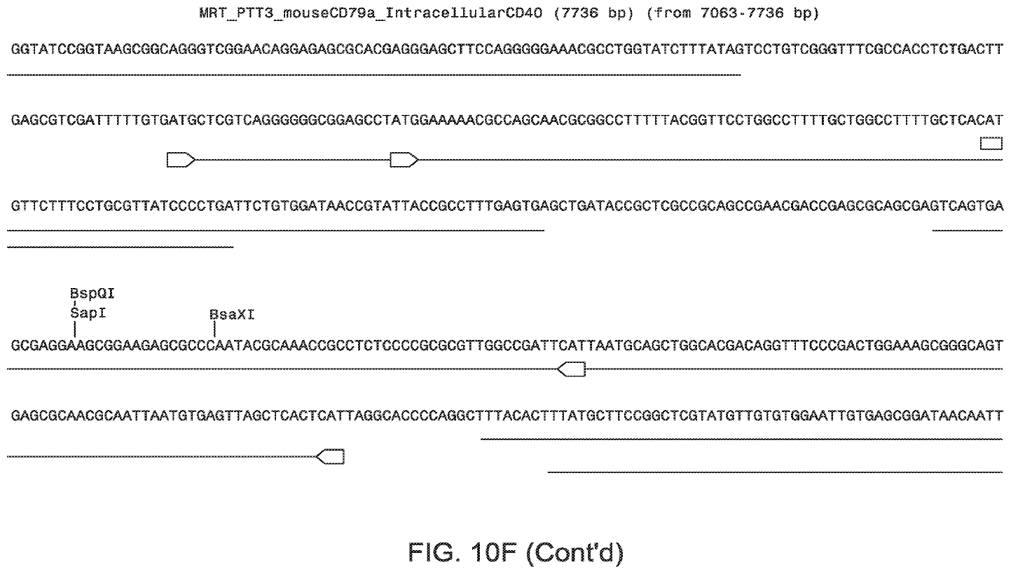

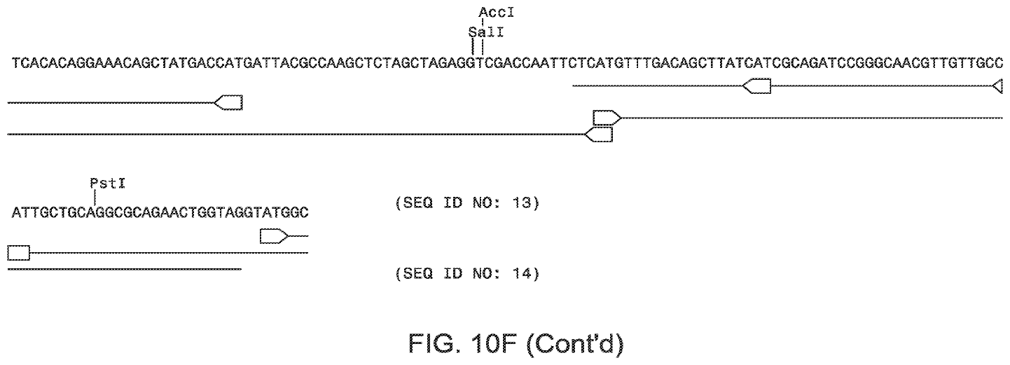

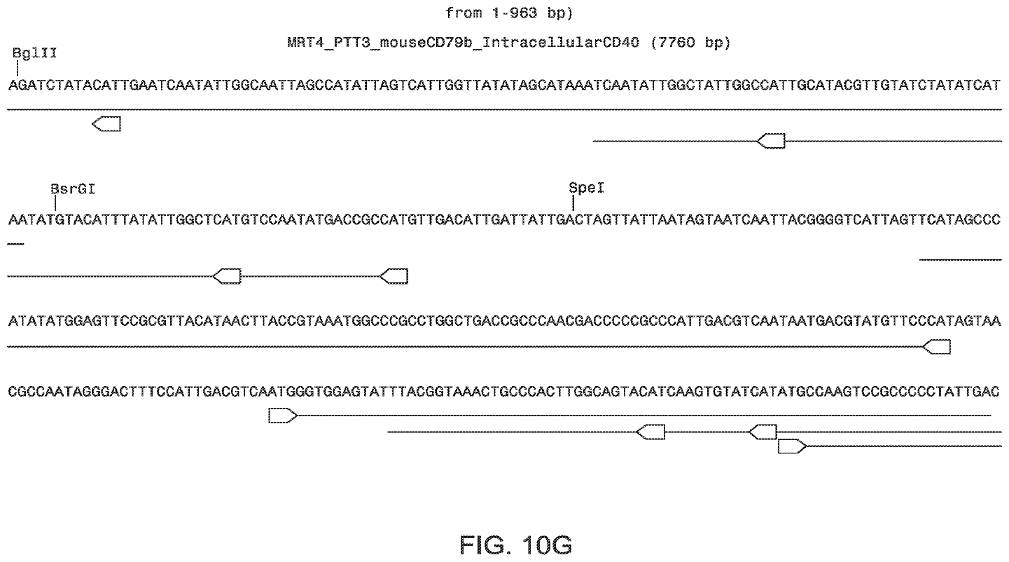

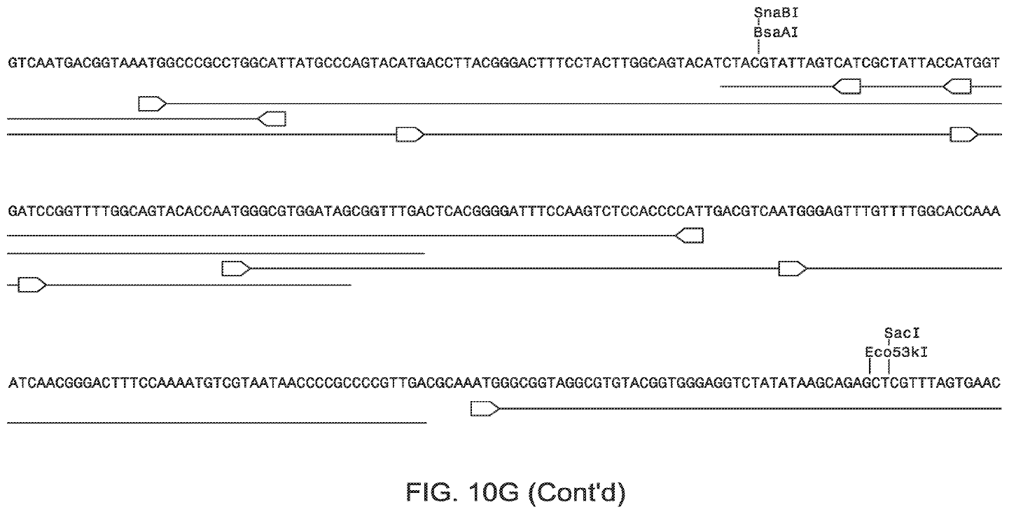

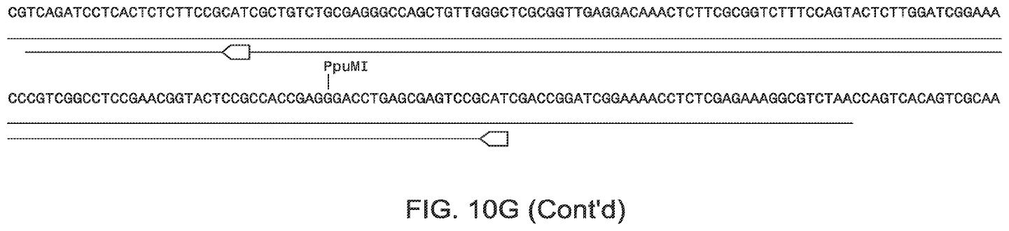

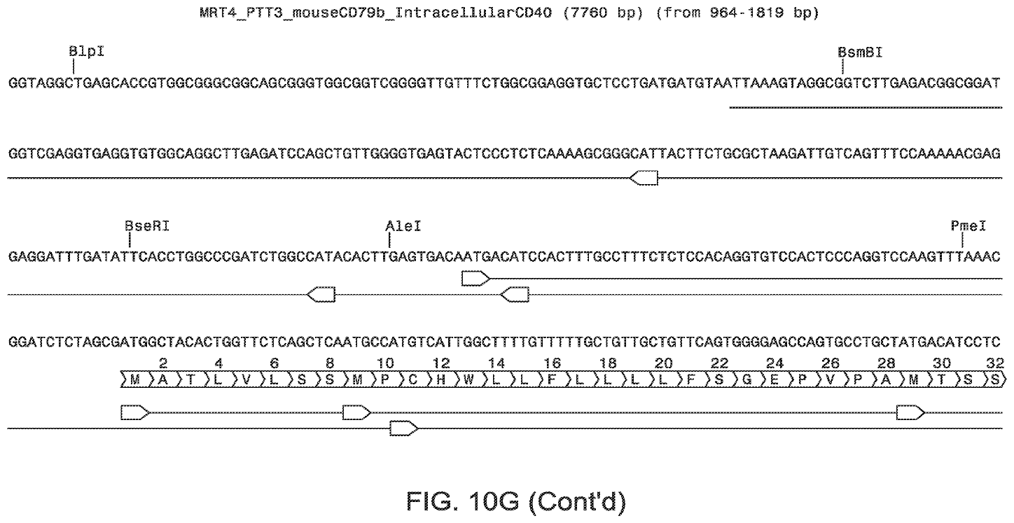

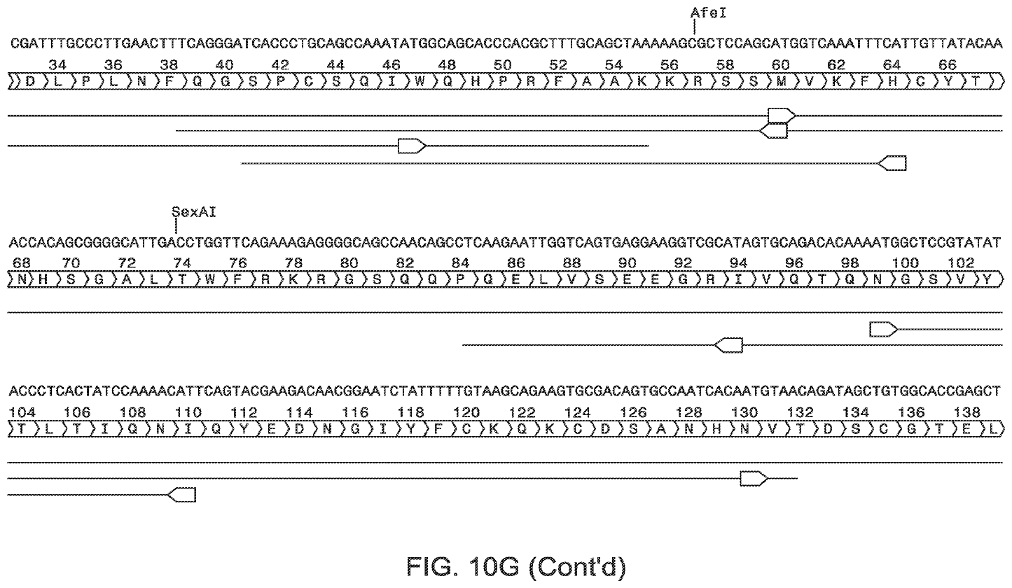

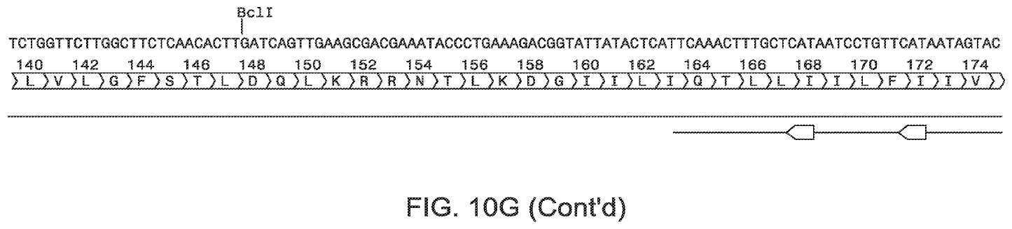

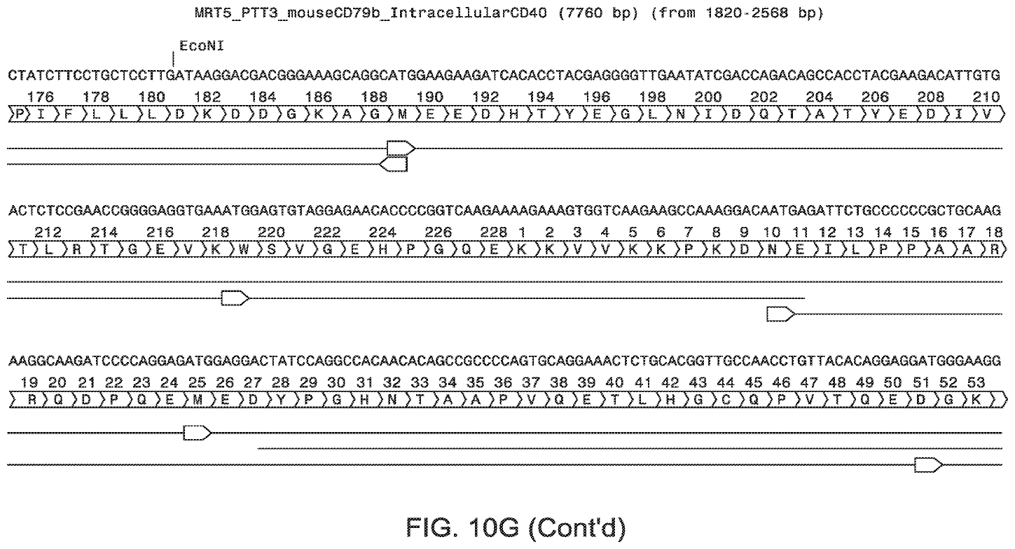

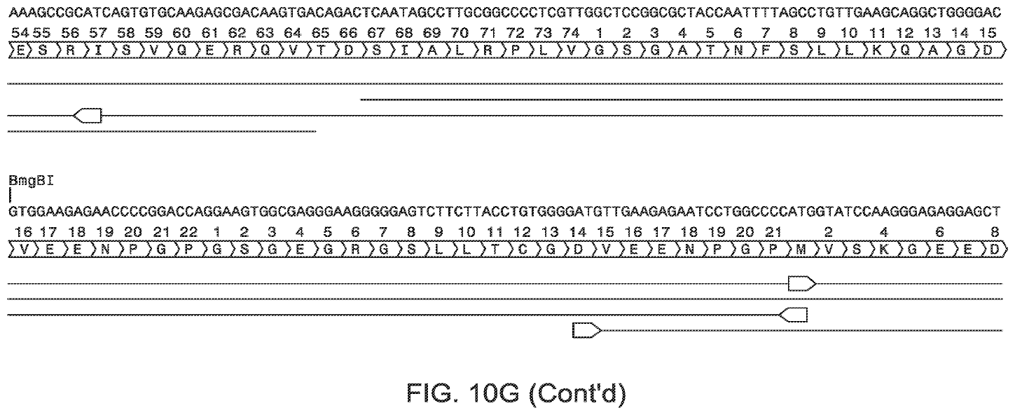

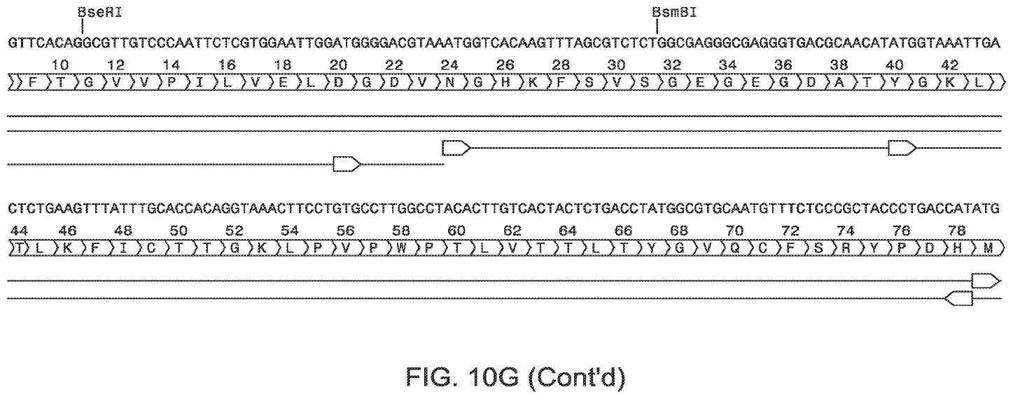

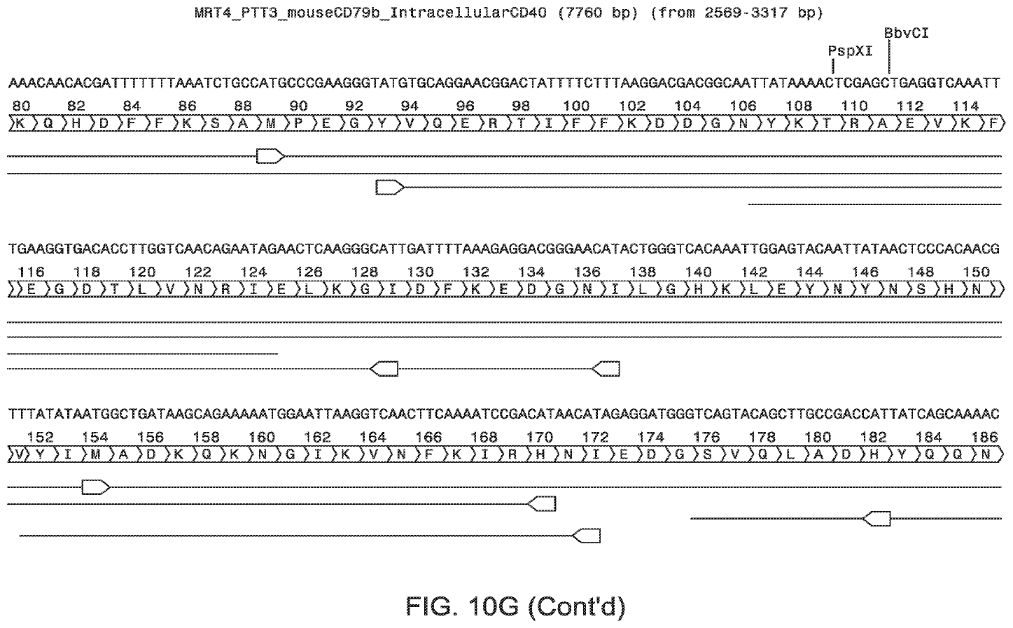

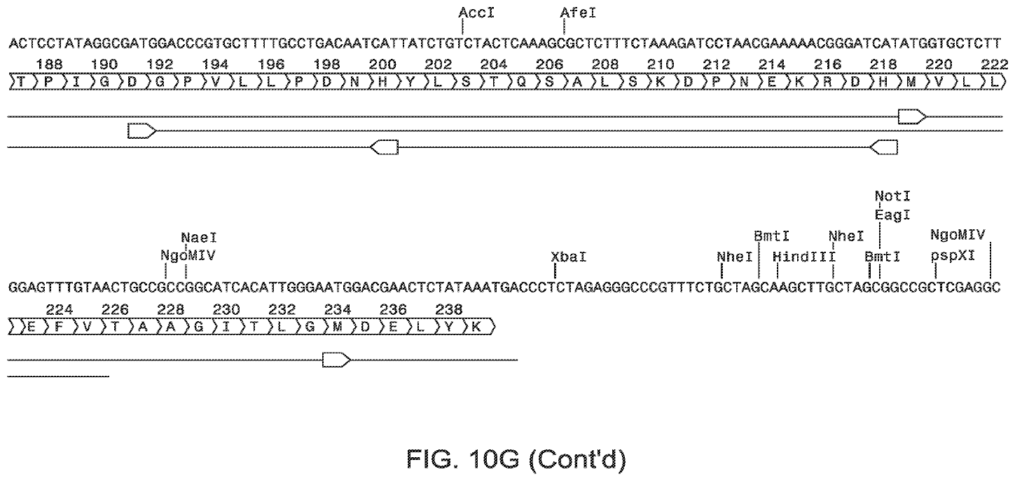

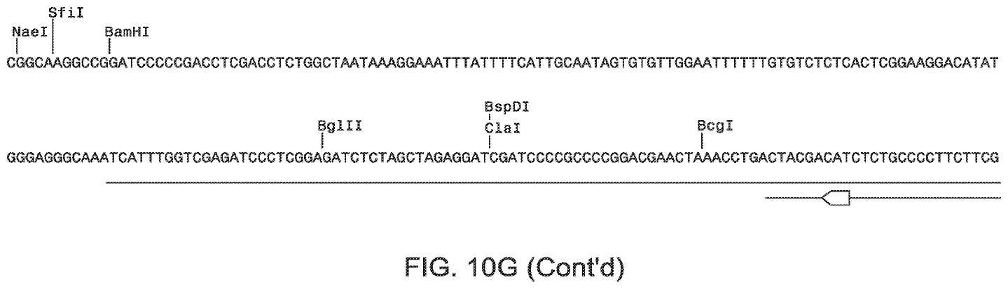

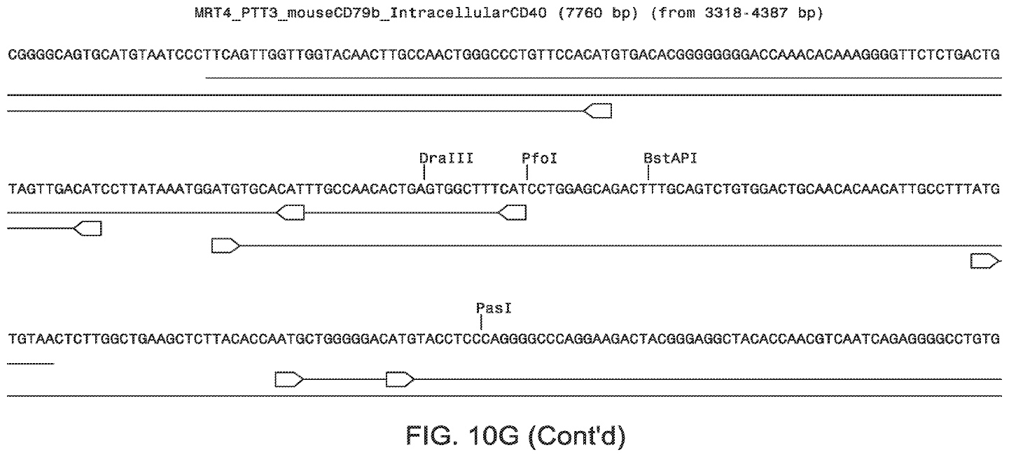

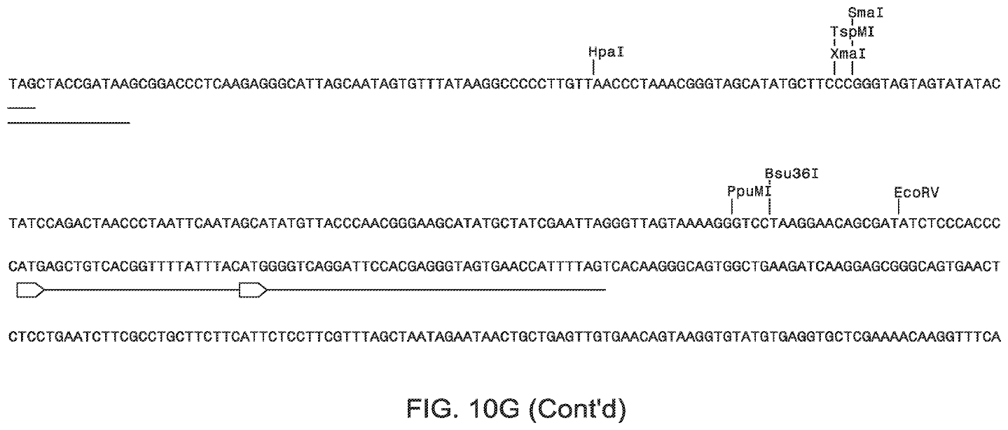

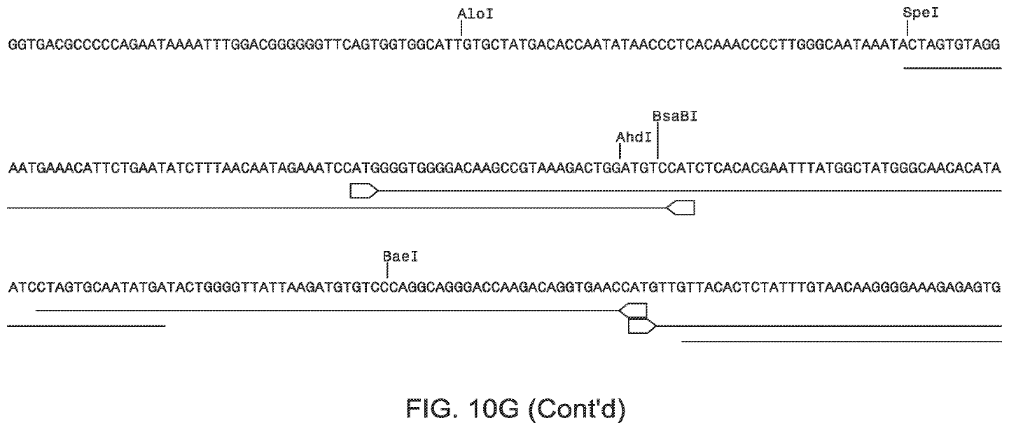

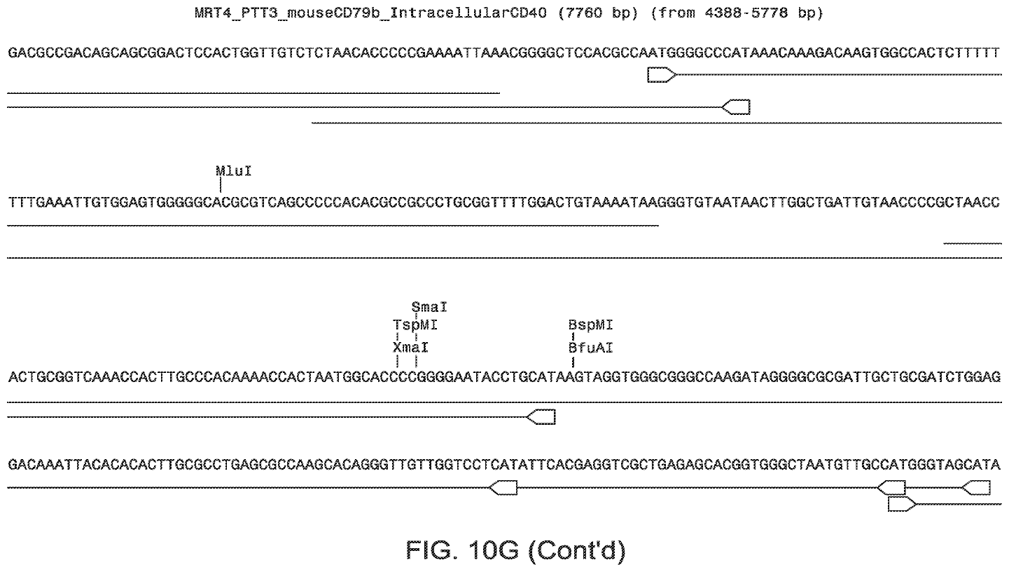

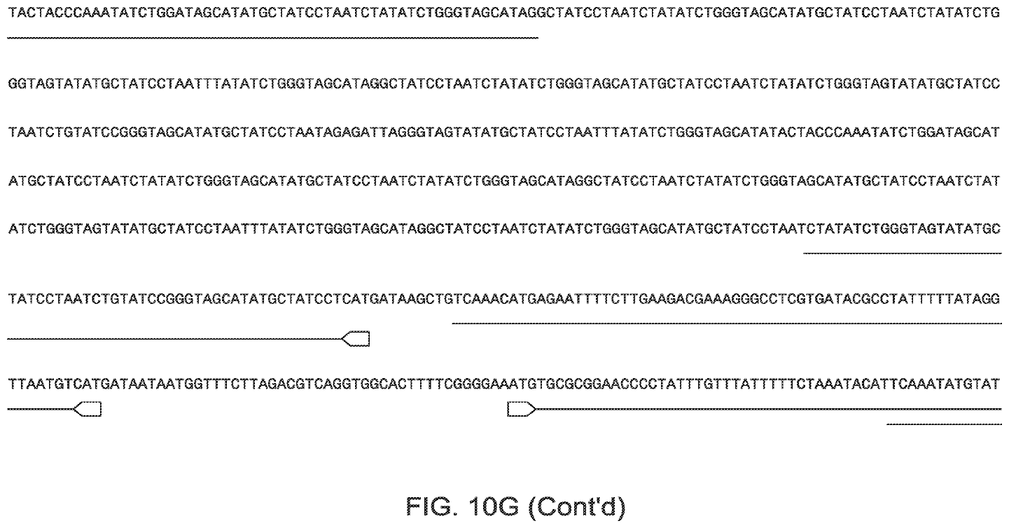

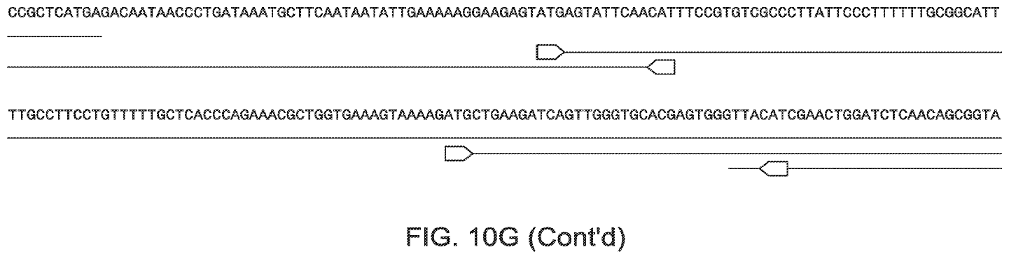

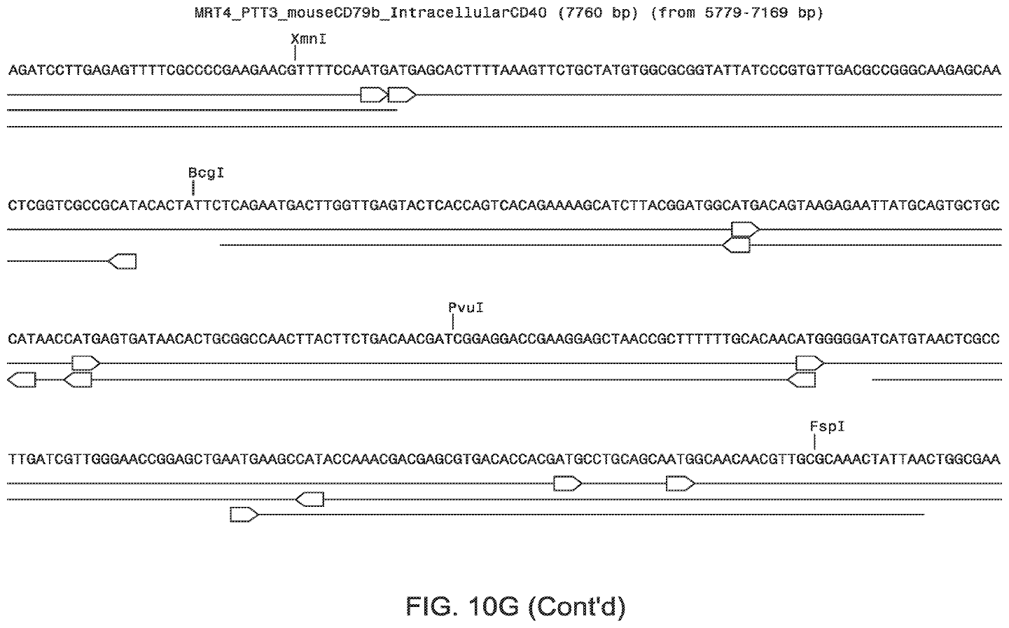

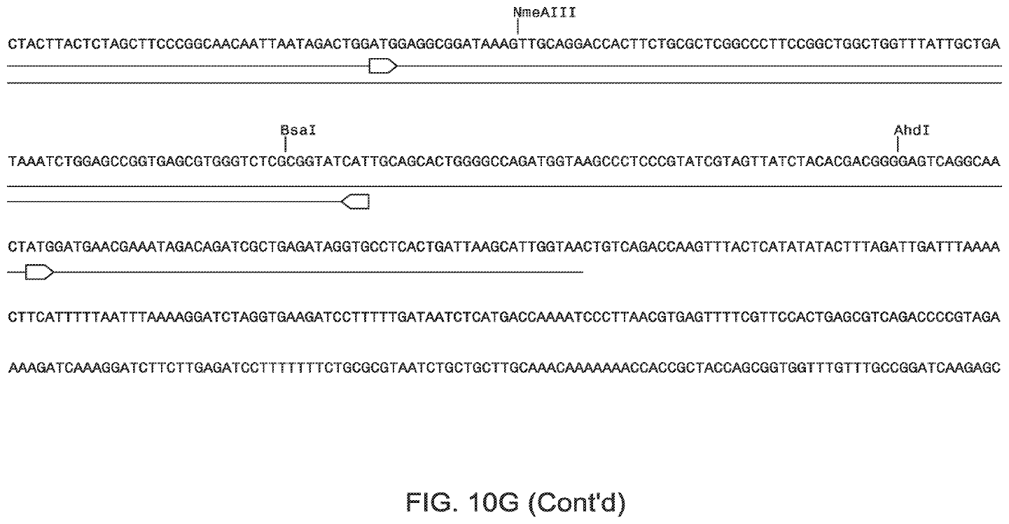

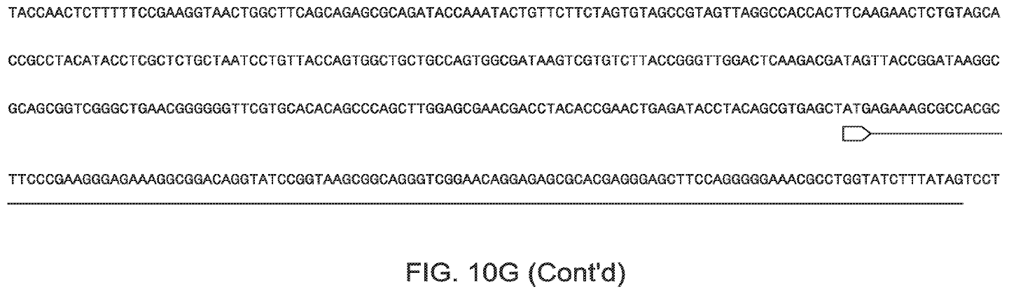

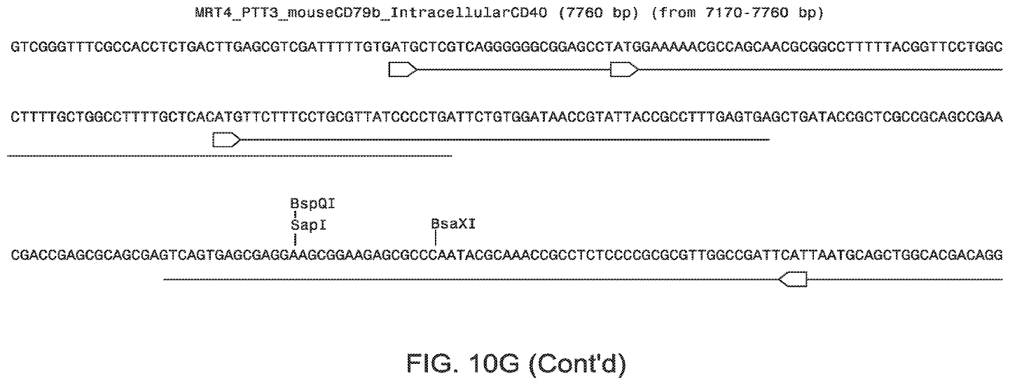

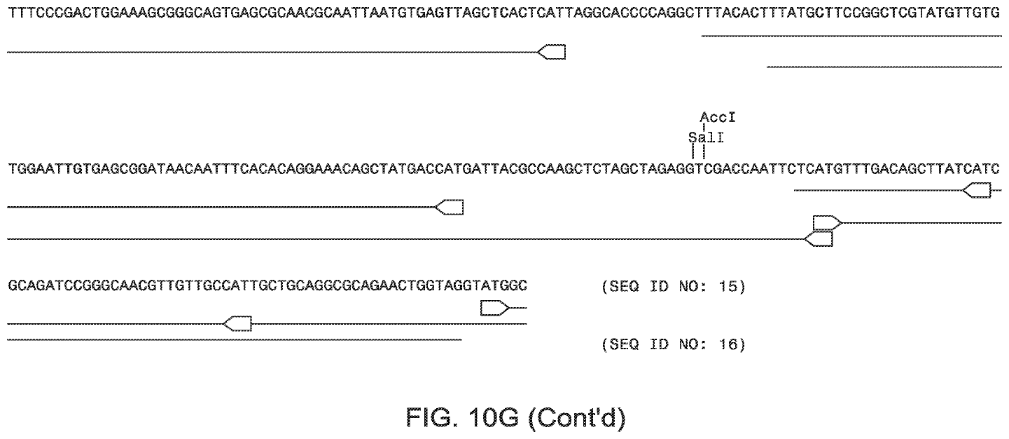

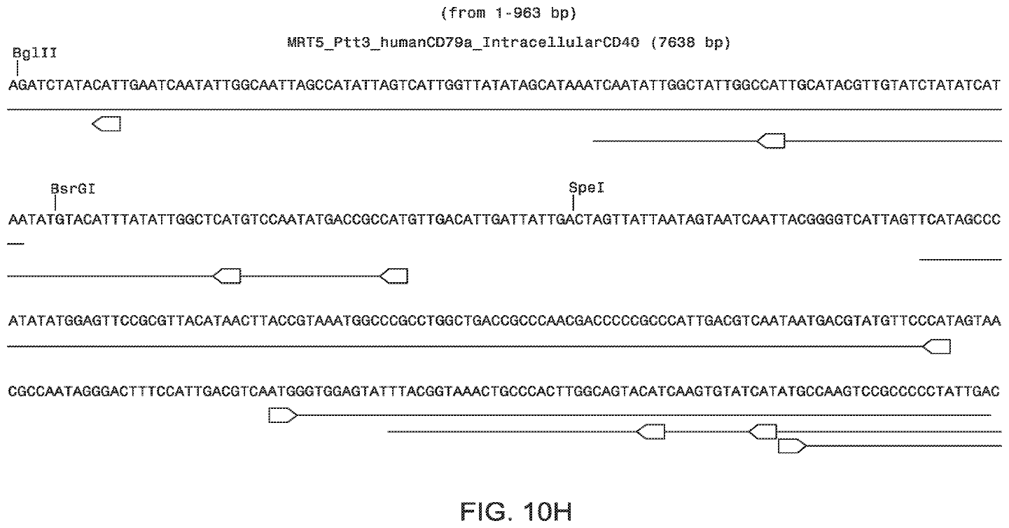

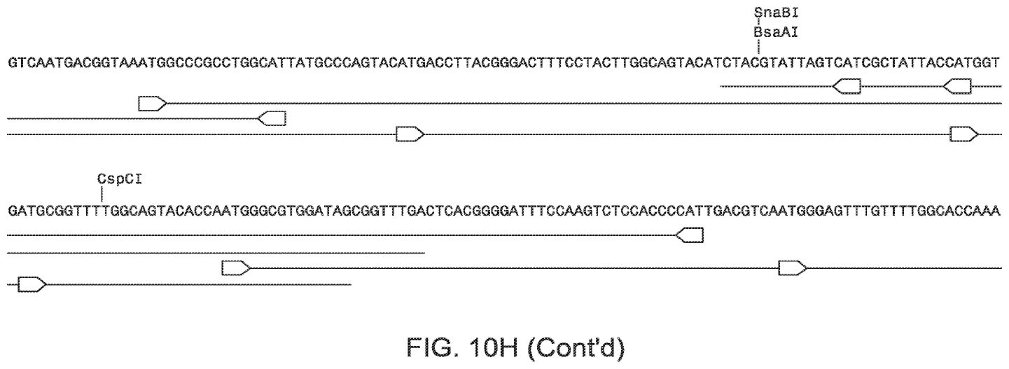

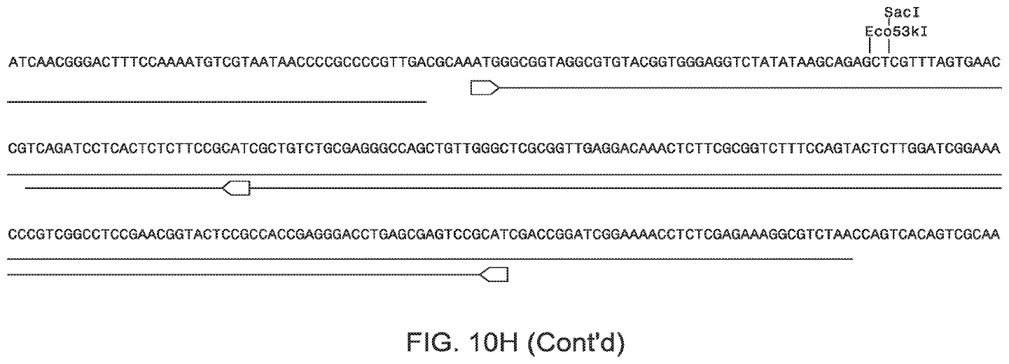

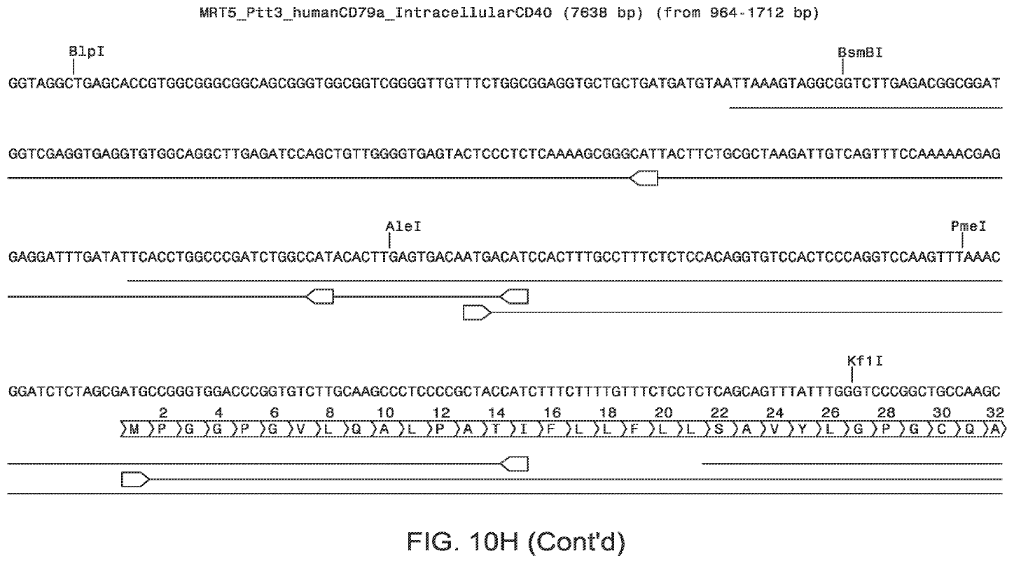

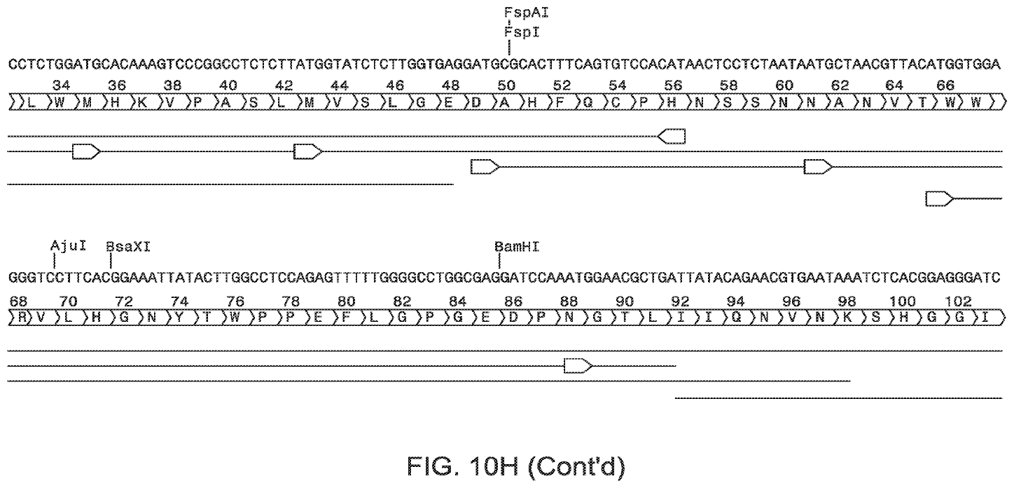

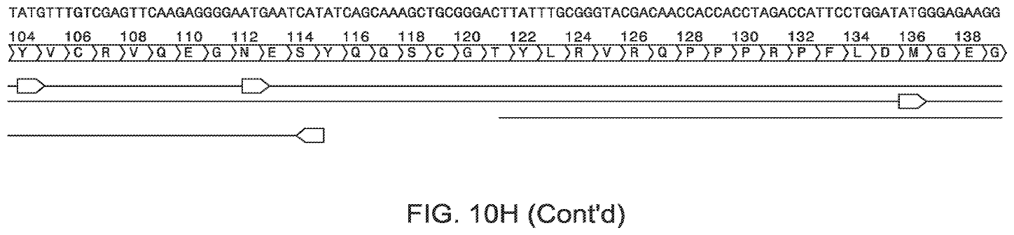

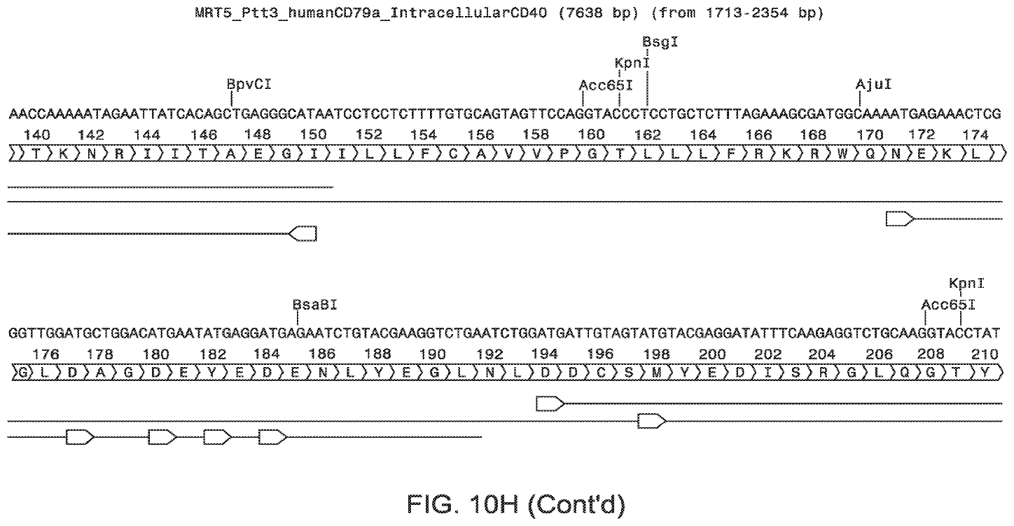

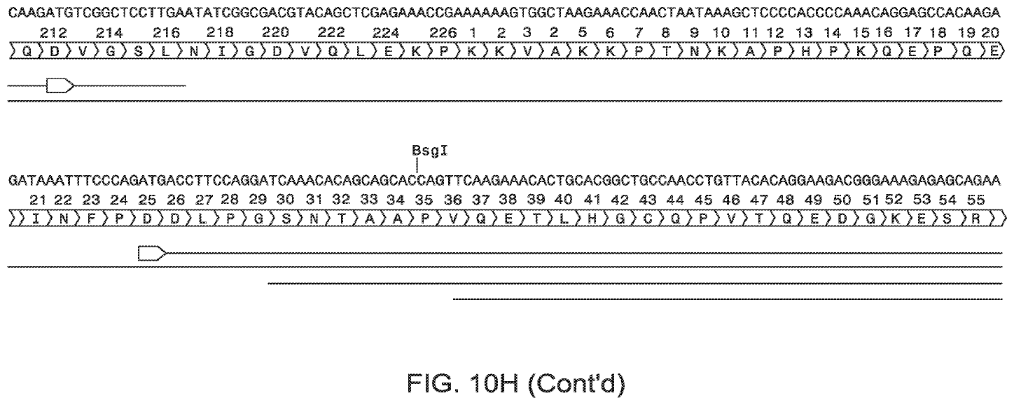

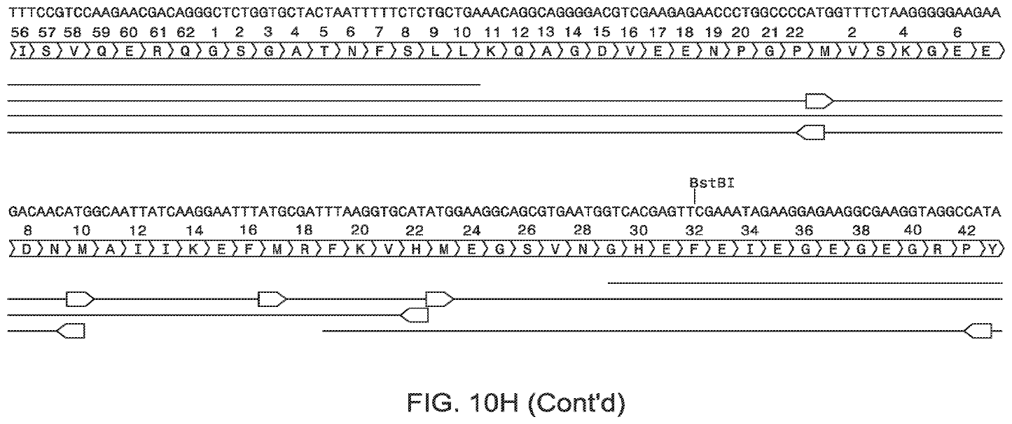

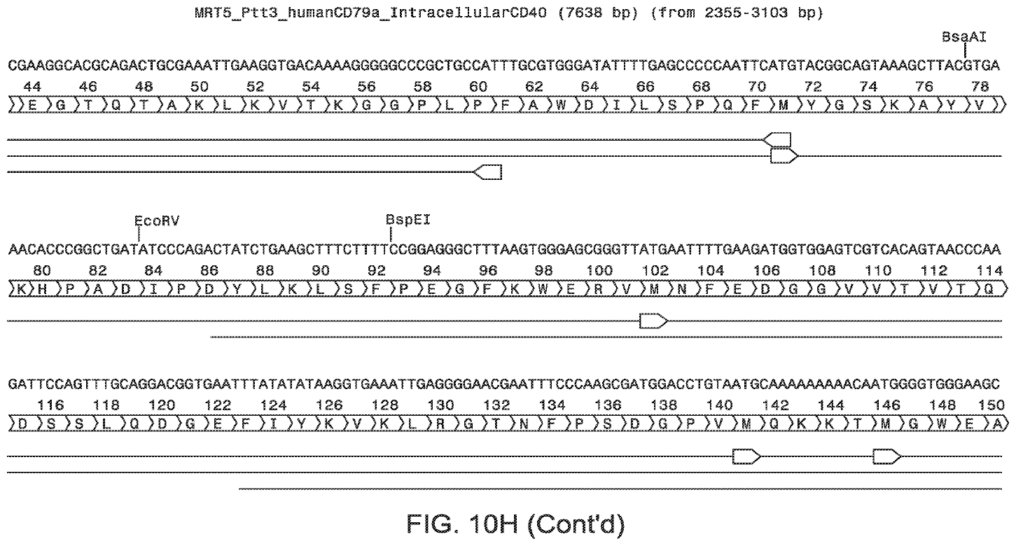

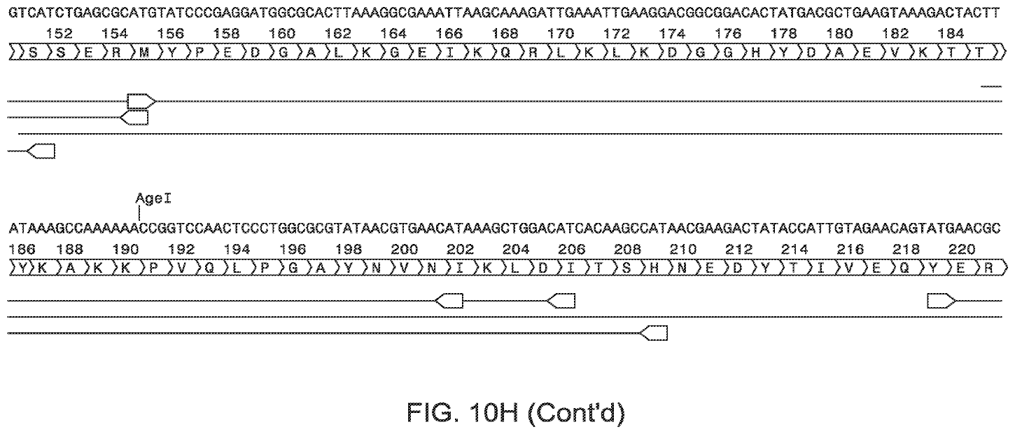

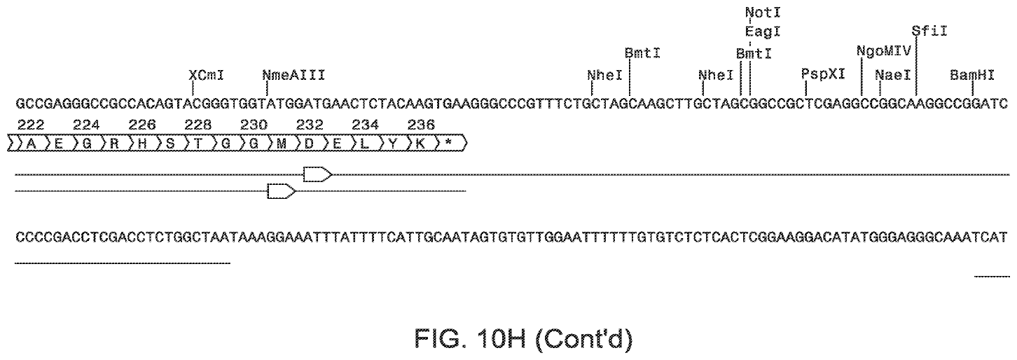

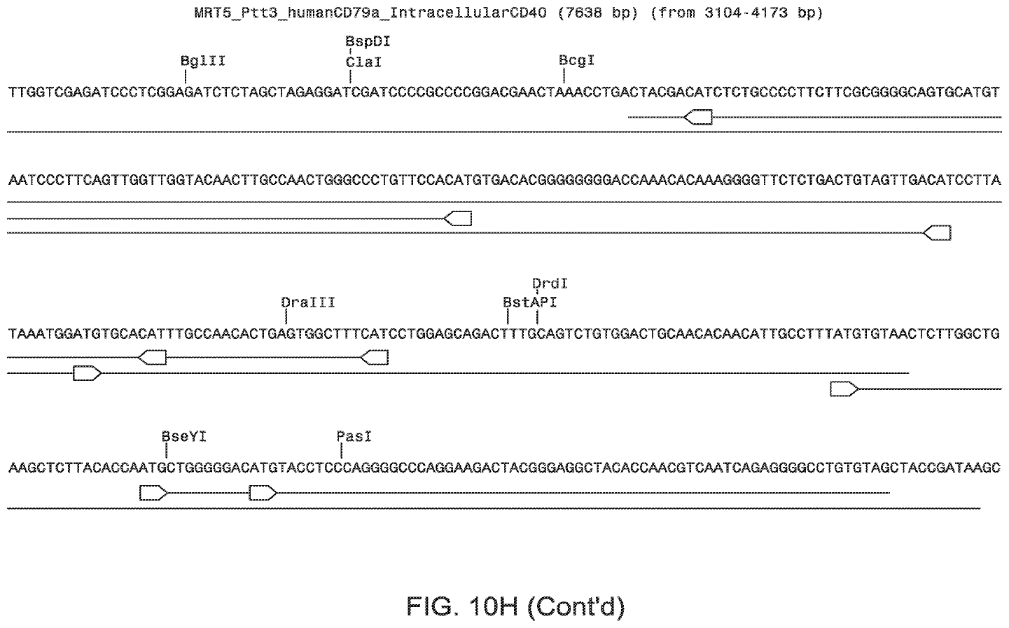

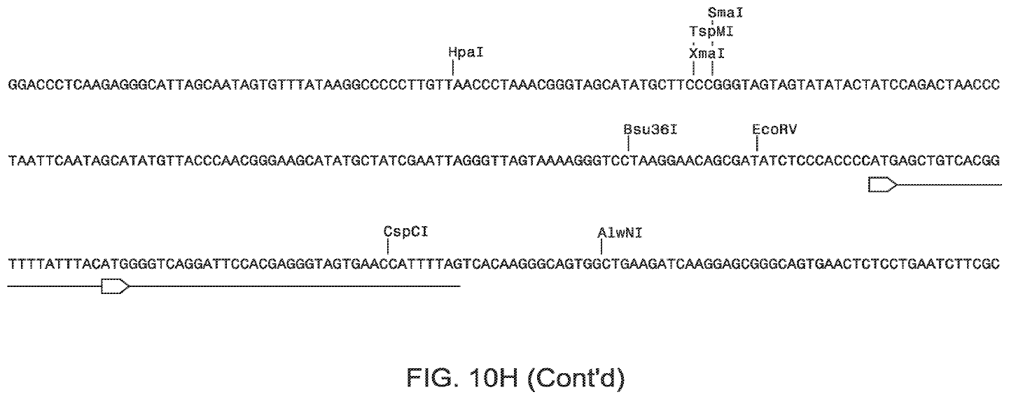

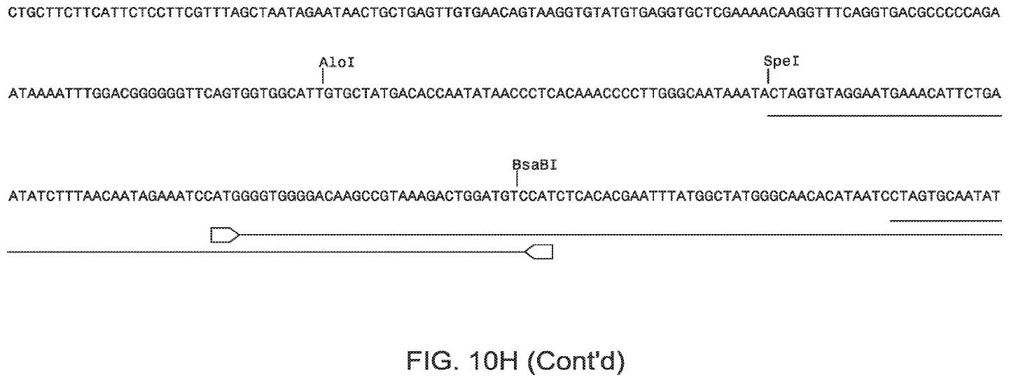

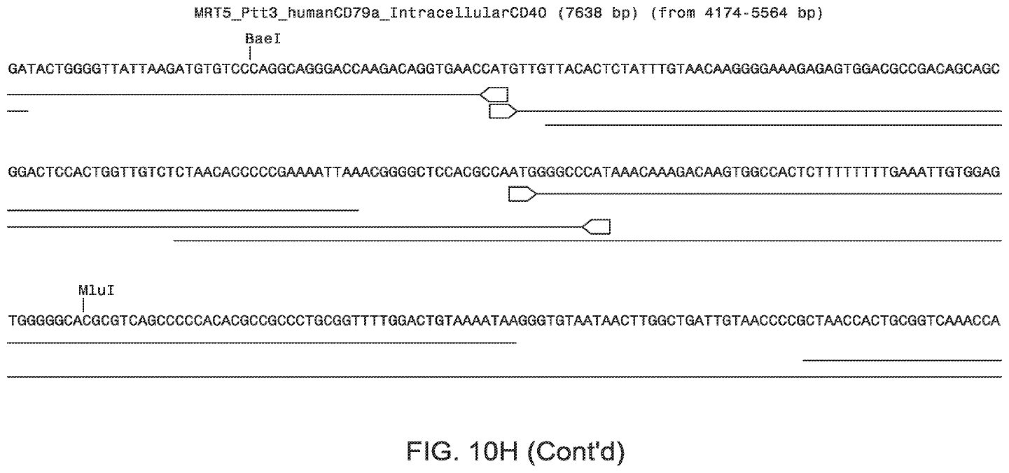

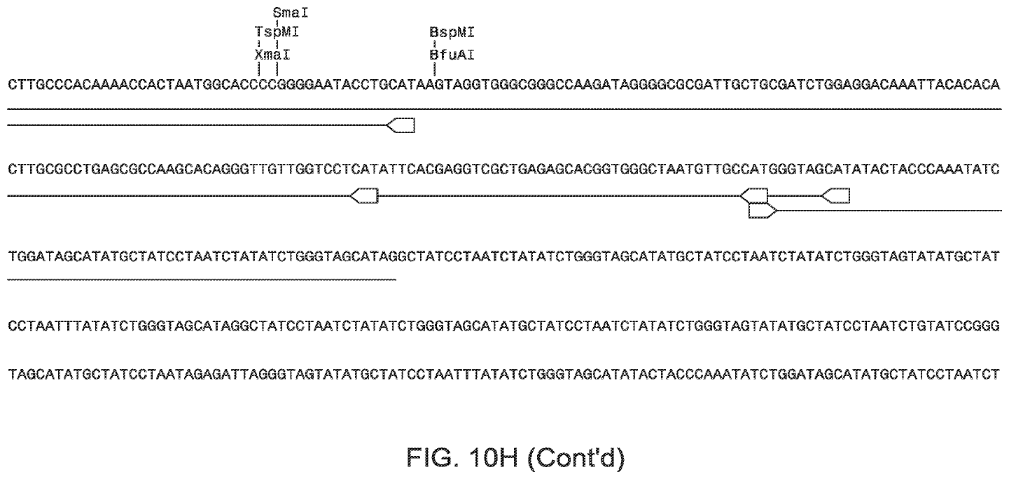

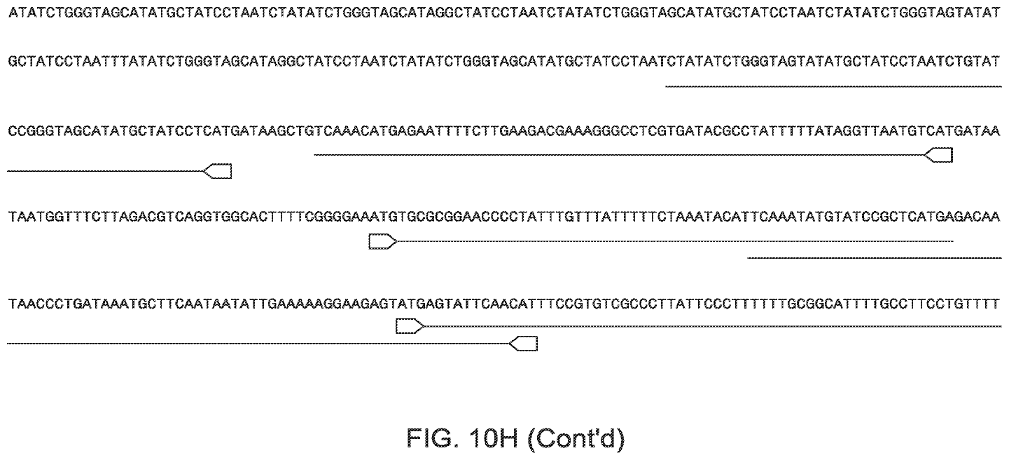

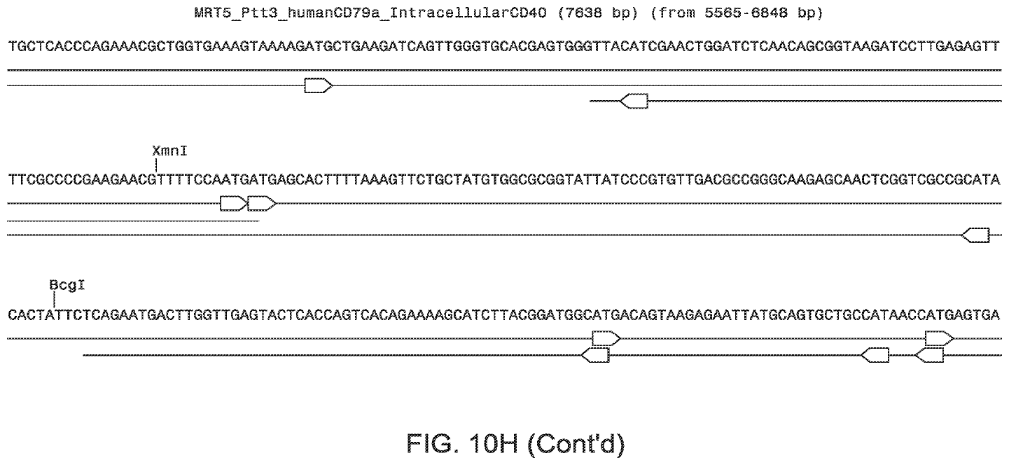

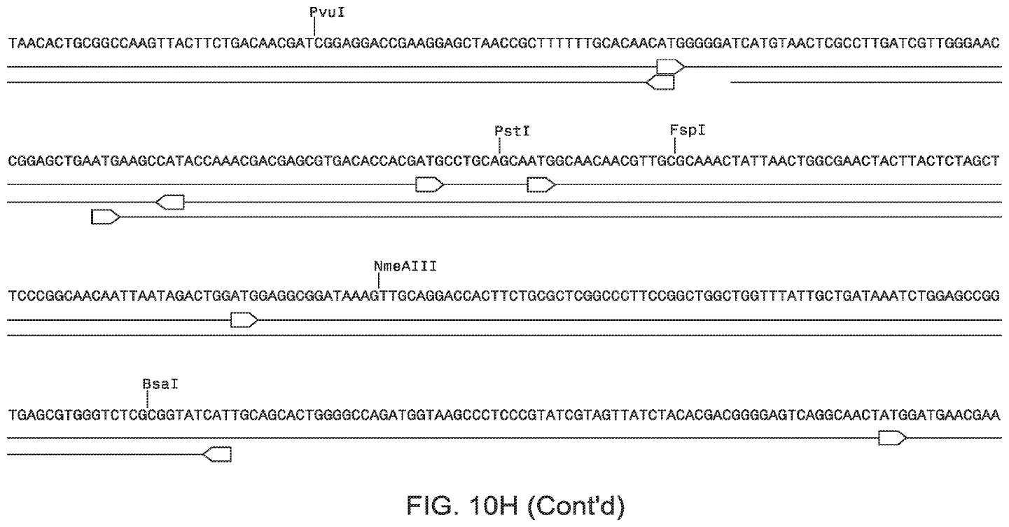

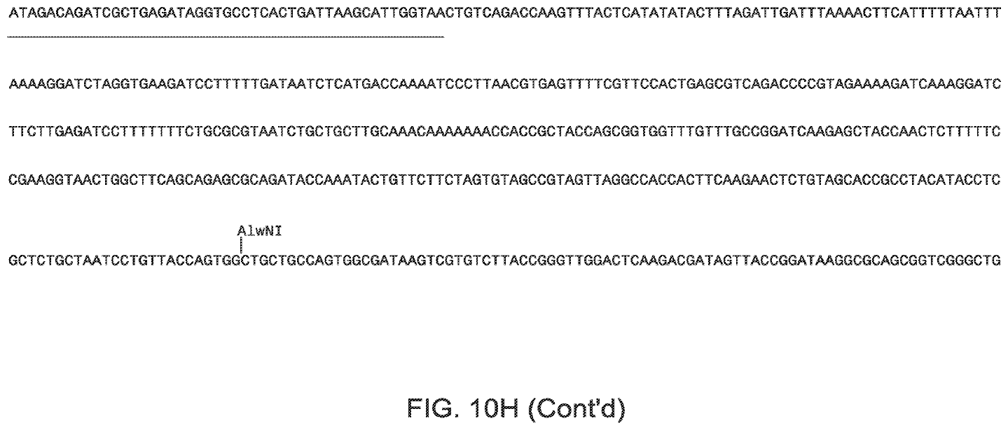

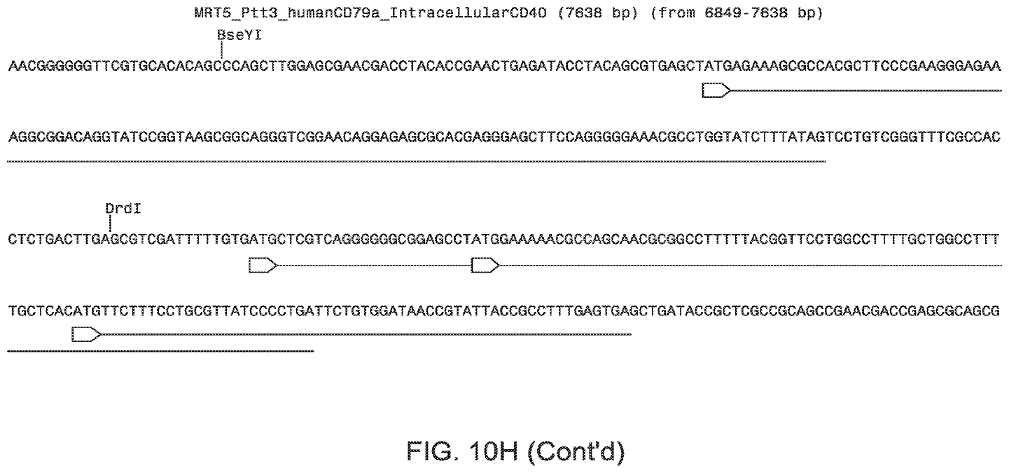

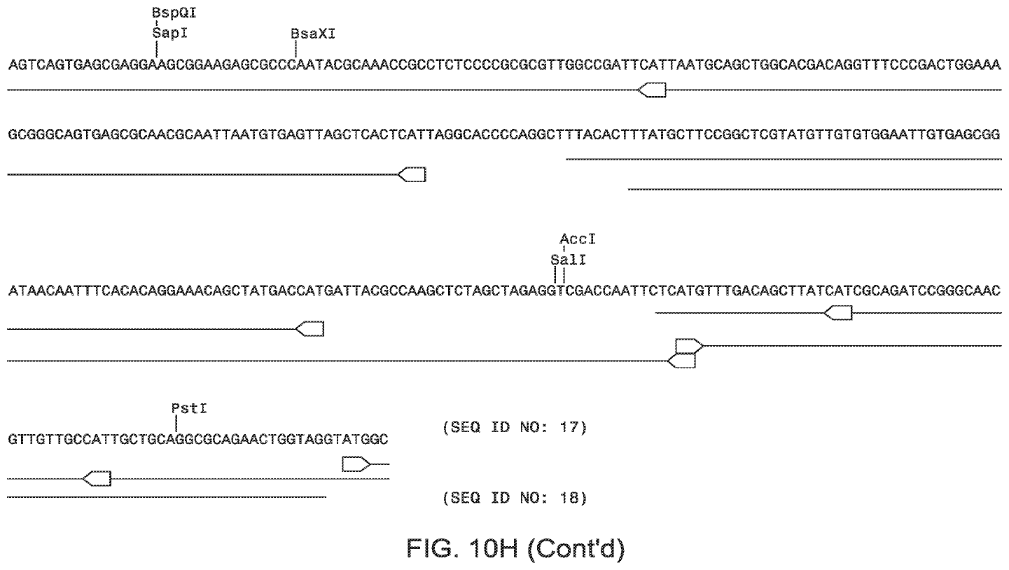

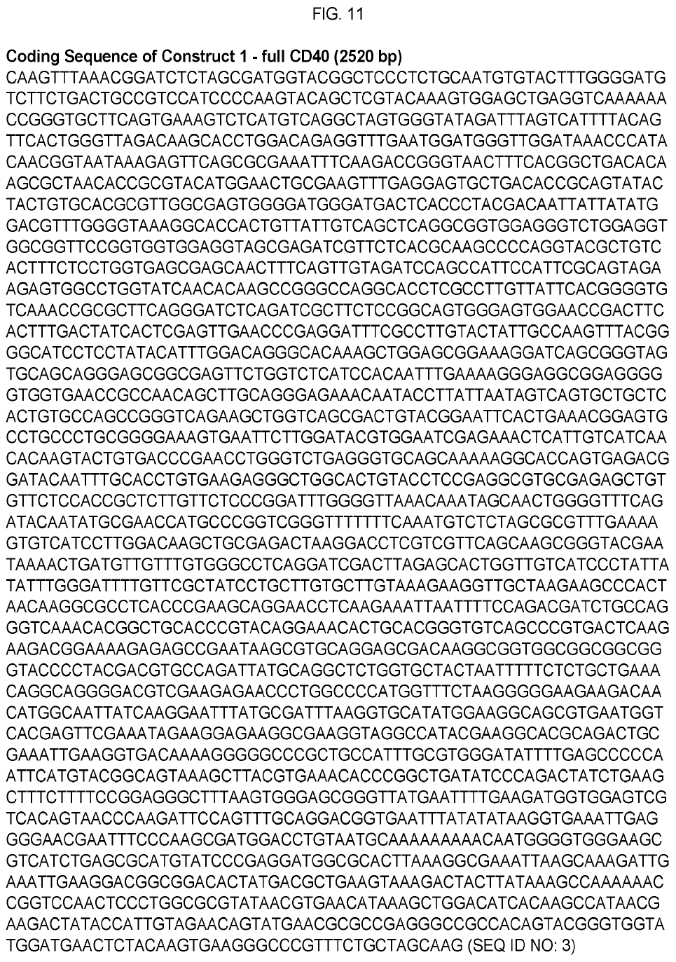

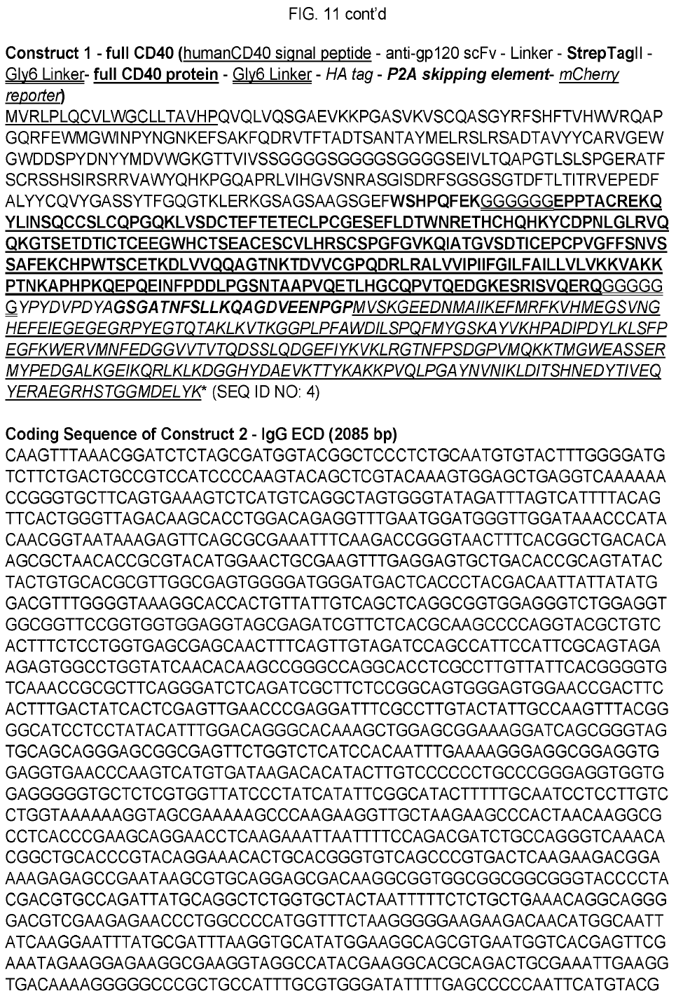

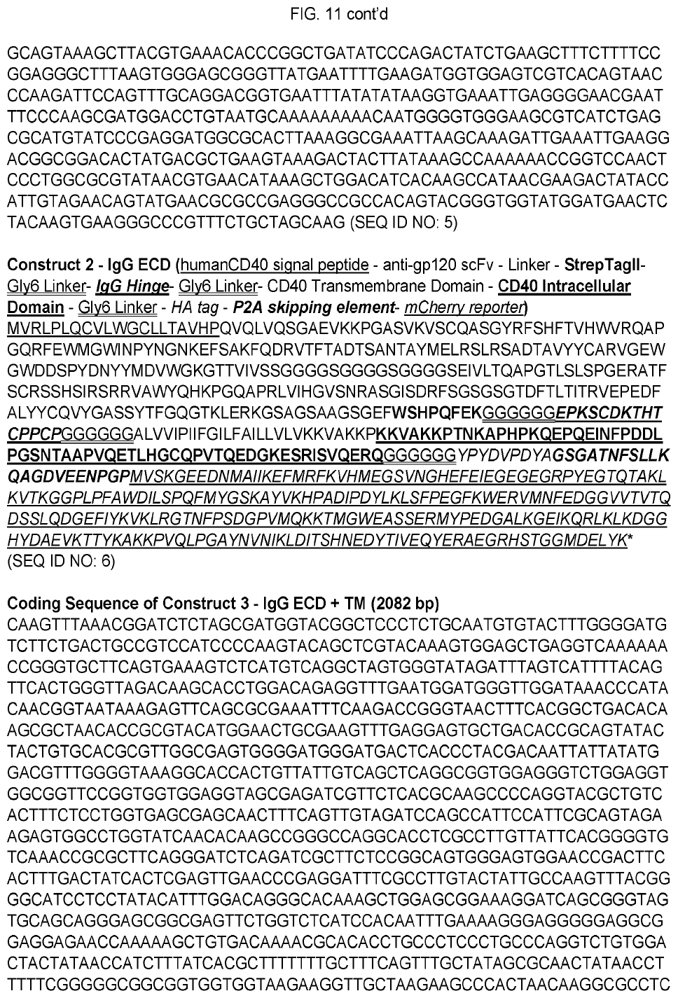

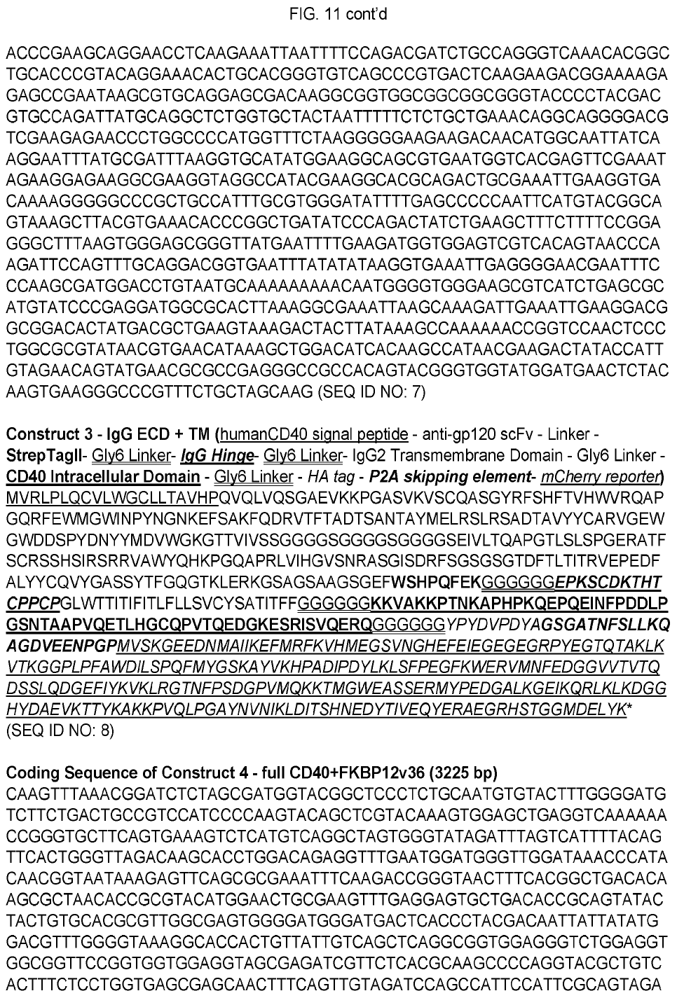

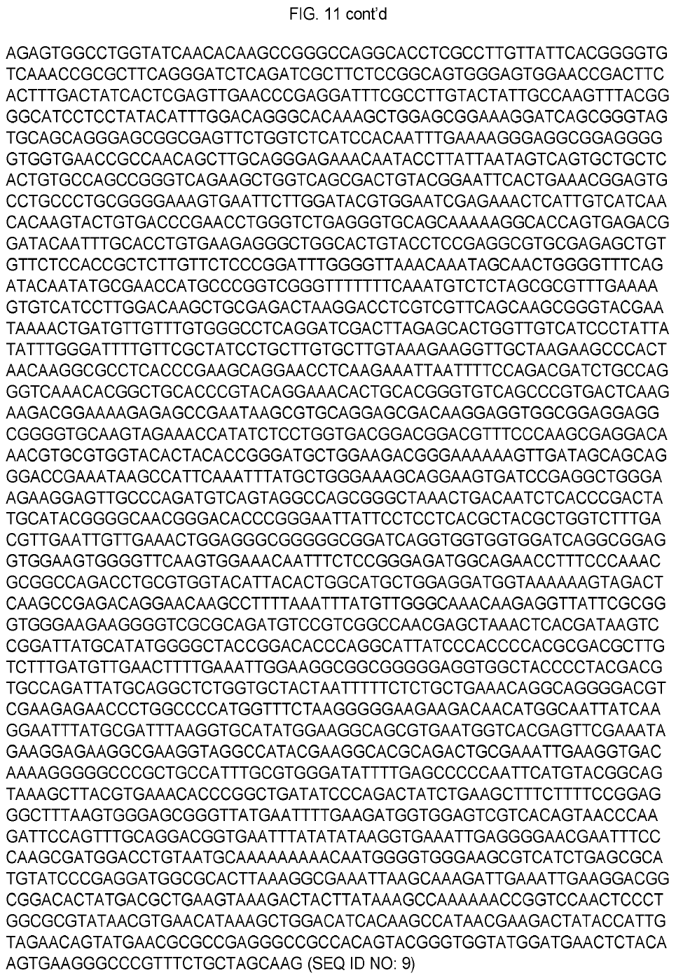

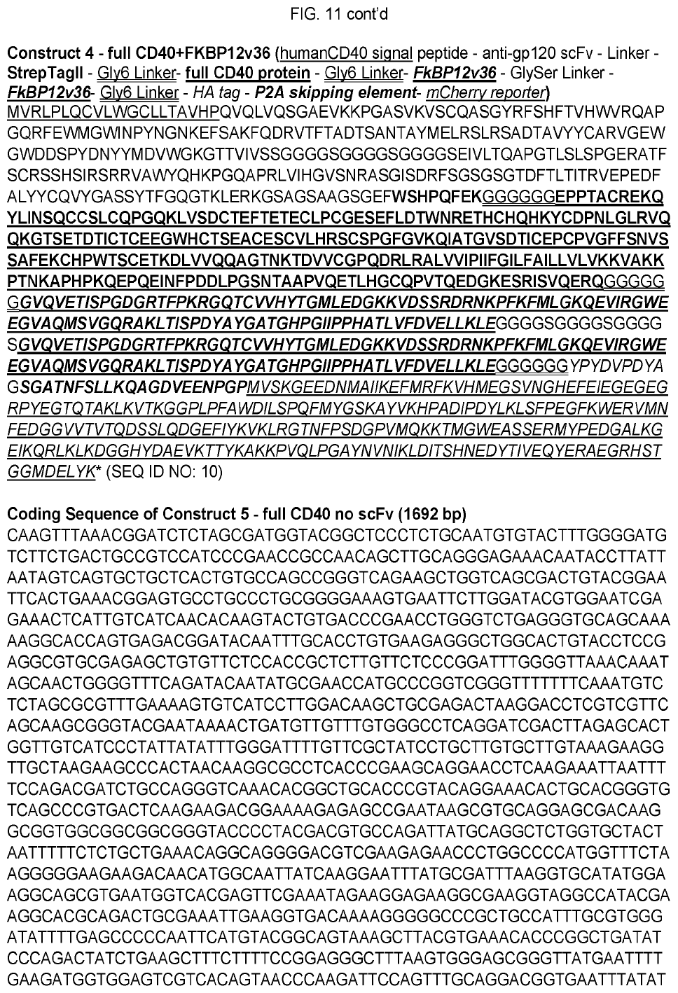

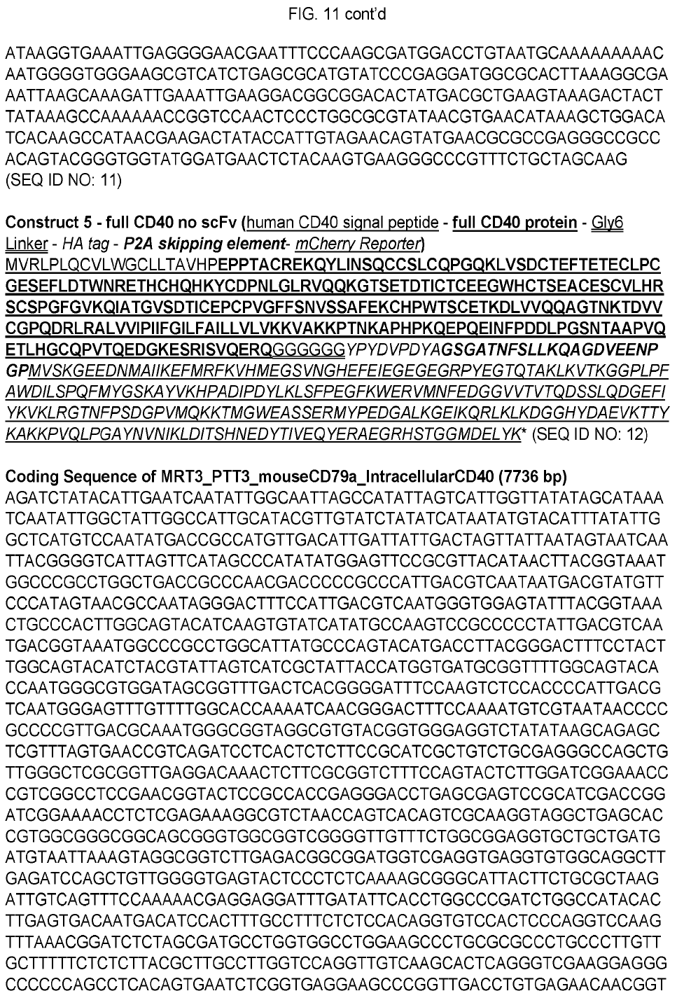

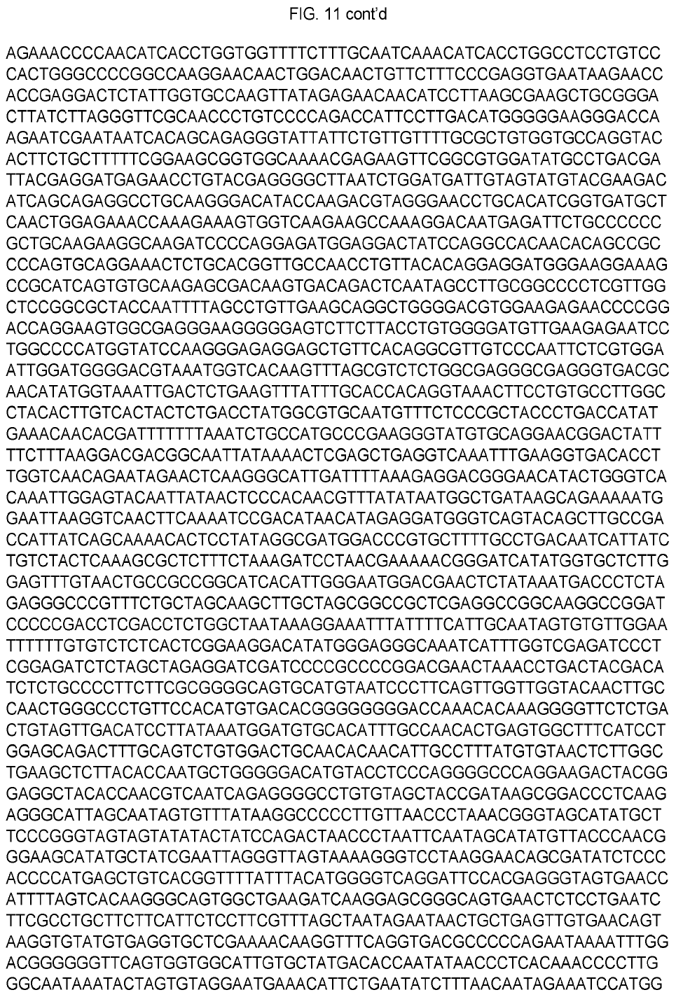

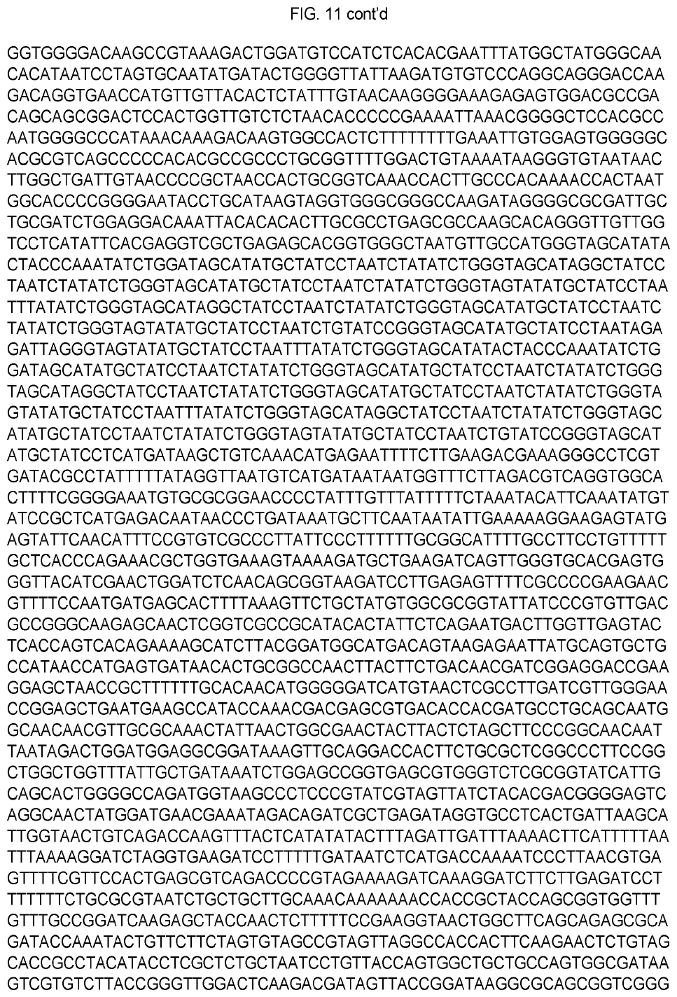

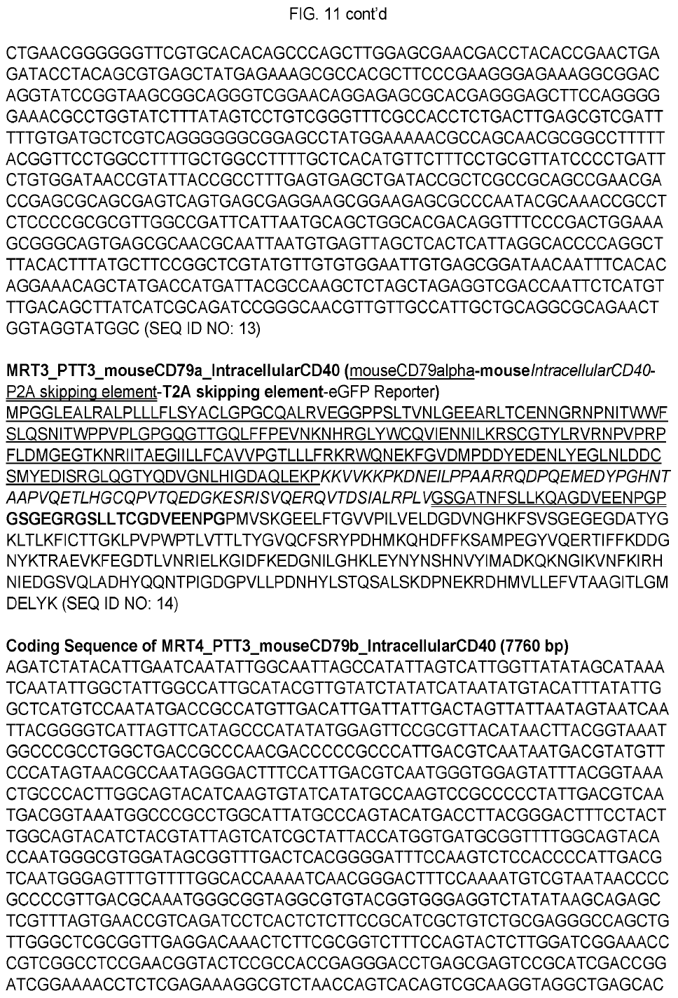

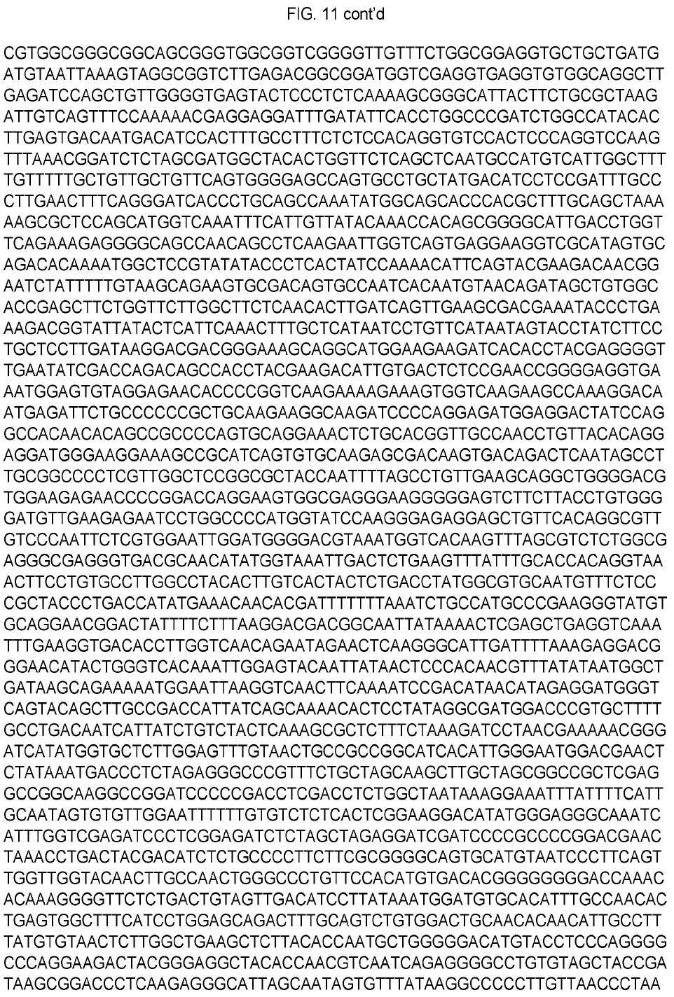

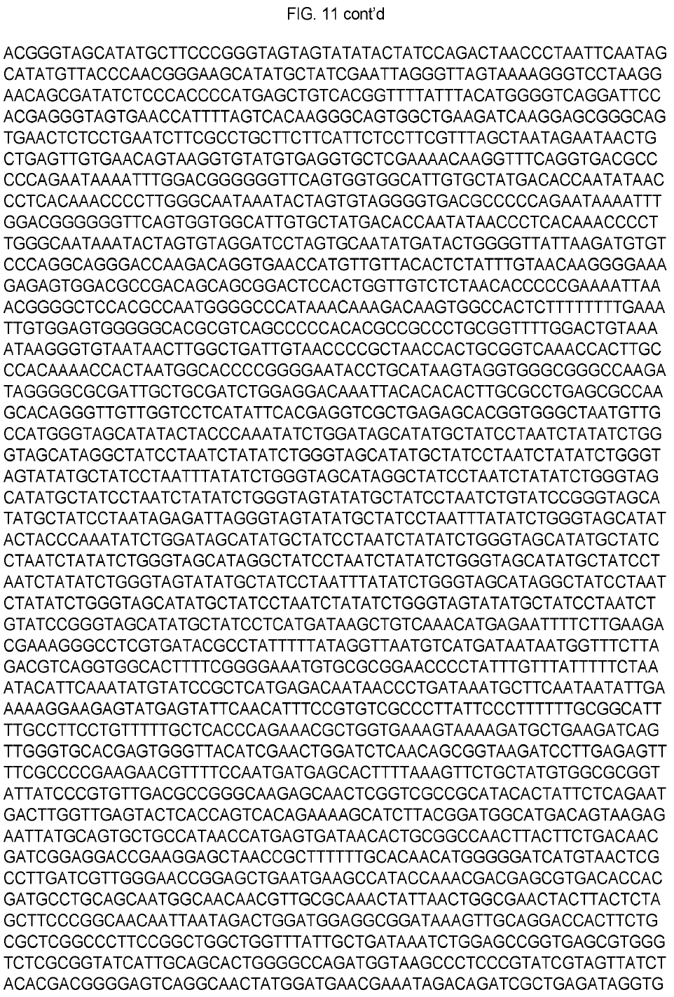

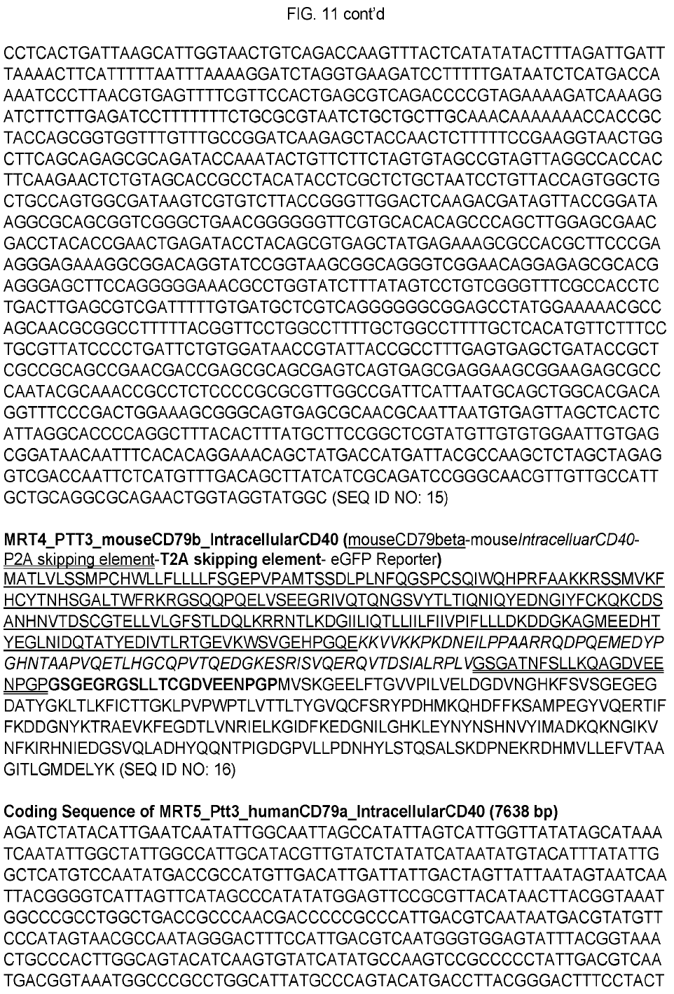

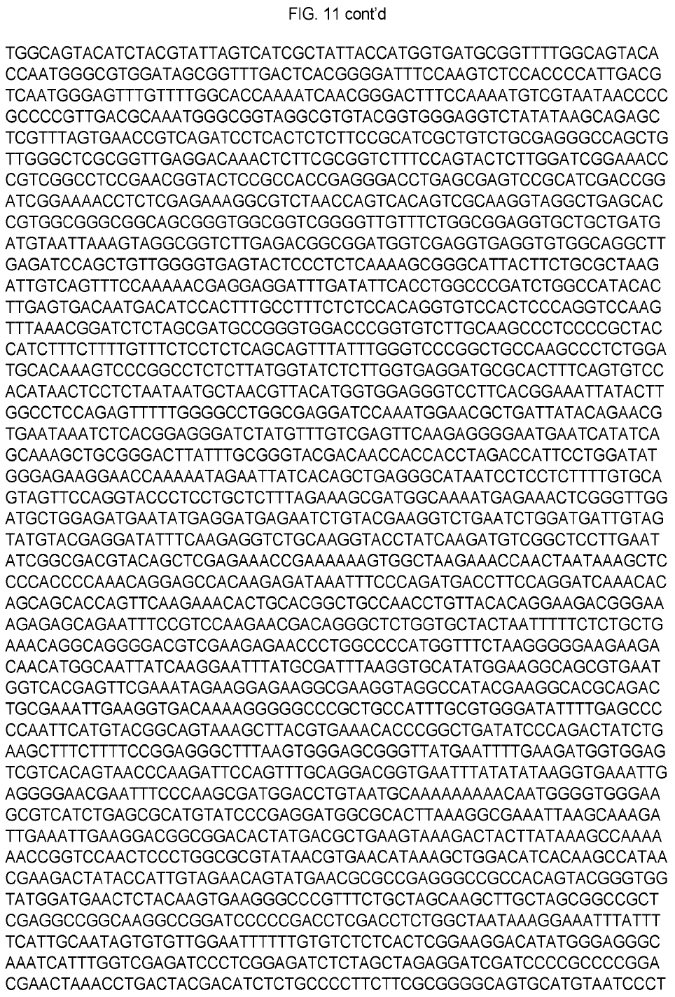

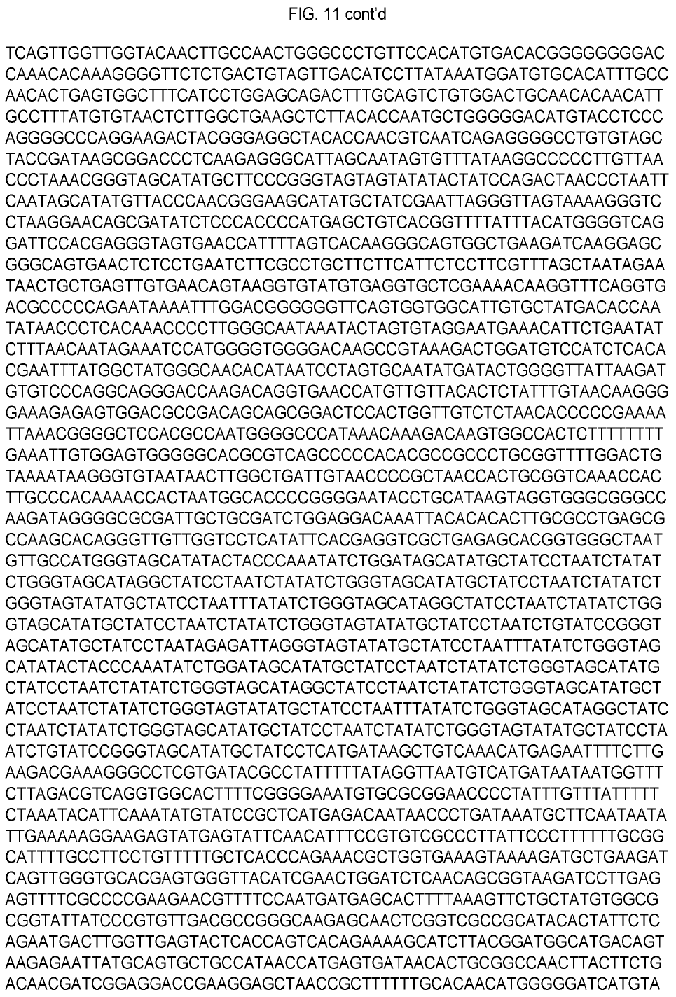

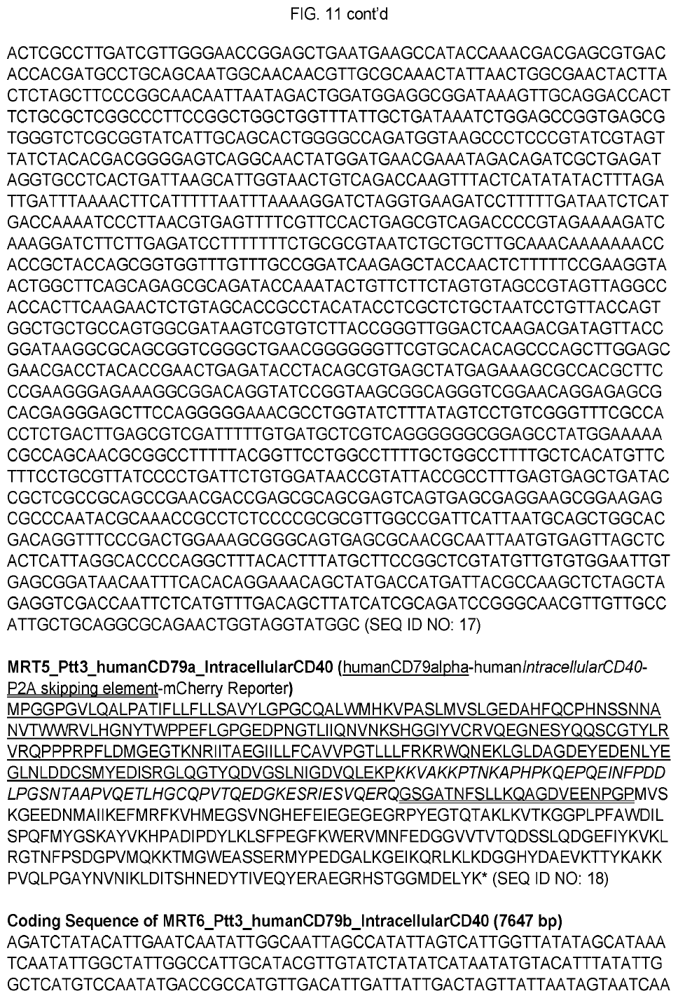

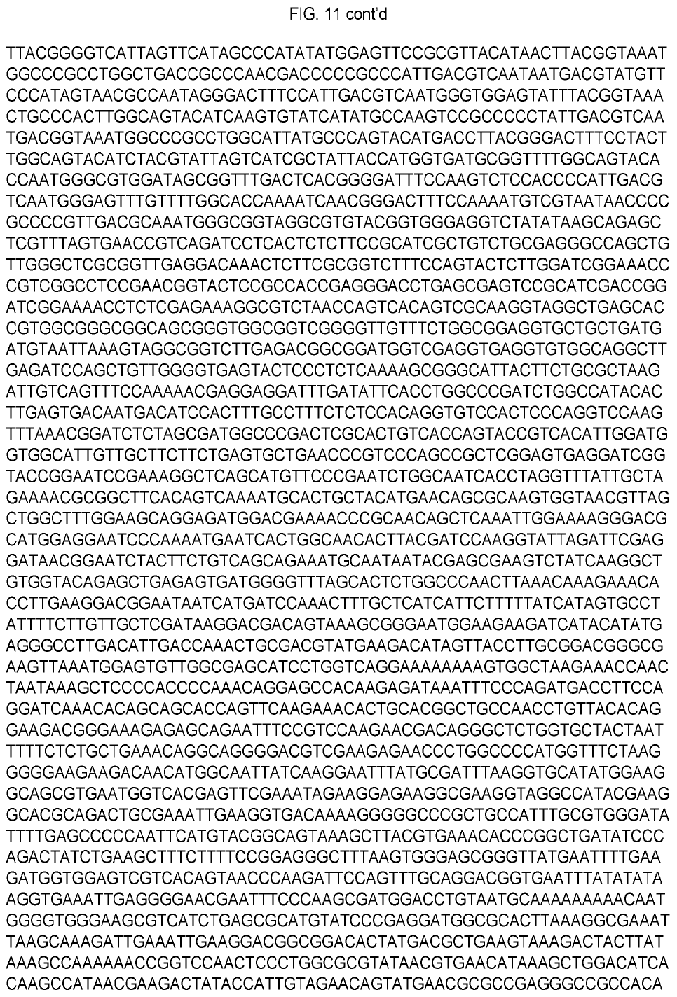

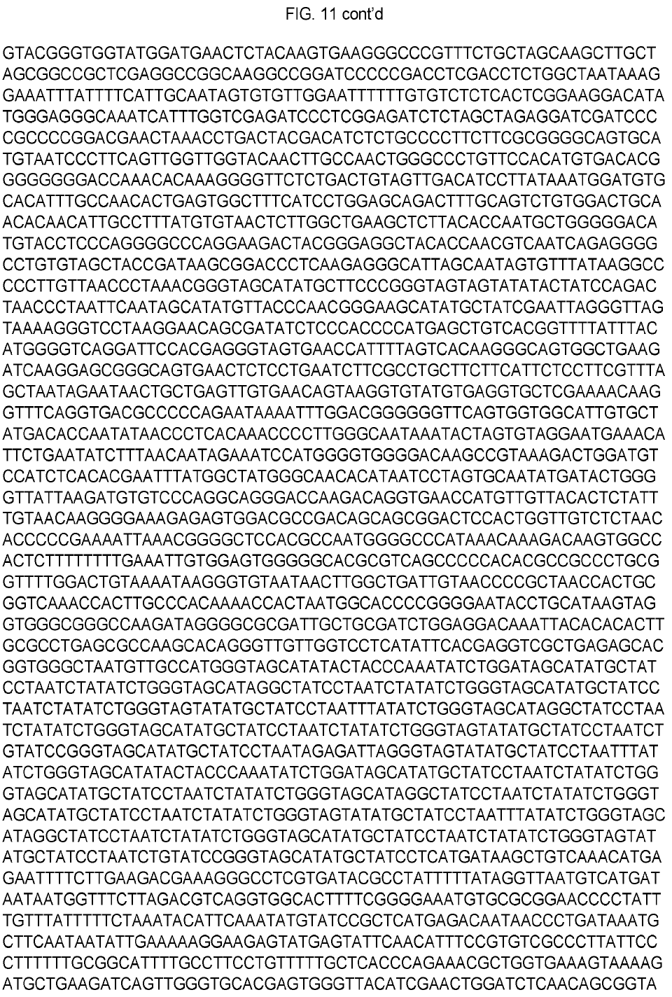

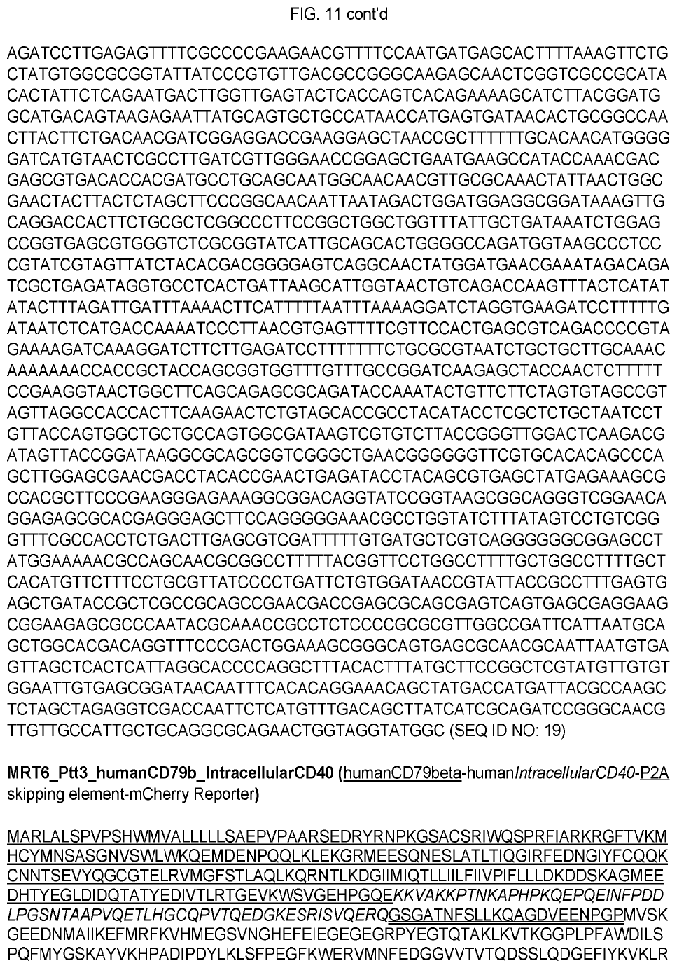

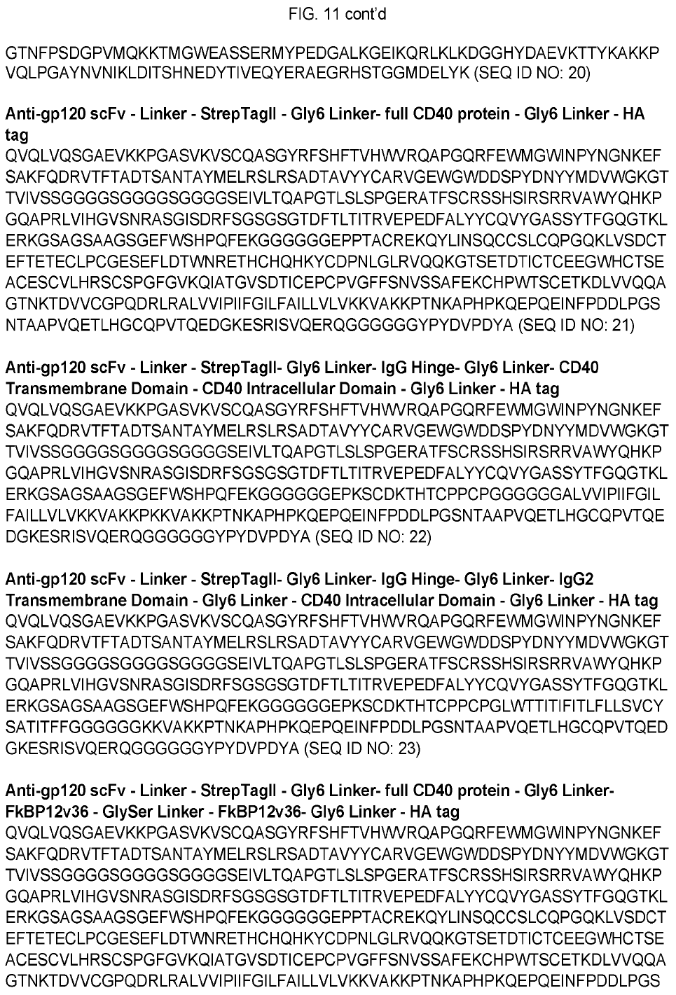

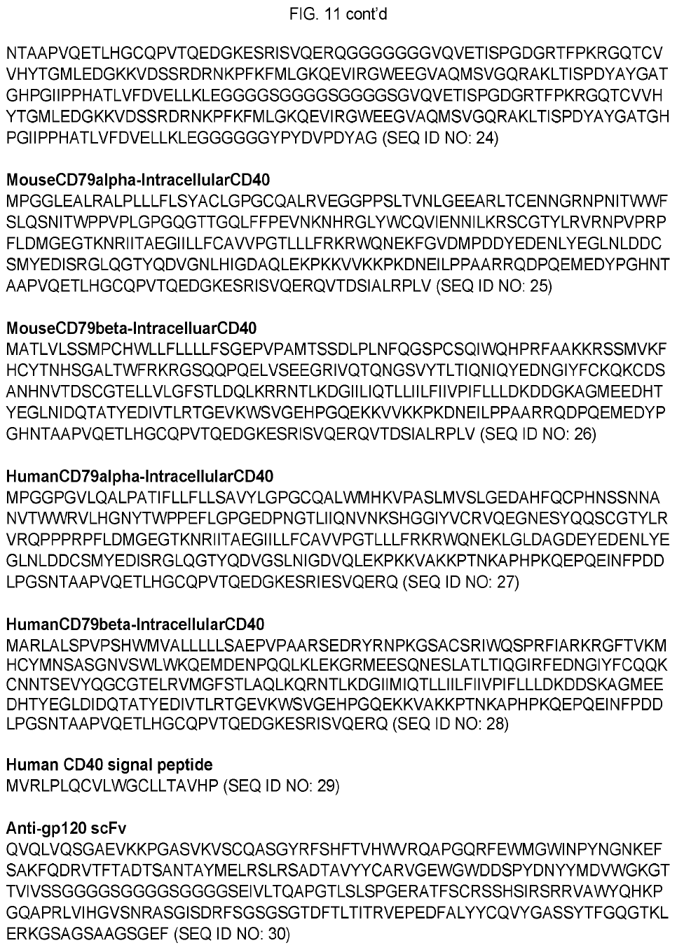

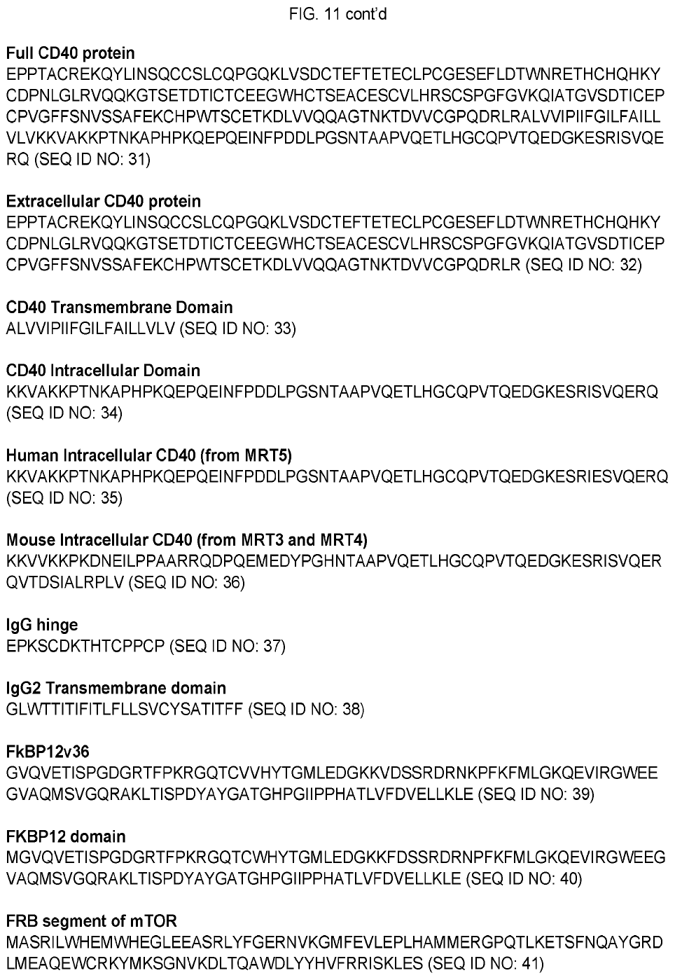

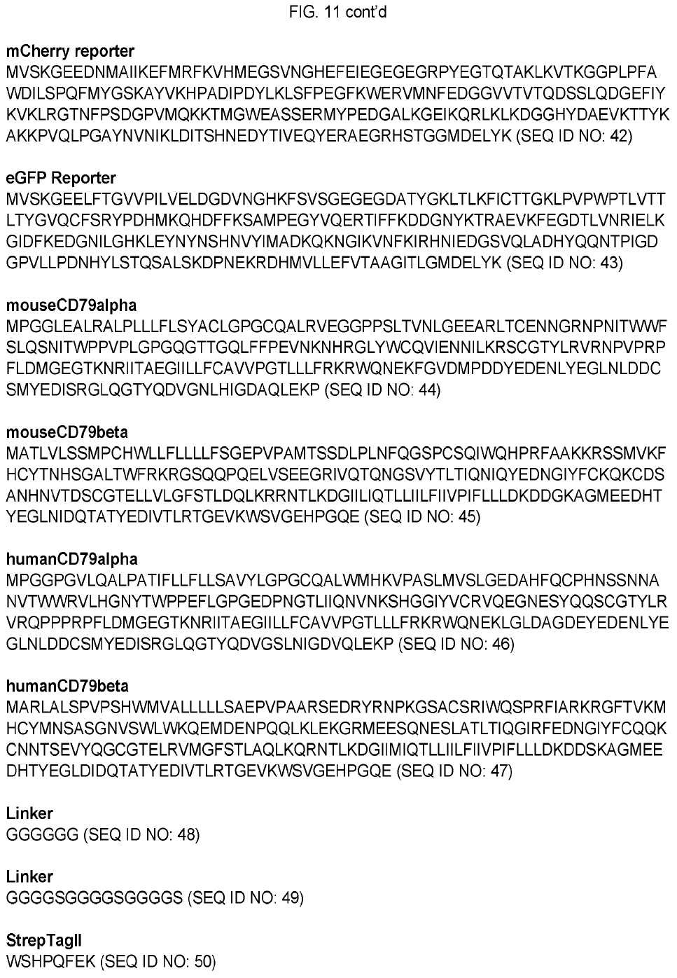

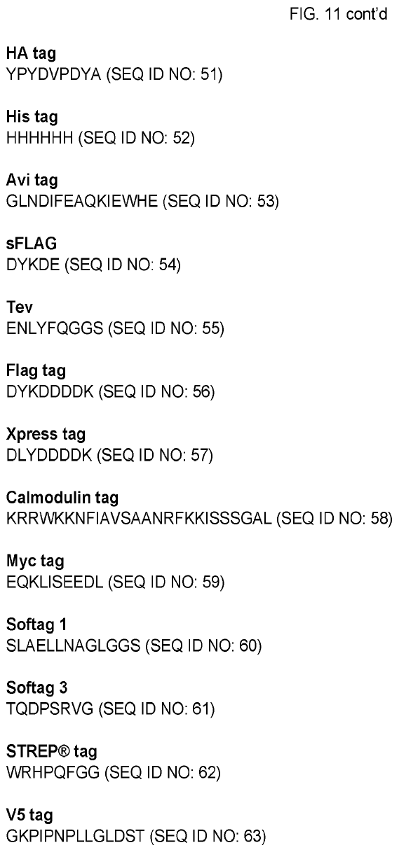

CD40 intracellular signaling domains utilized within embodiments disclosed herein result in activation of the B cell when bound by antigen. The term “CD40 intracellular signaling domain” is thus meant to include any portion of the intracellular domain sufficient to transduce an activation signal. A CD40 intracellular signaling domain can directly or indirectly promote a biological or physiological response in a cell when receiving the appropriate signal. Exemplary CD40 intracellular signaling domains are provided in FIGS. 10A-10H and 11. Functional variants and fragments of fusion proteins and intracellular signaling domains may also be used. Functional variants include one or more residue additions or substitutions that do not substantially impact the physiological effects of a protein. Functional fragments include one or more deletions or truncations that do not substantially impact the physiological effects of a protein. In particular embodiments, CD40 signaling through a fusion protein disclosed herein or a functional variant or fragment thereof is evidenced by an increase in one or more of: p38 phosphorylation, Erk1/2 phosphorylation, Stat3 phosphorylation, cytokine production (e.g., IL-2, IL-4, IL-21, IL-10, IFN-g, BAFF/BLyS production), B cell proliferation, plasma cell differentiation, and antibody production.

When a subject donates his or her own B cells for genetic engineering or is otherwise immunologically matched with a donor, no modifications regarding the B cell's MHC class II alleles are required. However, when B cells genetically engineered as disclosed herein are used as universal donor cells, MHC class II molecules should be deleted. MHC class II molecule expression can be deleted by reducing or eliminating activity of one or more transcription factors required for MHC class II molecule expression. Such transcription factors include CIITA, TRAC, TRBC, B2M, RFX5, and RFXAP. As used herein, “deleting” or “eliminating” does not require complete deletion or elimination but instead refers to reduced levels leading to clinically-acceptable use. “Clinically-acceptable use” means that research or clinical benefit outweighs potential risk, as determined by a relevant regulatory authority (e.g., IACUC or the FDA) and/or a treating researcher, physician, or veterinarian.

Aspects of the disclosure are now described with additional detail and options as follows: (i) Extracellular Antigen Binding Domains; (ii) Exemplary Anti-Viral Binding Domains; (iii) Spacers, Linkers, and Junction Amino Acids; (iv) Tags; (v) Multimerization Molecules; (vi) Transmembrane Domains; (vii) Skipping Elements; (viii) Reporters; (ix) Gene Editing Techniques and Cell Sorting; (x) Formulation of Modified B cells; (xi) Methods of Use; (xii) Exemplary Embodiments; (xiii) Experimental Examples; and (xiv) Closing Paragraphs. These headings do not limit the interpretation of the disclosure and are provided for organizational purposes only.

(i) Extracellular Antigen Binding Domains. Approaches to activate B cells through antigen binding, independently of interactions with CD40L utilize an extracellular antigen binding domain. The extracellular antigen binding domain can be an antigen binding domain such as an engineered binding domain (e.g., an engineered antibody binding domain) or a BCR.

One approach to activating CD40 signaling in B cells independently of interactions with CD40L includes expressing an engineered molecule that results in an extracellular antigen binding domain that binds an antigen of interest. In some examples, B cells expressing the engineered molecule that results in an extracellular antigen binding domain that binds an antigen of interest will also be genetically modified to express antibodies that bind the antigen of interest.

Engineered antigen binding domains can take many forms. Particular examples are binding domain fragments derived from antibody binding domains. For example, particular embodiments can include binding fragments of an antibody or engineered forms thereof, e.g., Fv, Fab, Fab′, F(ab′)₂, Fab′-SH, diabodies; and linear antibodies, single chain Fv fragments (scFvs) or any biologically effective fragments of an immunoglobulin that bind specifically to a targeted antigen. In other words, an “antibody fragment” denotes a portion of a complete or full-length antibody that retains the ability to bind to an epitope. Antibody fragments can be made by various techniques, including proteolytic digestion of an intact antibody as well as production by recombinant host-cells (e.g., mammalian suspension cell lines, E. coli or phage), as described herein. Antibody fragments can be screened for their binding properties in the same manner as intact antibodies. Engineered forms of antibody binding domains or fragments thereof re-arrange or newly-construct antibody sequences or formats into non-naturally occurring forms thereof.

A single chain variable fragment (scFv) is a fusion protein of the variable regions of the heavy and light chains of immunoglobulins connected with a short linker peptide. Fv fragments include the V_{L} and V_{H} domains of a single arm of an antibody but lack the constant regions. Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). For additional information regarding Fv and scFv, see e.g., Bird, et al., Science 242:423-426, 1988; Huston, et al., Proc. Natl. Acad. Sci. USA 85:5879-5883, 1988; Plueckthun, in The Pharmacology of Monoclonal Antibodies, vol. 113, Rosenburg and Moore (eds.), Springer-Verlag, New York), (1994) 269-315; WO 1993/16185; U.S. Pat. Nos. 5,571,894 and 5,587,458.

Linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. Linker sequences of scFv are commonly Gly-Ser linkers, described in more detail elsewhere herein.

Commonly used flexible linkers include linker sequence with the amino acids glycine and serine (Gly-Ser linkers). In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 65), (Gly₃Ser)ₙ(Gly₄Ser)ₙ (SEQ ID NO: 66), (Gly₃Ser)ₙ(Gly₂Ser)ₙ (SEQ ID NO: 67), and (Gly₃Ser)ₙ(Gly₄Ser)₁ (SEQ ID NO: 68). In particular embodiments, the linker is (Gly₄Ser)₄ (SEQ ID NO: 69), (Gly₄Ser)₃ (SEQ ID NO: 49), (Gly₄Ser)₂ (SEQ ID NO: 70), (Gly₄Ser)₁ (SEQ ID NO: 71), (Gly₃Ser)₂ (SEQ ID NO: 72), (Gly₃Ser)₁ (SEQ ID NO: 73), (Gly₂Ser)₂ (SEQ ID NO: 74) or (Gly₂Ser)₁, GGSGGGSGGSG (SEQ ID NO: 75), GGSGGGSGSG (SEQ ID NO: 76), or GGSGGGSG (SEQ ID NO: 77).

For additional examples of and information regarding linkers, see Chen et al., Adv Drug Deliv Rev. 2013 Oct. 15; 65(10): 1357-1369.

Additional examples of engineered antigen binding domain formats include scFv-based grababodies and soluble VH domain antibodies. These binding domains form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

A Fab fragment is a monovalent antibody fragment including V_{L}, V_{H}, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90:6444-6448, 1993. Dual affinity retargeting antibodies (DART™; based on the diabody format but featuring a C-terminal disulfide bridge for additional stabilization (Moore et al., Blood 117:4542-51, 2011)) can also be used. Engineered antibody fragments can also include isolated CDRs. For a review of antibody fragments, see Hudson, et al., Nat. Med. 9:129-134, 2003.

(ii) Exemplary Anti-Viral Binding Domains. Engineered antigen binding domains and engineered BCR can be derived from numerous antibodies, depending on the selected antigen of interest. For example, engineered antigen binding domains and engineered BCR can be prepared from an antibody that can provide a protective effect against a pathogen or condition (e.g., autoimmune disease). In particular embodiments, the engineered antigen binding domain is derived from an anti-RSV antibody, an anti-HIV antibody, an anti-Dengue virus antibody, an anti-Bordatella pertussis antibody, an anti-hepatitis C antibody, an anti-influenza virus antibody, an anti-parainfluenza virus antibody, an anti-metapneumovirus (MPV) antibody, an anti-cytomegalovirus antibody, an anti-Epstein Barr virus antibody; an anti-herpes simplex virus antibody, an anti-Clostridium difficile bacterial toxin antibody, or an anti-tumor necrosis factor (TNF) antibody.

In particular embodiments, engineered BCR are chimeric. In particular embodiments, chimeric BCR refer to a synthetic BCR that includes: (i) at least one portion that is encoded by a B cell's endogenous genome, and (ii) at least one portion that is encoded by an inserted nucleic acid. In particular embodiments, the chimeric BCR includes an endogenous heavy chain constant domain, an exogenous immunoglobulin variable and constant light chain, and an exogenous variable heavy chain. See, for example, WO 2019/079772.

The following antibodies and sequences are useful to generate engineered antigen binding domains and/or engineered BCR with targeted binding against pathogens or antigens of interest (unless noted, Kabat numbering is intended):

An exemplary anti-RSV antibody is palivizumab, which targets the RSV fusion protein and is used to prevent or reduce RSV infections.

In particular embodiments, an anti-RSV antibody is mouse palivizumab that includes a variable

heavy chain sequence including:
(SEQ ID NO: 78)
QVELQESGPGILQPSQTLSLTCSFSGFSLSTSGMSVGWIRQPSGEGLEWL
ADIWWDDKKDYNPSLKSRLTISKDTSSNQVFLKITGVDTADTATYYCARS
MITNWYFDVWGAGTTVTVSS;
and
a variable light chain sequence including:
(SEQ ID NO: 79)
DIQLTQSPAIMSASPGEKVTMTCSASSSVGYMHWYQQKLSTSPKLQIYDT
SKLASGVPGRFSGSGSGNSYSLTISSIQAEDVATYYCFRGSGYPFTFGQG
TKLEIK.

An additional exemplary anti-RSV antibody is human palivizumab and includes a variable

light chain sequence including:
(SEQ ID NO: 80)
DIQMTQSPSTLSASVGDRVTITCKCQLSVGYMHWYQQKPGKAPKLLIYDT
SKLASGVPSRFSGSGSGTEFTLTISSLQPDFATYYCFQGSGYPFTFGGGT
KLEIKR;
and
a variable heavy chain sequence including:
(SEQ ID NO: 81)
QVTLRESGPALVKPTQTLTLTCTFSGFSLSTSGMSVGWIRQPPGKALEWL
ADIWWDDKKDYNPSLKSRLTISKDTSKNQVVLKVTNMDPADTATYYCARS
MITNWYFDVWGAGTT.

Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3)

An additional exemplary anti-RSV antibody is described in U.S. Pat. No. 9,403,900. This anti-RSV antibody includes a variable heavy chain including a CDRH1 sequence including GASINSDNYYWT (SEQ ID NO: 82), a CDRH2 sequence including HISYTGNTYYTPSLKS (SEQ ID NO: 83), and a CDRH3 sequence including CGAYVLISNCGWFDS (SEQ ID NO: 84); and a variable light chain including a CDRL1 sequence including QASQDISTYLN (SEQ ID NO: 85), a CDRL2 sequence including GASNLET (SEQ ID NO: 86), and a CDRL3 sequence including QQYQYLPYT (SEQ ID NO: 87).

Exemplary anti-RSV antibodies also include AB1 128 (available from MILLIPORE) and ab20745 (available from ABCAM).

An example of an anti-HIV antibody is 10E8, which is a broadly neutralizing antibody that binds to gp41. The 10E8 anti-HIV antibody includes a variable heavy chain including a CDRH1 sequence including GFDFDNAW (SEQ ID NO: 88), a CDRH2 sequence including ITGPGEGWSV (SEQ ID NO: 89), and a CDRH3 sequence including TGKYYDFWSGYPPGEEYFQD (SEQ ID NO: 90); and a variable light chain including a CDRL1 sequence including TGDSLRSHYAS (SEQ ID NO: 91), a CDRL2 sequence including GKNNRPS (SEQ ID NO: 92), and a CDRL3 sequence including SSRDKSGSRLSV (SEQ ID NO: 93).

An additional example of an anti-HIV antibody is VRC01, which is a broadly neutralizing antibody that binds to the CD4 binding site of gp120. The VRC01 antibody includes a variable heavy chain including a CDRH1 sequence including GYEFIDCT (SEQ ID NO: 94), a CDRH2 sequence including KPRGGAVN (SEQ ID NO: 95), and a CDRH3 sequence including RGKNCDYNWDFEHW (SEQ ID NO: 96); and a variable light chain including a CDRL1 sequence including QYGS (SEQ ID NO: 97), a CDRL2 sequence including SGS, and a CDRL3 sequence including QQYEF (SEQ ID NO: 98).

Exemplary anti-HIV antibodies also include ab18633 and 39/5.4A (available from ABCAM); and H81E (available from THERMOFISHER).

An example of an anti-Dengue virus antibody is antibody 55 described in U.S. 20170233460 and includes a variable heavy chain including a CDRH1 sequence including EVQLHQSGAELVKPGASVKLSCTVSGFNIK (SEQ ID NO: 99), a CDRH2 sequence including WVKQRPEQGLEWI (SEQ ID NO: 100), and a CDRH3 sequence including ATIKADTSSNTAYLQLISLTSEDTAVYYCAF (SEQ ID NO: 101); and a variable light chain including a CDRL1 sequence including DIQMTQSPASLSVSVGETVTITC (SEQ ID NO: 102), a CDRL2 sequence including WYQQKQGKSPQLLVY (SEQ ID NO: 103), and a CDRL3 sequence including GVPSRFSGSGSGTQYSLKINSLQSEDFGTYYC (SEQ ID NO: 104).

An additional example of an anti-Dengue virus antibody is DB2-3 described in U.S. Pat. No. 8,637,035 and includes a variable heavy chain including a CDRH1 sequence including YTFTDYAIT (SEQ ID NO: 105), a CDRH2 sequence including GLISTYYGDSFYNQKFKG (SEQ ID NO: 106), and a CDRH3 sequence including TIRDGKAMDY (SEQ ID NO: 107); and a variable light chain including a CDRL1 sequence including RSSQSLVHSNGNTYLH (SEQ ID NO: 108), a CDRL2 sequence including KVSNRFS (SEQ ID NO: 109), and a CDRL3 sequence including SQSTHVPYT (SEQ ID NO: 110). Examples of anti-Dengue virus antibodies also include ab155042 and ab80914 (both available from ABCAM).

An example of an anti-pertussis antibody is described in U.S. Pat. No. 9,512,204 and includes a variable heavy chain including QVQLQQPGSELVRPGASVKLSCKASGYKFTS YWMHWVKQRPGQGLEWIGNIFPGSGSTNYDEKFNSKATLTVDTSSNTAYMQLSSLTSEDSAV YYCTRWLSGAYFDYWGQGTTVTVSS (SEQ ID NO: 111) and a variable light chain including QIVLTQSPALMSASPGEKVTMTCSASSSVSFMYWYQQKPRSSPKPWIYLTSNLPSGVPARFSG SGSGTSYSLTISSMEAEDAATYYCQQWSSHPPTFGSGTKLEIK (SEQ ID NO: 112).

An example of an anti-hepatitis C antibody includes a variable heavy chain including a CDRH1 sequence including SYGMHW (SEQ ID NO: 113), a CDRH2 sequence including VIWLDGSNTYYADSVKGR (SEQ ID NO: 114), and a CDRH3 sequence including ARDIFTVARGVIIYFDY (SEQ ID NO: 115); and a variable light chain including a CDRL1 sequence including RASQSVSSYLA (SEQ ID NO: 116), a CDRL2 sequence including DASNRAT (SEQ ID NO: 117), and a CDRL3 sequence including QQRSNWVT (SEQ ID NO: 118). Examples of anti-hepatitis C antibodies also include MAB8694 (available from MILLIPORE) and C7-50 (available from ABCAM).

An example of an anti-influenza virus antibody is described U.S. Pat. No. 9,469,685 and includes a variable heavy chain including a CDRH1 sequence including GMTSNSLA (SEQ ID NO: 119), a CDRH2 sequence including IIPVFETP (SEQ ID NO: 120), and a CDRH3 sequence including ATSAGGIVNYYLSFNI (SEQ ID NO: 121); and a variable light chain including a CDRL1 sequence including QTITTW (SEQ ID NO: 122), a CDRL2 sequence including KTS, and a CDRL3 sequence including QQYSTYSGT (SEQ ID NO: 123). An example of an anti-influenza virus antibody also includes C102 (available from THERMOFISHER).

An exemplary anti-MPV antibody includes MPE8.

Exemplary anti-CMV antibodies includes MCMV5322A, MCMV3068A, LJP538, and LJP539. RG7667 includes a mixture of MCMV5322A and MCMV3068A while CSJ148 includes a mixture of LJP538, and LJP539. See also, for example, Deng et al., Antimicrobial Agents and Chemotherapy 62(2) e01108-17 (February 2018); and Dole et al., Antimicrobial Agents and Chemotherapy 60(5) 2881-2887 (May 2016).

An example of an anti-EBV antibody includes a variable heavy chain including an AMMO1 CDRH1 sequence including YTFIHFGISW (SEQ ID NO: 124), an AMMO1 CDRH2 sequence including IDTNNGNTNYAQSLQG (SEQ ID NO: 125), and an AMMO1 CDRH3 sequence including RALEMGHRSGFPFDY (SEQ ID NO: 126); and a variable light chain including an AMMO1 CDRL1 sequence including GGHNIGAKNVH (SEQ ID NO: 127), an AMMO1 CDRL2 sequence including YDSDRPS (SEQ ID NO: 128), and an AMMO1 CDRL3 sequence including CQVWDSGRGHPLYV (SEQ ID NO: 129).

An example of an anti-HSV antibody includes HSV8-N and MB66.

Exemplary anti-Clostridium difficile antibodies include actoxumab and bezlotoxumab. See also, for example, Wilcox et al., N Engl J Med 376(4) 305-317 (2017).

Numerous additional antibody sequences are available and known to those of ordinary skill in the art that can be used within the teachings of the current disclosure. Sequence information for commercially available antibodies may be found in the Drug Bank database, the CAS Registry, and/or the RSCB Protein Data Bank. Moreover, nucleic acid sequences encoding portions of selected antibodies described herein can be easily derived by one of ordinary skill in the art.

As indicated previously, when an engineered antigen binding domain is derived from an antibody, it means that the engineered antigen binding domain includes the binding domain of an antibody. Including the binding domain of an antibody means that the engineered antigen binding domain has sufficient sequence similarity with the binding domain of the antibody that it has substantially similar binding properties. In certain examples, an engineered antigen binding domain derived from an antibody has the same CDRs of the antibody, as defined by any relevant CDR numbering scheme or prediction algorithm. Examples include Kabat et al. (1991) “Sequences of Proteins of Immunological Interest,” 5th Ed. Public Health Service, National Institutes of Health, Bethesda, Md. (Kabat numbering scheme); AI-Lazikani et al. (1997) J Mol Biol 273: 927-948 (Chothia numbering scheme); Maccallum et al. (1996) J Mol Biol 262: 732-745 (Contact numbering scheme); Martin et al. (1989) Proc. Natl. Acad. Sci., 86: 9268-9272 (AbM numbering scheme); North et al. (2011) J. Mol. Biol. 406(2):228-56 (North numbering scheme); Lefranc M P et al. (2003) Dev Comp Immunol 27(1): 55-77 (IMGT numbering scheme); and Honegger and Pluckthun (2001) J Mol Biol 309(3): 657-670 (“Aho” numbering scheme) and software programs such as ABodyBuilder. The boundaries of a given CDR or FR may vary depending on the scheme used for identification. In certain examples, an engineered antigen binding domain derived from an antibody has the same VL and VH chain as the antibody (i.e., 100% sequence identity) or can have up to 10 residue additions, deletions, or substitutions outside of CDR residues. In certain examples, an engineered antigen binding domain derived from an antibody has a VL with 90%, 91%, 92%, 93%, 94%, 95%, 96%, 97%, 98%, 99%, or 100% sequence identity to the VL of the antibody and a VH with 90%, 91%, 92%, 93%, 94%, 95%, 96%, 97%, 98%, 99%, or 100% sequence identity to the VH of the antibody.

(iii) Spacers, Linkers, and Junction Amino Acids. Various forms of fusion proteins described herein can include spacers, linkers, and/or junction amino acids that connect different portions of the fusion proteins.

Spacer regions are generally used to create appropriate distances and/or flexibility from other fusion protein sub-components. In particular embodiments, the length of a spacer region can be customized for binding different antigens (e.g., viral antigens).

Spacer regions typically include those having 35 to 250 amino acids, 35 to 200 amino acids, 35 to 150 amino acids, 35 to 100 amino acids, or 35 to 50 amino acids.

Exemplary spacer regions include all or a portion of an extracellular CD40 domain or an immunoglobulin hinge region. An extracellular CD40 domain or immunoglobulin hinge region may be a wild-type extracellular CD40 domain or immunoglobulin hinge region or an altered wild-type extracellular CD40 domain or immunoglobulin hinge region. In certain embodiments, an extracellular CD40 domain or immunoglobulin hinge region is a human or murine extracellular CD40 domain or immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region.

Other examples of hinge regions that can be used as spacers in fusion proteins described herein include the hinge region present in the extracellular regions of type 1 membrane proteins, such as CD8α, CD4, CD28 and CD7, which may be wild-type or variants thereof.

A linker can include any portion of a fusion protein that serves to connect two other subcomponents of the fusion protein. Some linkers serve no purpose other than to link components while many linkers serve an additional purpose. As used herein, linkers are shorter than spacers (i.e., less than 35 amino acid residues).

Linkers can be flexible, rigid, or semi-rigid, depending on the desired function of the linker. Gly-Ser linkers are described above in relation to scFv. In this scenario, Gly-Ser linkers provide flexibility and room for conformational movement between different components of fusion proteins. Linkers may also be Gly linkers (e.g., Gly₄₋₂₀ including particularly Gly₄, Gly₅, Gly₆, Gly₇, and Gly₈.

In some situations, flexible linkers may be incapable of maintaining a distance or positioning of fusion proteins needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or noncleavable linker). In some aspects, the linker is a procharged linker, a hydrophilic linker, or a dicarboxylic acid-based linker.

Junction amino acids can be a linker which can be used to connect sequences when the distance provided by a spacer region or larger linker is not needed and/or wanted. For example, junction amino acids can be short amino acid sequences that can be used to connect intracellular signaling components to tags, when present. In particular embodiments, junction amino acids are 3 amino acids or less. In particular embodiments, a glycine-serine doublet can be used as a suitable junction amino acid linker. In particular embodiments, a single amino acid, e.g., an alanine, a glycine, can be used as a suitable junction amino acid.

(iv) Tags. Tags can be used for multiple purposes in the fusion proteins described herein. In some embodiments, tags are used during cell manufacturing for the purposes of cell isolation or tracking. Tags also have in vivo uses, for example, for tracking, activating, or depleting cells once administered.

Exemplary tags include e.g., Strep tag (e.g., the original STREP® tag, STREP® tag II, or any variant thereof; see, e.g., U.S. Pat. No. 7,981,632), His tag, Flag tag, Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), Thioredoxin tag, or any combination thereof. Antibodies and binding domains to these tags are commercially available.

Tags may be present in multiple copies in fusion proteins. For example, a fusion protein can have one, two, three, four or five tags (e.g., Strep tag). In certain embodiments, a fusion protein can include one tag, two tags, three tags, four tags, or five tags. Each of the plurality of tags may be the same or different. Exemplary embodiments include a fusion protein having a Strep tag and an HA tag, a His tag and a Strep tag, or a Myc tag and a Strep tag. Alternatively, a fusion protein can have multiple tags of the same type or same amino acid sequence, such as two, three, four or five Strep tags (e.g., Strep tag II).

Tag binding molecules (e.g., receptor, protein, antibody) may be soluble, part of a matrix composition, or conjugated to a solid surface (e.g., plate, bead). Exemplary solid surfaces include beads and particles (e.g., micro and nano), such as magnetic beads and particles.

(v) Multimerization Molecules. In certain instances, it can be beneficial to include multimerization molecules within the disclosed fusion proteins. In particular embodiments, the multimerization molecules can be part of a chemically-induced dimerization (CID) system. These systems require that one fusion protein includes a chemical inducer of dimerization binding domain 1 (CBD1) and a second molecule (e.g., second fusion protein) includes a second chemical inducer of dimerization binding domain (CBD2), wherein CBD1 and CBD2 are capable of simultaneously binding to the chemical inducer of dimerization (CID). If the CID is rapamycin, CBD1 and CBD2 can be the rapamycin binding domain of FK-binding protein 12 (FKBP12) (SEQ ID NO: 40) and the FKBP12-Rapamycin Binding (FRB) domain of mTOR (SEQ ID NO: 41). If the CID is FK506/cyclosporin fusion protein or a derivative thereof, CBD1 and CBD2 can be the FK506 (Tacrolimus) binding domain of FK-binding protein 12 (FKBP12) and the cyclosporin binding domain of cylcophilin A. If the CID is estrone/biotin fusion protein or a derivative thereof, CBD1 and CBD2 can be an oestrogen-binding domain (EBD) and a streptavidin binding domain. If the CID is dexamethasone/methotrexate fusion molecule or a derivative thereof, CBD1 and CBD2 can be a glucocorticoid-binding domain (GBD) and a dihydrofolate reductase (DHFR) binding domain. If the CID is O⁶-benzylguanine derivative/methotrexate fusion molecule or a derivative thereof, CBD1 and CBD2 can be an O⁶-alkylguanine-DNA alkyltransferase (AGT) binding domain and a dihydrofolate reductase (DHFR) binding domain. If the CID is RSL1 or a derivative thereof, CBD1 and CBD2 can be a retinoic acid receptor domain and an ecodysone receptor domain. If the CID is AP1903 or a derivative thereof, CBD1 and CBD2 can be the FK506 binding protein (FKBP12) binding domains including a F36V mutation. Use of the CID binding domains can also be used to alter the affinity to the CID. For instance, altering amino acids at positions 2095, 2098, and 2101 of FRB can alter binding to Rapamycin: KTW has high, KHF intermediate and PLW is low (Bayle et al, Chemistry & Biology 13, 99-107, January 2006).

Examples of dimerization molecules that do not rely on CID include protein sequence motifs such as coiled coils, acid patches, zinc fingers, calcium hands, a CH1-CL pair, an “interface” with an engineered “knob” and/or “protruberance” (U.S. Pat. No. 5,821,333), leucine zippers (U.S. Pat. No. 5,932,448), SH2 and SH3 (Vidal et al., Biochemistry, 43:7336-44, 2004), PTB (Zhou et al., Nature, 378:584-592, 1995), WW(Sudol Prog Biochys MoL Bio, 65:113-132, 1996), PDZ (Kim et al., Nature, 378: 85-88, 1995; Komau et al., Science, 269:1737-1740, 1995) and WD40 (Hu et al., J Biol Chem., 273:33489-33494, 1998). Additional examples of molecules that contain dimerization domains/motifs are receptor dimer pairs such as the interleukin-8 receptor (IL-8R), integrin heterodimers such as LFA-I and GPIIIb/Illa, dimeric ligand polypeptides such as nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa et al., J Biol. Chem., 269:27833-27839, 1994; Radziejewski et al., Biochem, 32: 1350, 1993) and variants of some of these domains with modified affinities (PCT Publication No. WO 2012/001647).

(vi) Transmembrane Domains. Particular embodiments disclosed herein include a transmembrane domain. As indicated, transmembrane domains within fusion proteins serve to connect the extracellular component and intracellular component through the cell membrane. The transmembrane domain can anchor the expressed molecule in the modified cell's membrane.

In particular embodiments, a transmembrane domain has a three-dimensional structure that is thermodynamically stable in a cell membrane, and generally ranges in length from 15 to 30 amino acids. The structure of a transmembrane domain can include an a helix, a β barrel, a β sheet, α β helix, or any combination thereof.

The transmembrane domain can be derived either from a natural and/or a synthetic source. When the source is natural, the transmembrane domain can be derived from any membrane-bound or transmembrane protein. Transmembrane domains can include at least the transmembrane region of CD40. Transmembrane domains can also be derived from transmembrane region(s) of CD2, CD3, CD4, CD5, CD8, CD9, CD11a, CD16, CD18, CD19, CD22; CD27, CD28, CD29, CD33, CD37, CD45, CD49a, CD49d, CD49f, CD64, CD80, CD84, CD86, CD96, CD100, CD103, CD134, CD137, CD150, CD154, CD160, CD162, CD226, CD229, CD244, CD278, KIRDS2, OX40, LFA-1, GITR, BAFFR, HVEM (LIGHTR), SLAMF7, NKp80 (KLRF1), NKp44, NKp30, NKp46, IL2R3, IL2Ry, IL7R a, ITGA1, VLA1, ITGA4, IA4, ITGA6, VLA-6, ITGAD, ITGAE, ITGAL, ITGAX, ITGB1, ITGB2, ITGB7, TNFR2, CEACAMI, CRT AM, PSGL1, SLAMF6 (NTB-A, Ly108), BLAME (SLAMF8), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge.

A transmembrane domain can include one or more additional amino acids adjacent to the transmembrane region, e.g., one or more amino acid within the extracellular region of the fusion protein (e.g., up to 15 amino acids of the extracellular region) and/or one or more additional amino acids within the intracellular region of the fusion protein (e.g., up to 15 amino acids of the intracellular components). In one aspect, the transmembrane domain is from the same protein as the signaling domain (i.e., is derived from CD40). In another aspect, the transmembrane domain is derived from a protein that is not otherwise represented in any other segment of the fusion protein.

(vii) Skipping Elements. In any of the embodiments described herein, a nucleic acid can include a polynucleotide that encodes or includes a skipping element. In certain examples, the skipping element is placed between the polynucleotide segment encoding the portion of the fusion protein including the CD40 signaling domain and the polynucleotide segment encoding a reporter.

Exemplary skipping elements include self-cleaving peptides, such as the self-cleaving “2A” peptides. 2A peptides function by causing the ribosome to skip the synthesis of a peptide bond at a defined location, leading to production of two proteins from one mRNA. The 2A sequences are short (e.g., 20 amino acids), facilitating use in size-limited constructs, and proteins are produced at a 1:1 ratio. Exemplary self-cleaving 2A peptides include and/or are derived from porcine teschovirus-1 (P2A (GSG)ATNFSLLKQAGDVEENPGP (SEQ ID NO: 130)), Thosea asigna virus (T2A (GSG)EGRGSLLTCGDVEENPGP (SEQ ID NO: 131)), equine rhinitis A virus (E2A (GSG)QCTNYALLKLAGDVES NPGPP (SEQ ID NO: 132)), foot-and-mouth disease virus (F2A (GSG)VKQTLNFDLLKLAGDVESNPGP (SEQ ID NO: 133)), or variants thereof. Further exemplary nucleic acid and amino acid sequences of 2A peptides are set forth in, for example, Kim et al. (PLOS One 6:e18556 (2011)).

Internal ribosome entry site (IRES) sequences can also be used as skipping elements. IRES are non-coding structured RNA sequences that allow ribosomes to initiate translation at a second internal site on a mRNA molecule, leading to production of two proteins from one mRNA.

(viii) Reporters. As indicated, fusion proteins disclosed herein can include reporters, that, in certain instances are separated from the majority of the fusion protein after expression. Exemplary reporter genes/proteins include fluorescent proteins such blue fluorescent proteins (e.g. eBFP, eBFP2, Azurite, mKalamal, GFPuv, Sapphire, T-sapphire); cyan fluorescent proteins (e.g. eCFP, Cerulean, CyPet, AmCyanl, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen), CopGFP, AceGFP, avGFP, ZsGreenl, Oregon Green™(Thermo Fisher Scientific)); Luciferase; orange fluorescent proteins (mOrange, mKO, Kusabira-Orange, Monomeric Kusabira-Orange, mTangerine, tdTomato, dTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowl); and tandem conjugates. EGFR and truncated EGFR (tEGFR) can also be used.

(ix) Genetic Modification and Cell Sorting. Any technique known to those of ordinary skill in the art can be utilized to introduce nucleic acids encoding fusion proteins into B cells for expression.

Coding sequences encoding particular examples of fusion proteins described herein are provided in FIGS. 10A-10H and 11. To the extent not explicitly disclosed, coding sequences can be obtained from publicly available databases, publications, and the knowledge of those or ordinary skill in the art. Coding sequences can further include sequence variants wherein the alterations do not affect (or significantly affect) the function of the encoded molecule. In certain examples, sequence variants are due to different codon optimization strategies. The terms “code”, “coding”, “encode” and “encoding” refer to a property of sequences of nucleic acids, such as a vector, a plasmid, a gene, cDNA, or mRNA, to serve as templates for synthesis of other molecules such as proteins.

Nucleic acids may include genes. The term “gene” may include not only coding sequences but also regulatory regions such as promoters, enhancers, insulators, and/or post-regulatory elements, such as termination regions.

In particular embodiments, nucleic acids including genes are provided to B cells within vectors. The term vector refers to a nucleic acid molecule capable of transferring or transporting another nucleic acid molecule, such as a gene. A vector may include sequences that direct autonomous replication in a cell or may include sequences that permit integration into host cell DNA. Useful vectors include, for example, plasmids (e.g., DNA plasmids or RNA plasmids), transposons, cosmids, bacterial artificial chromosomes, and viral vectors. Each of these may be used to deliver nucleic acids to B cells.

Gene editing systems allow control over target sites for genetic insertions or deletions in the genome. Within the teachings of the current disclosure, any gene editing system capable of precise sequence targeting and modification can also be used. These systems typically include a targeting element for precise targeting and a cutting element for cutting the targeted genetic site. Guide RNA is one example of a targeting element while various nucleases provide examples of cutting elements. Targeting elements and cutting elements can be separate molecules or linked, for example, by a nanoparticle. Alternatively, a targeting element and a cutting element can be linked together into one dual purpose molecule. When insertion of a therapeutic nucleic acid sequence is intended, the systems can also include homology-directed repair templates (i.e., homology arms as described above) associated with the nucleic acid intended for insertion.

Particular embodiments utilize zinc finger nucleases (ZFNs), transcription activator like effector nucleases (TALENs), MegaTALs, and/or CRISPR-based systems for genome editing.

For information regarding ZFNs, see Kim, et al. Proceedings of the National Academy of Sciences of the United States of America 93, 1156-1160 (1996); Wolfe, et al. Annual review of biophysics and biomolecular structure 29, 183-212 (2000); Bibikova, et al. Science 300, 764 (2003); Bibikova, et al. Genetics 161, 1169-1175 (2002); Miller, et al. The EMBO journal 4, 1609-1614 (1985); and Miller, et al. Nature biotechnology 25, 778-785 (2007).

For information regarding TALENs, see Boch, et al. Science 326, 1509-1512 (2009); Moscou, & Bogdanove, Science 326, 1501 (2009); Christian, et al. Genetics 186, 757-761 (2010); and Miller, et al. Nature biotechnology 29, 143-148 (2011).

The CRISPR nuclease system is a prokaryotic immune system that confers resistance to foreign genetic elements such as plasmids and phages and provides a form of acquired immunity. Within CRISPR systems, a nuclease enzyme can be programmed by a short guide RNA (gRNA) molecule to recognize a specific DNA target. Within CRISPR systems, gRNA can be referred to as crRNA.

At least three different Cas9 nucleases have been developed for genome editing. The first is the wild type Cas9 which introduces double strand breaks (DSBs) at a specific DNA site, resulting in the activation of DSB repair machinery. DSBs can be repaired by non-homologous end joining (NHEJ), homology-directed repair (HDR), or microhomology mediated repair (MMEJ). NHEJ can involve repair of a DSB with no homology (<5 bp) between the two ends joined during repair; HDR can involve repair of a DSB with a large region of homology between the ends joined during repair (100 or more nucleotides); and MMEJ can involve repair of a DSB with a small (5 to 50 bp) region of homology between the ends joined during repair. Another type of Cas9 includes a mutant Cas9, known as the Cas9D10A, with only nickase activity, which means that it only cleaves one DNA strand and does not activate NHEJ. Thus, the DNA repairs proceed via the HDR pathway only. The third is a nuclease-deficient Cas9 (dCas9) which does not have cleavage activity but is able to bind DNA. Therefore, dCas9 is able to target specific sequences of a genome without cleavage. By fusing dCas9 with various effector domains, dCas9 can be used either as a gene silencing or activation tool.

Cpf1 is another nuclease suitable for use within CRISPR-based systems (see Zetsche et al. (2015) Cell 163(3): 759-771). The Cpf1 nuclease particularly can provide added flexibility in target site selection by means of a short, three base pair recognition sequence (TTN), known as the protospacer-adjacent motif or PAM. Cpf1's cut site is at least 18 bp away from the PAM sequence, thus the enzyme can repeatedly cut a specified locus after indel (insertion and deletion) formation, increasing the efficiency of HDR. Moreover, staggered DSBs with sticky ends permit orientation-specific donor template insertion.

Additional information regarding CRISPR-Cas systems and components thereof are described in, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

In particular embodiments, nucleotide segments encoding scFv or CD40-CD79α/3 linked molecules can be inserted into the light chromosome locus, for example, to knockout endogenous expression.

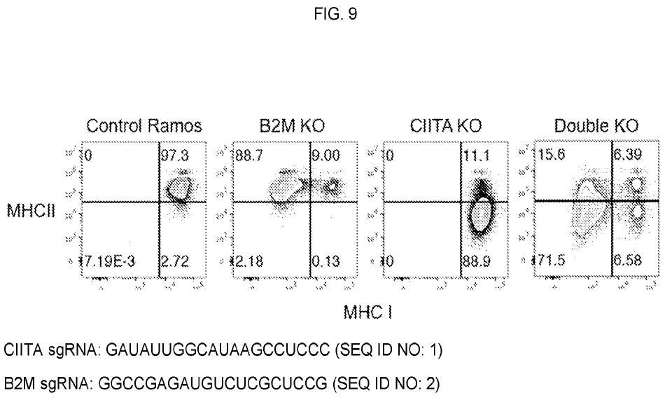

When genetic modifications reduce expression of a transcription factor to delete MHC class II molecules, gRNA molecules for CIITA deletion include: UCGAGUUGGAUGUGGAAGGU (SEQ ID NO: 134), UUUUCAUCCCCACUUCACAC (SEQ ID NO: 135), CCUCGGGGGAGAGAGAGGUG (SEQ ID NO: 136), UGGGCUCAGGUGCUUCCUCA (SEQ ID NO: 137), UCAAAGUAGAGCACAUAGGA (SEQ ID NO: 138), CCAUCAAAAGUCCUUUUUGG (SEQ ID NO: 139), GUGUCUACACUUAGCCUUUC (SEQ ID NO: 140), GGGUGAAAUUUCCCAACUUU (SEQ ID NO: 141), CCGGCCUUUUUACCUUGGGG (SEQ ID NO: 142), and UCUGCAGCCUUCCCAGAGGA (SEQ ID NO: 143). An sgRNA sequence for CIITA deletion is also provided in FIG. 9 as SEQ ID NO: 1.

gRNA molecules for TRAC deletion include: UUGCUCCAGGCCACAGCACU (SEQ ID NO: 144), UCGACCAGCUUGACAUCACA (SEQ ID NO: 145), AGAAUCAAAAUCGGUGAAUA (SEQ ID NO: 146), CAUGUGCAAACGCCUUCAAC (SEQ ID NO: 147), AAAGUUUAGGUUCGUAUCUG (SEQ ID NO: 148), UUUGAGAAUCAAAAUCGGUG (SEQ ID NO: 149), AUUCUCAAACAAAUGUGUCA (SEQ ID NO: 150), CUUUUAGAAAGUUCCUGUGA (SEQ ID NO: 151), AAAGCUUUUCUCGACCAGCU (SEQ ID NO: 152), and GAGUCUCUCAGCUGGUACAC (SEQ ID NO: 153).

gRNA molecules for TRBC deletion include: CAGAGGACCUGAAAAACGUG (SEQ ID NO: 154), AGGUCCUCUGGAAAGGGAAG (SEQ ID NO: 155), AGCCAUCAGAAGCAGAGAUC (SEQ ID NO: 156), GGUGUGGGAGAUCUCUGCUU (SEQ ID NO: 157), GCCCUAUCCUGGGUCCACUC (SEQ ID NO: 158), UUCCCCUGUUUUCUUUCAGA (SEQ ID NO: 159), UUUCAGACUGUGGCUUCACC (SEQ ID NO: 160), AGGCCUCGGCGCUGACGAUC (SEQ ID NO: 161), CAGGCCCCACUCACCUGCUC (SEQ ID NO: 162), and AGGCCCCACUCACCUGCUCU (SEQ ID NO: 163).

gRNA molecules for B2M deletion include: UGGCCUGGAGGCUAUCCAGC (SEQ ID NO: 164), CCGAUAUUCCUCAGGUACUC (SEQ ID NO: 165), GAGUACCUGAGGAAUAUCGG (SEQ ID NO: 166), CUCACGUCAUCCAGCAGAGA (SEQ ID NO: 167), CAUUCUCUGCUGGAUGACGU (SEQ ID NO: 168), ACUUUCCAUUCUCUGCUGGA (SEQ ID NO: 169), CUGAAUUGCUAUGUGUCUGG (SEQ ID NO: 170), AUCCAUCCGACAUUGAAGUU (SEQ ID NO: 171), AAUUCUCUCUCCAUUCUUCA (SEQ ID NO: 172), and AGCAAGGACUGGUCUUUCUA (SEQ ID NO: 173). An sgRNA sequence for B2M deletion is also provided in FIG. 9 as SEQ ID NO: 2.

For additional information regarding gRNAs to delete genes encoding CIITA, TRAC, TRBC, and B2M, see US 2020/0299661.

RNA molecule for RFX5 deletion include:

| (SEQ ID NO: 174) | |
|---|---|
| | AAAAUGUUUUAAUGUAUAUGUGUUA, |
| (SEQ ID NO: 175) | |
| | AAUGUUUUAAUGUAUAUGUGUUAU, |
| (SEQ ID NO: 176) | |
| | UAUUAUAAUUUCUUAAAAUCUGCUG, |
| (SEQ ID NO: 177) | |
| | UAUAAUUUCUUAAAAUCUGCUGUGG, |
| (SEQ ID NO: 178) | |
| | UCCCUGAAAGAAAAUCAGUGUUUCU, |
| (SEQ ID NO: 179) | |
| | CCCUGAAAGAAAAUCAGUGUUUCUU, |
| (SEQ ID NO: 180) | |
| | CCCUGACAGAAGCUACCCUAUGUAG, |
| (SEQ ID NO: 181) | |
| | AGCUACCCUAUGUAGAGGACAAAGU, |
| (SEQ ID NO: 182) | |
| | AAGUAGGUCUUCAAUAAAUAUUAGU, |
| | and |
| (SEQ ID NO: 183) | |
| | AUUAGUUGGUUUACUGCUUUUCCCA. |

gRNA molecules for RFXAP deletion include:

| (SEQ ID NO: 184) | |
|---|---|
| | CGCGGGCGUACACACGCUGACGCGC, |
| (SEQ ID NO: 185) | |
| | CGUACACACGCUGACGCGCAGGCUG, |
| (SEQ ID NO: 186) | |
| | GCUGACGCGCAGGCUGCGGUCGCGC, |
| (SEQ ID NO: 187) | |
| | CAGGCUGCGGUCGCGCAGGCGCAGU, |
| (SEQ ID NO: 188) | |
| | GGCUGCGGUCGCGCAGGCGCAGUCG, |
| (SEQ ID NO: 189) | |
| | CGCAGGCGCAGUCGGGGCGCCUUCC, |
| (SEQ ID NO: 190) | |
| | CGCAGUCGGGGCGCCUUCCCGGUAU, |
| (SEQ ID NO: 191) | |
| | UUUACCCCAGCGUGUCCUGAGUCUU, |
| | and |
| (SEQ ID NO: 192) | |
| | AGUCUUUGGUUCGCGAAGUGCCGUU |

For additional information regarding gRNAs to delete genes encoding RFX5 and RFXAP, see PCT/US2019/036,111.

gRNA sequences for RFXANK deletion include:

| (SEQ ID NO: 193) | |
|---|---|
| | CGGCTCCTCCATAGCGACCG, |
| (SEQ ID NO: 194) | |
| | CCTCCATAGCGACCGCGGCC, |
| (SEQ ID NO: 195) | |
| | GCGACCGCGGCCGAGCGAAC, |
| (SEQ ID NO: 196) | |
| | CGACCGCGGCCGAGCGAACC, |
| (SEQ ID NO: 197) | |
| | CGGCCGAGCGAACCGGGAAA, |
| (SEQ ID NO: 198) | |
| | AGCGAACCGGGAAACGGCAC, |
| (SEQ ID NO: 199) | |
| | GAACCGGGAAACGGCACCGG, |
| (SEQ ID NO: 200) | |
| | GGGAAACGGCACCGGAAGCC, |
| (SEQ ID NO: 201) | |
| | GAAACGGCACCGGAAGCCCG, |
| | and |
| (SEQ ID NO: 202) | |
| | AAACGGCACCGGAAGCCCGG. |

Additional gRNA molecules and targeting domains for RFXANK can be found in US 2019/0309259.

In particular embodiments, nucleic acids, and/or genome targeting and cutting elements can be administered through electroporation, nanoparticle-mediated delivery and/or viral vector delivery. Adeno-associated viral vectors include those derived from e.g., adenovirus 5 (Ad5), adenovirus 35 (Ad35), adenovirus 11 (Ad11), adenovirus 26 (Ad26), adenovirus 48 (Ad48) or adenovirus 50 (Ad50)), and adeno-associated virus (AAV; see, e.g., U.S. Pat. No. 5,604,090; Kay et al., Nat. Genet. 24:257 (2000); Nakai et al., Blood 91:4600 (1998)).

In particular embodiments, genome targeting and cutting elements can be administered through electroporation and nucleic acids for insertion can be administered through AAV-mediated delivery. In particular embodiments, genome targeting and cutting elements can be administered through nanoparticle-mediated delivery and nucleic acids for insertion can be administered through AAV-mediated delivery.

In particular embodiments, a nucleic acid for delivery to B cells can be mixed with a targeting element (e.g., gRNA) and a cutting element (e.g., Cas9 or cpf1) immediately or shortly before electroporation. Selected fusion protein expression can be confirmed later (e.g., 3 days later) by measuring cell binding to fluorescently tagged target proteins by flow cytometry. Enrichment and analysis methodologies for detecting and analyzing epitope-specific B cells can be used. Pape et al., Science. 2011; 331(6021):1203-7; Taylor et al., J Exp Med. 2012; 209(3):597-606; Taylor et al., J Exp Med. 2012; 209(11):2065-77; Haasken et al., J Immunol. 2013; 191(3):1055-62; Taylor et al., J Immunol Methods. 2014; 405:74-86; Nanton et al., Eur J Immunol. 2015; 45(2):428-41; Hamilton et al., J Immunol. 2015; 194(10):5022-34; Taylor et al., Science. 2015; 347(6223):784-7).

In particular embodiments, cells can be identified and/or sorted based on marker expression, before or after delivering the nucleic acids. For example, it may be useful to isolate a particular type of B cells (e.g., memory B cells, antibody-secreting B cells, naïve B cells, B1B cells, marginal zone B cells) from a sample prior to delivering nucleic acids. As another example, it may be useful to isolate B cells from other cells present in a blood sample. CD19 is an example of a protein expressed by B cells but few other cells of the body. By marking CD19 with a fluorescent molecule, B cells can be specifically identified. B220 is a useful marker to identify mouse B cells.

CD27 is an example of a protein expressed by memory but not naive human B cells. By marking CD27 with a fluorescent molecule, memory B cells can be identified.

CD21 is an example of a protein not expressed (or expressed to a low degree) by some memory human B cells with the capacity to quickly secrete antibody following infection. Low CD21 expression can be used to define B cells primed for plasma cell differentiation. By marking CD21 with a fluorescent molecule, these B cells can be specifically identified by for example, negative selection.

Human naïve B cells can be identified by the marker profile IgM+ IgD+CD27−. Mouse naïve B cells can be identified by the marker profile CD38+GL7− IgM+ IgD+. Human B1 B cells can be identified by the marker profile CD5+CD43+. Mouse B1 B cells can be identified by the marker profile CD43+B220LOW. Human marginal zone B cells can be identified by the marker profile CD21+++IgM++ IgD—CD27+. Mouse marginal zone B cells can be identified by the marker profile CD21+++IgM++ IgD−.

Particular embodiments may utilize the CD19⁺CD27⁺CD21^{lo} marker profile.

As indicated, in particular embodiments, cells may be identified and/or isolated using flow cytometry. Flow cytometry is a sensitive and powerful analysis approach that uses lasers to individually analyze the fluorescent molecules marking millions of individual cells. By analyzing the combination of fluorescent molecules each cell is marked with, different B cell subtypes can be identified.

In particular embodiments, methods of modifying B cells can include obtaining hematopoietic stem cells (HSC), and/or delivering the nucleic acids to HSC. HSC can refer to a type of stem cell that naturally produces B cells as well as all other cells of the immune system. HSC can be obtained, for example, from cord blood.

In particular embodiments, B cells may be obtained from a human subject and obtained B cells or a subset thereof may be modified ex vivo.

(x) Formulations of Modified B Cells. Once modified, B cells can be harvested from a culture medium, and washed and concentrated into a carrier in a therapeutically-effective amount.

Exemplary carriers include saline, buffered saline, physiological saline, water, Hanks' solution, Ringer's solution, Nonnosol-R (Abbott Labs), PLASMA-LYTE A® (Baxter Laboratories, Inc., Morton Grove, IL), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% hyaluronic acid sodium salt (HAS) or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Formulations can include, for example, greater than 10² modified B cells, greater than 10³ modified B cells, greater than 10⁴ modified B cells, greater than 10⁵ modified B cells, greater than 10⁶ modified B cells, greater than 10⁷ modified B cells, greater than 10⁸ modified B cells, greater than 10⁹ modified B cells, greater than 10¹⁰ modified B cells, or greater than 10¹¹ modified B cells.

B cells formulation can be allogeneic or autologous to a subject, depending on whether the B cells were derived from the subject and/or whether MHC class II molecules have been deleted from the B cells.

(xi) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with formulations disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of a condition's development, progression, and/or resolution.

A “prophylactic treatment” includes a treatment administered to a subject who does not display signs or symptoms of a condition or displays only early signs or symptoms of a condition such that treatment is administered for the purpose of diminishing or decreasing the risk of developing the condition. Thus, a prophylactic treatment functions as a preventative treatment against a condition. In particular embodiments, prophylactic treatments reduce, delay, or prevent the worsening of a condition. Particular embodiments include administration of a formulation described herein as prophylactic protection in the absence of a currently effective vaccine. Particular embodiments include administration of a formulation described herein as prophylactic protection as a replacement for conventional vaccination strategies. Particular embodiments include administration of a formulation described herein as prophylactic protection as a supplement to conventional vaccination strategies.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

In particular embodiments, the condition is an infection.

Function as an effective amount, prophylactic treatment or therapeutic treatment are not mutually exclusive, and in particular embodiments, administered dosages may accomplish more than one treatment type.

In particular embodiments, therapeutically effective amounts provide anti-pathogen effects. Anti-pathogen effects can include anti-infection effects. Anti-infection effects can include a decrease in the occurrence of infections, a decrease in the severity of infections, a decrease in the duration of infections, a decrease in the number of infected cells, a decrease in volume of infected tissue, an increase in life expectancy, induced sensitivity of infected cells to immune clearance, reduced infection-associated pain, and/or reduction or elimination of a symptom associated with the treated infection.

In particular embodiments, therapeutically effective amounts provide anti-inflammatory effects. Anti-inflammatory effects can include reduced inflammation-associated pain, heat, redness, swelling and/or loss of function.

In particular embodiments, therapeutically effective amounts provide anti-Crohn's disease effects or anti-ulcerative colitis effects. Anti-Crohn's disease effects or anti-ulcerative colitis effects can include reduced diarrhea, reduced rectal bleeding, reduced unexplained weight loss, reduced fever, reduced abdominal pain and cramping, reduced fatigue and feelings of low energy, and/or restored appetite.

In particular embodiments, therapeutically effective amounts provide anti-arthritis effects. Anti-arthritis effects can include reduced pain, stiffness, swelling, redness in the joints and/or a restored range of motion. Types of arthritis include rheumatoid arthritis (RA), ankylosing spondylitis, and psoriatic arthritis.

In particular embodiments, therapeutically effective amounts provide anti-plaque psoriasis effects. Anti-plaque psoriasis effects can include reduced red patches, scaling spots, itching, burning, soreness, nail bed abnormalities and/or swollen or stiff joints.

In particular embodiments, B cells may be obtained, a subset of the B cells may be modified ex vivo, and then the modified B cells may be formulated and administered a subject. Obtained B cells can be modified to express a fusion protein that allows CD40 signaling and activation independently of interactions with CD40L. Obtained B cells can also optionally be modified to delete MHC class II molecule expression.

In particular embodiments, a first set of B cells may be modified with nucleic acids to produce a selected antibody against a first pathogen, and a second set of B cells may be modified with different nucleic acids to produce a selected antibody against a second pathogen, thereby providing protective antibodies against two pathogens. The B cells may similarly be modified to express fusion proteins disclosed herein having engineered antigen binding domains that bind the same pathogens to trigger B cell activation independently of interactions with CD40L. Additionally or alternatively, the B cells may be modified to express fusion proteins linking CD40 signaling to CD79α and/or CD79β domains.

B cells against any number of pathogens can be formed and administered to a subject. In particular embodiments, the selected antibodies can be an anti-RSV antibody, an anti-HIV antibody, an anti-Dengue virus antibody, an anti-Bordatella pertussis antibody, an anti-hepatitis C antibody, an anti-influenza virus antibody, an anti-parainfluenza virus antibody, an anti-MPV antibody, an anti-cytomegalovirus antibody, an anti-Epstein Barr virus antibody; an anti-herpes simplex virus antibody, an anti-Clostridium difficile bacterial toxin antibody, and/or an anti-TNF antibody. In particular embodiments, the selected antibodies can be one or more of an anti-RSV antibody, an anti-influenza virus antibody, an anti-parainfluenza virus antibody, and/or an anti-MPV antibody. In particular embodiments, the selected antibodies can be an anti-RSV antibody, an anti-influenza virus antibody, an anti-parainfluenza virus antibody, and an anti-MPV antibody. In particular embodiments, the selected antibody is palivizumab. The B cells may similarly be modified to express fusion proteins disclosed herein having engineered antigen binding domains that bind the same pathogens to trigger B cell activation independently of interactions with CD40L. Additionally or alternatively, the B cells may be modified to express fusion proteins linking CD40 signaling to CD79α and/or CD79β domains.

In particular embodiments, B cells may be obtained from a bone marrow donor or a hematopoietic stem cell donor that has been immunologically matched to a recipient. As indicated, obtained B cells can be modified to express a fusion protein that allows CD40 activation independently of interactions with CD40L. In particular embodiments, a first subset of the donor's B cells may be modified with nucleic acids to produce a selected antibody against a first pathogen, and a second subset of the donor's B cells may be modified with different nucleic acids to produce a selected antibody against a second pathogen, thereby providing protective antibodies against two pathogens. The genetically-modified B cells can be administered to the recipient to provide protection against infection (e.g., an anti-infection effect) until the transplanted cells repopulate the recipient's own immune system. The administered B cells may similarly be modified to express fusion proteins disclosed herein having engineered antigen binding domains that bind the same pathogens to trigger B cell activation independently of interactions with CD40L. Additionally or alternatively, the B cells may be modified to express fusion proteins linking CD40 signaling to CD79α and/or CD79β domains.

In particular embodiments, the recipient is receiving bone marrow from a donor or a hematopoietic stem cell transplant as a treatment for a hematological malignancy. Examples of hematological malignancies include acute lymphocytic leukemia, B-cell prolymphocytic leukemia, Burkitt lymphoma/leukemia, chronic lymphocytic leukemia (CLL), diffuse large B-cell lymphoma, follicular lymphoma (grades I, II, Ill, or IV), Hodgkin's lymphoma, intravascular large B-cell lymphoma, lymphoma, lymphoplasmocytic lymphoma, mantle cell lymphoma, marginal zone lymphoma (extra-nodal and nodal), mediastinal (thymic) large B-cell lymphoma, multiple myeloma, non-Hodgkin's lymphoma, POEMS syndrome/osteosclerotic myeloma, primary effusion lymphoma, splenic marginal zone lymphoma, small lymphocytic lymphoma, smoldering multiple myeloma (SMM), and Waldenstrom's macroglobulinemia.

In particular embodiments, the recipient is receiving genetically-modified hematopoietic stem cells that provide a gene the recipient is lacking. These recipients may have a primary or secondary immunodeficiency that can be treated with the provision of a therapeutic gene through hematopoietic stem cells. More than 80 primary immune deficiency diseases are recognized by the World Health Organization. These diseases are characterized by an intrinsic defect in the immune system in which, in some cases, the body is unable to produce any or enough antibodies against infection. In other cases, cellular defenses to fight infection fail to work properly. Typically, primary immune deficiencies are inherited disorders. X-linked severe combined immunodeficiency (SCID-X1) is another example of a primary immune deficiency. X-linked SCID results in both a cellular and humoral immune depletion caused by mutations in the common gamma chain gene (γC), which result in the absence of T and natural killer (NK) lymphocytes.

Secondary, or acquired, immune deficiencies are not the result of inherited genetic abnormalities, but rather occur in individuals in which the immune system is compromised by factors outside the immune system. Examples include trauma, viruses, chemotherapy, toxins, and pollution. Acquired immunodeficiency syndrome (AIDS) is an example of a secondary immune deficiency disorder caused by a virus, the human immunodeficiency virus (HIV), in which a depletion of T lymphocytes renders the body unable to fight infection.

In particular embodiments, B cells may be obtained, a subset of the B cells may be modified ex vivo, and then the modified B cells may be formulated and administered to a subject. In particular embodiments, a first set of B cells may be modified with first nucleic acids to produce a selected antibody against an inflammatory molecule, such as an inflammatory cytokine, thereby providing antibodies that protect against inflammation. In particular embodiments, the selected antibodies can be anti-TNF antibodies and/or anti-IL-1 antibodies. In particular embodiments, the selected antibody is infliximab, adalimumab, and/or golimumab and/or an approved biosimilar thereof.

As indicated, in particular embodiments, modified B cells express a tag that allows, for example, tracking and/or elimination after administration to a subject.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including age, previous vaccinations (if any), target, body weight, severity of condition, type of condition, stage of condition, previous or concurrent therapeutic interventions, idiopathy of the subject and route of administration.

Exemplary doses can include greater than 10² modified B cells, greater than 10³ modified B cells, greater than 10⁴ modified B cells, greater than 10¹ modified B cells, greater than 10⁶ modified B cells, greater than 10⁷ modified B cells, greater than 10⁸ modified B cells, greater than 10⁹ modified B cells, greater than 10¹⁰ modified B cells, or greater than 10¹¹ modified B cells.

In particular embodiments, the effects of selected antibodies can be measured using viral titers. Viral titer refers to the amount of virus that can be detected. High viral titers mean high levels of infection. An optimal protective response is observed with titers that fall to zero.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (xii) Exemplary Embodiments

1. A fusion protein, the expression of which in a B cell, results in CD40 activation independently of CD40 ligand (CD40L) binding.

2. The fusion protein of embodiment 1, including an antigen binding domain linked to a transmembrane domain that is linked to an intracellular CD40 signaling domain.

3. The fusion protein of embodiment 2, wherein the antigen binding domain is an engineered antigen binding domain.

4. The fusion protein of embodiment 3, wherein the engineered antigen binding domain is a single chain variable fragment (scFv).

5. The fusion protein of embodiments 3 and 4, wherein the engineered antigen binding domain is linked to the transmembrane domain through a spacer.

6. The fusion protein of embodiment 5, wherein the spacer is an extracellular portion of CD40 or an IgG hinge region.

7. The fusion protein of any of embodiments 2-6, further including a tag.

8. The fusion protein of embodiment 7, wherein the tag has a sequence set forth in SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, or SEQ ID NO: 63.

9. The fusion protein of any of embodiments 2-8, further including at least two tags including (i) SEQ ID NO: 62 or SEQ ID NO: 50 and (ii) SEQ ID NO: 51.

10. The fusion protein of embodiments 7 and 8, wherein the tag links the antigen binding domain to the spacer.

11. The fusion protein of any of embodiments 3-10, wherein the engineered antigen binding domain is derived from the binding domain of an anti-Respiratory Syncytial Virus (RSV) antibody, an anti-human immunodeficiency virus (HIV) antibody, an anti-Dengue virus antibody, an anti-Bordatella pertussis antibody, an anti-hepatitis C antibody, an anti-influenza virus antibody, an anti-parainfluenza virus antibody, an anti-metapneumovirus (MPV) antibody, an anti-cytomegalovirus antibody, an anti-Epstein Barr virus antibody; an anti-herpes simplex virus antibody, an anti-Clostridium difficile bacterial toxin antibody, or an anti-tumor necrosis factor (TNF) antibody.

12. The fusion protein of any of embodiments 3-11, wherein the engineered antigen binding domain is derived from the binding domain of an anti-RSV antibody including a heavy chain including the sequence set forth in SEQ ID NO: 78 and a light chain including the sequence set forth in SEQ ID NO: 79; or an anti-RSV antibody including a heavy chain including the sequence set forth in SEQ ID NO: 80 and a light chain including the sequence set forth in SEQ ID NO: 81.

13. The fusion protein of any of embodiments 3-11, wherein the engineered antigen binding domain is derived from the binding domain of an anti-RSV antibody including a CDRH1 including the sequence set forth in SEQ ID NO: 82, a CDRH2 including the sequence set forth in SEQ ID NO: 83, a CDRH3 including the sequence set forth in SEQ ID NO: 84; a CDRL1 including the sequence set forth in SEQ ID NO: 85, a CDRL2 including the sequence set forth in SEQ ID NO: 86, and a CDRL3 including the sequence set forth in SEQ ID NO: 87.

14. The fusion protein of any of embodiments 3-11, wherein the engineered antigen binding domain is derived from the binding domain of an anti-HIV antibody including 10E8, VRC01, ab18633 or 39/5.4A.

15. The fusion protein of any of embodiments 3-11 and 14, wherein the engineered antigen binding domain is derived from the binding domain of an anti-HIV antibody including a CDRH1 including the sequence set forth in SEQ ID NO: 88, a CDRH2 including the sequence set forth in SEQ ID NO: 89, a CDRH3 including the sequence set forth in SEQ ID NO: 90, a CDRL1 including the sequence set forth in SEQ ID NO: 91, a CDRL2 including the sequence set forth in SEQ ID NO: 92, and a CDRL3 including the sequence set forth in SEQ ID NO: 93 or a CDRH1 including the sequence set forth in SEQ ID NO: 94, a CDRH2 including the sequence set forth in SEQ ID NO: 95, a CDRH3 including the sequence set forth in SEQ ID NO: 96, a CDRL1 including the sequence set forth in SEQ ID NO: 97, a CDRL2 including the sequence SGS, and a CDRL3 including the sequence set forth in SEQ ID NO: 98.

16. The fusion protein of any of embodiments 3-11, wherein the engineered antigen binding domain is derived from the binding domain of an anti-Dengue virus antibody including antibody 55, DB2-3, ab155042 or ab80914.

17. The fusion protein of any of embodiments 3-11 and 16, wherein the engineered antigen binding domain is derived from the binding domain of an anti-Dengue virus antibody including a CDRH1 including the sequence set forth in SEQ ID NO: 99, a CDRH2 including the sequence set forth in SEQ ID NO: 100, a CDRH3 including the sequence set forth in SEQ ID NO: 101; a CDRL1 including the sequence set forth in SEQ ID NO: 102, a CDRL2 including the sequence set forth in SEQ ID NO: 103, and a CDRKL3 including the sequence set forth in SEQ ID NO: 104 or a CDRH1 including the sequence set forth in SEQ ID NO: 105, a CDRH2 including the sequence set forth in SEQ ID NO: 106, a CDRH3 including the sequence set forth in SEQ ID NO: 107, a CDRL1 including the sequence set forth in SEQ ID NO: 108, a CDRL2 including the sequence set forth in SEQ ID NO: 109, and a CDRL3 including the sequence set forth in SEQ ID NO: 110.

18. The fusion protein of any of embodiments 3-11, wherein the engineered antigen binding domain is derived from the binding domain of an anti-pertussis antibody including a heavy chain including the sequence set forth in SEQ ID NO: 111 and a light chain including the sequence set forth in SEQ ID NO: 112.

19. The fusion protein of any of embodiments 3-11, wherein the engineered antigen binding domain is derived from the binding domain of an anti-hepatitis C antibody including MAB8694 or C7-50.

20. The fusion protein of any of embodiments 3-11, wherein the engineered antigen binding domain is derived from the binding domain of an anti-hepatitis C antibody including a CDRH1 including the sequence set forth in SEQ ID NO: 113, a CDRH2 including the sequence set forth in SEQ ID NO: 114, a CDRH3 including the sequence set forth in SEQ ID NO: 115, a CDRL1 including the sequence set forth in SEQ ID NO: 116, a CDRL2 including the sequence set forth in SEQ ID NO: 117, and a CDRL3 including the sequence set forth in SEQ ID NO: 118.

21. The fusion protein of any of embodiments 3-11, wherein the engineered antigen binding domain is derived from the binding domain of an anti-influenza virus antibody including C102.

22. The fusion protein of any of embodiments 3-11, wherein the engineered antigen binding domain is derived from the binding domain of an anti-influenza virus antibody including a CDRH1 including the sequence set forth in SEQ ID NO: 119, a CDRH2 including the sequence set forth in SEQ ID NO: 120, a CDRH3 including the sequence set forth in SEQ ID NO: 121, a CDRL1 including the sequence set forth in SEQ ID NO: 122, a CDRL2 including the sequence KTS, and a CDRL3 including the sequence set forth in SEQ ID NO: 123.

23. The fusion protein of any of embodiments 3-11, wherein the engineered antigen binding domain is derived from the binding domain of an anti-MPV antibody including MPE8.

24. The fusion protein of any of embodiments 3-11, wherein the engineered antigen binding domain is derived from the binding domain of an anti-CMV antibody including MCMV5322A, MCMV3068A, LJP538, or LJP539.

25. The fusion protein of any of embodiments 3-11, wherein the engineered antigen binding domain is derived from the binding domain of an anti-EBV antibody including a CDRH1 including the sequence set forth in SEQ ID NO: 124, a CDRH2 including the sequence set forth in SEQ ID NO: 125, a CDRH3 including the sequence set forth in SEQ ID NO: 126, a CDRL1 including the sequence set forth in SEQ ID NO: 127, a CDRL2 including the sequence set forth in SEQ ID NO: 128, and a CDRL3 including the sequence set forth in SEQ ID NO: 129.

26. The fusion protein of any of embodiments 3-11, wherein the engineered antigen binding domain is derived from the binding domain of an anti-HSV antibody including HSV8-N and MB66.

27. The fusion protein of any of embodiments 3-11, wherein the engineered antigen binding domain is derived from the binding domain of an anti-Clostridium difficile antibody including actoxumab or bezlotoxumab.

28. The fusion protein of any of embodiments 3-11, wherein the engineered antigen binding domain is derived from the binding domain of an anti-TNF antibody including infliximab, adalimumab, etanercept, certolizumab, or accepted biosimilars thereof.

29. The fusion protein of any of embodiments 4-28, wherein the scFv includes a Gly-Ser linker having 5-30 amino acids.

30. The fusion protein of embodiment 29, wherein the Gly-Ser linker has 15 amino acids.

31. The fusion protein of any of embodiments 2-30, further including a Gly linker.

32. The fusion protein of embodiment 31, wherein the Gly linker is Gly₆. 33. The fusion protein of embodiments 31 and 32, wherein the Gly linker links a tag to a spacer region or a tag to an intracellular CD40 signaling domain.

34. The fusion protein of any of embodiments 2-33, having a sequence set forth in SEQ ID NO: 4, 6, 8, or 10.

35. The fusion protein of any of embodiments 1-34, including CD79α linked to an intracellular CD40 signaling domain or CD79β linked to an intracellular CD40 signaling domain.

36. The fusion protein of any of embodiments 2-35, further including a multimerization domain.

37. The fusion protein of embodiment 36, wherein the multimerization domain includes FKBP12 or FKBP12v36.

38. The fusion protein of any of embodiments 2-37, having a sequence set forth in SEQ ID NO: 14, 16, 18, or 20.

39. A B cell expressing the fusion protein of any of embodiments 2-38.

40. A nucleic acid encoding the fusion protein of any of embodiments 2-38.

41. The nucleic acid of embodiment 40, wherein the nucleic acid further encodes a signal peptide.

42. The nucleic acid of embodiment 41, wherein the signal peptide is a CD40 signal peptide.

43. The nucleic acid of any of embodiments 40-42, wherein the nucleic acid further encodes a skipping element, and a reporter.

44. The nucleic acid of embodiment 43, wherein the skipping element is a self-cleaving peptide.

45. The nucleic acid of embodiment 44, wherein the self-cleaving peptide is a 2A self-cleaving peptide.

46. The nucleic acid of any of embodiments 43-45, wherein the reporter is a fluorescent protein or truncated epidermal growth factor receptor (tEGFR).

47. The nucleic acid of any of embodiments 40-46, having a sequence set forth in SEQ ID NO: 3, 5, 7, 9, 13, 15, 17, or 19.

48. A B cell including the nucleic acid of any of embodiments 40-47.

49. The B cell of embodiment 48, further including a nuclease and guide RNA (gRNA) that results in deletion of a gene encoding a transcription factor required for MHC class II molecule expression.

50. The B cell of embodiment 49, wherein the transcription factor includes CIITA, TRAC, TRBC, B2M, RFX5, RFXAP, or RFXANK.

51. The B cell of embodiments 49 and 50, wherein the nuclease is Cas9 or Cpf1.

52. The B cell of any of embodiments 49-51, wherein the gRNA has the sequence set forth in SEQ ID NO: 1, 2, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, or 202.

53. The B cell of any of embodiments 39 and 48-52, wherein the B cell is an antibody-secreting B cell, a memory B cell, a naïve B cell, a B1 B cell or a marginal zone B cell.

54. The B cell of any of embodiments 48-53, formulated for administration to a subject.

55. A kit including a nucleic acid encoding the fusion protein of any of embodiments 2-38.

56. The kit of embodiment 55, further including a nucleic acid vector.

57. The kit of embodiment 56, wherein the nucleic acid vector includes a plasmid, a transposon, a cosmid, or a viral vector.

58. The kit of any of embodiments 55-57, further including a nuclease.

59. The kit of embodiment 58, wherein the nuclease is Cas9 or Cpf1.

60. The kit of any of embodiments 55-59, further including guideRNA (gRNA).

61. The kit of embodiment 60, wherein the gRNA has a sequence set forth in SEQ ID NO: 1, 2, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, or 202.

62. The kit of any of embodiments 58-61, wherein the gRNA and nuclease are associated with a nanoparticle.

63. A method of genetically modifying B cells to express a fusion protein that allows CD40 activation of the B cell based on antigen binding and independently of CD40L binding wherein the method includes introducing the nucleic acid of any of embodiments 40-47 into the B cell.

64. The method of embodiment 63, further including genetically modifying the B cell to delete expression of a transcription factor required for MHC class II molecule expression.

65. The method of embodiment 64, wherein the transcription factor includes CIITA, TRAC, TRBC, B2M, RFX5, or RFXAP.

66. A method of providing an anti-infection effect in a subject in need thereof including administering a therapeutically effective amount of the B cell of any of embodiments 39 and 48-54 to the subject thereby providing an anti-infection effect.

67. The method of embodiment 66, wherein the providing obviates the need for a vaccination.

68. The method of embodiments 66 and 67, wherein the administering replaces a vaccination protocol.

69. The method of any of embodiments 66-68, wherein the subject is immune-suppressed.

70. The method of any of embodiments 66-69, wherein the subject is immune-suppressed as part of a treatment regimen including a bone marrow transplant, hematopoietic stem cell transplant, or administration of genetically modified hematopoietic stem cells.

71. A method of providing an anti-inflammatory effect in a subject in need thereof including administering a therapeutically effective amount of a B cell of any of embodiments 39 and 48-54 to the subject thereby providing an anti-inflammatory effect.

(xiii) Experimental Examples. Example 1. scFv CD40. The following constructs were generated using Gibson cloning:
Construct 1: humanCD40 signal peptide—anti-gp120 scFv—Linker—StrepTag—Gly6 Linker—full CD40 protein—Gly6 Linker—HA tag—P2A skipping element—mCherry reporter;
Construct 2: humanCD40 signal peptide—anti-gp120 scFv—Linker—StrepTagII—Gly6 Linker—IgG Hinge—Gly6 Linker—CD40 Transmembrane Domain—CD40 Intracellular Domain—Gly6 Linker—HA tag—P2A skipping element—mCherry reporter;
Construct 3: humanCD40 signal peptide—anti-gp120 scFv—Linker—StrepTagII—Gly6 Linker—IgG Hinge—Gly6 Linker—IgG2 Transmembrane Domain—Gly6 Linker—CD40 Intracellular Domain—Gly6 Linker—HA tag—P2A skipping element—mCherry reporter;
Construct 4: humanCD40 signal peptide—anti-gp120 scFv—Linker—StrepTag—Gly6 Linker—full CD40 protein—Gly6 Linker—FkBP12v36—GlySer Linker—FkBP12v36—Gly6 Linker—HA tag—P2A skipping element—mCherry reporter; and
Construct 5: human CD40 signal peptide—full CD40 protein—Gly6 Linker—HA tag—P2A skipping element—mCherry Reporter.

The constructs were amplified in E. Coli and the DNA was isolated by Qiagen Midi Prep kit. The DNA was then chemically transfected into HEK293E cells. After 36 hours incubation, the cells were harvested and stained with the following panel for flow cytometry analysis of expression.

| Antibody | Color | Function |
|---|---|---|
| p-p38 | PE-Cy7 | Phosphorylated downstream of CD40 signaling |
| p-ERk | AF700 | Phosphorylated downstream of CD40 signaling |
| p-Stat3 | 488 | Phosphorylated downstream of CD40 signaling |
| | mCherry | Plasmid in cell |
| SA | BV421 | Extracellular tag on construct |
| HA | AF647 | Intracellular tag on construct |
| Ghost Dye | V510 | Live/dead |

For functional testing, Ramos cells were electroporated via Neon protocol for the conditions outlined below:
1. Construct 1 stimulated w/HIV Env tetramer;
2. Construct 1 stimulated w/Streptactin-PE;
3. Construct 1 no stim;
4. Construct 2 stimulated w/HIV Env tetramer;
5. Construct 2 stimulated w/Streptactin-PE;
6. Construct 2 no stim;
7. Construct 3 stimulated w/HIV Env tetramer;
8. Construct 3 stimulated w/Streptactin-PE;
9. Construct 3 no stim;
10. Construct 4 stimulated w/AP1903; and
11. Construct 4 no stim
   After 24 hour incubation, the cells were harvested and stained for flow cytometry experiments.

Example 2. BCR-CD40 Complexes. The following constructs were created using Gibson cloning:
MRT3: mouseCD79α-IntracellularCD40-P2A skipping element —eGFP Reporter;
MRT4: mouseCD79β-IntracelluarCD40-P2A skipping element —eGFP Reporter;
MRT5: humanCD79α-IntracellularCD40-P2A skipping element —mCherry Reporter;
MRT6: humanCD79β-IntracelluarCD40-P2A skipping element —mCherry Reporter;
MRT7: humanCD79α-P2A skipping element —mCherry Reporter; and
MRT8: humanCD79β-P2A skipping element —mCherryReporter

These constructs were amplified in E. Coli and the DNA was isolated by Qiagen Midi Prep kit. The DNA was then chemically transfected into HEK293E cells for the conditions outlined below:

Conditions:
WT CD79α+WT CD79β+WT anti-huRSV;
Mutated CD79α+WT CD79β+WT anti-huRSV;
WT CD79α+Mutated CD79β+WT anti-huRSV; and
Mutated CD79α+Mutated CD79β+WT anti-huRSV

After 36 hours incubation, the cells were harvested and stained with the following panel for flow cytometry analysis of expression.

| Item | Fluorescence | Channel | Marker |
|---|---|---|---|
| monomer RSV tag | Blue | B510 | Fitc, AF488 |
| mCherry | Green | G610 | PE-eF610 |
| FVD | Violet | V510 | Ghost 510 |
| IgD | UV | U737 | BUV 737 |
| anti huCD79β | Red | R780 | APC Fire 750 |

For functional testing, Ramos cells were electroporated via Neon protocol for the following conditions (mock and sample 1-4 for each below):
IgG stimulation, 24 hours/24+ hours incubation, phospho-flow;
RSV-preF stimulation, 24 hours/24+ hours incubation, phospho-flow;
No stimulation, 48 total hours incubation, phospho-flow;
No stimulation, 24 total hours incubation, Calcium Flux; and
No stimulation, 48 total hours incubation, Calcium Flux

After 24-hour incubation, the cells were harvested and stained for flow cytometry experiments via the following panel:

| | | |
|---|---|---|
| Channel | Fluor | Target |

Intra-cellular Stain

| | | |
|---|---|---|
| B510 | AF488 | Stat3 |
| R660 | AF647 | CD79α |
| R710 | AF700 | ERK1/2 |
| G575 | PE | Syk |
| G610 | | mCherry |
| G780 | PE-Cy7 | P38 |
| | BV Buffer | |

Extracellular Stain

| | | |
|---|---|---|
| R780 | APC Fire 780 | antiCD79β |
| U737 | BUV 737 | IgD |
| V510 | Ghost508 | Live/Dead |

(xiv) Closing Paragraphs. Variants of protein and nucleic acid sequences described herein are also included. Variants of proteins can include those having one or more conservative amino acid substitutions or one or more non-conservative substitutions that do not adversely affect the binding or activity of the protein.

In particular embodiments, a conservative amino acid substitution may not substantially change the structural characteristics of the reference sequence (e.g., a replacement amino acid should not tend to break a helix that occurs in the reference sequence or disrupt other types of secondary structure that characterizes the reference sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden & J. Tooze, eds., Garland Publishing, New York, N.Y. (1991)); and Thornton et al., Nature, 354:105 (1991).

In particular embodiments, a “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gln); Group 4: Arginine (Arg), Lysine (Lys), Histidine (His); Group 5: Isoleucine (lie), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and lie. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, lie, Val, and Cys; and large aromatic residues: Phe, Tyr, and Trp. Additional information is found in Creighton (1984) Proteins, W. H. Freeman and Company.

In the fusion proteins disclosed herein, amino acid substitutions, deletions or additions can be made in linker sequences and spacer regions. They may also be made within CD40 signaling domains so long as the substitution, deletion, or addition does not adversely affect CD40 activation signals.

In particular embodiments, a V_{L} region can be derived from or based on a disclosed V_{L} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the disclosed V_{L}. An insertion, deletion or substitution may be anywhere in the V_{L} region, including at the amino- or carboxy-terminus or both ends of this region, provided that each CDR includes zero changes or at most one, two, or three changes and provided an antibody including the modified V_{L} region can still specifically bind its target epitope with an affinity similar to the wild type binding domain.

In particular embodiments, a V_{H} region can be derived from or based on a disclosed V_{H} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V_{H} disclosed herein. An insertion, deletion or substitution may be anywhere in the V_{H} region, including at the amino- or carboxy-terminus or both ends of this region, provided that each CDR includes zero changes or at most one, two, or three changes and provided an antibody including the modified V_{H} region can still specifically bind its target epitope with an affinity similar to the wild type binding domain.

In particular embodiments, a variant includes or is a sequence that has at least 90%, at least 91%, at least 92%, at least 93%, at least 94%, at least 95%, at least 96%, at least 97%, at least 98%, at least 99%, at least 99.5% sequence identity to a sequence disclosed herein. In particular embodiments, a variant includes or is a sequence that has at least 90%, at least 91%, at least 92%, at least 93%, at least 94%, at least 95%, at least 96%, at least 97%, at least 98%, at least 99%, at least 99.5% sequence identity to a light chain variable region (V_{L}) and/or to a heavy chain variable region (V_{H}), or both, wherein each CDR includes zero changes or at most one, two, or three changes, from the reference antibody disclosed herein or fragment or derivative thereof that binds the relevant antigen.

Each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in CD40 signaling following antigen binding, independently of interactions with CD40L through T cell interaction.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; +19% of the stated value; ±18% of the stated value; +17% of the stated value; +16% of the stated value; ±15% of the stated value; +14% of the stated value; ±13% of the stated value; +12% of the stated value; +11% of the stated value; +10% of the stated value; ±9% of the stated value; ±8% of the stated value; +7% of the stated value; ±6% of the stated value; ±5% of the stated value; +4% of the stated value; ±3% of the stated value; +2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006) or Janeway's Immunobiology, 9th Edition (Murphy & Weaver, Garland Science, 2017).

What is claimed is:

1. A fusion protein, the expression of which in a B cell, results in CD40 activation independently of CD40 ligand (CD40L) binding.

2. The fusion protein of claim 1, comprising an antigen binding domain linked to a transmembrane domain that is linked to an intracellular CD40 signaling domain.

3. The fusion protein of claim 2, wherein the antigen binding domain is an engineered antigen binding domain.

4. The fusion protein of claim 3, wherein the engineered antigen binding domain is a single chain variable fragment (scFv).

5. The fusion protein of claim 3, wherein the engineered antigen binding domain is linked to the transmembrane domain through a spacer.

6. The fusion protein of claim 5, wherein the spacer is an extracellular portion of CD40 or an IgG hinge region.

7. The fusion protein of claim 2, further comprising a tag.

8. The fusion protein of claim 7, wherein the tag has a sequence set forth in SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, or SEQ ID NO: 63.

9. The fusion protein of claim 2, further comprising at least two tags comprising (i) SEQ ID NO: 62 or SEQ ID NO: 50 and (ii) SEQ ID NO: 51.

10. The fusion protein of claim 7, wherein the tag links the antigen binding domain to the spacer.

11. The fusion protein of claim 3, wherein the engineered antigen binding domain is derived from the binding domain of an anti-Respiratory Syncytial Virus (RSV) antibody, an anti-human immunodeficiency virus (HIV) antibody, an anti-Dengue virus antibody, an anti-Bordatella pertussis antibody, an anti-hepatitis C antibody, an anti-influenza virus antibody, an anti-parainfluenza virus antibody, an anti-metapneumovirus (MPV) antibody, an anti-cytomegalovirus antibody, an anti-Epstein Barr virus antibody; an anti-herpes simplex virus antibody, an anti-Clostridium difficile bacterial toxin antibody, or an anti-tumor necrosis factor (TNF) antibody.

12. The fusion protein of claim 3, wherein the engineered antigen binding domain is derived from the binding domain of an anti-RSV antibody comprising a heavy chain comprising the sequence set forth in SEQ ID NO: 78 and a light chain comprising the sequence set forth in SEQ ID NO: 79; or an anti-RSV antibody comprising a heavy chain comprising the sequence set forth in SEQ ID 46 NO: 80 and a light chain comprising the sequence set forth in SEQ ID NO: 81.

13. The fusion protein of claim 3, wherein the engineered antigen binding domain is derived from the binding domain of an anti-RSV antibody comprising a CDRH1 comprising the sequence set forth in SEQ ID NO: 82, a CDRH2 comprising the sequence set forth in SEQ ID NO: 83, a CDRH3 comprising the sequence set forth in SEQ ID NO: 84; a CDRL1 comprising the sequence set forth in SEQ ID NO: 85, a CDRL2 comprising the sequence set forth in SEQ ID NO: 86, and a CDRL3 comprising the sequence set forth in SEQ ID NO: 87.

14. The fusion protein of claim 3, wherein the engineered antigen binding domain is derived from the binding domain of an anti-HIV antibody comprising 10E8, VRC01, ab18633 or 39/5.4A.

15. The fusion protein of claim 3, wherein the engineered antigen binding domain is derived from the binding domain of an anti-HIV antibody comprising a CDRH1 comprising the sequence set forth in SEQ ID NO: 88, a CDRH2 comprising the sequence set forth in SEQ ID NO: 89, a CDRH3 comprising the sequence set forth in SEQ ID NO: 90, a CDRL1 comprising the sequence set forth in SEQ ID NO: 91, a CDRL2 comprising the sequence set forth in SEQ ID NO: 92, and a CDRL3 comprising the sequence set forth in SEQ ID NO: 93 or a CDRH1 comprising the sequence set forth in SEQ ID NO: 94, a CDRH2 comprising the sequence set forth in SEQ ID NO: 95, a CDRH3 comprising the sequence set forth in SEQ ID NO: 96, a CDRL1 comprising the sequence set forth in SEQ ID NO: 97, a CDRL2 comprising the sequence SGS, and a CDRL3 comprising the sequence set forth in SEQ ID NO: 98.

16. The fusion protein of claim 3, wherein the engineered antigen binding domain is derived from the binding domain of an anti-Dengue virus antibody comprising antibody 55, DB2-3, ab155042 or ab80914.

17. The fusion protein of claim 3, wherein the engineered antigen binding domain is derived from the binding domain of an anti-Dengue virus antibody comprising a CDRH1 comprising the sequence set forth in SEQ ID NO: 99, a CDRH2 comprising the sequence set forth in SEQ ID NO: 100, a CDRH3 comprising the sequence set forth in SEQ ID NO: 101; a CDRL1 comprising the sequence set forth in SEQ ID NO: 102, a CDRL2 comprising the sequence set forth in SEQ ID NO: 103, and a CDRKL3 comprising the sequence set forth in SEQ ID NO: 104 or a CDRH1 comprising the sequence set forth in SEQ ID NO: 105, a CDRH2 comprising the sequence set forth in SEQ ID NO: 106, a CDRH3 comprising the sequence set forth in SEQ ID NO: 107, a CDRL1 comprising the sequence set forth in SEQ ID NO: 108, a CDRL2 comprising the sequence set forth in SEQ ID NO: 109, and a CDRL3 comprising the sequence set forth in SEQ ID NO: 110.

18. The fusion protein of claim 3, wherein the engineered antigen binding domain is derived from the binding domain of an anti-pertussis antibody comprising a heavy chain comprising the sequence set forth in SEQ ID NO: 111 and a light chain comprising the sequence set forth in SEQ ID NO: 112.

19. The fusion protein of claim 3, wherein the engineered antigen binding domain is derived from the binding domain of an anti-hepatitis C antibody comprising MAB8694 or C7-50.

20. The fusion protein of claim 3, wherein the engineered antigen binding domain is derived from the binding domain of an anti-hepatitis C antibody comprising a CDRH1 comprising the sequence set forth in SEQ ID NO: 113, a CDRH2 comprising the sequence set forth in SEQ ID NO: 114, a CDRH3 comprising the sequence set forth in SEQ ID NO: 115, a CDRL1 comprising the sequence set forth in SEQ ID NO: 116, a CDRL2 comprising the sequence set forth in SEQ ID NO: 117, and a CDRL3 comprising the sequence set forth in SEQ ID NO: 118.

21. The fusion protein of claim 3, wherein the engineered antigen binding domain is derived from the binding domain of an anti-influenza virus antibody comprising C102.

22. The fusion protein of claim 3, wherein the engineered antigen binding domain is derived from the binding domain of an anti-influenza virus antibody comprising a CDRH1 comprising the sequence set forth in SEQ ID NO: 119, a CDRH2 comprising the sequence set forth in SEQ ID NO: 120, a CDRH3 comprising the sequence set forth in SEQ ID NO: 121, a CDRL1 comprising the sequence set forth in SEQ ID NO: 122, a CDRL2 comprising the sequence KTS, and a CDRL3 comprising the sequence set forth in SEQ ID NO: 123.

23. The fusion protein of claim 3, wherein the engineered antigen binding domain is derived from the binding domain of an anti-MPV antibody comprising MPE8.

24. The fusion protein of claim 3, wherein the engineered antigen binding domain is derived from the binding domain of an anti-CMV antibody comprising MCMV5322A, MCMV3068A, LJP538, or LJP539.

25. The fusion protein of claim 3, wherein the engineered antigen binding domain is derived from the binding domain of an anti-EBV antibody comprising a CDRH1 comprising the sequence set forth in SEQ ID NO: 124, a CDRH2 comprising the sequence set forth in SEQ ID NO: 125, a CDRH3 comprising the sequence set forth in SEQ ID NO: 126, a CDRL1 comprising the sequence set forth in SEQ ID NO: 127, a CDRL2 comprising the sequence set forth in SEQ ID NO: 128, and a CDRL3 comprising the sequence set forth in SEQ ID NO: 129.

26. The fusion protein of claim 3, wherein the engineered antigen binding domain is derived from the binding domain of an anti-HSV antibody comprising HSV8-N and MB66.

27. The fusion protein of claim 3, wherein the engineered antigen binding domain is derived from the binding domain of an anti-Clostridium difficile antibody comprising actoxumab or bezlotoxumab.

28. The fusion protein of claim 3, wherein the engineered antigen binding domain is derived from the binding domain of an anti-TNF antibody comprising infliximab, adalimumab, etanercept, certolizumab, or accepted biosimilars thereof.

29. The fusion protein of claim 4, wherein the scFv comprises a Gly-Ser linker having 5-30 amino acids.

30. The fusion protein of claim 29, wherein the Gly-Ser linker has 15 amino acids.

31. The fusion protein of claim 2, further comprising a Gly linker.

32. The fusion protein of claim 31, wherein the Gly linker is Gly₆.

33. The fusion protein of claim 31, wherein the Gly linker links a tag to a spacer region or a tag to an intracellular CD40 signaling domain.

34. The fusion protein of claim 2, having a sequence set forth in SEQ ID NO: 4, 6, 8, or 10.

35. The fusion protein of claim 1, comprising CD79α linked to an intracellular CD40 signaling domain or CD79β linked to an intracellular CD40 signaling domain.

36. The fusion protein of claim 2 or 35, further comprising a multimerization domain.

37. The fusion protein of claim 36, wherein the multimerization domain comprises FKBP12 or FKBP12v36.

38. The fusion protein of claim 2, having a sequence set forth in SEQ ID NO: 14, 16, 18, or 20.

39. A B cell expressing the fusion protein of claim 2 or 35.

40. A nucleic acid encoding the fusion protein of claim 2 or claim 35.

41. The nucleic acid of claim 40, wherein the nucleic acid further encodes a signal peptide.

42. The nucleic acid of claim 41, wherein the signal peptide is a CD40 signal peptide.

43. The nucleic acid of claim 40, wherein the nucleic acid further encodes a skipping element, and a reporter.

44. The nucleic acid of claim 43, wherein the skipping element is a self-cleaving peptide.

45. The nucleic acid of claim 44, wherein the self-cleaving peptide is a 2A self-cleaving peptide.

46. The nucleic acid of claim 43, wherein the reporter is a fluorescent protein or truncated epidermal growth factor receptor (tEGFR).

47. The nucleic acid of claim 40, having a sequence set forth in SEQ ID NO: 3, 5, 7, 9, 13, 15, 17, or 19.

48. A B cell comprising the nucleic acid of claim 40.

49. The B cell of claim 39 or 48, further comprising a nuclease and guide RNA (gRNA) that results in deletion of a gene encoding a transcription factor required for MHC class II molecule expression.

50. The B cell of claim 49, wherein the transcription factor comprises CIITA, TRAC, TRBC, B2M, RFX5, RFXAP, or RFXANK.

51. The B cell of claim 49, wherein the nuclease is Cas9 or Cpf1.

52. The B cell of claim 49, wherein the gRNA has the sequence set forth in 1, 2, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, or 202.

53. The B cell of claim 39 or 48, wherein the B cell is an antibody-secreting B cell, a memory B cell, a naïve B cell, a B1 B cell or a marginal zone B cell.

54. The B cell of claim 48, formulated for administration to a subject.

55. A kit comprising a nucleic acid encoding the fusion protein of claim 2 and/or claim 35.

56. The kit of claim 55, further comprising a nucleic acid vector.

57. The kit of claim 56, wherein the nucleic acid vector comprises a plasmid, a transposon, a cosmid, or a viral vector.

58. The kit of claim 55, further comprising a nuclease.

59. The kit of claim 58, wherein the nuclease is Cas9 or Cpf1.

60. The kit of claim 55, further comprising guideRNA (gRNA).

61. The kit of claim 60, wherein the gRNA has a sequence set forth in SEQ ID NO: 1, 2, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, or 202.

62. The kit of claims 58 and 60, wherein the gRNA and nuclease are associated with a nanoparticle.

63. A method of genetically modifying B cells to express a fusion protein that allows CD40 activation of the B cell based on antigen binding and independently of CD40L binding wherein the method comprises introducing the nucleic acid of claim 40 into the B cell.

64. The method of claim 63, further comprising genetically modifying the B cell to delete expression of a transcription factor required for MHC class II molecule expression.

65. The method of claim 64, wherein the transcription factor comprises Cl TA, TRAC, TRBC, B2M, RFX5, or RFXAP.

66. A method of providing an anti-infection effect in a subject in need thereof comprising administering a therapeutically effective amount of the B cell of claim 39 or 48 to the subject thereby providing an anti-infection effect.

67. The method of claim 66, wherein the providing obviates the need for a vaccination.

68. The method of claim 66, wherein the administering replaces a vaccination protocol.

69. The method of claim 66, wherein the subject is immune-suppressed.

70. The method of claim 66, wherein the subject is immune-suppressed as part of a treatment regimen comprising a bone marrow transplant, hematopoietic stem cell transplant, or administration of genetically modified hematopoietic stem cells.

71. A method of providing an anti-inflammatory effect in a subject in need thereof comprising administering a therapeutically effective amount of a B cell of claim 39 or 48 to the subject thereby providing an anti-inflammatory effect.
